SEQUENCE LISTING (149 sequences)

SEQ ID NO: 1 - DNA, 11922 bp, Artificial
  synthetic
ctgagcgcacaacagcgagtcgcatggcaccggacgacatgagcgagatttagatcggagggtgcggacatggggcaacctgcgcagctaacgcagggatccacacgaccaccaacgaagccaagcccgggcacgtccccaggcaggttgggccctggttccaccagcggatgcatgcagtgaagcggggacggagagacaagccgagggcgcgggtgggaatggcgtccgggaggacgagtggaggagaagaatctagaggcatcgagattcgagaagccgacggagacaagattcgtgtggggggagacaaaccgcggggctgagcgccgttgatatgggatcagacggtgtggataaaaaaagtgacgttgatagaacgtctggccagtgaaaaaacaaaacaactccaacaaaatactttaaaagctcttataccctaaatgtaggggatcaaacacgtctctacactatttagcagcgtcctctaaatgatcctctaaatttagagaacgctactagattctctatatatagtttctctaaacgatcttttatccatttaaatactttaaataaccggtttaacaaaactaaaatatatacaatacatttgagagtatgacaaatacgtatgtataaaaataaaaaataaaataatgtattagtctactttgaatcttcttttcttcataatataatgatgtatagctctcatgtgcgttgagaaaaaagttagagctagacgtttaatgtgtagtgacagtcttcgacgaaatctccctaatgagatgaattactggaggttccatcagaaagtcccctgaaaagaggcatttatttagtttagtcagcaatttctgggaacacaaatattcttttgttatcaccactattaaaaatctatggttataacttataataacatgaaaaaataatttagcatcccatatatataaaaactgaaggaagccatatatactaacataagttaggagaaactaagaaggttgtgcaaagcttgcactgctccaaaatactgcaaacaaccactctcctctaccaaccaaagaaactcatgtactccctccgttcttttttatttgtcgcattttagtttaaaaatgaactagcagtcgacaaatattcgagaacagatatagtatatactaacataacttaggagatactaagaaagttgcgcagagctttcactgttccaaattactgcaaagcctctcccctctgccagtacatctacgagatgtttcagttaaacaaagattcagacaagtgatgagccacttcttgtcatagattgtgtggtcaaccaacccattgatgccacggtttttgtgcatccatgcttttgtattaaaacatcagttatgtttaccatgtccgatatgctctacataatgacaatcaacttggtgttcattatatttacaatgttaggaatttcaatagctacgaacacttcaatagaagtgcctttgtgggatcaccttaatgtgttgttgatgtaaggagaagaatcttaatttactcttgctaaatttgaactacacaaaaccactgcactgaggattgtcctaataaattactgctcatacacgttagcatctgttcagatactgagctaatccctaggattaaaggatttgtaaaagatatgcccaatcattcattttagttatttatttcttagttatccacttgaagatttacatacatttgaaataaatttcttagaggtaaagtgaaaatcagttatttaaatacattttagttatttattttcttctttttcctaatttttccttgtatttgaagtctgaaaagataactttgcccttatacatattttatcttctacgtacgcatctgaacaacgtctctttgtcccctgatcgtgcagcaattagtgctatgaatcgcgtttaagcgctgcaaaatcatggctggggcttcgtcctcgagtcgtcctgctgctcgatgtcacctcgagtcccgcaccgacctcagtgcttgttcttgttggagccacctctctcggacgatcgccaaagacggataaggccgaagccgtcacttcagaccgcgctcatgcgccgtagcagactcctacatagcagggccagggtatgtggacctttgcaagtttaggattggaaccagcgaccagaatccacaagattggagcaaacgaccaaaaattcacaaggattggcggctgacattgccagcgcgggatcgcatgcggcggcggcggccggggcgagcacgggagcaggcgacagtcgagctccattggaacgtagaaatacttaagggcaaggtctccaaatacttgaaaaaataggaaaaagaagaaaatacatgaaatgatattgaaatcaattggaagatgttatgaatcttgtttttgcaaagcgaacgattcagatggcaaaactatgaatctttttgtttgaagtcccaaatataaaattttctcgtactcaccaacattggtgcgcacctgtgattggctcataaaaattcttggagggacggaagaaagagtgaagggataagcaagtaaaagcgctcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagcttactagtagcgctgtttaaacgctcttcaactggaagagcggttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatgtaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccacacgacaccatgtccgcccgcgaggtgcacatcgacgtgaacaacaagaccggccacaccctccagctggaggacaagaccaagctcgacggcggcaggtggcgcacctccccgaccaacgtggccaacgaccagatcaagaccttcgtggccgaatccaacggcttcatgaccggcaccgagggcaccatctactactcaattaatggcgaggccgagatcagcctctacttcgacaacccgttcgccggctccaacaaatacgacggccactccaacaagtcccagtacgagatcatcacccagggcggctccggcaaccagtcccacgtgacctacaccatccagaccacctcctcccgctacggccacaagtcctgagtcatgagtcatgagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgcggaccgaattggggatctgcatgaaagaaactgtcgcactgctgaaccgcaccttgtcactttcatcgaacacgacctgtgcccaagatgacggtgctgcggtctaagtgaggctgaattgccttggacagaagcggactccctacaattagttaggccaaacggtgcatccatgtgtagctccgggctcgggctgtatcgccatctgcaatagcatccatggagctcgttccatgtagttggagatgaaccaatgatcgggcgtgtggacgtatgttcctgtgtactccgatagtagagtacgtgttagctctttcatggtgcaagtgaaatttgtgttggtttaattacccctacgttagttgcgggacaggagacacatcatgaatttaaaggcgatgatgtcctctcctgtaatgttattcttttgatgtgatgaatcaaaatgtcatataaaacatttgttgctctttagttaggcctgatcgtagaacgaaatgctcgtgtagcggggctacgagcctatgacgcaataacactggtttgccggcccggagtcgcttgacaaaaaaaagcatgttaagtttatttacaattcaaaacctaacatattatattccctcaaagcaggttcacgatcacacctgtacctaaaaaaaacatgaagaatatattactccattattatgagatgaaccacttggcaagagtggtaagctatataaaaaaatgaacattattacgagatgttatatgccattatattgattcgaagatatatgtttctttctcccacgggcacctaacggatacatgataaggccaaggcagatcacgggaaattattcgaatacatgttacgccctattgccggaaaaaaaatgcagggcaggtgttggccgtagcgatttaagcacttaagctggaggttgccacacttggatgcaagcgtctgacccttctaaaacatcggcggctttgtccgtatccgtatcccctatccgacatctagctggccacacgacggggctgggcagatcgtggatgccgggtcgacgtcgatcgtcagccatcatagaccaatcgaccatctgttatggatgcttgctagctagactagtcagacataaaatttggatactttctcccaactgggagacggggactgatgtgcagctgcacgtgagctaaatttttccctataaatatgcatgaaatactgcattatcttgccacagccactgccacagccagataacaagtgcagctggtagcacgcaacgcatagctctggacttgtagctaggtagccaaccggatccacacgacaccatgctcgacaccaacaaggtgtacgagatcagcaaccacgccaacggcctctacgccgccacctacctctccctcgacgactccggcgtgtccctcatgaacaagaacgacgacgacatcgacgactacaacctcaagtggttcctcttcccgatcgacgacgaccagtacatcatcacctcctacgccgccaacaactgcaaggtgtggaacgtgaacaacgacaagattaatgtgtcaacctactcctccaccaactccatccagaagtggcagatcaaggccaacggctcctcctacgtgatccagtccgacaacggcaaggtgctcaccgccggcaccggccaggccctcggcctcatccgcctcaccgacgagtcctccaacaacccgaaccagcaatggaacctgacgtccgtgcagaccatccagctcccgcagaagccgatcatcgacaccaagctcaaggactacccgaagtactccccgaccggcaacatcgacaacggcacctccccgcagctcatgggctggaccctcgtgccgtgcatcatggtgaacgacccgaacatcgacaagaacacccagatcaagaccaccccgtactacatcctcaagaagtaccagtactggcagagggccgtgggctccaacgtcgcgctccgcccgcacgagaagaagtcctacacctacgagtggggcaccgagatcgaccagaagaccaccatcatcaacaccctcggcttccagatcaacatcgacagcggcatgaagttcgacatcccggaggtgggcggcggtaccgacgagatcaagacccagctcaacgaggagctcaagatcgagtattcacatgagacgaagatcatggagaagtaccaggagcagtccgagatcgacaacccgaccgaccagtccatgaactccatcggcttcctcaccatcacctccctggagctctaccgctacaacggctccgagatccgcatcatgcagatccagacctccgacaacgacacctacaacgtgacctcctacccgaaccaccagcaggccctgctgctgctgaccaaccactcctacgaggaggtggaggagatcaccaacatcccgaagtccaccctcaagaagctcaagaagtactacttctgagtcatgagtcatgagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacagggtacccggggatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgacctgcaggcatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagggcgagctcggtacccggggatcctctagagtcgacctgcaggcatgcccgcggatatcgatgggccccggccgaagcttcggtccgggccatcgtggcctcttgctcttcaggatgaagagctatgtttaaacgtgcaagcgctcaattcgccctatagtgagtcgtattacaatcgtacgcaattcagtacattaaaaacgtccgcaatgtgttattaagttgtctaagcgtcaatttttcccttctatggtcccgtttgtttatcctctaaattatataatccagcttaaataagttaagagacaaacaaacaacacagattattaaatagattatgtaatctagatacctagattatgtaatccataagtagaatatcaggtgcttatataatctatgagctcgattatataatcttaaaagaaaacaaacagagcccctataaaaaggggtcaagtggacacttggtcactcatttaatccctccctctcctcttttatccctctttttggtgtattcaccaatagtggtgtgcacctgtgattggctcgtaaaaattcttggacggatggaagagtgaagagataagcaagtcaaagaaaagtaacaacgaagcttcatcagctacaaattttggcccaactggttgcaccagcaccaaacttacgtatacatgattatctctgtttccctcatttcgaagaaaaaaacgggtttcaaaacccactgctttcaggagtaaaaaaagataataatctgaaacattgcttccaccttggcccttatttggttacgttgcaattcaccccaatccacatgtggattgagatggattgcagtgtagctagacaaacccttaggccctgtttgcataggaatacaccaggaattattccagctaatcaaaatttatataaatgagagaaacaattcggataggaattgttccaggacttcattctgcagtaaccgaacggccccttaatccaccccaatacacgtggattggagtggattgaggtacagccaaacaaggcctaagtgcagatcaaataaatcacccgtcatattcttctacctacaaaaacagcaataaacacctgaatgaagttctaatttgcacagtgtaggtaggatgaaaatagttacctcctcatggtcagtaactcttggcacacaacttcacatgtaatcgatgtaccacttggctcttgcctgaaacccaatacatctttagcataagaataatattatgatggcaaggcatgatcaccagcactcctttattgtttagtaagtctatcactccccaaaacaattcaaatgaacagagatgcattgcccccaatgaattctatttcaattagccggaaaattctacttcatcagaagcatccaaattgccagcatccctactagactgaccatgaccaggctgccgcagatgcctctttttctgtcctctcctctttgccttgagtttctcttcaagatccctcaccccacgtctcttatacatcttaaagctaacatgtctctcctccgccatcttcctaaccttctcagtaatctcagcagcaatctgacggttgtacaacttcttcagccccttcatcaactttgcaaatgtgtcaggctgtggcatcagtcctgcctctagcatgtctaagcaatacaggcaggcctccttgacatgtttcttcgcaaacagtgcatgaatccagatagtccatgcactcacattgagctcacagcctttgctcacaatacatttccaaacatcctttgcaagctcaagtttctcatctctgaccaacgcattgaggaggtccttcagcaccccatattgcggtaccacaaagagccccctcccaaccatgtctttaaaataactacatgcctcaatcagcaaaccctgcccaacaaggccactcaccacgatagcaaatgtatcgaccacaggactgagcccagcactttccatctcattccacaatgtcatggcttgcttggtctccccaagcctgcaggccaaccgaatcaccacattgtatatcttgagatctggtggacaccggcactcccgcatcctctccatcagctccaagcactcctcaagctgctccttcttctcgtgtgctacaaagaaaccatggtacacggcagcgtccacccgcaggccatccctcgacatagcatccaagaactcgtacccctgggat

SEQ ID NO: 2 - DNA, 16752 bp, Artificial
  synthetic
gagcatatccagcaccagctggtaccaaggtcgggtctctgtgctagtgctattagctagtgtaaggagcgagtaggtcagttaaggctggtgcgtcgtgagggctgtcttgtgtgtagctacagcagacggttcatcagaaggattattcgtgcagtatatacagtacaactagacaatgatgttgatgattggtctagagctagaggcctatagccctatactactgtgtattgtccgccgttttagttttttggtcccatcccatcaatgcaaccgccttgttttgctccaattgtcccgttcctgcgcctcgcttttgctctgtcgcatcgcatacaaaaaaaaaaacgccgcgccggctttgaatcgcgccccccaactgctccaaccaggcaacggacacggccaccgtccgtgtcgcgagcaaaaaaacaaaaagaggaacgcgtccaggacgaagcagtccactgccgctgtggccggcaaaagatctggttgagcacatggagattggagaaggttggttggttcttctggaaacgccaatgaatgggggcactgacatgtactcttaacatgtagtgcaatccagagatcggatatccagacactggcagcacgatcgcctcgcgccgtagatcacgcacgcaaattactgaagaccattcacaaaaaaaaaaaaacacacaggggctagcgtgccccacaccaaacccaagtgctgcgttgcacgcaggggagcgaaaaaaaacaataatgctcactgtcacgtcgcgtatccaaccccgcggacgtctcggctctcagcagcagcacacggggcacctcacgatgccgttctcgttgcactccgtgcaccgccggaacccgccgccgcattcgtcgccctcctcctcctcctccgcctcgtcttcgtcacccacgtacaccttgcagctgcccgagcagacatcgcagagcacgaaccgcatgtccccgcaggcctcgcacgcgccggcgtcgccgccgtgtgggccggccgtcgacgcagcgctctcgcacccggccagcctcggcgcgagctccccggcctcgtgcagccgcttcagctcctcggcgttgcccacgagctccccgtccacgaagaggctggggagggcggcgggcgtgccgccggcttggccgagcccgaggccgagaaggccgcggagctcgtcccggaacccgcggtgcatggacacgtcgcgctcgtcgaggcgcacgccgtagcccttgaggatggcgcgcgccaggcagcagtcctcgtgcgtggcgcgcacgccgcgcagcgacgtgaagtagagcaccgccctccgcggcggcagcgccttcccctccccgccgctcgtcggggcggcgtcgggccgaggcatcggcatcggcagcggcgtcaccttggcggacgccgcgaggtcctgcgcaggcgccgtggcgaccgggaacgagaaggagtggcgcccgaacggcgcgcccagcagcggggagcggtcctcgaggccggccatgagcgcccacgcgtcgatgtcctcgggctcgttgggcggcgtcatggtgggcgtgcgcggcgccagcctcgtgggcgcgggctccggcgcccgcggcagggccttgtccagctccagggacccgagcgtggacgacgtgagccgcaccacgtggacgccgacgtcgctggggcaccgagccgggaacgactggctgcgcggcagcggtgacgggcagtaccggaggtcgtgacgggcctgccttgaggtggtgcaccccatggcaccaatgtacacacacggccaaagcgccaagtgggctgcagactgcctgccaatgtgatcaagcagccaggagcagagacggatctctggggatcggggtttctggggtttaggatctttatactactctgtcattggggatataaaactaggagtgtggttaattaggactcgatagataagtttaccacaagcgcgtgaaatggtctacccgatgatgtgattggcctaaaaagaacaagaagagtatttggagctactgaacattctcttttcctgaagataactaatttttggaacattcagacttgggagtctggacttttggagggaagttcaaattgtggtctgcctctgccatgtgttgttttttagtcggagagtggccctcattttttttgtcctgtttagctttatagtcgtagcagctagtagcgaaatttaaccttggattatggccgtgttagtcaaacaatcattgatttatttcctccctttcgcgctgcttttcctgtacgcatctccgctgcccttgattcgaggaccctgttcacaacacagggctctggctttggagcctctcgtttgtagcacttgcacgtagttacccggaccgaagcttcaacacagatctgatagtttaaacgctcttcaactggaagagcggttacccggaccgaagcttcggccggggcccatcgatatccgcgggcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatggagaacaacatacagaatcagtgcgtcccctacaactgcctcaacaatcctgaagtagagattctcaacgaagagaggtcgactggcagattgccgttagacatctccctgtcccttacacgtttcctgttgtctgagtttgttccaggtgtgggagttgcgtttggcctcttcgacctcatctggggcttcatcactccatctgattggagcctctttcttctccagattgaacagttgattgaacaaaggattgagaccttggaaaggaatcgggccatcactacccttcgtggcttagcagacagctatgagatctacattgaagcactaagagagtgggaagccaatcctaacaatgcccaactgagagaagatgtgcgtatacgctttgctaacacagatgatgctttgatcacagccatcaacaacttcacccttaccagcttcgagatccctcttctctcggtctatgttcaagctgctaacctgcacttgtcactactgcgcgacgctgtgtcgtttgggcaaggttggggactggacatagctactgtcaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacatagttgctctctttccgaactacgatgttcgtacctatccgattcaaacgtcatcccaacttacaagggagatctacaccagttcagtcattgaagactctccagtttctgcgaacatacccaatggtttcaacagggctgagtttggagtcagaccaccccatctcatggacttcatgaactctttgtttgtgactgcagagactgttagatcccaaactgtgtggggaggacacttagttagctcacgcaacacggctggcaatcgtatcaactttcctagttacggggtcttcaatcccgggggcgccatctggattgcagatgaagatccacgtcctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcagattcccttggtgaaggcacacacacttcagtcaggaactacagttgtaagagggccggggttcacgggaggagacattcttcgacgcactagtggaggaccattcgcgtacaccattgtcaacatcaatgggcaacttccccaaaggtatcgtgccaggatacgctatgcctctactaccaatctaagaatctacgttacggttgcaggtgaacggatctttgctggtcagttcaacaagacaatggataccggtgatccacttacattccaatctttctcctacgccactatcaacaccgcgttcacctttccaatgagccagagcagtttcacagtaggtgctgataccttcagttcaggcaacgaagtgtacattgacaggtttgagttgattccagttactgccacactcgagtaaggatccgtcgacctgcagccaagctttcgcgagctcgagatccccgacatatgccccggtttcgttgcgactaacatgagttcttggacaaatttgattggacctgatgagatgatccaacccgaggatatagcaaagctcgttcgtgcagcaatggaacggccaaaccgtgcttttgtccccaagaatgaggtgctatgcatgaaggaatctacccgttgatgtccaacagtctcagggttaatgtctatgtatcttaaataatgttgtcggtattttgtaatctcatatagattttcactgtgcgacgcaaaaatattaaataaatattattattatctacgttttgattgagatatcatcaatattataataaaaatatccattaaacacgatttgatacaaatgacagtcaataatctgatttgaatatttattaattgtaacgaattacataaagatcgaatagaaaatactgcactgcaaatgaaaattaacacatactaataaatgcgtcaaatatctttgccaagatcaagcggagtgagggcctcatatccggtctcagttacaagcacggtatccccgaagcgcgctccaccaatgccctcgacatagatgccgggctcgacgctgaggacattgcctaccttgagcatggtctcagcgccggctttaagctcaatcccatcccaatctgaatatcctatcccgcgcccagtccggtgtaagaacgggtctgtccatccacctctgttgggaattccggtccgggtcacctttgtccaccaagatggaactgcggccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccacacgacaccatgtccgcccgcgaggtgcacatcgacgtgaacaacaagaccggccacaccctccagctggaggacaagaccaagctcgacggcggcaggtggcgcacctccccgaccaacgtggccaacgaccagatcaagaccttcgtggccgaatccaacggcttcatgaccggcaccgagggcaccatctactactcaattaatggcgaggccgagatcagcctctacttcgacaacccgttcgccggctccaacaaatacgacggccactccaacaagtcccagtacgagatcatcacccagggcggctccggcaaccagtcccacgtgacctacaccatccagaccacctcctcccgctacggccacaagtcctgagtcatgagtcatgagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgcggaccgaattggggatctgcatgaaagaaactgtcgcactgctgaaccgcaccttgtcactttcatcgaacacgacctgtgcccaagatgacggtgctgcggtctaagtgaggctgaattgccttggacagaagcggactccctacaattagttaggccaaacggtgcatccatgtgtagctccgggctcgggctgtatcgccatctgcaatagcatccatggagctcgttccatgtagttggagatgaaccaatgatcgggcgtgtggacgtatgttcctgtgtactccgatagtagagtacgtgttagctctttcatggtgcaagtgaaatttgtgttggtttaattacccctacgttagttgcgggacaggagacacatcatgaatttaaaggcgatgatgtcctctcctgtaatgttattcttttgatgtgatgaatcaaaatgtcatataaaacatttgttgctctttagttaggcctgatcgtagaacgaaatgctcgtgtagcggggctacgagcctatgacgcaataacactggtttgccggcccggagtcgcttgacaaaaaaaagcatgttaagtttatttacaattcaaaacctaacatattatattccctcaaagcaggttcacgatcacacctgtacctaaaaaaaacatgaagaatatattactccattattatgagatgaaccacttggcaagagtggtaagctatataaaaaaatgaacattattacgagatgttatatgccattatattgattcgaagatatatgtttctttctcccacgggcacctaacggatacatgataaggccaaggcagatcacgggaaattattcgaatacatgttacgccctattgccggaaaaaaaatgcagggcaggtgttggccgtagcgatttaagcacttaagctggaggttgccacacttggatgcaagcgtctgacccttctaaaaaatcggcggctttgtccgtatccgtatcccctatccaacatctagctggccacacgacggggctgggcagatcgtggatgccgggtcgacgtcgatcgtcagccatcatagaccaatcgaccatctgttatggatgcttgctagctagactagtcagacataaaatttggatactttctcccaactgggagacggggactgatgtgcagctgcacgtgagctaaatttttccctataaatatgcatgaaatactgcattatcttgccacagccactgccacagccagataacaagtgcagctggtagcacgcaacgcatagctctggacttgtagctaggtagccaaccggatccacacgacaccatgctcgacaccaacaaggtgtacgagatcagcaaccacgccaacggcctctacgccgccacctacctctccctcgacgactccggcgtgtccctcatgaacaagaacgacgacgacatcgacgactacaacctcaagtggttcctcttcccgatcgacgacgaccagtacatcatcacctcctacgccgccaacaactgcaaggtgtggaacgtgaacaacgacaagattaatgtgtcaacctactcctccaccaactccatccagaagtggcagatcaaggccaacggctcctcctacgtgatccagtccgacaacggcaaggtgctcaccgccggcaccggccaggccctcggcctcatccgcctcaccgacgagtcctccaacaacccgaaccagcaatggaacctgacgtccgtgcagaccatccagctcccgcagaagccgatcatcgacaccaagctcaaggactacccgaagtactccccgaccggcaacatcgacaacggcacctccccgcagctcatgggctggaccctcgtgccgtgcatcatggtgaacgacccgaacatcgacaagaacacccagatcaagaccaccccgtactacatcctcaagaagtaccagtactggcagagggccgtgggctccaacgtcgcgctccgcccgcacgagaagaagtcctacacctacgagtggggcaccgagatcgaccagaagaccaccatcatcaacaccctcggcttccagatcaacatcgacagcggcatgaagttcgacatcccggaggtgggcggcggtaccgacgagatcaagacccagctcaacgaggagctcaagatcgagtattcacatgagacgaagatcatggagaagtaccaggagcagtccgagatcgacaacccgaccgaccagtccatgaactccatcggcttcctcaccatcacctccctggagctctaccgctacaacggctccgagatccgcatcatgcagatccagacctccgacaacgacacctacaacgtgacctcctacccgaaccaccagcaggccctgctgctgctgaccaaccactcctacgaggaggtggaggagatcaccaacatcccgaagtccaccctcaagaagctcaagaagtactacttctgagtcatgagtcatgagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattatcgatgggccccggccgaagctggccgcggaccgaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacagggtacccggggatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgacctgcaggcatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagggcgagctcgaattcgagctcgagcccgggtggatcctctagagtcgacctgcagaagcttcggtccggcgcgcctctagttgaagacacgttcatgtcttcatcgtaagaagacactcagtagtcttcggccagaatggcctaactcaaggccatcgtggcctcttgctcttcaggatgaagagctatgtttaaacgtgcaagcgctactagacaattcagtacattaaaaacgtccgcaatgtgttattaagttgtctaagcgtcaatttggaacaagtggctatcgccagatataagaacttcgatccgaaatatcgtttcaaaactagaaaacagcgcggctttggctaagccgcgcactatataggattttgggcaccttttgatggaacgtgaaagcgtactgcgcactagttatttaggttgaaccttggatatacggttctcactgcgccaatgcaaggcttgaaacttggttagtaatacgtactccctccgtttctttttatttgtcgctggatagtgcaattttgcactatcgagcgacaaataaaaagaaacggagggagtatatgattgtcagatgtagatatgtttatttatatatcacatacagatatataaaacagatcactttttcagatatacagttccaatgtcagccctgatcaccctgtcataaattgcacgtttctaattgatgttgcttcatggtcgtcatgagaaccttctgaagaaatcgatgaaggttgccaacctttcaaagtttcagaaaccactttgcatgtacactaagggctggtttggcagcccaaaaccagccagcgttttcctggtcttttctcccgggagaaagcccatgcatagattgtccctggattatttatctgtgtcctttggctaaaaattcgtcccaatttcctgtaggaaactacctcggccttgggaggccaggcgattctccaccgcctcgtctcgtccatccttcgatgctcacgcgtgcctcctcggatgctatcctcaggcgattctccgtcgtctcgtctcatccatcctcacgcgcgcctcctccgacgctatccccaggcgattctccaccgtctcgtctcatccatcctcatgtacgcctcgtccgatgctatccccagacgattttccgtcgtctcatctccttcatgctcgcgcgcgcctcctccgacgctatccccaggcgatttttctgccgtctcgtctccttcatgcccgcgcgcgcctcctccgacgctatccccaggcgattttccgccgtctcgtctccttcatgcccgcgcgtgcctcctccgacgctattcccacgagcgcctccgccgccgctatccccagacgattttccgctgtctcgtctccttcatgcccgcgcgcccctcctccgacgctatccccacgagcgcctccgccgccgctccaccgtcttccccgccgccatccccttaattcctatagatctggaccccgctctactttcgttggcatacttttgcttggtgtgcgcgggctggagtggaaggttgcgcattcgatcacgggggagaagtggatcttgggtcttggcaggctagggcggttgccaggacgccgtggtgtgcattcatgggtcctataaatctttatcattaccgccttaggagctagttgtagttcacacatcatatccttttctgctcgacatcgtctggggatgccctaggtgccctaccgaccctacggcattgtcttgacctctattagactctatgtcatctagagccttcttgggtggccttttgaccccaaagcgaccctatgatcttaccctaacgaggtctcccttggtggggcaagatccactttgtccacttaactgaagatctgatcctcatcttgaaatctttaatcccaaggtgactctacgtcgtatgtggatgctccgggtaacctgccaacccggatcaccctaagatctctttcctaaggggcgagatctaggttcctacgagaaagaagacgaccctgcaccattgcggtccgtccggtccagagtgcgaacgtccggatgcgacacagggaaggagtcgctcctgcagcgaggtcgcagactgtccacacagcctcagaaggcaccgccagacaatacatgtaatacccactctgtaagaaaaacctaaaaggagaaagtatattcctttatctatatgtgtgttatatttctactcaccatcacatgtgaacatctcacttacacaaataaataattaacaaaagacactcaaataaattatgcatcatgctcgaccttattttgtgtgcattctgttacaatataaaaataatataaaaaacatatattaatatcaaaatttggagatttaaccctaatatgcaaatcggagtttagaggaaagaaagaaaaatgctatacaaaataaaggaataaatatataaataaaggtaaaactattaatactggtatattaatttgaacagttgacctaattatgaatatcacaactggtttgaattcaaatatgaaatccaagaatttggaaataggaaaaatggagataagaataaaggaaaagaattcttaactcggatgggcctgggaaacgaatttcggcccacttcctgtgtccttagctgtgcggctcagtccagtg

SEQ ID NO: 3 - DNA, 12248 bp, Artificial
  synthetic
ccctagcgttgggcccaactagtcagtctgccttccctcccagtcgctgacctccttggtccacttgtcagctttcgcgctcagatctaatctcaactgtcgatctgtgatcgggtggccgagatcacccgatgcccgttcgtttgcaaaagttgttaaaagaccctctgtttcttagaaaataacctacattcatgtttcttgcgattaggcccctggtttcttgtagagaagccccttgctttattttaatcacaaaaataaatctaatttagtgttttgaattctaaaacttgtgaatttcatatcttttgcatatgaactctaaattgggtggtttaaattgcaaaatgatcataatattattctctatctgtttaaattataatatttcactgtctacatgtatgtattttatgactagacaataggttcatttaaagtgatggattatttattaaaaggaaaataaaaaggcaaaacactaatgaatagttaagtggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccaccactcgaggtcgaggtaccgttgtcaatcaattggcaagtcataaaatgcattaaaaaatattttcatactcaactacaaatccatgagtataactataattataaagcaatgattagaatctgacaaggattctggaaaattacataaaggaaagttcataaatgtctaaaacacaagaggacatacttgtattcagtaacatttgcagcttttctaggtctgaaaatatatttgttgcctagtgaataagcataatggtacaactacaagtgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcaaactcagtaggattctggtgtgtgcgcaatgaaactgatgcattgaacttgacgaacgttgtcgaaaccgatgatacgaacgaaagctaggcctcagcgagtaccgctggcgatctaatccatgatatcgtgaacatcatctacattcaaattcttatgagctttcttaagggcatctgcagcatttttcatagaatctaatacagcagtatttgtgctagctccttcgagggcttccctctgcatttcaatagttgtaagggttccatctatttgtagttgggtcttttccaatcgtttcttctttttgagggcttggagtgcaactcttttatttttcgacgcatttttctttgcgctcctgcaggcggccgcgtggatgaggagttaatcggtcgtgtgagagtagtgatcgagtggatgtcgtcgagagtgatgagtgttgatgttgttagtgatatgtggtagaaggtatcgtgataaagcgttaacgcgatcgcagtacttgcaaagaaaaatgcgtcgaaaaataaaagagttgcactccaagccctcaaaaagaagaaacgattggaaaagacccaactacaaatagatggaacccttacaactattgaaatgcagagggaagccctcgaaggagctagcacaaatactgctgtattagattctatgaaaaatgctgcagatgcccttaagaaagctcataagaatttgaatgtagatgatgttcacgatatcatggatggtatcgcacagcgactgctgagggacgtcgagctcccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaatcggacctgcagcctgcaggctagcggcgcgccacaaatcacaggccatgaaccctactcatgcttcgatttgtccaacacacacttaccaaaactcaaatcatgtccttgacagtcactcgggactcataacatgggtacgtatcgactatgtcaactatatgtgttctcatcagattatagattggcctagtacgtagtgatatttccactagcactgtggttatggctgtacctgatagtgatatcagcaccgggtcatggctctactaccaggtagtgagagtgacctttatactgtcagactgtaactaaggatttccaatcactgttcggatcctaggcttagaattaagtaaaactctatcactataggctgcagcacactcggtatatattgatgggccaacagaaattgtgcgtactatgcgcgatgtaaaatggacataaaccctacccatatacaatgcaataacttttgtccggtctgggccaccggttagcagaggtcctgatttcggtggtagtggtagcttgatctggtcgtcgtatcgtagagggatatataaaatcatgtcacttttgaagggagcgctcacagaaataataggtattcgcgggagccgcccccgcagaacacaaaataaggcgagcacgcacacgcatcagtttcgataaaataataatagcgccagctgatcggaacaattccagctagcactaatgtatttctgcattgatctgtttatacaacatgctacctcgttgagtgattttgacatgatttgtcaacttgctccgatcctatatctcgatcgatctccacatgacgatggttgttgtcctgtatcccatgacaaccaggcaacgctcaaagcacacatgcgttgccgattacccgtgcatgccgccaagcacgaaagcacctccctccacaccgtccatcagctataaaaaccatgccaagcaccctgtgaaaagccccgggaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggccaaccccaacaatcgctccgagcacgacacgatcaaggtcacccccaactccgagctccagaccaaccacaaccagtacccgctggccgacaaccccaactccaccctggaagagctgaactacaaggagttcctgcgcatgaccgaggactcctccacggaggtcctggacaactccaccgtcaaggacgccgtcgggaccggcatctccgtcgttgggcagatcctgggcgtcgttggcgtccccttcgcaggtgctctcacctccttctaccagtccttcctgaacaccatctggccctccgacgccgacccctggaaggccttcatggcccaagtcgaagtcctgatcgacaagaagatcgaggagtacgccaagtccaaggccctggccgagctgcaaggcctgcaaaacaacttcgaggactacgtcaacgcgctgaactcctggaagaagacgcctctgtccctgcgctccaagcgctcccaggaccgcatccgcgagctgttctcccaggccgagtcccacttccgcaactccatgccgtccttcgccgtctccaagttcgaggtcctgttcctgcccacctacgcccaggctgccaacacccacctcctgttgctgaaggacgcccaggtcttcggcgaggaatggggctactcctcggaggacgtcgccgagttctaccgtcgccagctgaagctgacccaacagtacaccgaccactgcgtcaactggtacaacgtcggcctgaacggcctgaggggctccacctacgacgcatgggtcaagttcaaccgcttccgcagggagatgaccctgaccgtcctggacctgatcgtcctgttccccttctacgacatccgcctgtactccaagggcgtcaagaccgagctgacccgcgacatcttcacggaccccatcttcctgctcacgaccctccagaagtacggtcccaccttcctgtccatcgagaactccatccgcaagccccacctgttcgactacctccagggcatcgagttccacacgcgcctgaggccaggctacttcggcaaggactccttcaactactggtccggcaactacgtcgagaccaggccctccatcggctcctcgaagacgatcacctcccctttctacggcgacaagtccaccgagcccgtccagaagctgtccttcgacggccagaaggtctaccgcaccatcgccaacaccgacgtcgcggcttggccgaacggcaaggtctacctgggcgtcacgaaggtcgacttctcccagtacgatgaccagaagaacgagacctccacccagacctacgactccaagcgcaacaatggccacgtctccgcccaggactccatcgaccagctgccgcctgagaccactgacgagcccctggagaaggcctactcccaccagctgaactacgcggagtgcttcctgatgcaagaccgcaggggcaccatccccttcttcacctggacccaccgctccgtcgacttcttcaacaccatcgacgccgagaagatcacccagctgcccgtggtcaaggcctacgccctgtcctcgggtgcctccatcattgagggtccaggcttcaccggtggcaacctgctgttcctgaaggagtcctcgaactccatcgccaagttcaaggtcaccctgaactccgctgccttgctgcaacgctaccgcgtccgcatccgctacgcctccaccacgaacctgcgcctgttcgtccagaactccaacaatgacttcctggtcatctacatcaacaagaccatgaacaaggacgatgacctgacctaccagaccttcgacctcgccaccacgaactccaacatgggcttctcgggcgacaagaatgaactgatcattggtgctgagtccttcgtctccaacgagaagatctacatcgacaagatcgagttcatccccgtccagctgtgataggaactctgattgaattctgcatgcgtttggacgtatgctcattcaggttggagccaatttggttgatgtgtgtgcgagttcttgcgagtctgatgagacatctctgtattgtgtttctttccccagtgttttctgtacttgtgtaatcggctaatcgccaacagattcggcgatgaataaatgagaaataaattgttctgattttgagtgcaaaaaaaaaggaattagatctgtgtgtgttttttggatcccattttcgacaagcttgcctcgagacaacaacatgcttctcatcaacatggagggaagagggagggagaaagtgtcgcctggtcacctccattgtcacactagccactggccagctctcccacaccaccaatgccaggggcgagctttagcacagccaccgcttcacctccaccaccgcactaccctagcttcgcccaacagccaccgtcaacgcctcctctccgtcaacataagagagagagagaagaggagagtagccatgtggggaggaggaatagtacatggggcctaccgtttggcaagttattttgggttgccaagttaggccaataaggggagggatttggccatccggttggaaaggttattggggtagtatctttttactagaattgtcaaaaaaaaatagtttgagagccatttggagaggatgttgcctgttagaggtgctcttaggacatcaaattccataaaaacatcagaaaaattctctcgatgaagatttataaccactaaaactgccctcaattcgaagggagttcaaaacaattaaaatcatgttcgaattgagtttcaatttcactttaacccctttgaaatctcaatggtaaaacatcaacccgtcaggtagcatggttctttttattcctttcaaaaagagttaattacaaacagaatcaaaactaacagttaggcccaaggcccatccgagcaaacaatagatcatgggccaggcctgccaccaccctccccctcctggctcccgctcttgaatttcaaaatccaaaaatatcggcacgactggccgccgacggagcgggcggaaaatgacggaacaacccctcgaattctaccccaactacgcccaccaacccacacgccactgacaatccggtcccacccttgtgggcccacctacaagcgagacgtcagtcgctcgcagcaaccagtgggcccacctcccagtgagcggcgggtagatctggactcttacccacccacactaaacaaaacggcatgaatattttgcactaaaaccctcagaaaaattccgatattccaaaccagtacagttcctgaccgttggaggagccaaagtggagcggagtgtaaaattgggaaacttaatcgagggggttaaacgcaaaaacgccgaggcgcctcccgctctatagaaaggggaggagtgggaggtggaaaccctaccacaccgcagagaaaggcgtcttcgtactcgcctctctccgcgccctcctccgccgccgctcgccgccgttcgtctccgccgccaccggctagccatccaggtaaaacaaacaaaaacggatctgatgcttccattcctccgtttctcgtagtagcgcgcttcgatctgtgggtggatctgggtgatcctggggtgtggttcgttctgtttgatagatctgtcggtggatctggccttctgtggttgtcgatgtccggatctgcgttttgatcagtggtagttcgtggatctggcgaaatgttttggatctggcagtgagacgctaagaatcgggaaatgatgcaatattaggggggtttcggatggggatccactgaattagtctgtctccctgctgataatctgttcctttttggtagatctggttagtgtatgtttgtttcggatagatctgatcaatgcttgtttgttttttcaaattttctacctaggttgtataggaatggcatgcggatctggttggattgccatgatccgtgctgaaatgcccctttggttgatggatcttgatattttactgctgttcacctagatttgtactcccgtttatacttaatttgttgcttattatgaatagatctgtaacttaggcacatgtatggacggagtatgtggatctgtagtatgtacattgctgcgagctaagaactatttcagagcaagcacagaaaaaaatatttagacagattgggcaactatttgatggtctttggtatcatgctttgtagtgctcgtttctgcgtagtaatcttttgatctgatctgaagataggtgctattatattcttaaaggtcattagaacgctatctgaaaggctgtattatgtggattggttcacctgtgactccctgttcgtcttgtcttgataaatcctgtgataaaaaaaattcttaaggcgtaatttgttgaaatcttgttttgtcctatgcagcctgatccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggcgatgggcgcccgcatccgtaaggaaggcgacacctggatcatcgatggcgtcggcaatggcggcctcctggcgcctgaggcgccgctcgatttcggcaatgccgccacgggctgccgcctgacgatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctgatgagctccagggttcttgcctggtgccttggcaatgcttgattactgctgctatcctatgatctgtccgtgtgggcttctatctatcagtttgtgtgtctggttttgaaaaacatttgcttttcgattatgtagggtttgcttgtagctttcgctgctgtgacctgtgttgtttatgtgaaccttctttgtggcatctttaatatccaagttcgtggtttgtcgtaaaacgaagcctctacttcgtaaagttgtgtctatagcattgaaatcgtttttttgctcgagaataattgtgacctttagttggcgtgaaactagttttggatatctgattctctggttcgcaatcttgagatcgtcgctgcttaggtgagctaagtgatgttcctaagtaaatgctcctcaccagaatacgtagctgtgtgaaaagagaacgcgtgaatacgtagctgtgtaaagattgtgtcccaagtaaacctcagtgatttttgtttggatttttaatttagaaacattcgactgggagcggctagagccacacccaagttcctaactatgataaagttgctctgtaacagaaaacaccatctagagcggccgcgtttaaactatcagtgtttagagaatcacaaacctctagatgtattaatctaccctagaactagttcacttttgtgtgcatacttttctattgaactggtgttcactttgttgcatatgttttgtgtactgtttatttgtcattgcccaaatgtgtttaatgagtgattgctttgcgtagacaacgagcagttcaaggtttccgagtgtgttgcaaaagacttccctgagcagcaacctggtgaaggtaagtgtcctctgacccattatgtcatatttactttataattatatccttaacaatatgattaaagattaacaccaaattatatacatacatatgttaaaattttaaatgtcaaataattcagatgtttagaatgcatccctaagacggccagtgcaggctcgtacgatgcatacgaaaacctatccctagtgttcgcttcgaattaatgccgacagtttaaactactgcatctgcaatctatagagacaaaaacactatgaaaatagtaga

SEQ ID NO: 4 - DNA, 12282 bp, Artificial
  synthetic
aatatttaaaaatggaagtaatactatattaaaatgattcatgtggaactcctgcgcttctttttgaagtttcaaaagggagctttcagggtcgcttagagtttgtttggttggaaatacaagcgaaaagagagctaatgagggggacatccatattttctatggtgtttgaataagagtcacgcgggaataagatgaacaccgaaacaatttttttgtagctacgtggttccaaaaaatcgagtagacggtgtcgcttccacctcatactacttcaacctcaaaccacacatccttacgcgcccgccggtgtctccgtcgtctcaagttctcaacatacctacacatgtaaccactaccggtctttgtcatgtttcgaaagaagattacaggtcctctagaagagaggacgcggggtggcgagaaagctggggaagaaaaaggctagtacatatgattggtctgtgaacctgtgaggtgggtaggtaggtaggtggagatttttgttaactggtgttgttgacggactcgaacggggccgggcgtgtggtgtggctagctgtggtggtttgctcgccagccagccagccacacatcagcgagcatgcagagcttaagcatgtatgtacggatcggcttgcttagcggttaagggtgtgtttggtttggcttttggctttggcttttgccccctaaaagccaaaagccaaccaagggtgtatttggtttgacttttggcttttggcttttgtcccctaaaagccaaaagccaaacaaagggttagatctaggaagcagctttttctaaaagctggctttctcacagtgcaaatctgaaagcacccctgaacctgcttttagtggcttttcgaatggaactgtgaaaacatatatcgaagaacttttaacgacttttagtggtttccaccaaacagtttagctttttaacggcttacagcctacaacagctttttccacagctcacagcccacagcaacttttttcacagccacagcccaaccaaacagaccccaaagggctgaatccaggaagcagctttttctaaaagccgactttctcgtagtgtaaaactgaaaacacccctggacctgcttttagtggcttttggatggaactgtgaaaacatatatcgaagaacttttaacgacttttagtggtttccaccaaacgatttctagctttttaacagcacacagcctacaacagcttttttcacagctcacagcccacaacaactttttctacagccacaacccaaccaaacggaccctaaggcggccgagcgagcgcaaagcgtcgtcagctttgattgccatgccatctcctgctccacttgtctctctggccgtcgtcagccaccatccaacaaggccggtgctactggcggctcctaggtagacgacgacgacgacgacacctccaccgttcgccgccgtccactcaccaatcaacacggaacgcccaaaacacacacacacgcacgctggggagggaaaaaaaggcagagacatatgcgtgcgtcgctgcattattgtacgcgatcgaatggcatctctctcactctctctcctccctttattaatctggtactggctagctggtccggcgacaccgacgtgtcagctccgtcgcgcgcgtccgtccgtccgctggagcggacacggaccgcggcgtctgtcgatcgccggctcgccgcagcgcagctacctagcacgctcacgcatgctacactgcctacacgcacacggccggcccaaaagcgttccctgccgcctgccggccggcttttttattattattggaacatgaggctatttctcctcccacacgggctacgacgtgagcacgagtactgggatccccggatccgccctctctgtccctgctgctactccagccactgaaatgttgtcagatgaaacagcagagccgatctccgcacggaaacccatgcacggccattcaaattcaggtgcccacgtacgtcagggtgctgctgctactactatcaagccaataaaaggatggtaatgagtatgatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatttggcgcgccaaagcttggtcgagtggaagctagctttccgatcctacctgtcacttcatcaaaaggacagtagaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatcctctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggacaacaacccaaacatcaacgagtgcatcccgtacaactgcctcagcaaccctgaggtcgaggtgctcggcggtgagcgcatcgagaccggttacacccccatcgacatctccctctccctcacgcagttcctgctcagcgagttcgtgccaggcgctggcttcgtcctgggcctcgtggacatcatctggggcatctttggcccctcccagtgggacgccttcctggtgcaaatcgagcagctcatcaaccagaggatcgaggagttcgccaggaaccaggccatcagccgcctggagggcctcagcaacctctaccaaatctacgctgagagcttccgcgagtgggaggccgaccccactaacccagctctccgcgaggagatgcgcatccagttcaacgacatgaacagcgccctgaccaccgccatcccactcttcgccgtccagaactaccaagtcccgctcctgtccgtgtacgtccaggccgccaacctgcacctcagcgtgctgagggacgtcagcgtgtttggccagaggtggggcttcgacgccgccaccatcaacagccgctacaacgacctcaccaggctgatcggcaactacaccgaccacgctgtccgctggtacaacactggcctggagcgcgtctggggccctgattctagagactggattcgctacaaccagttcaggcgcgagctgaccctcaccgtcctggacattgtgtccctcttcccgaactacgactcccgcacctacccgatccgcaccgtgtcccaactgacccgcgaaatctacaccaaccccgtcctggagaacttcgacggtagcttcaggggcagcgcccagggcatcgagggctccatcaggagcccacacctgatggacatcctcaacagcatcactatctacaccgatgcccaccgcggcgagtactactggtccggccaccagatcatggcctccccggtcggcttcagcggccccgagtttacctttcctctctacggcacgatgggcaacgccgctccacaacaacgcatcgtcgctcagctgggccagggcgtctaccgcaccctgagctccaccctgtaccgcaggcccttcaacatcggtatcaacaaccagcagctgtccgtcctggatggcactgagttcgcctacggcacctcctccaacctgccctccgctgtctaccgcaagagcggcacggtggattccctggacgagatcccaccacagaacaacaatgtgccccccaggcagggtttttcccacaggctcagccacgtgtccatgttccgctccggcttcagcaactcgtccgtgagcatcatcagagctcctatgttctcttggatacaccgtagtgctgagttcaacaacatcattgcatccgacagcattactcaaatacccttggtgaaagcacatacacttcagtcaggtactactgttgtcagaggtccagggtttacaggaggagacattcttcgtcgcacaagtggaggaccctttgcttacactattgttaacatcaatggccaattgccccaaaggtatcgtgcaagaatccgctatgcctctactacaaatctcaggatctacgtgactgttgcaggtgaaaggatctttgctggtcagttcaacaagactatggataccggtgaccctttgacattccaatcttttagctacgcaactatcaacacagcttttacattcccaatgagccagagtagcttcacagtaggtgctgacactttcagctcagggaatgaagtttacatcgacaggtttgaattgattccagttactgcaaccctcgaggctgagtacaaccttgagagagcccagaaggctgtgaacgccctctttacctccaccaatcagcttggcttgaaaactaacgttactgactatcacattgaccaagtgtccaacttggtcacctaccttagcgatgagttctgcctcgacgagaagcgtgaactctccgagaaagttaaacacgccaagcgtctcagcgacgagaggaatctcttgcaagactccaacttcaaagacatcaacaggcagccagaacgtggttggggtggaagcaccgggatcaccatccaaggaggcgacgatgtgttcaaggagaactacgtcaccctctccggaactttcgacgagtgctaccctacctacttgtaccagaagatcgatgagtccaaactcaaagccttcaccaggtatcaacttagaggctacatcgaagacagccaagaccttgaaatctactcgatcaggtacaatgccaagcacgagaccgtgaatgtcccaggtactggttccctctggccactttctgcccaatctcccattgggaagtgtggagagcctaacagatgcgctccacaccttgagtggaatcctgacttggactgctcctgcagggatggcgagaagtgtgcccaccattctcatcacttctccttggacatcgatgtgggatgtactgacctgaatgaggacctcggagtctgggtcatcttcaagatcaagacccaagacggacacgcaagacttggcaaccttgagtttctcgaagagaaaccattggtcggtgaagctctcgctcgtgtgaagagagcagagaagaagtggagggacaaacgtgagaaactcgaatgggaaactaacatcgtttacaaggaggccaaagagtccgtggatgctttgttcgtgaactcccaatatgatcagttgcaagccgacaccaacatcgccatgatccacgccgcagacaaacgtgtgcacagcattcgtgaggcttacttgcctgagttgtccgtgatccctggtgtgaacgctgccatcttcgaggaacttgagggacgtatctttaccgcattctccttgtacgatgccagaaacgtcatcaagaacggtgacttcaacaatggcctcagctgctggaatgtgaaaggtcatgtggacgtggaggaacagaacaatcagcgttccgtcctggttgtgcctgagtgggaagctgaagtgtcccaagaggttagagtctgtccaggtagaggctacattctccgtgtgaccgcttacaaggagggatacggtgagggttgcgtgaccatccacgagatcgagaacaacaccgacgagcttaagttctccaactgcgtcgaggaagaaatctatcccaacaacaccgttacttgcaacgactacactgtgaatcaggaagagtacggaggtgcctacactagccgtaacagaggttacaacgaagctccttccgttcctgctgactatgcctccgtgtacgaggagaaatcctacacagatggcagacgtgagaacccttgcgagttcaacagaggttacagggactacacaccacttccagttggctatgttaccaaggagcttgagtactttcctgagaccgacaaagtgtggatcgagatcggtgaaaccgagggaaccttcatcgtggacagcgtggagcttctcttgatggaggaataatgagatctatcgattctagaaggcctgaattctgcatgcgtttggacgtatgctcattcaggttggagccaatttggttgatgtgtgtgcgagttcttgcgagtctgatgagacatctctgtattgtgtttctttccccagtgttttctgtacttgtgtaatcggctaatcgccaacagattcggcgatgaataaatgagaaataaattgttctgattttgagtgcaaaaaaaaaggaattagatctgtgtgtgttttttggatccccggggcggccgcgttaacaagcttgagctcaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaaccaatccttctaggatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcctctagaggatcagcatggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccggtgcatgcaggtaacaaatgcatcctagctagtagttctttgcattgcagcagctgcagctagcgagttagtaataggaagggaactgatgatccatgcatggactgatgtgtgttgcccatcccatcccatttcccaaccccaaacgaaccaaaacacacgtactacgtgcaggtgtggccggcctacggcaacaagaagttcgagacgctgtcgtacctgccgccgctgtcgaccggcgggcgcatccgctgcatgcaggccatggacaactccgtcctgaactctggtcgcaccaccatctgcgacgcctacaacgtcgcggcgcatgatccattcagcttccagcacaagagcctcgacactgttcagaaggagtggacggagtggaagaagaacaaccacagcctgtacctggaccccatcgtcggcacggtggccagcttccttctcaagaaggtcggctctctcgtcgggaagcgcatcctctcggaactccgcaacctgatctttccatctggctccaccaacctcatgcaagacatcctcagggagaccgagaagtttctcaaccagcgcctcaacactgatacccttgctcgcgtcaacgctgagctgacgggtctgcaagcaaacgtggaggagttcaaccgccaagtggacaacttcctcaaccccaaccgcaatgcggtgcctctgtccatcacttcttccgtgaacaccatgcaacaactgttcctcaaccgcttgcctcagttccagatgcaaggctaccagctgctcctgctgccactctttgctcaggctgccaacctgcacctctccttcattcgtgacgtgatcctcaacgctgacgagtggggcatctctgcagccacgctgaggacctaccgcgactacctgaagaactacaccagggactactccaactattgcatcaacacctaccagtcggccttcaagggcctcaatacgaggcttcacgacatgctggagttcaggacctacatgttcctgaacgtgttcgagtacgtcagcatctggtcgctcttcaagtaccagagcctgctggtgtccagcggcgccaacctctacgccagcggctctggtccccaacaaactcagagcttcaccagccaggactggccattcctgtattcgttgttccaagtcaactccaactacgtcctcaacggcttctctggtgctcgcctctccaacaccttccccaacattgttggcctccccggctccaccacaactcatgctctgcttgctgccagagtgaactactccggcggcatctcgagcggcgacattggtgcatcgccgttcaaccagaacttcaactgctccaccttcctgccgccgctgctcaccccgttcgtgaggtcctggctcgacagcggctccgaccgcgagggcgtggccaccgtcaccaactggcaaaccgagtccttcgagaccacccttggcctccggagcggcgccttcacggcgcgtggaaattctaactacttccccgactacttcatcaggaacatctctggtgttcctctcgtcgtccgcaacgaggacctccgccgtccactgcactacaacgagatcaggaacatcgcctctccgtccgggacgcccggaggtgcaagggcgtacatggtgagcgtccataacaggaagaacaacatccacgctgtgcatgagaacggctccatgatccacctggcgcccaatgattacaccggcttcaccatctctccaatccacgccacccaagtgaacaaccagacacgcaccttcatctccgagaagttcggcaaccagggcgactccctgaggttcgagcagaacaacaccaccgccaggtacaccctgcgcggcaacggcaacagctacaacctgtacctgcgcgtcagctccattggcaactccaccatcagggtcaccatcaacgggagggtgtacacagccaccaatgtgaacacgacgaccaacaatgatggcgtcaacgacaacggcgcccgcttcagcgacatcaacattggcaacgtggtggccagcagcaactccgacgtcccgctggacatcaacgtgaccctgaactctggcacccagttcgacctcatgaacatcatgctggtgccaactaacatctcgccgctgtactgataggagctctgatccccatgggaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatatccccggggcggccgcggggaattcggtaccaagctttggcgcgccaaatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgcatccccggaaattatgtttttttaaaaaccacggtattatagataccgtgttattttttgagtattggaaatttcatttcaacccaaagtttcttcatggcacatctagcttttgcctaataccatgtagggctacatcttaaaaatctatactactatattaaagctgcaggggtagcctgtctccacctggttctgcctcgagccaatctaaaccgtccatctatatccatcaaatcagcaccgtccggtccgtgcgcacctcctctcccgctattcagttgcatacttgcagcaggttctccctcctcaccatttcctctgcctcctctctcgctcactggtcagattcatcctgcctctcccgcatgcgctccctccccatgccccgtctcgcactatcgccacacctcaccgcggggagacgaagacggtggacgcatcctcacctcctccgctagttgtcgctcttccatcctcttcaacaacttctacatagggagaggcggttcggcgtcccgacgccgccgcttctcccctccccatggaggacgagaacatcgacctcggcggcgggggcgatgcctccgctctgcatagaggagggttgtagtggcaagcagcaatgccaacaccgaggcgggccaagactaggcaacaataggacggcacgcccggttgtcagcgaggtggcggcatcgtgtgccgctaccgaacaacatctccggcgctggagtcggtgagttactgcgccacccggacgccctcaatgcactgatatctacccggtctccatcgccgcccttcctcccttccctctccctgtgcctccctctcttgccctctcccttccaactgctcccgccccagccctagcccaaccacctcccgcgcagggtcaccaa

SEQ ID NO: 5 - DNA, 10579 bp, Artificial
  synthetic
tcctgtgttgttggaacagacttctgtctcttctggtgatcataaatatttaaatgaaccagttgtgttggaaaatgttgttttcttttgtctctagactggaaagcggagttctcgtcaacacggttctttcaactagggatgaaagtggtaatccgaattgttagtacaaatttaatattttaaaatagatatgtataaaattttatgttgatcttttttatgttatcaagcacattagtataaattagtataaatatgaataaaatattacataaaatgttttatgtattatttggtccctacaacataaatagttgaaaaaattactaaatttgttttcgaatctatatcgaagtttatatctattatttaagaaaaatataggatgaaaaggtttatcttttatgaatctttacaagctggatcttataaacaagaaaataaatttatattgtagattttatatcctatttattcgcaatcaaagaaaagcgactaaaaaactgattaccgagtaaatactgtttccaaccgttttcgtccctactatcaacgccttctcccaaccgcagtcgatctgtccgtctgtatcaggcgcagcggcacccctgctgttcgactatctagaccatagaatattttaggtatacaataattttagttccacgctagaacattttagttagaataataacaagatttgctattgatgtaggactcgcccgtcactgtctaaaaaagcattctgtcggtcttattctttaggcatcagcgggtgtactatctcatttttcctatcatattcctcagtactctgttaagtataaatggtctattttacatgatgaactaataaaactaattaaggatcctaactttttgtgaaggtaatttggatcattatgcattaccatcctacgtatacctgctgcagcagcatctgcgtaagcacagcctagatatatgcttctgtgtggactgaaaggagactttgtttatcaattagtatactcccaaaaaactgatgacaccaatgatgcaaataggctgggaatagtctgtctaatagtttgagtgaatcatgtcactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtcgaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggccgcggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggagctctagatctgttctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccaggggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccccgggtctagacaattcagtacattaaaaacgtccgccatggtctgaaggcaacagataaggcatactgggccttgtggtagttgttttactgggcctttttgtatgatctataaaattcactgggatcaacccggagaggaatggcagcagatgcagtccccagggtcctccgtcgccgcctgagcacccggcacccgcgctgaaccggagagggacgcgcggacgccgtgcagctggtgcggagggggctgtggcagatgaggatgagacgcgtacgtggctgggaaggccagcaggccaccgggtcttcgtccagcccggcgcgagtggacaggactagagatggcaacggttacaaacccgctgggttttaccgtcccaaacccgtacccgtgaaaaatatctatgcccattaaaaaacccgtacccatgacgggtttgagattttgcccaaacccgtacccatcgggttaacgggtacccatgggttacccgcgggtttcatctccaatatacctgttcttctcataatcaataagtatcgtaatgattaatgatatcatgatccaaaatctatgtaatgaacaacgagttcatgatttggtataaaaattattagtagagagaatgaaatacaaataataagttgtataattaagtgaccttgcactaagttatccatccatcacatatataacgctagtaaaaactataatatcaagcaagcaacactctcaccgactactgatacattcaccaattgataaaaaatatgaagtaaataaggaataacaagtttgttgttcgtttataaaataaaatgacaatatgcactaggtttggtcgggtttaaaaaacccacgggttcacgggtttgggtactataggaacaaacccgtacccataaacccattgggtacagatttatgcccgttaacaaacccatgggtatgaaaattgacccaaacctataccctaatggggtaaaaacccatcgggtttcggatttcgggtacccattgccatctctagacaggacaacctcggccggtcctgtatgtaggccaccagcatcggccagttggtacatccagccggggtcaggtcacttttactcgtctcaatcagacaatcaccgtccaccaacgaacgccaacgttgtcacttgtcaggtcggttgagacttgtatttttttttgtcctccgtaaaaatcggttcaccag

SEQ ID NO: 6 - DNA, 10547 bp, Artificial
  synthetic
  misc_feature (3541)..(3549): n is a, c, g, or t
  misc_feature (5347)..(5396): n is a, c, g, or t
  misc_feature (5650)..(5664): n is a, c, g, or t
  misc_feature (7658)..(7663): n is a, c, g, or t
  misc_feature (8840)..(8889): n is a, c, g, or t
  misc_feature (9143)..(9160): n is a, c, g, or t
  misc_feature (10382)..(10382): n is a, c, g, or t
  misc_feature (10470)..(10470): n is a, c, g, or t
ccccgcgtttcgttgcccctggccggtacccatttggcgccgattcttttcttgccccccggccggccgctcgctcgcctttggattcttccaaagccgctgatgggatggtggcgaacacacccaccacccgtctttgcccaaagcgacccggcacaggccgcgccggcttcactaaccactagcgcttgtactaataaaatggtttctagcgtttgttgctctcctttttcttttttcgccggttcttcggagccgtgtggacactggacagcgtccagtccagcaggcatagggtggtctcggcggcggtcgtccgacgacgatcgatctccatgagattccgcgacaggccaggacggaaagctgggcccttctcaccaattcgcgtcggagccggaacaagattccctcccccaatcatttcgacgcgccctttcttcgccacccctcgtggccgtgtttcgcggccggcccttatctccttcccgtgacgcgttcttttgtagcttagcggccggcacgttgctaaccaggctagcttcgttcgtttttaatctgcctatcgagaagagaagaaaaattcgtccatggggccacggcctcttctgcaggcatttggcatgtgaaggaacccgaaccagtgaatggagatggacggatgctgctcagatacgcagtcaaacctgccggcgaaattacggggggagctggctggctggctggctggacgccagatcacacatggatgacgcggcacggcagctagccgagcaggcgctctgcgcacgcaattcaacagaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtacaagcttgcacatgacaacaattgtaagaggatggagaccacaacgatccaacaatacttctgcgacgggctgtgaagtatagagaagttaaacgcccaaaagccattgtgtttggaatttttagttattctatttttcatgatgtatcttcctctaacatgccttaatttgcaaatttggtataactactgattgaaaatatatgtatgtaaaaaaatactaagcatatttttgaagctaaacatgatgttatttaagaaaatatgttgttaacagaataagattaatatcgaaatggaaacatctgtaaattagaatcatcttacaagctaagagatgttcacgctttgagaaacttcttcagatcatgaccgtagaagtagctctccaagactcaacgaaggctgctgcaattccacaaatgcatgacatgcatccttgtaaccgtcgtcgccgctataaacacggataactcaattccctgctccatcaatttagaaatgagcaagcaagcacccgatcgctcaccccatatgcaccaatctgactcccaagctctgtttcgcattagtaccgccagcactccacctatagctaccaattgagacctttccagcctaagcagatcgattgatcgttagagtcaaagagttggtggtacgggtactttaactaccatggaatgatggggcgtgatgtagagcggaaagcgcctccctacgcggaacaacaccctcgccatgccgctcgactacagcctcctcctcgtcggcgccacaacgagggagcccgtggtcgcagccaccgaccagcatgtctctgtgtcctcgtccgacctcgacatgtcatggcaaacagtcggacgccagcaccagactgacgacatgagtctctgaagagcccgccacctagaaagatccgagccctgctgctggtagtggtaaccattttcgtcgcgctgacgcggagagcgagaggccagaaatttatagcgactgacgctgtggcaggcacgctatcggaggttacgacgtggcgggtcactcgacgcggagttcacaggtcctatccttgcatcgctcggcgcggagtttacggggacttatccttacgacgtgctctaaggttgcgataacgggcggaggaaggcgtgtggcgtgcggagacggtttatacacgtagtgtgcgggagtgtgtttcgtagacgcgggaaagcacgacgacttacgaaggttagtggaggaggaggacacactaaaatcaggacgcaagaaactcttctattatagtagtagagaagagattataggagtgtgggttgattctaaagaaaatcgacgcaggacaaccgtcaaaacgggtgctttaatatagtagatatatatatatagagagagagagaaagtacaaaggatgcatttgtgtctgcatatgatcggagtattactaacggccgtcgtaagaaggtccatcatgcgtggagcgagcccatttggttggttgtcaggccgcagttaaggcctccatatatgattgtcgtcgggcccataacagcatctcctccaccagtttattgtaagaataaattaagtagagatatttgtcgtcgggcagaagaaacttggacaagaagaagaagcaagctaggccaatttcttgccggcaagaggaagatagtggcctctagtttatatatcggcgtgatgatgatgctcctagctagaaatgagagaagaaaaacggacgcgtgtttggtgtgtgtcaatggcgtccatccttccatcagatcagaacgatgaaaaagtcaagcacggcatgcatagtatatgtatagcttgttttagtgtggctttgctgagacgaatgaaagcaacggcgggcatatttttcagtggctgtagctttcaggctgaaagagacgtggcatgcaataattcagggaattcgtcagccaattgaggtagctagtcaacttgtacattggtgcgagcaattttccgcactcaggagggctagtttgagagtccaaaaactataggagattaaagaggctaaaatcctctccttatttaattttaaataagtagtgtatttgtattttaactcctccaacccttccgattttatggctctcaaactagcattcagtctaatgcatgcatgcttggctagaggtcgtatggggttgttaatagcatagctagctacaagttaaccgggtcttttatatttaataaggacaggcaaagtattacttacaaataaagaataaagctaggacgaactgctggattattactaaatcgaaatggacgtaatattccaggcaagaataattgttcgatcaggagacaagtggggcattggaccggttcttgcaagcaagagcctatggcgtggtgacacggcgcgttgcccatacatcatgcctccatcgatgatccatcctcacttgctataaaaagaggtgtccatggtgctcaagctcagccaagcaaataagacgacttgtttcattgattcttcaagagatcgagcttcttttgcaccacaaggtcgagnnnnnnnnnatgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagnnnnnnnnnnnnnnnnnnnnnnnnnnnnnnnnnnnnnnnnnnnnnnnnnngatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcnnnnnnnnnnnnnnnctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagnnnnnnatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaannnnnnnnnnnnnnnnnnnnnnnnnnnnnnnnnnnnnnnnnnnnnnnnnngatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcnnnnnnnnnnnnnnnnnngatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagagttggtggtacgggtactttaactaacgaggtgtgtcgcgcagcgctcctgcacggatgtagctttggattgctggataatgtctcgcgcaagcgtcgtatttatttatttatttattacagcctccaccgccgtgcgtgctccgtttcggattataataaaactaatattaaataaaaaaatcggattaaaggatgtttccgaaataaagatctccaccacaggagcgaaagaaaaaaaaagagaaacgggctatggagaaatggtgttgcgagtatacggcggctccgtcgtcgtcggatcgacatgtacaaagtaggtgcacaaaaggcaaagcaaaatcacctcatcaaagaccaaaagcggagcaaagaatcgatactaaatccacatgttttttttgttcctgtctactacgtgctgtgcctgtgcgtgaagcacgattagtacgtgtactcactcttgtcatattctttttagtgtcttgtcactagtcacatggagtagcaaccatggctggcgatacccgcgataaataaaaaaaagagagagggagtaatatattagatactcacccattataaattataaaatattttagagtttgaataggtagttcttgtatatttatttatagaccttcaagtttgtccgcctctcgagagccgaactttgttgcccatgcttccccggctcaggtcatgccacctccttcaccaagggcacacggaagatctggtgaagcttgtcatcaccccgcgcccttcaaacatgtgaggatgcgtcgtcgctggcactagtagcactcattgtaggcactactttgacagtttcctccaaatatgtagtgaggaaacacttgaacaacacgtttgggattacttatgatgtttggttngtccatcaatgataattccttcttcttgcttaatgattggctctagaaccgatacttggcacatttcatcaggaagggcgcatgcacnaatttaacctgttatcgatgttccggttcctaagttgaagaaaacaatggctaacaattagcccatgtgagcataac

SEQ ID NO: 7 - DNA, 7584 bp, Artificial
  synthetic
agagcctcacgtttccagggtgaagtatcagaggatttaccgcccatgccttttatggagacaagaaggggaggaggtaaacagatcagcatcagcgctcgaaagtttcgtcaaaggatgcggaactgtttccagccgccgtcgccattcggccagactcctcctctctcggcatgagccgatcttttctctggcatttccaaccctagagacgtgcgtccctggtgggctgctcggccagcaagccttgtagcggcccacgcgtggtaccaagcttgatatccctagggcggccgcgttaacaagcttactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctcccccttcgccgccgccgcgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggcgatgggcgcccgcatccgtaaggaaggcgacacctggatcatcgatggcgtcggcaatggcggcctcctggcgcctgaggcgccgctcgatttcggcaatgccgccacgggctgccgcctgacgatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctgatgagctcgaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatagcttctgcaggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctaggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcggcctctagaggatccaggagcaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggcgatgggcgcccgcatccgtaaggaaggcgacacctggatcatcgatggcgtcggcaatggcggcctcctggcgcctgaggcgccgctcgatttcggcaatgccgccacgggctgccgcctgacgatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtctccccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccctaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctgatgagctcgaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatatccccggggaattcggtaccaagcttttataatagtagaaaagagtaaatttcactttgggccaccttttattaccgatattttactttataccaccttttaactgatgttttcacttttgaccaggtaatcttacctttgttttattttggactatcccgactctcttctcaagcatatgaatgacctcgagtaagcttgttaacgcggccgccctagggatatcaagcttggtaccacgcgacacacttccactctagtgtttgagtggatcctgttatctcttctcgaaccataacagactagtattatttgatcattgaatcgtttatttctcttgaaagcggtttcatttttttttacagacgtctttttttaggaggtcgacatccattatgcggcataggtgttacatcgcgtatacaacttaaccgtacaccacttttagcaatggctcgtaatgcggcatctcttccgctaccagcaccttttaccataacttctgctcgttgcaaacccactgtacgaatagcatctactgctgttctgctgactttattttttttaataaagtgaaaaaccataaaatggacaacaacaccctgcccttcactaccggtcggagcgacgccgaagatggggtt

SEQ ID NO: 8 - DNA, 9067 bp, Artificial
  synthetic
agccaatcacggcaaaatacgcaagcataacgcgcggctcacgtctccccacccgatccccggccccacgctaccatttatgaagctctttcattcatgttacgtatgcataaacgccggtacctgcgcgcggtatacccaccacctgccacgtcgacccgcggcccgcacacctagccacgccgggggccggggctctctctagtaatggaagccctgggcttccattacctcgtccctgcgtgcagctctcttctgtactaagatttcatccctgatcctgcgcgggtcatcatcaatacgagacggccatctcatgcatccgtcctcctctccgatccctgatctgatggatcattccaacaatatttaatatactactctactagcgtcgtgtgtacatgtgccttgtagtactgtactgtacagctagctagctagctccgagcagcaaagcaggctacaggctaggcagggcttgaagtcgcattgcattgcatcgcatgcgcatcgcagcatgcatcaattcctgcccagagagcgaagtggattaattaattagcaccaaggactgtggtacgtactgctgctaaaaactaaaaaggtttgaaatgctgagagagagagaaagagagagagtacatgcatgcaatgcaatgcaccatgcatctttcaattccatggcgccgtagcatctctctctctctatagccttctctttccatcaacctgagcaaaagaaagctggtttcctgcaggctgcgtgcgcccggcggccgtctctgctgcgtgcatgcatcagcatgcgcgcagtaactccacacagccagcctctcctacaaaagacatgtactacagtaccgctaccgagctagtactaggtacacacagtgtgtgtgtgtggctggctactagtgtttcttcttgaacgatgaatatacagcacaatcctcctgctctcttgctcttgctcttgcgctgcatgctcttccttccccgcactccctaatagctaggcatgcattgtccattgaattgaagctctctctctctctctctctctctctctctctctctctctgccaagccatgcccatgcaagtcgtcgtctctgttccagctgaattcatgcccatgcaaatgcaagtcgtacgtctctctttccagaattcagcagcagtactctccacgcactgatcgccgtcagagagctaggtcagtggctgcagtgagtgggcatgatgagtgcgtgatgagggctcttttggagctactactactagcgtgtgtattcattcactctcactggccgataaactgaccatctatttatctccaatcgatcgaattcatcagaccagattctcttttatggccggccggccggccctgctgactgctgacgcggccaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtacaagcttcgatcatccaggtgcaaccgtataagtcctaaagtggtgaggaacacgaaacaaccatgcattggcatgtaaagctccaagaatttgttgtatccttaacaactcacagaacatcaaccaaaattgcacgtcaagggtattgggtaagaaacaatcaaacaaatcctctctgtgtgcaaagaaacacggtgagtcatgccgagatcatactcatctgatatacatgcttacagctcacaagacattacaaacaactcatattgcattacaaagatcgtttcatgaaaaataaaataggccggacaggacaaaaatccttgacgtgtaaagtaaatttacaacaaaaaaaaagccatatgtcaagctaaatctaattcgttttacgtagatcaacaacctgtagaaggcaacaaaactgagccacgcagaagtacagaatgattccagatgaaccatcgacgtgctacgtaaagagagtgacgagtcatatacatttggcaagaaaccatgaagctgcctacagccgtctcggtggcataagaacacaagaaattgtgttaattaatcaaagctataaataacgctcgcatgcctgtgcacttctccatcaccaccactgggtcttcagaccattagctttatctactccagagcgcagaagaacccgatcgacaggatccaccatgagggtgttgctcgttgccctcgctctcctggctctcgctgcgagcgccaccagcgctaagtacctggagctggaggagggcggcgtgatcatgcaggcgttctactgggacgtcccgagcggaggcatctggtgggacaccatccgccagaagatccccgagtggtacgacgccggcatctccgcgatctggataccgccagcttccaagggcatgtccgggggctactcgatgggctacgacccgtacgactacttcgacctcggcgagtactaccagaagggcacggtggagacgcgcttcgggtccaagcaggagctcatcaacatgatcaacacggcgcacgcctacggcatcaaggtcatcgcggacatcgtgatcaaccacagggccggcggcgacctggagtggaacccgttcgtcggcgactacacctggacggacttctccaaggtcgcctccggcaagtacaccgccaactacctcgacttccaccccaacgagctgcacgcgggcgactccggcacgttcggcggctacccggacatctgccacgacaagtcctgggaccagtactggctctgggcctcgcaggagtcctacgcggcctacctgcgctccatcggcatcgacgcgtggcgcttcgactacgtcaagggctacggggcctgggtggtcaaggactggctcaactggtggggcggctgggcggtgggcgagtactgggacaccaacgtcgacgcgctgctcaactgggcctactcctccggcgccaaggtgttcgacttccccctgtactacaagatggacgcggccttcgacaacaagaacatcccggcgctcgtcgaggccctgaagaacggcggcacggtggtctcccgcgacccgttcaaggccgtgaccttcgtcgccaaccacgacacggacatcatctggaacaagtacccggcgtacgccttcatcctcacctacgagggccagcccacgatcttctaccgcgactacgaggagtggctgaacaaggacaagctcaagaacctgatctggattcacgacaacctcgcgggcggctccactagtatcgtgtactacgactccgacgagatgatcttcgtccgcaacggctacggctccaagcccggcctgatcacgtacatcaacctgggctcctccaaggtgggccgctgggtgtacgtcccgaagttcgccggcgcgtgcatccacgagtacaccggcaacctcggcggctgggtggacaagtacgtgtactcctccggctgggtctacctggaggccccggcctacgaccccgccaacggccagtacggctactccgtgtggtcctactgcggcgtcggctccgagaaggacgagctgtgataggtaacgaaactagagctctagatctgttctgcacaaagtggagtagtcagtcatcgatcaggaaccagacaccagacttttattcatacagtgaagtgaagtgaagtgcagtgcagtgagttgctggtttttgtacaacttagtatgtatttgtatttgtaaaatacttctatcaataaaatttctaattcctaaaaccaaaatccaggggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatttcaagtcatctgcatgtgaaataaacatcttgtccctcctcgatgatccacctctctctctctctctctctctctctctctctctctctctctctctcatgctcaattgctcatgcatatgcacgcgcgcgtttgctcgcccattgtttgtttgcttttgcttttatgttcgatcgatggatgtatgtgtatataggcgagaaggggcaccagctgctgcatgcctgtttcttacagtcttgtattgtatgcatgcaaaagagagaagctagactagaccagacaacactagcgattctgacaggctgacttttgagcagttgactactcgctctacatacagccttgtattattgtatagttttctttaatgcctgacgttcacgtgttcctatgcattgcagtgtatagcgacctgcctatcgtggacctttgttttttttcaattctatttgtttaatcagatactgtacagtaaagatgcagactcacaaataaacacgcatgaaaaaaaaggtgctatccgtctgaacttctgatctacactagtacacagaagctctatagtggcagttgaaaacgtatttacactggcgtttttcagttccgccagtgctaggggccagtggaaatcagcatttccactggcggttattttggaaccgccagtggaaagtgcattttcactggcggttttcttaaggaaaccgccagtgaaaagtgcattttcaaccgccagtgaaaatgcactttacactggcggtttcctttataaaaccgccagtggaaatgcacttttcactggcggttttctttatttagccgccagtggaagttttcccgccttttttcaaaatttcaaacaatactgaattatagatatatttatttacacacacacaaacatatatatatatagatctatattgatattgaaagcaacatggaattaaattctatcatacatttatatacatcaaagtattctgtttacaaccatatatgcttcatgcattctatacatcaaaagttttcacctaagttctaataactatctcggctaagagataatctactaatttttgttagtattctaaactctggcaaagctaatgttccggaagcatcgtgatattttccttctccgggaatgacctctttcaatatgaatgtgcagaggtcctcgactatgccatacaatgcagcttcggtcaagttctccgggtttcctcgttgaaattgctgtaaaggaattttataaacatcatctatttatactcaataataacacatttgcatctttaatgacataaatacatacttgactattactaataataccttgtcagggttcgtgatgtatcgtccgttcactctcatgaactcgcacgcatagaatccacataggaccgatccttgtggttgcttgtggcactacataacgggagattggttatttagttgcaacattgtgtatgatatgtattcataaaatcacatacttaccggccagtgatgatggatgtctagtggcacgcgttgtttcgacaggtcgta

SEQ ID NO: 9 - DNA, 11361 bp, Artificial
  synthetic
actagtttcctagcccgcgtcgtgcccctaccccaccgacgtttatggaaggtgccattccacggttcttcgtggccgcccctaaggatgtaaatggtcggtaaaatccggtaaatttccggtaccgtttaccagatttttccagccgttttcggatttatcgggatatacagaaaacgagacggaaacggaataggttttttttcgaaaacggtacggtaaacggtgagacaaacttaccgtccgttttcgtatttctcgggaaactctggtatattcccgtatttgtcccgtattttcccgacccacggacctgccaatcaaccatcagccagtcagcccatccccacagctatggcccatggggccatgttggccacatgcccacgcaacgcaaggcagtaaggctggcagcctggcacgcattgacgcatgtggacacacacagccgccgcctgttcgtgtttctgtgccgttgtgcgagactgtgactgcgagtggcggagtcggcgaacggcgaggcgtctccggagtctggactgcggctgtggacagcgacgctgtgacggcgactcggcgaagccccaagctaccaagcccccaagtccccatccatctctgcttctctggtcatctccttcccctggtcgatctgcaggcgccagaccggccgaagcatcacgaaacgcactaagacctcgaaggagtcaaaccactcctccgaggcctcgggggctacacccggcgggtgcgctcgcgcgcacccaccggaacaaaatgtaaccgagaaaggtcggtccccttgcaaaaaaagtgcgacaaaagcctccaagcgagtattaacactcactttgaggctcgggggctactgtcggggaccataattaggggtacccccaagactcctaatctcagctggtaacccccatcagcacaaagctgcaaaggcctgatgggtgcgattaagtcaaggctcggtccactcaagggacacgatctcgcctcgcccgagcccagcctcgggcaagggcggccgaccccgaggattcacgtctcgcccgagggccccctcaagcgacgggcacaccttcggctcgcccgaggcccattcttcgccgagaagcaaccttggccagatcgccacaccgaccgaccgtatcgcaggagcatttaatgcgaggatcgcctgacaccttatcctgacgcgcgctcttcagtcgacagagccgaagtgaccgcaatcacttcgccgctccactgaccgacctgacaagaagacagcgccgcctgcgtcgctccgactgctgtgccactcgacagagtgaggctgacagcagccaagtccggcctcgggcgccataggaagctccgcctcgcccgaccctagggctcggactcggcctcggctccggaagacgacgaactacgcttcgcccgaccccagggcttggactcagcctcggctccggaagacgacgaattccgcctcgcccgaccccagggctcggactcggcctcggctccagaagacgacgaactccgcctcgcccgaccccagggctcggactcagcctcggctccggaagacgacgaactccgcctcgcccgaccccagggctcggactcagcctcggcctcagacgatggtctccgcctcgcccgacccggggctcggactcgacctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcattcttcaagaactgcatatcttgccgagatcctcatccctaaaggtacttgacaatagtattattggagtcgatacacaactcacaaaaaatacaagaagtcgactaggtggattggtccgagtgaagagaaaaaaaagccatacagaactcaaaatcttttccggagatattcattttcctgaagaggcggataagatattaggtggcagtttgataccaccagaaagagaaaaaaaagattctaaggaatcaaaaaaaaggaaaaattgggtttatgttcaacggaaaaaatttctcaaaagcaaggaaaagtattgtggctatttatctatccgtgcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaaccctcaacctcagcaaccaaccaatggtatctatcttgcaacctctctagatcatcaatccactcttgtggtgtttgtggctctgtcctaaagttcactgtagacgtctcaatgtaatggttaacgatatcacaaaccgagagaagagggatctcgaagcttcggccggggcccatcgatatccgcgggcatgcctgcagtgcagcgtgacccggtcgtgcccctctctagagataatgagcattgcatgtctaagttataaaaaattaccacaactggaagagcggttacccggaccgaagcttcggccggggcccatcgatatccgcgggcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatggagaacaacatacagaatcagtgcgtcccctacaactgcctcaacaatcctgaagtagagattctcaacgaagagaggtcgactggcagattgccgttagacatctccctgtcccttacacgtttcctgttgtctgagtttgttccaggtgtgggagttgcgtttggcctcttcgacctcatctggggcttcatcactccatctgattggagcctctttcttctccagattgaacagttgattgaacaaaggattgagaccttggaaaggaatcgggccatcactacccttcgtggcttagcagacagctatgagatctacattgaagcactaagagagtgggaagccaatcctaacaatgcccaactgagagaagatgtgcgtatacgctttgctaacacagatgatgctttgatcacagccatcaacaacttcacccttaccagcttcgagatccctcttctctcggtctatgttcaagctgctaacctgcacttgtcactactgcgcgacgctgtgtcgtttgggcaaggttggggactggacatagctactgtcaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacatagttgctctctttccgaactacgatgttcgtacctatccgattcaaacgtcatcccaacttacaagggagatctacaccagttcagtcattgaagactctccagtttctgcgaacatacccaatggtttcaacagggctgagtttggagtcagaccaccccatctcatggacttcatgaactctttgtttgtgactgcagagactgttagatcccaaactgtgtggggaggacacttagttagctcacgcaacacggctggcaatcgtatcaactttcctagttacggggtcttcaatcccgggggcgccatctggattgcagatgaagatccacgtcctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcagattcccttggtgaaggcacacacacttcagtcaggaactacagttgtaagagggccggggttcacgggaggagacattcttcgacgcactagtggaggaccattcgcgtacaccattgtcaacatcaatgggcaacttccccaaaggtatcgtgccaggatacgctatgcctctactaccaatctaagaatctacgttacggttgcaggtgaacggatctttgctggtcagttcaacaagacaatggataccggtgatccacttacattccaatctttctcctacgccactatcaacaccgcgttcacctttccaatgagccagagcagtttcacagtaggtgctgataccttcagttcaggcaacgaagtgtacattgacaggtttgagttgattccagttactgccacactcgagtaaggatccgtcgacctgcagccaagctttcgcgagctcgagatccccgacatatgccccggtttcgttgcgactaacatgagttcttggacaaatttgattggacctgatgagatgatccaacccgaggatatagcaaagctcgttcgtgcagcaatggaacggccaaaccgtgcttttgtccccaagaatgaggtgctatgcatgaaggaatctacccgttgatgtccaacagtctcagggttaatgtctatgtatcttaaataatgttgtcggtattttgtaatctcatatagattttcactgtgcgacgcaaaaatattaaataaatattattattatctacgttttgattgagatatcatcaatattataataaaaatatccattaaacacgatttgatacaaatgacagtcaataatctgatttgaatatttattaattgtaacgaattacataaagatcgaatagaaaatactgcactgcaaatgaaaattaacacatactaataaatgcgtcaaatatctttgccaagatcaagcggagtgagggcctcatatccggtctcagttacaagcacggtatccccgaagcgcgctccaccaatgccctcgacatagatgccgggctcgacgctgaggacattgcctaccttgagcatggtctcagcgccggctttaagctcaatcccatcccaatctgaatatcctatcccgcgcccagtccggtgtaagaacgggtctgtccatccacctctgttgggaattccggtccgggtcacctttgtccaccaagatggaactgcggccgcggaccgaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacagggtacccggggatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgacctgcaggcatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtggcgagctcgaattcgagctcgagcccgggtggatcctctagagtcgacctgcagaagcttcggtccggcgcgcctctagttgaagacacgttcatgtcttcatcgtaagaagacactcagtagtcttcggccagaatggcctaactcaaggccctcactccgcttgatcttggcaaagatatttgacgcatttattagtatgtgttaattttcatttgcagtgcagtattttctattcgatctttatgtaattcgttacaattaataaatattcaaatcagattattgactgtcatttgtatcaaatcgtgtttaatggatatttttattataatattgatgatatctcaatcaaaacgtagataataataatatttatttaatatttttgcgtcgcacagtgaaaatctatatgagattacaaaataccgacaacattatttaagaaacatagacattaaccctgagactgttggacatcaacgggtagattccttcatgcatagcacctcattcttggggacaaaagcacggtttggccgttccattgctgcacgaacgagctttgctatatcctcgggttggatcatctcatcaggtccaatcaaatttgtccaagaactcatgttagtcgcaacgaaaccggggcatatgtcgggtatctcgagctcgcgaaagcttggctgcaggtcgacggatccttcaacaaaagggtacctgtacccgaaaccgacacaggtgggtaggtagagaatacctaggggcgcgagacaactctctctaaggaactcggcaaaatagccccgtaacttcgggagaaggggtgccccccgctaacaataaacgaatacggtttatgtatggattccggtaaaataccggtactcgatttcataagagtcgaataggaagttaagatgagggtggtatcatcataaaaatggagtagtatcctaaattatactaatccacgtatgatatgtatgcctttccttatcaaccggaagtagtgcaaaaaaaattctatactgcactgctctctttttactgagaaatgcaaaaaaataaaagtgaagtaagggtgccccatagatatttgatcttgcctcctgtcccccccccccttttttcatcaaaaatttccatgaaaaaagaaaagatgaatttgtccattcattgaaccctagttcgggactgacggggctcgaacccgcagcttccgcctgttcctagccttccagggcccagcgtaagcaataccagccacagcaccctcaacctcagcaaccaaccaagggtatctatcttgcaacctctctagatcatcaatccactcttgtggtgtttgtggctctgtcctaaagttcactgtagacgtctcaatgtaatggttaacgatatcacaaaccgcggaacacaagaacgaaagcaccttttcattctttcatatactaggggtttttacttggaaaagacaatgttccatactaaaggatagctgcagaagccgccaccgtcttgaggaccttccggggagccagaccggtcgaaccgtgcctccacttgctaaggagaaagggaaaatcagggccaggacatacgaaggaggagccagaacgaagatatcctaagatacttactcgctccgggccatgatcaatcatgcctgtggggaggtctctcgcacctcgatccatgaaggtaccaccgaggtctgccccgccgccggcttcggtaccgtcctcgccttgggcgcccgaggcacccgggggatggactgcccaggcgcagccacgacgacccaaggatcaccctcctgcgcagtcggcacgagcaatagttctcggggaacaggcagcttggcctgactccccggggtcacctcaactacctcggccgaggggtcaagtaccccctcagtccgcccccgctcttcggaccgggaccccgacgtcccggccccggataccgacggcaccagcccgctcgggggctggcttgacgacccctggcccagcctcagatctgggctgaggccgaggcaggcggccatgtcgtcgtcttcatcatcgtcttcatcatcgtcgtcgtcatcaggcgtctccggcgacggctcccttgggagcccctccctctcctgccgacgacgaagcctttccaaggcatcccgagcccacgtccgctcgtgggcccgagccttctttgcgtccttcttctccttcctcttctccgcggtgaccctccgcgcagctcggtccaccgcatcctccgggactggtggcagggaaggcttgtgatgccctacctcctggagacagacgaaaagtctcagctatgagaaccgagggcaatctgacgcaagaaggaagaaggagcggatactcaccagagacacgcacccgcgatcgggacgcattaagggctgggaaaaagtgccggcctctaatttcgctaccgtgccgtccacccacctgtggaggtcatcgatgggaaggggaa

SEQ ID NO: 10 - DNA, 8865 bp, Artificial
  synthetic
tacaagaatattgagacgtgagtacatagcattggcattttcattagcaagcatttcaaaagaatttaattttctcatagcaatgtgatatctctcctcacgctcaattctagttccttcatgtagagcacatatgtccatccacaaatcatgacaatttttatggtttctaactctattaaacacatctttgcaaaggcctctaaaaagggtgtttttggccttagcattccatttctcatagttcaactcttcacctacaagatttgtgggatctctaggttcggggaatctttgtgtggcggctttgtagacaccaatgtctatagcctctaaatatgcttccatacgaattttccaatatggaaaatcgtcaccataaaaaacgggagaaggtccatccccaccggacatcgttactctagcggttaagctaatctaagagcaacaaggctcttataccaattgaaaggatcacgatgcccaagagggggggttgaattgggcttttctaaaaatcaacactaactaaaatctaagcaagagcccaacttcaccccgacaactagcactaagagaataatactagaaatacaacaatgctaagataatacttcaaatacttgctaaacaaatacacaatgtaaaatacttgaattaagtgcggaatgtaaagcaaggtttagaagactcctccaatttttctagaggtatcaaagagtcggcactctcccctagtcctcgttggagcacctgcgtaagggtatcgctctcccttggtcatcgcaagaaccaagtgctcacaacgagatgatcctttgccactccggcgcggtggatccctcacgaccgcttacaaacttgagtcgggtcaccaacaagatctccacggtgatcaccgagctcccaacgccaccaagccgtctaggtgatgccgatcaccaagagtaataagccatagactttcacttgaccaagagaagcctaatgcatgcggtgtgtgctctaggtggctctcgctagcgttaatgaggtccaaatgcgggattaagattctcaagtcacctcactaggctttgtggtgcttgcaatgctctaccaatgtgtaggagtaaatgtgggcagcaagaccatcaatatggtaggtggatggggtataaatagccctcacccaccaactagccattaccaggaatctgctgcgcatgggcgcaccggacagtccggtgtgccaccggtgcgccaacggtcgactcaaacggctagttctgacagctagccgttggacagatggcataccggacagtccgatacgctgtccggtgtgcctctaaaattcaactcacgaactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagctgccatttaaatcaattgggcgcgccgaattcgagctcggtacaagcttctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaggatccaccatgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctgagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatataccctcttccctgggccaggctgggcccactggcaaagggtgcaccggacagtccggtgccccaaagccagaaaccctagcttctgttttgtgctgttttttcaatttggtttttgttctaacttgtgagtatgttctagagttacacctagcactatatgtgagtgtgaatatgcaccaacactacactagaactcttttggtcaaactacttatcgacaacccctctttatagtacggctaaaacaaaataaaagacctaactatatcacgagtgtccgcaactccttgacactcggaatacgaagaccttcactttttgtttcgtcgctttagccgttgcttcaagtttttatctccgggattgttttcaccattgtagtacatctacctgtaatgcgacctaacttaccatttgcctctgcaaaacacatgttagtcacatataaaattacgttgtcattaatcactaaaaccaaccaggggcctagatgctttctagtttaaatccccaacaagtcaaaattctttctatttttttttgcaagttccaattgacatctgaaaggttgtaaggtacacgtttggctctcattgataacgggggaaagatacagtgcaaaccaccatataatgacccacttctaatcgaatggacctgtaacgacgaaataccctgtgagaactatggttcactcatgttaattcattgaaattgttgtagtgaattgacatggttgggagcctgcttagagagtatagattgtcacttttttttggaccgcaacttatttttaaaagatattgcgatcgcttgtttagtagctgtttcaggccccaatgcagtttctatcgtgatccatttaagtcactcaacattctcatacttctcattttgcattaattcattccaatctccactactataaaatactagcttcgatggtcgtcatacgccatgcacgaagcatgtagatcaatccgcataccagtgggcatctatagataggctgtgaaaaccacccaaatccctactagtggacattttatctatagatggaccgtgagaaaccacacaagtctaacacgacagg

SEQ ID NO: 11 - DNA, 13659 bp, Artificial
  synthetic
ccgctgaagaagatcaagtgtgtgaaccaaagtgaaaataatgtttagaagccaacacagtggacaattttgatttctggttaatgggtgtattaaaaatatcagaaaactttcaaatatctcgagcaggcagttgggttgcctaatccatcacttgatctcaaagattcttgttgtcaagcttcgatgagtagcgaaacctaccgtgtgggctctaagctttaggttgccattgacgatcttgacacgacatatgacctatgatagcaattcaaacatggttcatatcagctgagttgcagacttagctgctagcatttcacgttgtgaatgcaagaggaaaaatacatagaaaaagagggaacaaaattgttaaccctccatatgtacaggttttagcctcaattttacccattgattgatttattattgaaaagtaatcacttatcagaaccagagtttgtaattcagcttgtatcgtaccatctaatatcttagtcaaatttgtgtataataagtatttaaatgtgagtctcttttatttaattagtttaaataattggaatacaattggcaaatgggcacttcaactataattagtgaattgatttagttgactgttatgaagtattttacttaagttagtaatagtagagtgatgtttgatgaattaaacttaagattggttgaagttattgatctcactggatccatagtttggtctgtgggattgcatctgaaacggatcatatggttttgttttgtgactgaattgtggcaatgtaacacctggactttttcacaactattgtataaatccagtatatctcacgtgaatctgaaattagtagcatgcttaacatataagtatcgatttatctaatcagtttccatatttatgaaaactgcactgttgaaaattgtgcaagcttaacatacaagtaatgtaatccacagtacgaaaaatgtgcaggttcttatttgtgctccataattgtttcttgattccgatcaaagcaagagcatccagtctcaaaattttgtcttctcaattcactcattcatcaaaatcagcagttttatgcatcaacaagcatggaatgttgaaccacccatgattaagccccatatcgttgtgttgagataactatcacctgaagttgtcttataaaaaacacatctgaatacttttataatcatacctttctcggccttttggctaagatcaagtgtagtatctgttcttatcagtttaatatctgatatgtgggtcattggcccacatgatattaaatttattttttgaagggtggggcctgacatagtagcttgctactgggggttcttaagcgtagcctgtgtcttgcactactgcatgggcctggcgcaccctacgattcagtgtatatttatgtgtgataatgtcatgggtttttattgttcttgttgtttcctctttaggaacttacatgtaaacggtaaggtcatcatggaggtccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccgacgaatgtccccgatcaaatctgagggacgttaaagcgatgataaattggaaccagaatatagaatctttgttctgctctagcttttcttctgtacattttttacgattagactatgattttcattcaataaccaaaattctgaagtttgtcatcaagttgctcaatcaaacttgtaccggtttgtttcggttttatatcagctcactgttacactttaaccaaaatcggtttatgtcttaataaaggaattgagtcggtttaactcatatccgtaccaatgcgacgtcgtgtccgcgtttcagtagctttgctcattgtcttctacgggaactttcccggacataggaaccgccctttcgttatcctcatccatcgtgaaatcaggaaataaatgttcgaagatttgaggtcaaaagtcgaatttcatgttgtctcttctatttagatacaaaattgaagcaattttcaccaatttaatgccaaaatttaaaacaacgctgataaagtgaaacttgattcgatttatatttcaaccgaaactgctgaagcaagaagaaaaagcgtaattacacataacaagaacgctaccgcaaactactaaacgccaaacccaatacaaaagtaaaacgcagacgcttaagtgagaaacccagaaaacacaaacgcggatcgggggatccactagttctagagcttaattcttgacgaaagtgctcagcacatcgaagtagtcggggaaggtcttccgggtgcacccagggtcccggatggtgacggggacctcggcacaggcggcaagggagaaagccatcgccatcctgtggtcgtcgtacgtgtcgatcgccgtcacgttcagcttctccggcggcgtgatgatgcagtagtccggcccttcctcaacagatgctcccagcttggttagctccgtccggatcgcaaccatcctctcggtctcctttactctccaggaagccacgtctctgatggctgtcgggccatcggcaaagagggcaaccacagcaagagtcatggcgacatcaggcatcttgttcatgttgacatcaatcgccttgaggtgtttcctcccaaatggctcccgcggtgggccagtaacagttacgctagtctcggtccatgtaaccttcgctcccatcatctccagtacctcagcaaacttcacatcaccctgcaaactggtggtgccacaaccttccacagtcacagtccctccagtaattgcagcaccagccaagaaatagcttgcgcttgaggcatcaccttcaacataggcatttttaggggacttgtatttttgacctcccttaatgtagaatctgtcccagctatcagaatgctctgctttcacaccaaaacgctccatcaatctcaatgtcatttcgacgtacggaatggagattaatttatcaatgatttcaatctccacatccccaagagccaaaggagcagccatcagcaaggcactcaagtactgactgctgatggagccagacagcttgaccttgccaccaggtagccctccgattccattgacacgaacaggtgggcagtcagtgccaaggaaacaatcaacatctgcaccaagctgcttcaatccgacaaccaagtcgccaatgggtctctccctcattcttggtactccatcaagcacgtaagttgcatttccaccagcagcagtaacagctgctgtcaaggaccgcattgcgattccagcattccccaagaagagctgcacttcctctttagcatcctcaactgggaactttccaccacagccaacaactacagctcttttggcagctttgtccgcttcgacagagagaccaagagtcctcaaggccccgagcatgtagtggacatcctcactgttcagcaggttatcaaccactgttgtcccctcggacagggcggcgagtaggaggatccggttggaaagcgacttggaccccggcagcttgacggtgccggagatctccttgatgggctgcagcacgatctcctcggcgccggccatgcaccggatccttccgccgttgctgacgttgccgaggcttctggaggagcggcgggcgacggggaggctggcggtggacttgagcccctggaacggagcgacggcggtggccgacgaggccatcatcacggtgggcgccatagacagcggcggcaggtacgacagcgtctcgaacttcttgttgccgtaggccggccacacctgcatacattgaactcttccaccgttgctgggaagggtggagaagtcgttagccttcttggtggtggggaaggcggcgttggacttaaggccggtgaacggagccaccatgttggcctgagcaggggcggtccggctaacggtcgcgactgaggaggagatcgaagccatggggatctgcgcatttaacaagaaattgaacagtcaattggggattttcattatccataactaaattttgaagaaattggaatactaaacgtcaccacttaaaaccctaatccagatgaatcgttatcgaaccagatataaccaaaaggggcaaaattgactcgaaaaccctagttctcgatacacggctaggtaatgacaatcgcacacagacaaatctggttatacagaacttcgaagcaagaaaaaaacgatgaagaatggatcatccaataaatcgactagactcaatcttcacaggtttatcgatccagcaaacttaaaagacggacctttattttcaaactggaatgggacaaaacccgaaactctattgtcgtaaaatcagatcgcggagacagtaacagaaaaaacattaaaaagtaatggaaagacctaaacccctgatctaattacaaacaaatcatacctgttcttcgcctgaggggttcgaaatcgataagcttggatcctctagagtcgagagaaattgatgtctgtagaagaagaagaacggttaagagtagatttgggtgagaaagatgtgaaattgtttttataggcaaagacggagagtctattttttgagcaatcagatcgcatattaaatctaacggctgagatatcgatccgtgtgtacaataaaatgatgtataaaccgtcgatctgttttaatcgacggttcatattagtgatccgcgtgatggcagtgatagccactaagaatcgtcttttgttttacatgtggcgccacaaattagggtaatgaagcggcaatattttggaactcggaaaataaaattgcgccatcacattatttgaaaattttcacatgcttttattttaaaaacccacgaattacaagttacaaccgaaaaagatttataatatagtgatttatactaattttgtagtagcttaatgtatattgatactggaaaaacaatgacaatcatatgttagtattatcaagttatcgtattgatattgatattggaacatacaatgggtattgccttctttcgaccataaatatcaccaaatttacaaagtttgtgtataccaagttatcaattgtaaatgggatgtcaacattttaatttccctttgagaaactatagaccacaagaacacacttcaatagataaagtaactatttacataagaggttttaaaatcacattaacaaaaataattaccaaccggcactcacaaatacaaacagagcacacgacatgtcaaagccacaagtaaattcgttgagtggtggtttcattacaattgtgtcacttgcagcacaaactatcttgctctgggaatcatctcagcatcaaagatcatgctcacttcaggggaacttagtgtatccatgcctcgactcatatttctcctcgacatgcatcctgcaggggcgcgccatgcccgggcaagcggccgcacaagtttgtacaaaaaagcaggctccgcggtgactgactgaaaagcttgtcgacctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacagagatctccatggctcagaccactctccaaatcacacccactggtgccaccttgggtgccacagtcactggtgttcaccttgccacacttgacgatgctggtttcgctgccctccatgcagcctggcttcaacatgcactcttgatcttccctgggcaacacctcagcaatgaccaacagattacctttgctaaacgctttggagcaattgagaggattggcggaggtgacattgttgccatatccaatgtcaaggcagatggcacagtgcgccagcactctcctgctgagtgggatgacatgatgaaggtcattgtgggcaacatggcctggcacgccgactcaacctacatgccagtcatggctcaaggagctgtgttcagcgcagaagttgtcccagcagttgggggcagaacctgctttgctgacatgagggcagcctacgatgcccttgatgaggcaacccgtgctcttgttcaccaaaggtctgctcgtcactcccttgtgtattctcagagcaagttgggacatgtccaacaggccgggtcagcctacataggttatggcatggacaccactgcaactcctctcagaccattggtcaaggtgcatcctgagactggaaggcccagcctcttgatcggccgccatgcccatgccatccctggcatggatgcagctgaatcagagcgcttccttgaaggacttgttgactgggcctgccaggctcccagagtccatgctcaccaatgggctgctggagatgtggttgtgtgggacaaccgctgtttgctccaccgtgctgagccctgggatttcaagttgccacgtgtgatgtggcactccagactcgctggacgcccagaaactgagggtgctgccttggtttgagtagttagcttaatcacctagagctcggtcaccagcataatttttattaatgtactaaattactgttttgttaaatgcaattttgctttctcgggattttaatatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttgcggccgcgcgccgacccagctttcttgtacaaagtggttgcggccgcttaattaaatttaaatgcccgggcgtttaaacgcggccgcttaattaaggccggcctgcagcaaacccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatctccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaggtaccctgagcttgagcttatgagcttatgagcttagagctcggatccactagtaacggccgccagtgtgctggaattcgcccttgactagataggcgcccagatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacatagcggccgggtttctagtcaccggttaggatccgtttaaactcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgccccagccagccaacagctcgatttacagagaacgaatgtcgtgtgatatgtggaacaaggcaacgacaacaacatacatgaatctcacaatagagtcggggtcgccgagttgtgatgtaatccatggcatggacatggtggccgatcgaaaaagaaaaaagaaatgcatgtatatgtgtgaaaatgagagttttttttatccaaataataaaaaaaaattaattatttacccaaaaaattatttacatgaccgatacgtacacttttttccttagttaagaaacaccgatttcttaattacatttttttatacatttagaaattggtttccttggaaccgatttcaaaatgttcattttttttttcaaaaccaagttaagaaatcggttccttggaaaacgacttcttaattgcttttttttttgttttgttttaaaattgtttgtatttttattttttttgttattaattgtctatatttgtgttctgtttaaattgaaaacaatattatttttcatatgttgttaattcttaatttcttatgcatattttatgttttatcattttttaagagttgaaatcctttgtatttttattttatttgattattataatacataattaaacaacaacttaattgaaattaaaaaatatatatttaactgacaagttgacttgaactaaaatatttaaattacaaaatagatatgaaattacaaacaatagaacaaaatatttaaatttgaaataatacaacaaaaattttaaaatacaaacaatatggcataaaattaatgttgttggcctgagcctacacaatggggggaatgcgacacatggaacatcattttggtttacctgattcttggatatccattttggtgtgtatacgagaggagacatgacaacctttagaatttcttttcatttttgggttggggcaaattcttggcttgtgacatggtgaccaacatgcttcattgcacaatttcccaagtaatcatttgtacatgttatagatacttttcagcgtatacacaagatgtatgtagttcctatactcatggtgagcatgcttataaacaacaacatgagaacaaaacatgtgcattttttgaaatttgtcacagtcacactacctttcatctagcttgaccatgaaatttctcactagttgaatctctctctccagttaattgtctcctagactaagaattgtatgtcgcattaatcgaactcaagaacaatgtgagagtttgcctttttctgtatgtctttcatagccttgtttaatacttttgtataaacttcaccaaatgtaatcactcttgcgacttctctccccctttggttgaacaatgcattacacctagtataggttgattttaccaacacacattgccagattttgtgttttcttgagtaccaaattaattaactcagtgtccccatcgccagccaccatcccatgcgagagtccacttttcttgtggaatcttcctaagctaattaattgttagttcagcttggccttaaaacacacatgacaaatatttttattataaaacaaacacccagtgatacaacaatgaacaacacacatgacaactacaaatcattaaataatacaattacaatcaaataatttggggagggggtcttcaaaacttgattcaaggttcattgtatcatcgaggtattcacccaaaccctccaaattcaaagagcttgcagaaactggtggtggttgtgcatatgtttatgtgggtgtgtctacggtgataacataaaactcaaggcatgtgaagttgaagagtttcccaaatatagaaaacatagtaattatttcctcatcatcttgcagcatacatgccctgtactgaaaacaatcaccaacaaatgatacgggaca

SEQ ID NO: 12 - DNA, 10212 bp, Artificial
  synthetic
ctgtcgttggattcacagaacattgacgccagttttcacttcgttatctttgaattcattaaaatcgaatctctcacctatacccccccatttttctaatccatcataatcaaaattcataaatgaatcagttaccattaccataatacctttttgaaaatgagtttgaataatcagtatctttagaaaactaattaagaaattaaataaaaaatatttatcatgaagatgagtgtaagaaaaattatgaaaagtataactttatacatttctataaaattattttttcttttaatttcttaattaatatcctaagtaaatgagttaatatttatctttcaaaaattcttatagtcgccaattaattttcccatgcaatgacaacttgtccgtattctacgtggtaggttaggctacctgccgagacaaattgccttgagacaaattcaatagagaacccttccaagggaccattataaatagagaactttcattaaccgataagccacaccctttcaatcaaacacaaacacttgaagtactaagttagtgtgtttgagcaaattaactatggcttcgttttgttctagattgacaatttgtttggctctgtttgtcctcatatgggggagtgccaatgcacaactttctacaaacttttactaccattcatgtccaaacctcttctcctctgtgaaatccacagtgcaatctgccatatctaaggagacccgcatgggtgcttctctccttcgcttgttcttccacgattgctttgtcaatgtaatttatttgcaccttctcccacttacatacaaatatgctaagcttacatatagctcctctttctaccacttgcatgcatcatctaattttgtttgaaacaacacttgttccttttattatacacatcatctttgataaaattttgtcgtgtgcaacttttttttagtgtgttaatcagttctatgatgatactattagttaagaaattttaatgcacttaataaaccattttaagtactttaaccgttcaatgatattatatatttaaagataataaatatttctgcttttgtttctatattagtgtagttaagaaccttcttacttcttagctagctaaatattaatgagtaaacattaacaaatgcagggatgtgatggttcaattctattggatgacacatcaagcttcaccggagagaagaacgcaaaccccaacaggaactctgctcgtggattcgaggttattgacaacattaaatcagccgtggagaaagtgtgtccaggagttgtttcctgcgcagatatccttgccatcgctgccagagactctgttcagattgtaagtggtcaaacaaccaacaaaaacacattaaactaaatcattaaattgtacatatcaaaattaattaccaatttagtaccacacatgcaattaaagagaacattttgttgattttgatcaatatagcttggaggccctacatggaatgttaaacttggaagaagagacgctagaactgctagccaatctgctgctaacaatggcatccctgcacccacttcaaaccttaaccaactcatctcaagatttagcgctcttggactttccaccaaggacttggtcgccttgtccggtacaaaacatatatcacataattttccaattaattacatttcaatcatatagtaaaatttctcaattaattaggaacatgagaaacttatagtcacacgttcttttgttgaggaatattgcatggtttaattttgctttcattaggtggtcacacaattggacaagcaaggtgcacaaacttcagagcccgcatctacaacgagaccaacatagaaaccgcatttgcaaggactaggcagcaaagctgccctagaacatcagggtcaggggacaacaatctggcaccacttgatcttcaaactccaaccagctttgacaactactacttcaagaacctcgttcagaagaagggtctcctccactctgatcagcaactgttcaacggtgggtccaccgactccattgtgcgtggctacagcaccaacccgggcaccttctcctctgatttcgccgccgccatgatcaagatgggagacattagtcctctcactggctccaatggagaaatcaggaagaattgtagaaggattaactaatttgattcagtcttgaatattaagggtcctacacatacgcaagcaatttaattgtgtttaataagttgttaaaacatgttttggttgtattttggattcctagtgtagtttcggtgatcaatgccgtctactttagtgtgttctacttccctttatttttgtttcttttttactttttccttaactatattgtaggaaaaaaaaaatcctttatcaagcatttatcaagaacggagtttgctttttaattttcccttcataacattccatcagaattcagttttgcttttgcttctaaattacgttcaaatcagggatgataatcggttaggtaatatatacagtaccccttgcatagtcacgtttgaaaaatataatcatacttagttcggtaacaatttaaattatcattctcgtaatcattagctacttatgcactcatatccgtatccgctacttgctcttgtcgtaagtcaataaattaatataaaaaaatacttaaaacttgttacaactaaattaaaaatttatttttaaatcattcaagcaccagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcacaagtttgtacaaaaaagcaggctccgcggtgactgactgaaaagcttgtcgacctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacagagatctccatggctcagaccactctccaaatcacacccactggtgccaccttgggtgccacagtcactggtgttcaccttgccacacttgacgatgctggtttcgctgccctccatgcagcctggcttcaacatgcactcttgatcttccctgggcaacacctcagcaatgaccaacagattacctttgctaaacgctttggagcaattgagaggattggcggaggtgacattgttgccatatccaatgtcaaggcagatggcacagtgcgccagcactctcctgctgagtgggatgacatgatgaaggtcattgtgggcaacatggcctggcacgccgactcaacctacatgccagtcatggctcaaggagctgtgttcagcgcagaagttgtcccagcagttgggggcagaacctgctttgctgacatgagggcagcctacgatgcccttgatgaggcaacccgtgctcttgttcaccaaaggtctgctcgtcactcccttgtgtattctcagagcaagttgggacatgtccaacaggccgggtcagcctacataggttatggcatggacaccactgcaactcctctcagaccattggtcaaggtgcatcctgagactggaaggcccagcctcttgatcggccgccatgcccatgccatccctggcatggatgcagctgaatcagagcgcttccttgaaggacttgttgactgggcctgccaggctcccagagtccatgctcaccaatgggctgctggagatgtggttgtgtgggacaaccgctgtttgctccaccgtgctgagccctgggatttcaagttgccacgtgtgatgtggcactccagactcgctggacgcccagaaactgagggtgctgccttggtttgagtagttagcttaatcacctagagctcggtcaccagcataatttttattaatgtactaaattactgttttgttaaatgcaattttgctttctcgggattttaatatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttgcggccgcgcgccgacccagctttcttgtacaaagtggttgcggccgcttaattaaatttaaatgcccgggcgtttaaacgcggccgcttaattaaggccggcctgcagcaaacccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatctccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaggtaccctgagcttgagcttatgagcttatgagcttagagctcggatccactagtaacggccgccagtgtgctggaattcgcccttgactagataggcgcccagatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacatagcggccgggtttctagtcaccggttaggatccgtttaaactcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatattttaattcttaacaatcaatattttaattcttaaactttattaaatctaacaataaactgtaagaactaattcttaaacttcaataaacaatactgcgttttagtaattaaattaataatatatagatatagatatataatttgtcaacatattcttacctatttttccattgaaatatgttagcaagttcaaaaaaagttttgacaaaaaactctactatcttttgtttcatttactttatgtgagggatataatagtaatataacatttagtttatttaaagaaaataaaaaagttaatttctctttctgccactgatactctatggtggagagatccgatgcagtggtggagcctggcctcgacacataagtgtgacgacgcagctgttgaagagatctgattcgacggtggggtaatgcatggtggttgacaggttgatgggtggagaagacgtaattgctaccgccgtcaacggaggaaggagcaaagatgtctcgtatgtgaaaattatgcggttgagatgccgtttcattccctttaaaaaaatcccttgatggttgcaatgcaaattaaaaattgaaaaaataattaattgttcaaattaaagatttagcatgaaaaaaaaaacacttaattgtgcccatgactccatgacctgcgtaacttgggaaggaaaggaatttttttgctaaaggaaggcatgggaagatgagagaggagagagaatcagtggaagtgagagaaattaactttttgttttttaaaaactaaatattatattactattatatatatatatatatatatataaaagattttttagctggattcttgatataaaaaatttctcaccatatttattattatatatttttttggagatctcaaaaaaggaagttggatttcttctcaataactctaaaaaattattcctatttcaaaaaatattttttatgtctttctctaattgatgaataatatctatttaagtatattttattgtgaaatccacaaaagtgactgataaatctaatttaggatctaccattagagaaaaataaataaattcttatattatatgtgat

SEQ ID NO: 13 - DNA, 15294 bp, Artificial
  synthetic
ttaacaatgaccaagatttatgctatatagaagacttggagggcttaaggctatgatatattatggatgatatggttctgatttgtgtagtttcgaaggatcaaatcaaccatttgttggtacaatgggaagaaaaaatgttttcatcattccactctattgaaaaagatccaacaattgtaacaccccgacgaatcacaccggaaagagaagaatccaaagattgtgtaggtatgagactgtatagttgatgaaaacttaaaaaaattaattggtactacttataccaacaagatgcatatatttttcgatagcctatcacataagaacttcatagttaagggtgcttaacttggagtagttatgaaatgagtgaccttttaaaataattattgtcttaggttattgtatgaaaataaaaaataataataaatatacataaaaaataataattttataaaattaaccttatattatcattaatttatttttagattttgttattcattattaatatatgaggtataaatgaaaaatataattaatgtcacattaaaaaattaaaatgataattattttgaaacaaattatttatttttatacgacaattataatagaaatttgagagtaaaaaaaaattgaaaattcataaaatatatgaatatattcatttctcctatccgtcaaataaatctgctccataatttatctaagcattggtcttgtagttcagagtaataaaattttagcaattattagttagtacagatacatttaaagaaataatatattttagcaactagaagtttataaaaagttttaaattataaagacttatatataaatttagtaaaactagatggatgtcccaagtaatttttatataactattctcgtacaacattaatgaaaatcttgtttctattatttatatgtatattattattttattttggaacaatatgggattaaaaactcttataaattaaatcttagaataagttttcctaacatgttttttttatggatgttttcctaacatgtttggttatcttagttttgctttaattttgtcggattatttttggactttattaggtaattttgataaaacttttagttgatgttagtagtttactcttacataatgatttgatattgaatgtgtataattggaaggcaataaatgaagatcaagcgtacaagagttcgccaatcaagaggatttgaagagagtaaaatattatgcgaagtcccatgtgaagaaaatccaaccattggaataaaaaataaagttttttctttggaattgctaatgctacagcacttattggtacttgtcctaaaaatgaaactctagctatatttagcacttgatattcatgaatcaaacttctctatgaaataaccgcggtgcgcatcggtgcctgttgatcccgcgcaagttgggatcttgaagcaagttccgctcatcactaagtcgcttagcatgtttgaccttctcggacaactccttcttctctttaattgatcaacagtcagcatcatcacaccaaaagttaggcccgaatagtttgaaattagaaagctcgcaattgaggtctacaggccaaattcgctcttagccgtacaatattactcaccggatcctaaccggtgtgatcatgggccgcgattaaaaatctcaattatatttggtctaatttagtttggtattgagtaaaacaaattcggcgccatgcccgggcaagcggccgcacaagtttgtacaaaaaagcaggctccgcggtgactgactgaaaagcttgtcgacctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacagagatctccatggagaacaatatccagaaccagtgtgtcccatacaattgcctcaacaatcctgaagttgagatcctcaacgaagagaggagcactggacgccttccccttgacatctccctctccctcacaaggttccttttgtctgagtttgttcctggtgtgggtgtggcctttggcctctttgacctcatctggggcttcatcaccccatctgattggagcctcttccttctccagattgaacaattgattgagcagaggattgagacccttgaaaggaacagagccatcaccacacttcgtggccttgctgacagctatgaaatctacattgaagcactccgtgagtgggaagccaatcccaacaatgctcaactccgtgaagatgtgaggattcgctttgccaacacagatgacgctttgatcacagccatcaacaatttcaccctcaccagctttgagatccctttgctctcagtctatgttcaagctgcaaacctccacttgagcttgcttagggatgctgtgtccttcggacaaggttggggacttgacatagccactgtcaacaatcactacaacagactcatcaacttgattcatcgctacaccaaacattgcttggacacctacaatcaaggattggagaacctcagaggcaccaacactcgccaatgggcaaggttcaaccagtttagaagggatctcacactcactgtgcttgacatagttgctctcttccccaactatgatgttcgcacctacccaattcaaaccagctcccaacttacaagggaaatctacacctcctcagtcattgaggacagcccagtttctgccaacatacccaatggtttcaaccgtgctgagtttggtgtcagaccaccccatctcatggacttcatgaactccttgtttgtgactgccgagactgttaggtcccaaactgtgtggggaggccaccttgttagctcccgcaacaccgctggcaaccgcatcaacttcccatcctatggggttttcaatcctggtggagccatctggattgcagatgaggacccaaggcctttctacagaaccttgtcagatcctgtctttgtcagaggaggctttggcaatccacactatgttcttggtttgaggggagtggcttttcagcagactggcaccaatcacacccgcacattcagaaacagcggcaccattgacagccttgatgagatcccacctcaagacaacagcggagcaccctggaacgactactcccatgtgctcaatcatgtcacctttgtgcgctggcctggtgagatcagcggttcagattcttggagagcaccaatgttctcatggacccatcgctctgccacacccacaaacaccattgatccagagagaatcacccagattcccttggtgaaggcacacacacttcagtctggaaccacagttgtcagagggcctgggttcactggtggagacattctcagacgcacctctggagggccatttgcttacaccattgtcaacatcaatgggcaacttccccagcgttaccgtgccagaatccgctatgcttccaccactaacttgagaatctatgtcacagttgctggtgaaaggatctttgctggtcagttcaacaagacaatggacactggtgatccattgacattccagtcattctcctatgccaccatcaacactgcattcacctttccaatgagccagtccagcttcacagtgggtgcagataccttcagctccggcaatgaggtgtacattgaccgctttgagttgattccagtgactgccacacttgaggctgagtctgacttggagcgtgctcagaaggccgtgaatgctctcttcacctcttcaaatcagattgggctcaagacagatgtgactgactaccatatagaccgtgtttccaatcttgttgagtgcctctctgatgagttctgcttggatgagaagaaagagttgtcagagaaggtcaagcacgccaagaggctctctgatgagaggaacttgcttcaagatcccaacttcagagggatcaaccgtcaattggatcgtggatggaggggatcaactgacataaccattcaaggaggtgacgatgtgttcaaggagaactatgtcacactcttggggacctttgatgagtgctacccaacatacctttaccagaagatagacgaaagcaagctcaaggcctacacaagataccagttgagaggttacattgaggactctcaagaccttgaaatctacctcatcagatacaacgccaaacatgagacagtcaatgtgcctgggactggttcactctggccactttcagccccaagccccattggcaagtgtgcccatcactcacatcacttctccttggacatagatgttggctgcactgacttgaatgaggaccttggtgtgtgggtgatcttcaagatcaagacccaagatggccatgcaaggttgggcaatcttgagtttcttgaagagaaaccacttgttggagaagcccttgccagagtgaagagggctgagaagaaatggagggacaagagagagaagttggagtgggaaacaaacattgtgtacaaagaagccaaagaatcagttgatgctttgtttgtgaactcccaatatgataggctccaagctgacaccaacatagcaatgattcatgctgcagacaaaagggttcacagcattcgtgaagcataccttcctgaactctcagtgattcctggggtcaatgctgcaatctttgaagagcttgaaggacgcatcttcactgccttctccttgtatgatgcaaggaatgtcatcaagaatggtgacttcaacaatggcctttcctgctggaatgtgaaagggcacgtggatgttgaagagcagaacaatcaccgctctgtccttgttgtccctgagtgggaagctgaagtttcacaagaagttcgtgtctgccctggtcgtggctacattcttcgtgtgactgcttacaaagaaggctatggagaaggttgtgtcaccatccacgagatagagaacaatactgatgaattgaagttcagcaactgtgttgaggaagaggtctacccaaacaatactgtcacttgcaatgactacactgcaactcaagaagagtatgagggcacttacacttctcgcaaccgtggctatgatggagcctatgagagcaactcatctgtgcctgctgactatgcttcagcctatgaagagaaggcatacactgatggaaggcgtgacaatccttgtgaaagcaacagaggctatggggactacacacccctcccagctggctatgtgaccaaagagttggagtactttcctgaaactgacaaggtttggattgagataggagaaactgaaggcacattcatagttgactctgtggagcttttgctcatggaagagtgagtagttagcttaatcacctagagctcggtcaccagcataatttttattaatgtactaaattactgttttgttaaatgcaattttgctttctcgggattttaatatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttgcggccgcgcgccgaaaacaactttgtatacaaaagttgccgcggtgactgactgaactaaacccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatccaacaatggacaacaatcccaacatcaacgagtgcattccttacaactgcctgagcaaccctgaggttgaggtgctgggtggagaacggattgagactggttacacacctatcgacatctcgttgtcacttacccaattccttttgtcagagttcgtgcccggtgctggattcgtgcttggacttgtcgatatcatttggggaatctttggtccctctcaatgggacgcctttcttgtacagatagagcagttaattaaccaaagaatagaagaattcgctaggaaccaagccatctcaaggttagaaggcctcagcaacctttaccagatttacgcagaatcttttcgagagtgggaagcagacccgaccaatcctgccttaagagaggagatgcgcattcaattcaatgacatgaacagcgcgctgacgaccgcaattccgctcttcgccgttcagaattaccaagttcctcttttatccgtgtacgtgcaggctgccaacctgcacttgtcggtgctccgcgatgtctccgtgttcggacaacggtggggctttgatgccgcaactatcaatagtcgttataatgatctgactaggcttattggcaactataccgattatgctgttcgctggtacaacacgggtctcgaacgtgtctggggaccggattctagagattgggtcaggtacaaccagttcaggcgagagttgacactaactgtcctagacattgtcgctctctttcccaactacgactctaggcgctacccaatccgtactgtgtcacaattgacccgggaaatctacacaaacccagtcctcgagaacttcgacggtagctttcgaggctcggctcagggcatagagagaagcatcaggtctccacacctgatggacatattgaacagtatcacgatctacaccgatgcgcaccgcggttattactactggtcagggcatcagatcatggcatcacccgttgggttctctggaccagaattcactttcccactttacgggactatgggcaatgcagctccacaacaacgtattgttgctcaactcggtcagggcgtgtatagaaccttgtccagcactctatataggagacctttcaacatcggcatcaacaatcaacaattgtctgtgcttgacgggacagaatttgcctatggaacctcctcaaatctgccatccgctgtctacagaaagagcggaacagttgatagcttggatgagatccctccacagaacaacaacgttccacctaggcaagggtttagccatcgccttagccatgtgtccatgttccgttcaggctttagtaatagcagcgttagtatcatcagagctccgatgttctcttggatacatcgtagtgctgagtttaacaacataattgcatccgatagcattactcagatcccagctgtcaaggggaactttctctttaatggttctgtcatttcaggaccaggattcactggaggcgacttggttaggctgaattcttccggcaacaacatccagaatagagggtatattgaagtgcccattcacttcccatcgacatctaccagatatcgtgttcgtgtaaggtatgcctctgttacccctattcacctcaacgtcaattggggtaattcctccatcttttccaatacagtaccagcgacagctacatccttggataatctccaatctagcgatttcggttacttcgaaagtgccaatgccttcacctcttccctaggtaacatagtaggtgttagaaatttctccggaaccgccggagtgataatcgaccgcttcgaattcattcccgttactgcaacgctcgaggcagagtctgacttggaaagagcacagaaggcggtgaatgctctgttcacttcgtccaatcagattgggctcaagacagatgtgactgactatcacatcgatcgcgtttccaaccttgttgagtgcctctctgatgagttctgtttggatgagaagaaggagttgtccgagaaggtcaaacatgctaagcgacttagtgatgagcggaacttgcttcaagatcccaactttcgcgggatcaacaggcaactagatcgtggatggaggggaagtacggacatcaccattcaaggaggtgatgatgtgttcaaggagaactatgttacgctcttgggtacctttgatgagtgctatccaacatacctgtaccagaagatagatgaatcgaaactcaaagcctacacaagataccagttgagaggttacatcgaggacagtcaagaccttgagatctacctcatcagatacaacgccaaacatgagacagtcaatgtgcctgggacgggttcactctggccactttcagccccaagtcccatcggcaagtgtgcccatcactcacaccacttctccttggacatagacgttggctgtaccgacctgaacgaagacctcggtgtgtgggtgatcttcaagatcaagactcaagatggccatgccaggctaggcaatctggagtttctagaagagaaaccacttgttggagaagccctcgctagagtgaagagggctgagaagaagtggagggacaagagagagaagttggaatgggaaacaaacattgtgtacaaagaagccaaagaaagcgttgacgctctgtttgtgaactctcagtatgataggctccaagctgataccaacatagctatgattcatgctgcagacaaacgcgttcatagcattcgggaagcttaccttcctgaacttagcgtgattccgggtgtcaatgctgctatctttgaagagttagaagggcgcatcttcactgcattctccttgtatgatgcgaggaatgtcatcaagaatggtgacttcaacaatggcctatcctgctggaatgtgaaagggcacgtagatgtagaagaacagaacaatcaccgctctgtccttgttgttcctgagtgggaagcagaagtttcacaagaagttcgtgtctgtcctggtcgtggctacattcttcgtgttaccgcgtacaaagaaggatacggagaaggttgcgtcaccatacacgagattgagaacaacaccgacgagctgaagttcagcaactgcgtcgaggaggaagtctacccaaacaacaccgtaacttgcaatgactacactgcgactcaagaggagtatgagggtacttacacttctcgcaatcgaggatacgatggagcctatgagagcaactcttctgtacccgctgactatgcatcagcctatgaggagaaggcttacaccgatggacgtagggacaatccttgcgaatctaacagaggctatggggactacacaccgttaccagccggctatgtcaccaaagagttagagtactttccagaaaccgacaaggtttggattgagattggagaaacggaaggaacattcattgttgatagcgtggagttacttctgatggaggaatgagtagttagcttaatcacctagagctcggttacctatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttgcggccgcgcgccgacccagctttcttgtacaaagtggttgcggccgcttaattaaatttaaatgcccgggcgtttaaacgcggccgcttaattaaggccggcctgcagcaaacccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatctccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaggtaccctgagcttgagcttatgagcttatgagcttagagctcggatccactagtaacggccgccagtgtgctggaattcgcccttgactagataggcgcccagatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacatagcggccgggtttctagtcaccggttaggatccgtttaaactcgaggctagcgcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatatgatattcattaattttatattatctaaaagagttaaaagagaaaaaagaaatatgacaatttttttctttcacatcttctaacctaaaagtatgactctatggaggctaagtttagaaaaagatacggatctagggtgtggaaacatcaatggtcaactccttttatatttcaatcaattgggttttgctttatctttacattttctccttttattttccacgtctattcaaatctacttgttagcgggtgattactcttttttcttttatagatgccaattatttctctcctatgtattaaattagagtatattgtcttgaaagtgacttagtattttagtttatagtctcttaaagaacgacaccttttattcttaactctctttatcaagttttaatttaaaattattttaaattaagtatgcatacatatcttaatatttttcttaattatttttaaattccctaaatttaatgttttcatacaatgtaagagatatacatattaattatatttaaagataaaacttactttcctgcaataaaataaagaaaaggacagtcatacaattatataattaatccagaatatttatagcttttaaacatttattttctatcaattaagtaataactttaaataaaattaagagtacttttttatactccaaagaatttatttattttcaacaaaatcgtctgactgtttcaattgatcattatcagcctagcataacctaaatttcattttcaaacataacttttggcaccaaatcacccggcattgcaaaaaagtcttttgcgatatgaccctccacgacgcagaaccactgttattcattaccatcacttttaatcctaatttcccatacacttaccctttccatgacatcttcaaagcctttattttgcttttcttgtttaagctgttttaacctaatttcatgcatataaacaaagagtaaagcaaaggcaaatatttgtacgtatagtttttagacagaaaaggaaagtaaattatagagataatgaagtttgctcttttaaattcgtcgtgatgttatccatcatatctaaatgcttattcctgtttttgtcttttttctcttttaccggagtttattttatataattaattaaagttagtagatctatattctttttcatagataatccatcttctttggaggcacatcgatcattaatcatagagttttgagaagcattatcactaaagcttcaattaattatatccaataaacggtattggtgtatgatgttatgatagcaaatagataatctaatctatacgagccacaaaaggggcatgaactctatctcgaagaaattggagatgaagggattgagattggcaccttgtgctattattgcccactaatcatt

SEQ ID NO: 14 - DNA, 14416 bp, Artificial
  synthetic
gggaaaatccctcttccatattaagaacataaaaatcaacaggaaaaataagttcaccaacccgaaccagcacatcctctatgaaacctgcggggtaagcagcacttctattttccaaatgaatcaccacatctgtagattgcaaaggtccaagagataaagaattgaaaatggacagaggggtgacactaactgatgctcctagatctagcattgtattatcaaatttactgttcccaataatgcaaggtatacagaaagtacctgggtccttacatttctcaggaatgtaaggaacaaatttacctatcaatgctgacacatttctgcccatgctaatcctttcattgcctttgagcttccttttgtgggtgcacaactcctttagaaacttgacacatcttggaatctgcttgatggcatctagcagaggtatgttcacctctactttcctgaaggtctccaagatctccttttctgcttcttccattttttttgtttggaattgctcaaggttggaatggaagagggataagaggctgcggtaagtcagaattactagaagaaggtccacctgcatgaaaatttttgttaggaagctttctcttttgtgcaactatctcatcctctttttcaggtgtagaatgaagcttgacaggttcaggtgcgggtgctgctactggtggaggtacttgaatttggttgtcagacctcaaggtgatgacactcacatttttcggattttgcacagtttgtgaaggcaatttgtcagaattttgggaatgagcttggttcaactgagtagccatccgccccatctgatttgtcagactctgaatgaaggctcttgtctcttgctgaaattgcatattctggatggtcatttgcctcactaactcttctaaggaaggttaaggaggagtctcagttgcttgttgtctttgttgtgactgttgttgttgttgctgctgtattggaggaggaacatatggtttgcttggaccagcaacattctggaaaggagggacagactgttgttgttgtgaaggacttgcccatctcatatttggatgatttctccaacctggattgtatctgttgcttggaagatcataattattttgctattgttggttttgctgttgagggggtctattataaatgtttgcagcataagcttcaggttgttcattgactccagattactgcaaagaaggacaaagatctgtatggtgatctgcagaagaacatataccacagactcttgtaacaggtgcaaatttctgattcatggcaagctgagttactaggttgaccaaggcatcaagttttccctcaagctttttattttcagtagataaagatgaatctgtggccacctcatcgactcctctaaggacaatagcatcatttcttgcactgaattgttgggagttggaagccttcttctcaatcaaattcctagcctcagcaggggtcatatcacgaagagctccaccactggcagcatcaatcatactcctctccatgttgctaagtccctcatagaaatattgaagaaggagttgctcagaaatctggtggtgaggacagcttgcacacaatttcttgaatctttctcagtactcatacaagctctctccactaagttgcctgatgcctgaaatttcttttctgatggcagtggtcctagatgcagggaagaatttctccaagaacaccctcttaaggtcatcccagctgaaaatggacctgggagcaaggtagtagagccaatcttttgtcactacctccagagaatgaggaaaagcctttagaaagatatgatcttcttggacatcagggggcttcatggtggaacaaacaatatggaactccttaagatgcttatgaggatcttcacctagaagaccatgaaacttgggtagcaaatgtattagtccagtcttgagaacatatggaacaccctcatcaggatattgaatgcacaagttttcataagtgaaatcaggtgcagccatctccctaagagtcctctcacgaggtggaggtttagccatgttctcagtatgaaaattagtagttgaatgctcaaaatcagaatattcagaatcaccagaaacaaaatactcagaatgctcaaaatgctcaaaatgcacataatgattaggatgcacactatgcctaactaatctatgaaaggttctatctatttcaggatcgaagggttataaatcacctagattgcccctagtcatgcactatatgtagcaaataatgtgttctcaaacaagcaccaagggagggttaaaactacaactatagtcaaatgatatccaaatgagttgaaattttgtgagcagcaccctaaaatcatgaaaagatagcacaaaaaatttcaaacgaaaattcaaagtctaactatgaaaactacttaagaaaagtttagaaaaataggacaataatacttgaaaaataaaaaaaaacatagtaaacagctgatttttcgagtttgggagactccaaccggctaaaacgggttgccacaatatgagaaatttttttctaccccaaatgccacaatatgagaaagttttgctaaaatctagttcccaaaatttttgtctctctcaaattcaaccacaccaagtgctcctagtatttttcacacaaaaaatcagccaaaaataacaactctaactatcaaaacaaaaacagctaattaaattgcaaaatcagtcgctaattcctagtcactaatcactgttcacagcaaaacaccaactgaatcagtcgctaaacagtcgctaaacaggagacgcaactgaaatgcaaaacagaatgctacacaaaacaaaacaactaaacactattatgaacctttggcccactgctccccgacaacggcgccaaatttgatcgaggtcgtacccgaatcaaataaacattaaaaatgcagtatctaggaagtgatcctaggtcatctcccaacgagcaatggtcaaccaatgttcataatagatagtgataaaacaataacgaattggggggggggggtatttgtttttgtaatttaaacaacaagcaaattttaattagaaaataacagaattaaaacatgttatttccccttgattcataagcaagtctcttatcctaggttaggaggatttatccctaaccagttcaaccacttaatccaaccctaaattaaattactaagcgaaaattaacataaggttgtctttatatgattaagcaacacatacaccaattaatcatgaacaaaatcgatcattaagcatcaacataaattaagcgcaaagataattaatcaagcactaagcatgcatggattagtagcaacaaatacagagtaattggtggagatgaaaaactgatcaatattcaatagtaataacaaaacctcaaagagagttgtgcttgattctcaagagaaaacaacgctggagacttagccttccattaatcagtagaaaacgaaattgtagaaaacgaattttattctatgtgaacaatgtgcatgaacagtaataaaaactggaattgcaaaaccctaaaattattcttctctccaaaaaaactccctaaactaaaaccctggtgctattatataggtcctcagccccaaagcttacaaatctattttcagtccaaacccataaacgaaataaaataaaatctggacaagataagataagattggatgaaataaaatctggacgaaataaaatctggataagataagatttgataaaataaaattgtctgctcttttcaagtccaagcccaattccggattcaagcccaattttttataattcttctgaaattaaattaaaaatacgaaattagtcaagtaggcccaaatgataaaactgcataattaatttgacaattaaggctaatcagtaattaaaatagtgacaaaaagggttaagaaataggagaataatgacacatcacccatatggggagcaattctaaaatgcatttgagttctttaacctgagacacagtgcagtagagtctccaaggattcattgtgccttttattttatatgatggggtcactacattggccttgtcaaagaaactgaatttgggggattaaagaaacacaaaataaaaacaaatgaaactagttaatagaaatgttgcctattgcttcttggaaaaagtccaaccatttgtgatttggataaaattcatattacccacttgtagcttgttcaatcaaacactagatttggataaaatctcactcctagatatacctcaagggataatatgaccaacattagtcatttttagaaagtaaagtggacaaatttgagatttcattccttaatgacattataaacatgtattttttccatgaccctttttcaatgtaagtacaatttatcccttagtttagatactctatatatgcatgttacgtagttgatgaaaacatacctaagttgttgtgtatggttaagtttgcgactacctctgatatcaaactcctcatctccaatctcatacaaaagatacttgtcacttggtacctgaaccttgtcagtttgcagttgtgagtttcttctgaagccacacgcttgtatagtaaccagaagccaggagggagtcctctaaggctctaactcgtattttccgtggaagtacattttttttcttaaagaaaacagagatagtttaccaatgataatatttctttagccaaataggaccatcatagaaaacaaaactcttcttctaagtatttaatgcaactacatatttagggtgcgtttgattcgctaaaaaataagggtctagacaacacaaaaatatttttccaacgtttgattttaaaaatggctgagagacaatacaaaataaagaatgatgaactggacaaaaacctaaaaacttgtaactcactgaatctcatacaactttttgttcagtgtctaaaaaaagtaaaaatacaatattattcctattttttactttgattatctcacaccttctttctactcatttgtttcacttcacctctccagtgggcaccttggtttgtcggcgagagtcgtatggacttttgttgtttcctttttgctcattatttctttcttttcattgttaatttattcaaatgttcccatcatcatcttactccttcttgttatgttttttttctttggccaactccaacgaggccgtgccgcgaccaccatcatcacgaccttatggcggcctcacgccgcaaggccctgcacccagtggcatcaggggtcatgcctccttcttaaaggtgtctctcttttgttatgtcgtcaaagtgttgctaattcacctagaatttttcaatgaatccctttacttgtgggttagtctaggtcgctctgcccggttccaaccctagcccaaaaaaaaatgaaatgggtaggaaaggcgggcctagtttgaattaaaataaatcatgctaagatattgataactgctatgtataggtatattttgggattaaattatataggaattagtaatttttctctcttatttcttcctttttgttcaaataattggaattctaacatcatttaagtttttatgtagaaaatattaaaagttgatgaatttatgatacttagtgaataattagagtagaaaaataaagtaaagcccaaaaaagaaaattggtgatatgaagatacatgcttagcatgccccaggcacgcttagtgtgtgtgtcaaacactgatagtttaaactgaaggcgggaaacgacaatctgatccccatcaagcttgatatcgaattcctgcagcccgggggatccactagttctagagcggccgcgttaactgcaggtcgacggatccccgggtaccgagctcgaattcaaatttattatgtgttttttttccgtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgtttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacctcagtcacacaaagagtaaagaagaacaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccggtggtcgcgtcaactgcatgcaggccatggacaacaacccaaacatcaacgaatgcattccatacaactgcttgagtaacccagaagttgaagtacttggtggagaacgcattgaaaccggttacactcccatcgacatctccttgtccttgacacagtttctgctcagcgagttcgtgccaggtgctgggttcgttctcggactagttgacatcatctggggtatctttggtccatctcaatgggatgcattcctggtgcaaattgagcagttgatcaaccagaggatcgaagagttcgccaggaaccaggccatctctaggttggaaggattgagcaatctctaccaaatctatgcagagagcttcagagagtgggaagccgatcctactaacccagctctccgcgaggaaatgcgtattcaattcaacgacatgaacagcgccttgaccacagctatcccattgttcgcagtccagaactaccaagttcctctcttgtccgtgtacgttcaagcagctaatcttcacctcagcgtgcttcgagacgttagcgtgtttgggcaaaggtggggattcgatgctgcaaccatcaatagccgttacaacgaccttactaggctgattggaaactacaccgaccacgctgttcgttggtacaacactggcttggagcgtgtctggggtcctgattctagagattggattagatacaaccagttcaggagagaattgaccctcacagttttggacattgtgtctctcttcccgaactatgactccagaacctaccctatccgtacagtgtcccaacttaccagagaaatctatactaacccagttcttgagaacttcgacggtagcttccgtggttctgcccaaggtatcgaaggctccatcaggagcccacacttgatggacatcttgaacagcataactatctacaccgatgctcacagaggagagtattactggtctggacaccagatcatggcctctccagttggattcagcgggcccgagtttacctttcctctctatggaactatgggaaacgccgctccacaacaacgtatcgttgctcaactaggtcagggtgtctacagaaccttgtcttccaccttgtacagaagacccttcaatatcggtatcaacaaccagcaactttccgttcttgacggaacagagttcgcctatggaacctcttctaacttgccatccgctgtttacagaaagagcggaaccgttgattccttggacgaaatcccaccacagaacaacaatgtgccacccaggcaaggattctcccacaggttgagccacgtgtccatgttccgttccggattcagcaacagttccgtgagcatcatcagagctcctatgttctcttggatacatcgtagtgctgagttcaacaacatcatcgcatccgatagtattactcaaatccctgcagtgaagggaaactttctcttcaacggttctgtcatttcaggaccaggattcactggtggagacctcgttagactcaacagcagtggaaataacattcagaatagagggtatattgaagttccaattcacttcccatccacatctaccagatatagagttcgtgtgaggtatgcttctgtgacccctattcacctcaacgttaattggggtaattcatccatcttctccaatacagttccagctacagctacctccttggataatctccaatccagcgatttcggttactttgaaagtgccaatgcttttacatcttcactcggtaacatcgtgggtgttagaaactttagtgggactgcaggagtgattatcgacagattcgagttcattccagttactgcaacactcgaggctgagtacaaccttgagagagcccagaaggctgtgaacgccctctttacctccaccaatcagcttggcttgaaaactaacgttactgactatcacattgaccaagtgtccaacttggtcacctaccttagcgatgagttctgcctcgacgagaagcgtgaactctccgagaaagttaaacacgccaagcgtctcagcgacgagaggaatctcttgcaagactccaacttcaaagacatcaacaggcagccagaacgtggttggggtggaagcaccgggatcaccatccaaggaggcgacgatgtgttcaaggagaactacgtcaccctctccggaactttcgacgagtgctaccctacctacttgtaccagaagatcgatgagtccaaactcaaagccttcaccaggtatcaacttagaggctacatcgaagacagccaagaccttgaaatctactcgatcaggtacaatgccaagcacgagaccgtgaatgtcccaggtactggttccctctggccactttctgcccaatctcccattgggaagtgtggagagcctaacagatgcgctccacaccttgagtggaatcctgacttggactgctcctgcagggatggcgagaagtgtgcccaccattctcatcacttctccttggacatcgatgtgggatgtactgacctgaatgaggacctcggagtctgggtcatcttcaagatcaagacccaagacggacacgcaagacttggcaaccttgagtttctcgaagagaaaccattggtcggtgaagctctcgctcgtgtgaagagagcagagaagaagtggagggacaaacgtgagaaactcgaatgggaaactaacatcgtttacaaggaggccaaagagtccgtggatgctttgttcgtgaactcccaatatgatcagttgcaagccgacaccaacatcgccatgatccacgccgcagacaaacgtgtgcacagcattcgtgaggcttacttgcctgagttgtccgtgatccctggtgtgaacgctgccatcttcgaggaacttgagggacgtatctttaccgcattctccttgtacgatgccagaaacgtcatcaagaacggtgacttcaacaatggcctcagctgctggaatgtgaaaggtcatgtggacgtggaggaacagaacaatcagcgttccgtcctggttgtgcctgagtgggaagctgaagtgtcccaagaggttagagtctgtccaggtagaggctacattctccgtgtgaccgcttacaaggagggatacggtgagggttgcgtgaccatccacgagatcgagaacaacaccgacgagcttaagttctccaactgcgtcgaggaagaaatctatcccaacaacaccgttacttgcaacgactacactgtgaatcaggaagagtacggaggtgcctacactagccgtaacagaggttacaacgaagctccttccgttcctgctgactatgcctccgtgtacgaggagaaatcctacacagatggcagacgtgagaacccttgcgagttcaacagaggttacagggactacacaccacttccagttggctatgttaccaaggagcttgagtactttcctgagaccgacaaagtgtggatcgagatcggtgaaaccgagggaaccttcatcgtggacagcgtggagcttctcttgatggaggaataatgagatcccgtcctttgtcttcaattttgagggctttttactgaataagtatgtagtactaaaatgtatgctgtaatagctcatagtgagcgaggaaagtatcgggctatttaactatgacttgagctccatctatgaataaataaatcagcatatgatgcttttgttttgtgtacttcaactgtctgcttagctaatttgatatggttggcacttggcacgtataaatatgctgaagtaatttactctgaagctaaattaactagattagatgagtgtattatatacaaaaggcattaaatcagatacatcttagacaaattgtcacggtctaccagaaaagaaattgcatttgtttttgggtctttcagactgacaagatcgatctgaagtctaaacaattctaagaggtatcatgtagcaatgtcctgccacaatattgaattgacctgcagcccgggcggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatcaaaaagtaggactaatttaggaaagcaagctaattcaagaaagtgaaggcacgcttagtgtgagacacgtgttgagcgcgattactgccactcactaaccacacaagtgcactcagtgcgaaggttgcttaaaaattaagttgattcgcacttataaaagaaggatagagatgaaggaaaaaacacagaaaatacaattccttatagaagacaaaggctagaagaagcaaacgcaaacattagaagtcattccttccctcaattccctttttcaatttccccttttactaaatattctcctcttgcaattataaagcctcctatgacaatgacaagctaaactctcctttgttgggaacttatcagtcaactgctcttaatataatttctcttcctatctattatgaatattcactacaagaaatatgcccatttgccagggatttttgacagggacattaacccctggcaaatttcccagggactaagccaaggaaacccctggcaaaatgacatttgagaaggctgggaccacttacatttacacaggggtttgtccctcgcaaaaatacaaaagccttggcaaaaaaaagagcgggaaatgaattttaaaacagcatgttgttttcacacagccaaacacacgggtatgccctcgttttctgtaaagctgacggaatcttcccataagtcaacacgacatgaccatgcactgcaaaaagctgtgcggcccagacgtgacaggggtgttacccctcggaaatggcttgcagcccctggcaaaaaggaatccctgctttcctagctacaccgttctgctcatatagctgaagctaggaggttagcctttgactctgttgttttgcgaggggcattccgtgagttattccctgggtttttttacactatatagccaaaccgcgtgtttatcctcatgctcagtgttgtgtttttgaaacttagaaaaattttcggtttccatttccatcctcaccagttcattttcagtccattatcattcagttcatacacttgttctataatttggtaacactcttttcacttattatatttttctgtttttatttgttactacttattaacataaatattttttattgtatcagtgtccaaatttgcctcctcctgctgctccttgctctctgaatttgttctcttaagcttcaacaagttagtaatttttctacttataattttagatatatgatgtttatatatatgatgttataattttgcatgatctgtcaaagaaaatatgatgtttctacttgcatgatgtgttataatatatgatgtttatatatatttcgaattttgttgttaataaaactgtttaattagaaactgtataatttttttgtttaataaaactgtttaattttgcatgatctgtttaataaaactgtttatataaaactgtttatatataatatatgatgttaacatttttaaaactgtttataaaacagtttagttagaaaaaatgttaaaactagagaaaaaaatgtataataaaactgtgtcagtacagcagcgcgtcagaaaagtgtgcagatgcgtcagtgagaagacaggggctaagacagggattttgacagggaattttgccagggattttgccagggtcagcccctcgtttttttgccaggggtgaaatccctggcaaactgatttgcgatgggcgtttttcccagggattcagcccctggcaaaatccctggcaaacgtccatttcccagggctttttgttcttttcccagggaatccgcccctggcaaacgagcttgtttcttgtagtgattacttttgcattagtttttcctgtatttaattttattgtttatggcttgattacccatttgcattataagttttaggggtagcgttgaaaagtgttattctctaatagaactggaaaagagtatttaaataacttcatcactagggatacattgattttatttagcttattatatatctctattattaatgtaatttaactattttatctctgcaaagtgatttgggagagaagatagataagttagactctttcactcgaggctgagtacaaccttgagagagcccagaaggctgtgaacgccctctttacctccaccaatcagcttggcttgaaaactaacgttactgactatcacattgaccaagtgtccaacttggtcacctaccttagcgatgagttctgaaggg

SEQ ID NO: 15 - DNA, 5946 bp, Artificial
  synthetic
gtacaaataaaactttatctgtacatttcgttagttaaattatattttgtccatcaaattgttcctttaaattaaaatctaaaaactaactttaccgtaaagaaatatgcattcatgtataccaaataaaattttgacaagagtttaagttttatattatgattcttaaggaaatcattatcattataatttataaaaataaataaattgataattatatgctatatcaatttatgatttgatgagtatgtatgttttaaatgcgagattctgccgccgttcgatatagttagcagtagagccctgttctcaccctcacacctgctcagtgtgaactttaaaagggactttgttgacaaatgttaggatcgtcgtcttcttttgcaataaaaaattttcatctgtttaaaacgtttttatagtaaaattataaatagaaaatttagttgtaaaatttgaaatataaatttaattagaatgcattcacattgtaattcttttacattattatttattacctaattataaattatcaacaataaaatctgacatagtatatgtttagattaaaatttgtaaatgtaagtttgaattaaaaaagtatattataaacttgagtttggtataatattttttatcatcagactttggtataatatgagttgatctaaaagtaagttgaaggataccaaagggtaatctaaacatgcatgagaaatgttggggaatatctttagtgtaaacaaaaagccttataaattatcatgtcatactatatatgacaaatgttggtttttgtaataatcattttaaatatgaacaacaggattttctttttaccgtcaaaatatgaataaaagttaaactcttaaattattgatcataggtttgcaatttttttttatagagaggtttgcaatttctgagttctcaataactgatgattaaatgcgcatcgtttgcatgcatgaaaataaatttaagaggtaacatttgaagtctcaccttcatgtccggggctgccaggaaagcttagatctccatgagcatccacgagcttatccacgagcatccacgagcttatccgatttgagcattgatctccatgagccatttagtctcaccttcaaacactgatagtttaaactgaaggcgggaaacgacaatctgatccccatcaagctagcttctgcaggtcctgctcgagcggccgcagatcttgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgacccaacgagatcttgagccaatcaaagaggagtgatgtagacctaaagcaataatggagccatgacgtaagggcttacgcccatacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacggtcgagaagacacggccataagcttggatcctcgagaattctcaacacaacatatacaaaacaaacgaatctcaagcaatcaagcattctacttctattgcagcaatttaaatcatttcttttaaagcaaaagcaattttctgaaaattttcaccatttacgaacgatagccatggcttctatgatatcctcttccgctgtgacaacagtcagccgtgcctctagggggcaatccgccgcaatggctccattcggcggcctcaaatccatgactggattcccagtgaggaaggtcaacactgacattacttccattacaagcaatggtggaagagtaaagtgcatgcaggtgtggcctccaattggaaagaagaagtttgagactctttcctatttgccaccattgacgagagattcccgggccatggccaccttcgtccgcaatgcctggtatgtggcggcgctgcccgaggaactgtccgaaaagccgctcggccggacgattctcgacacaccgctcgcgctctaccgccagcccgacggtgtggtcgcggcgctgctcgacatctgtccgcaccgcttcgcgccgctgagcgacggcatcctcgtcaacggccatctccaatgcccctatcacgggctggaattcgatggcggcgggcagtgcgtccataacccgcacggcaatggcgcccgcccggcttcgctcaacgtccgctccttcccggtggtggagcgcgacgcgctgatctggatctgtcccggcgatccggcgctggccgatcctggggcgatccccgacttcggctgccgcgtcgatcccgcctatcggaccgtcggcggctatgggcatgtcgactgcaactacaagctgctggtcgacaacctgatggacctcggccacgcccaatatgtccatcgcgccaacgcccagaccgacgccttcgaccggctggagcgcgaggtgatcgtcggcgacggtgagatacaggcgctgatgaagattcccggcggcacgccgagcgtgctgatggccaagttcctgcgcggcgccaatacccccgtcgacgcttggaacgacatccgctggaacaaggtgagcgcgatgctcaacttcatcgcggtggcgccggaaggcaccccgaaggagcagagcatccactcgcgcggtacccatatcctgacccccgagacggaggcgagctgccattatttcttcggctcctcgcgcaatttcggcatcgacgatccggagatggacggcgtgctgcgcagctggcaggctcaggcgctggtcaaggaggacaaggtcgtcgtcgaggcgatcgagcgccgccgcgcctatgtcgaggcgaatggcatccgcccggcgatgctgtcgtgcgacgaagccgcagtccgtgtcagccgcgagatcgagaagcttgagcagctcgaagccgcctgaaccggcttatgctgcacgggcggggcggggcggtttcgatcggctcgcctgtcccggcgatattctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgcggccgctctagaactagtggatccccccctttaagggggctgcaggaattcgatatcaagctttggcgcgccaaatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggactttagctcaaaatgcatgtatttattagcgttctgtcttttcgttaatttgttctcatcataatattgtgacaaaaatatagctaggaaagctttccatgcatattttgtaagcaatgaagtatatagtggatgcaatgtctctatatattcactagtcgagaaaattgcggacagttctgagattgattggcttcatggccctacggtgcatctattaccattgtctttgcatttgtcatacaaaaaggtggaccatattcatgttatgtaaaaacaaacaaaatcacgcagtgcacatcttctgcagaatgtgtaggttaaccttattacacttgattaagttaagtgtcatgccattagtttgagattgaacttaaaatctttaatcaagatcttagatatggaaaaaattgtaattccattaaagataataagatttttggatagaaattaattatcaattttacattaataacataataatttgaagaaaaaaagtaagggtcataatcatactaaccagagtaatttgacacgtgaaggggacactatgaaagcaaattacttttggttcctaaaggttaggcaagggaaagaaagaatttgcacttaattagcactattttcaaaattattatgtttcttttccttatcttgcttaaaatttgcttattgtgttattattattattattgttatgcatgatcaattattcatcaaagatcgatctccaacctgccaggaaatccgctgatttgtttgcttccaatgtgagagatccaagatcagaattctggaaggtagtgctgactaccaaggtagcaaaataatgatattggggaaggtgaaaaatatgtagtactagtacttctactacaaaatttcaaaaagggttttgtgatttgtgcataagaatctttttgcatttgtctgtaagcttgaaaattacacgtggcacaagtcacttgcagccaaagaacctttctgtgaccaattatgttccctgagctgaatagtggttcttattctaatctcatcaatatctaattacctagtgaatatactactagactattgcagtgttattaatatcttaatgatagactattgcagcagacagaaattacaggtattattatatactaatatacaattctgcattttccacacttttcccctgcccatgcttccatgccactgaagtctgaaaccacattggcagattttgctatctagaaattaaataacaatataagtttgtatatttatatttcatatttttttagtacatttttattttgcacactctataattccatgattccttgattatcggagaatgatgtgatatgcaaaccacgagttagaaccatcaaatcaagcaaagatatggatggaatgcctttaatggaaagattaattcaaaggggcagaaactggtaattttttcttcaactgaatgctatgcagtatgcagcagatctttcatttacagaatatctgcaaaaccttgtgttggagatcttacctattgaataatgatataggtaaaataaagtatttaatttcaccataacttttaagatgatgttaaaatgatctatgcaattcatgttggatcgaatattaaagatgtcacatctaatgatactatgataaaaataaagtataatttctgatcttataagtcaaaataaatcatgtaaatataaattaattctcttcttataaattaattttatataattaagatagatccaatgtgaactctaagaccatgcatatataaaaatcattatcaagtgaatatgcaac

SEQ ID NO: 16 - DNA, 6466 bp, Artificial
  synthetic
tgggggctgcctgtacttcccaaaacttcgcttccctgacccatcatatccaggactggacgattggcttgattgataccagatgggtgagtcgagtccacctcggtagcggcatttatggcaacgattgcagccacgttgacctccatcattttttctcatgctcatcatggcctccatcatggtggtcatttggtctttcatggcctccatgtcggccttcatctgctcttgaacttcatctatctcactcatgattctagccttggcacgtgtttggtaagggtaccgtaaagcgcgttcgttcttttttattactatgattacattttgacgatgatgatgattgtaggaaagaatgaaatgagtaatgaaacaactaaataaacgtgaatgcatgacaatgataagttgctgaagtattataaatttacataggacattcagtggaacgtagggtcgaatcaaatcctatttcattaaaaacaatattgttcatcttgacagagccaaagcataactagaaatacaacatggacacatcagcgattcctaattatgtgggtcattagttcgaccatgtgttggcagtaacttgaaagactatgaacttcatcgggagcagagtatgtgtcagtcaccgccttggctctggctaacaaccttgggatctcttggctctcatttagagtaagagcaaatttgtccatccatttcatggcttctttatgcaataactctatcaccccttctcttgcttccctttcaacctgcaaggtcgacacttttgcctgttcgtcttctagccttcgcccatgactagcagctaggttcaccttctcttcatattggtcaatgattatcaacatattttcttttgttttgctcaactgttctctcaaacttctcttcgatctctgacaactctttaacttatcctctaacatcaggttttccatacttgatttgtccctcttggcttttctaagtttgagctcgttactgctgccccacaaagcccctcgaaacttgttcctgctccactcttccttttgggcttttttgtttcccgctctagcgcttcaatcgtggttatcaagctccaaacactgatagtttaaactgaaggcgggaaacgacaatctgatccccatcaagctctagctagagcggccgcgttatcaagcttctgcaggtcctgctcgagtggaagctaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaagaattcgatatcaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccagagatctaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttgatgagctcaagaattcgagctcggtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctcagactctagtgactaccaccttcactctcctcaagcatttcagcctcttccccgctcagactccttagctttgggagccaaattatcccttacgttctcgacttcaaccatatgtgatagctgcctatgataccatggctacttccccttagttctttatctttcctttccgctttattccatgccttaccgatcctctgaagtgtctttgcattagcttcattgaaacctcacgcgatgaaaggtgtgatggtctcctccgatggcgcacttctcatagggtaacctaattgtcttacgaccaacataggattataattaatacaacccctcgtccctataaaagggacatttggaaatccttcacataagcataacactcctacccctctttctttccactgtgggaaccaactaatggacgctcctatcatgcctgccaagagttcttcccaatttgcctcgtcctttcctgagcacatgcgatgaccttgtatggggtagacagatctactttcatgattgaagacgtgggataccaaccacacataaagagcaggcgcacaacagaaaatcctcgtagtgctcttcttgcatcttaagtcaaatgtatcatacacttatgctaaaacaacaatgatcgggctttccttgctatggtgataagcaagaaaagcatcgattgctactagatccaccaactcgtctacattcgaaaatagtactatcccaaacactagcagtgctaatacgtcgatgaatgatgcccactctccttggctggccagagtttccgccttctcctccaatcacttccttggtattccccctaccctattcctactttgcttcactcagtctaattctcatttcgagatcttgacaactcctgctattctcgccatagaaggatagtacccagaaaaaaggtatggcttccttcctcctatcgggcatcctaagatcccttcgaactcctctatggttggtgctaactgaaagtccccaaaagtgaagcatctgagtgattggtcatagtattgggtgagagatgcgatg

SEQ ID NO: 17 - DNA, 9382 bp, Artificial
  synthetic
  misc_feature (556)..(556): n is a, c, g, or t
accaattattatcgtcttttttaattattccaacctttaactattatcctgccttaaaattcgaatacatttattatctataaactatccgaatattattatctaaatcctaattaaatactattttttatcgagtattcgtatccgccaaggaaatccatctccaaattttcaattatttttcagatatctaaatctgtaaaatttcaaattcaagtacgttacaattctttataaataatccaaattataaatattttataactattaattcataaattaaaatttattattcaaatattcgaataatctatttttaagacgtaaagtattacatcgaagggttactttcaaagggtagtgtatttccatttcaattattcagaacgttgtcgttttgttccggtcatagaaaagggctctggaagagaagaaaatgacttgacttttcaatttcatgctcatccactcgtttcaattactgtttactaaaaaaataataaaataaaatattaacaatgcattgagtatgatgtccgggaaatctacatggatnagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggccacttggctgcagccaagctttcgcgagctcgagatccccgacatatgccccggtttcgttgcgactaacatgagttcttggacaaatttgattggacctgatgagatgatccaacccgaggatatagcaaagctcgttcgtgcagcaatggaacggccaaaccgtgcttttgtccccaagaatgaggtgctatgcatgaaggaatctacccgttgatgtccaacagtctcagggttaatgtctatgtatcttaaataatgttgtcggtattttgtaatctcatatagattttcactgtgcgacgcaaaaatattaaataaatattattattatctacgttttgattgagatatctagatctcgaggtgtggaagatatgaatttttttgagaaactagataagattaatgaatatcggtgttttggttttttcttgtggccgtctttgtttatattgagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgtctagggtcgaggggggatccgtcgagggggtccaccaaaaacgtaagcgcttacgtacatggtcgagggggtccaccaaaaacgtaagcgcttacgtacatggtcgagggggtccaccaaaaacgtaagcgcttacgtacatggtcgagggggtccaccaaaaacgtaagcgcttacgtacatggtcgactagagcgtgacgctcgcggtgacgccatttcgccttttcagaaatggataaatagccttgcttcctattatatcttcccaaattaccaatacattacactagcatctgaatttcataaccaatctcgatacaccaaatcgcggatccgtcgacctgcaggtcgacatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgacggatccccgacatatgccccggtttcgttgcgactaacatgagttcttggacaaatttgattggacctgatgagatgatccaacccgaggatatagcaaagctcgttcgtgcagcaatggaacggccaaaccgtgcttttgtccccaagaatgaggtgctatgcatgaaggaatctacccgttgatgtccaacagtctcagggttaatgtctatgtatcttaaataatgttgtcggtattttgtaatctcatatagattttcactgtgcgacgcaaaaatattaaataaatattattattatctacgttttgattgagatatcatcaatattataataaaaatatccattaaacacgatttgatacaaatgacagtcaataatctgatttgaatatttattaattgtaacgaattacataaagatcgaatagaaaatactgcactgcaaatgaaaattaacacatactaataaatgcgtcaaatatctttgccaagatcaagcggagtgagggcctcatatccggtctcagttacaagcacggtatccccgaagcgcgctccaccaatgccctcgacatagatgccgggctcgacgctgaggacattgcctaccttgagcatggtctcagcgccggctttaagctcaatcccatcccaatctgaatatcctatcccgcgcccagtccggtgtaagaacgggtctgtccatccacctctgttgggaattctgatcttggcgcgcatgcggatcctcattcctccatcagaagtaactccacgctatcaacaatgaatgttccttccgtttctccaatctcaatccaaaccttgtcggtttctggaaagtactctaactctttggtgacatagccggctggtaacggtgtgtagtccccatagcctctgttagattcgcaaggattgtccctacgtccatcggtgtaagccttctcctcataggctgatgcatagtcagcgggtacagaagagttgctctcataggctccatcgtatcctcgattgcgagaagtgtaagtaccctcatactcctcttgagtcgcagtgtagtcattgcaagttacggtgttgtttgggtagacttcctcctcgacgcagttgctgaacttcagctcgtcggtgttgttctcaatctcgtgtatggtgacgcaaccttctccgtatccttctttgtacgcggtaacacgaagaatgtagccacgaccaggacagacacgaacttcttgtgaaacttctgcttcccactcaggaacaacaaggacagagcggtgattgttctgttcttctacatctacgtgccctttcacattccagcaggataggccattgttgaagtcaccattcttgatgacattcctcgcatcatacaaggagaatgcagtgaagatgcgcccttctaactcttcaaagatagcagcattgacacccggaatcacgctaagttcaggaaggtaagcttcccgaatgctatgaacgcgtttgtctgcagcatgaatcatagctatgttggtatcagcttggagcctatcatactgagagttcacaaacagagcgtcaacgctttctttggcttctttgtacacaatgtttgtttcccattccaacttctctctcttgtccctccacttcttctcagccctcttcactctagcgagggcttctccaacaagtggtttctcttctagaaactccagattgcctagcctggcatggccatcttgagtcttgatcttgaagatcacccacacaccgaggtcttcgttcaggtcggtacagccaacgtctatgtccaaggagaagtggtgtgagtgatgggcacacttgccgatgggacttggggctgaaagtggccagagtgaacccgtcccaggcacattgactgtctcatgtttggcgttgtatctgatgaggtagatctcaaggtcttgactgtcctcgatgtaacctctcaactggtatcttgtgtaggctttgagtttcgattcatctatcttctggtacaggtatgttggatagcactcatcaaaggtacccaagagcgtaacatagttctccttgaacacatcatcacctccttgaatggtgatgtccgtacttcccctccatccacgatctagttgcctgttgatcccgcgaaagttgggatcttgaagcaagttccgctcatcactaagtcgcttagcatgtttgaccttctcggacaactccttcttctcatccaaacagaactcatcagagaggcactcaacaaggttggaaacgcgatcgatgtgatagtcagtcacatctgtcttgagcccaatctgattggacgaagtgaacagagcattcaccgccttctgtgctctttccaagtcagactctgcctcgagcgttgcagtaacgggaatgaattcgaagcggtcgattatcactccggcggttccggagaaatttctaacacctactatgttacctagggaagaggtgaaggcattggcactttcgaagtaaccgaaatcgctagattggagattatccaaggatgtagctgtcgctggtactgtattggaaaagatggaggaattaccccaattgacgttgaggtgaataggggtaacagaggcataccttacacgaacacgatatctggtagatgtcgatgggaagtgaatgggcacttcaatataccctctattctggatgttgttgccggaagaattcagcctaaccaagtcgcctccagtgaatcctggtcctgaaatgacagaaccattaaagagaaagttccccttgacagctgggatctgagtaatgctatcggatgcaattatgttgttaaactcagcactacgatgtatccaagagaacatcggagctctgatgatactaacgctgctattactaaagcctgaacggaacatggacacatggctaaggcgatggctaaacccttgcctaggtggaacgttgttgttctgtggagggatctcatccaagctatcaactgttccgctctttctgtagacagcggatggcagatttgaggaggttccataggcaaattctgtcccgtcaagcacagacaattgttgattgttgatgccgatgttgaaaggtctcctatatagagtgctggacaaggttctatacacgccctgaccgagttgagcaacaatacgttgttgtggagctgcattgcccatagtcccgtaaagtgggaaagtgaattctggtccagagaacccaacgggtgatgccatgatctgatgccctgaccagtagtaataaccgcggtgcgcatcggtgtagatcgtgatactgttcaatatgtccatcaggtgtggagacctgatgcttctctctatgccctgagccgagcctcgaaagctaccgtcgaagttctcgaggactgggtttgtgtagatttcccgggtcaattgtgacacagtacggattgggtagcgcctagagtcgtagttgggaaagagagcgacaatgtctaggacagttagtgtcaactctcgcctgaactggttgtacctgacccaatctctagaatccggtccccagacacgttcgagacccgtgttgtaccagcgaacagcataatcggtatagttgccaataagcctagtcagatcattataacgactattgatagttgcggcatcaaagccccaccgttgtccgaacacggagacatcgcggagcaccgacaagtgcaggttggcagcctgcacgtacacggataaaagaggaacttggtaattctgaacggcgaagagcggaattgcggtcgtcagcgcgctgttcatgtcattgaattgaatgcgcatctcctctcttaaggcaggattggtcgggtctgcttcccactctcgaaaagattctgcgtaaatctggtaaaggttgctgaggccttctaaccttgagatggcttggttcctagcgaattcttctattctttggttaattaactgctctatctgtacaagaaaggcgtcccattgagagggaccaaagattccccaaatgatatcgacaagtccaagcacgaatccagcaccgggcacgaactctgacaaaaggaattgggtaagtgacaacgagatgtcgataggtgtgtaaccagtctcaatccgttctccacccagcacctcaacctcagggttgctcaggcagttgtaaggaatgcactcgttgatgttgggattgttgtccattgttggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcgcgccttaattaaggaattcctcgagtttaaacggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtagacctcaattgcgagctttctaatttcaaactattcgggcctaacttttggtgtgatgatgctgactggcttacgtgtggaaaaaatttgcaatctatgtagtctttaactaatgtttttttctttaaaaaaaaagtcattatttttggtttgattaatatatttggtttaaattaaataaaatattaaaaagtttagttaaatcatctatttaaacgatttgtactgatttgtgatctattaattttttaacttaatctagaccagggtactagttggtccgatcccatcttgaaaacactatctttagcttgctggtaggttccagggtagaaggcagagacttttttggagggtttttattattaaatttatatttttataatttttaaatgattaaaataaaaatttattattttaagaggagataaagtgcaattttaccatatattaatttaaaattttataaatttaaaaaagaaaaaaactaaaattttaattttataggttctaaaataataaatataacttactgagtttttttaagctt

SEQ ID NO: 18 - DNA, 15490 bp, Artificial
  synthetic
aagcttgcttaaaagtatcacaagccatgatccttataaaaatgatatctgacactatgcttctttgcacattcttcactatgctttctcatttgagcaatggtgggatttgctctcaaatttggtggccctatgtcagtattcaaaaattttcataggtcaaaggcttgaagataagccttcatttttaccacccatatgtagtaaatttcaccgatgaatacaagaggtggaggtggtgagaaactagatgaatacattcttgcagaaaacgtccctcaaagatcaaaatggctctcaataccaattcttggtgttttcagactaagagagaaagcaaaacgagacaatgaacccacaagatgaattaatttcaatacatttttttaaatttcatttcaaacggttacaatatataccttttgttttccaaacaactcaactgatccaactaacatcttggaaataggaaaacttaacttgtacacaaactgattgtgaaatcaacacctaaacacatcaagtcattaccaacttagtttattcctagctaagtatcttaacataaggattgaacaaaaggttaaaccaaaactttgatttttttgctctaggaagacagcttgcacttcatcatacagttcgccttattgttaatcaactcacactttggctgtccgttgatatgctggcagttaaaatgttcaccaatgtgcttagacaatccgccaagcctatcgtgtaaaaactctttgcatgacagttcacctattgacagtccgccagtcttttaatgctcagactgtctaccatgtgacttagagacggaattgtttgccatatgtgttcgccaactagattgttcaccatttgtgttgtttaccatgtgtgttcgctaatgcatggttggccatgtcgcaaaacaaatttttggatgggccaaaattggaattttttcatttgaccaaatataaaaaaacgaattgaacaaattacttttagagggattaaaaattaaaattatactatttatcgaggggagtcaaggctcctatcttcttcgcttcttctattgtttagattaagactaaaattttaaaatttatagaaattaaaattgatgaaattaaaatacaaaattaaatatataattcagttaggtttaaccattttttaatgttgcttagctttaatgtttgggatttggctactttcagtcgttatgcagttatgctcagacaaatttgttctctttctgtcttatcaactactcaaaatctcagtatagttatgtcatttaatctcttcatcgtagatgttatattggtgaaaatggggccaagaaatccacattcaatgactttgaaagaatatatattgttagttgcacattcccttattcaatcacagttgcttgtttctgagtctatagaatcatgatatttgtaaatcttatataaagtaagagtatatggctagacagtctggccctgtcggctgaaggtagggaggaattaatcaatcacagttgcttgtttctgagtctatagaatcatgaattttaaatttatggaatgcattttttcgaagatattgtatgcattaagtgtaattttagtttcaatatgaaatttgagatttatatatatacttacataaaaccctcctttactgaattagtgccatggataaaagaccaattaagcaatccttccaacacgtgcatgcactggattttcatcgcctcgtccattgttaaattgataggttaataagaacaattagttggctactgattatatggattctgggttaaaagtatttaggtttactgttacatacatggaggatctacatctattttcacttttgtttaattaatttaagttagttttgatgagtttaaggattgtactagccaatagtagtacataaaggagatagagtaccaaaacaaagaaaaagccgaaaggtgttaatgctaaattgtaaaagaaagttaaaataagagactcgaattataatatgattctctggcgcactaattaagctactatatattgtcaatagtattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggccacttggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctccttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgacatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgagtcgacggatccccgacatatgccccggtttcgttgcgactaacatgagttcttggacaaatttgattggacctgatgagatgatccaacccgaggatatagcaaagctcgttcgtgcagcaatggaacggccaaaccgtgcttttgtccccaagaatgaggtgctatgcatgaaggaatctacccgttgatgtccaacagtctcagggttaatgtctatgtatcttaaataatgttgtcggtattttgtaatctcatatagattttcactgtgcgacgcaaaaatattaaataaatattattattatctacgttttgattgagatatcatcaatattataataaaaatatccattaaacacgatttgatacaaatgacagtcaataatctgatttgaatatttattaattgtaacgaattacataaagatcgaatagaaaatactgcactgcaaatgaaaattaacacatactaataaatgcgtcaaatatctttgccaagatcaagcggagtgagggcctcatatccggtctcagttacaagcacggtatccccgaagcgcgctccaccaatgccctcgacatagatgccgggctcgacgctgaggacattgcctaccttgagcatggtctcagcgccggctttaagctcaatcccatcccaatctgaatatcctatcccgcgcccagtccggtgtaagaacgggtctgtccatccacctctgttgggaattctgatcttggcggtacccggggatcctcattcctccatcagaagtaactccacgctatcaacaatgaatgttccttccgtttctccaatctcaatccaaaccttgtcggtttctggaaagtactctaactctttggtgacatagccggctggtaacggtgtgtagtccccatagcctctgttagattcgcaaggattgtccctacgtccatcggtgtaagccttctcctcataggctgatgcatagtcagcgggtacagaagagttgctctcataggctccatcgtatcctcgattgcgagaagtgtaagtaccctcatactcctcttgagtcgcagtgtagtcattgcaagttacggtgttgtttgggtagacttcctcctcgacgcagttgctgaacttcagctcgtcggtgttgttctcaatctcgtgtatggtgacgcaaccttctccgtatccttctttgtacgcggtaacacgaagaatgtagccacgaccaggacagacacgaacttcttgtgaaacttctgcttcccactcaggaacaacaaggacagagcggtgattgttctgttcttctacatctacgtgccctttcacattccagcaggataggccattgttgaagtcaccattcttgatgacattcctcgcatcatacaaggagaatgcagtgaagatgcgcccttctaactcttcaaagatagcagcattgacacccggaatcacgctaagttcaggaaggtaagcttcccgaatgctatgaacgcgtttgtctgcagcatgaatcatagctatgttggtatcagcttggagcctatcatactgagagttcacaaacagagcgtcaacgctttctttggcttctttgtacacaatgtttgtttcccattccaacttctctctcttgtccctccacttcttctcagccctcttcactctagcgagggcttctccaacaagtggtttctcttctagaaactccagattgcctagcctggcatggccatcttgagtcttgatcttgaagatcacccacacaccgaggtcttcgttcaggtcggtacagccaacgtctatgtccaaggagaagtggtgtgagtgatgggcacacttgccgatgggacttggggctgaaagtggccagagtgaacccgtcccaggcacattgactgtctcatgtttggcgttgtatctgatgaggtagatctcaaggtcttgactgtcctcgatgtaacctctcaactggtatcttgtgtaggctttgagtttcgattcatctatcttctggtacaggtatgttggatagcactcatcaaaggtacccaagagcgtaacatagttctccttgaacacatcatcacctccttgaatggtgatgtccgtacttcccctccatccacgatctagttgcctgttgatcccgcgaaagttgggatcttgaagcaagttccgctcatcactaagtcgcttagcatgtttgaccttctcggacaactccttcttctcatccaaacagaactcatcagagaggcactcaacaaggttggaaacgcgatcgatgtgatagtcagtcacatctgtcttgagcccaatctgattggacgaagtgaacagagcattcaccgccttctgtgctctttccaagtcagactctgcctcgagtgtggcagtaactggaatcaactcaaacctgtcaatgtacacttcgttgcctgaactgaaggtatcagcacctactgtgaaactgctctggctcattggaaaggtgaacgcggtgttgatagtggcgtaggagaaagattggaatgtaagtggatcaccggtatccattgtcttgttgaactgaccagcaaagatccgttcacctgcaaccgtaacgtagattcttagattggtagtagaggcatagcgtatcctggcacgatacctttggggaagttgcccattgatgttgacaatggtgtacgcgaatggtcctccactagtgcgtcgaagaatgtctcctcccgtgaaccccggccctcttacaactgtagttcctgactgaagtgtgtgtgccttcaccaagggaatctgagtgattctctctggatcaatggtgtttgtgggggtagcgctacgatgcgtccaagagaacattggtgctctccatgagtcggaacctgagatctcacctggccagcgcacaaaggtaacatgattcagcacatgggagtagtcattccaaggtgcgccgctgttgtcttgaggtggtatctcatctagagagtcaatggtcccggagttcctgaatgtgcgggtgtgattcgtaccagtttgttgaaaggccactcccctaagaccgagtacatagtgaggattgccaaagcctcctcggacgaagacaggatctgacaaggtccgatagaaaggacgtggatcttcatctgcaatccagatggcgcccccgggattgaagaccccgtaactaggaaagttgatacgattgccagccgtgttgcgtgagctaactaagtgtcctccccacacagtttgggatctaacagtctctgcagtcacaaacaaagagttcatgaagtccatgagatggggtggtctgactccaaactcagccctgttgaaaccattgggtatgttcgcagaaactggagagtcttcaatgactgaactggtgtagatctcccttgtaagttgggatgacgtttgaatcggataggtacgaacatcgtagttcggaaagagagcaactatgtctaacacagtaagtgtaaggtctctcctgaactgattgaacctggcccattggcgagtgttagtacctctcaggttctccaatccctgattgtaggtatccaaacaatgtttcgtgtatcgatgaatcagattgatgagtctgttgtagtgattgttgacagtagctatgtccagtccccaaccttgcccaaacgacacagcgtcgcgcagtagtgacaagtgcaggttagcagcttgaacatagaccgagagaagagggatctcgaagctggtaagggtgaagttgttgatggctgtgatcaaagcatcatctgtgttagcaaagcgtatacgcacatcttctctcagttgggcattgttaggattggcttcccactctcttagtgcttcaatgtagatctcatagctgtctgctaagccacgaagggtagtgatggcccgattcctttccaaggtctcaatcctttgttcaatcaactgttcaatctggagaagaaagaggctccaatcagatggagtgatgaagccccagatgaggtcgaagaggccaaacgcaactcccacacctggaacaaactcagacaacaggagacgtgtaagggacagggagatgtctaacggcaatctgccagtcgacctctcttcgttgagaatctctacttcaggattgttgaggcagttgtaggggacgcactgattctgtatgttgttctccattgttggatccgcgatttggtgtatcgagattggttatgaaattcagatgctagtgtaatgtattggtaatttgggaagatataataggaagcaaggctatttatccatttctgaaaaggcgaaatggcgtcaccgcgagcgtcacgctctagtcgaccatgtacgtaagcgcttacgtttttggtggaccccctcgaccatgtacgtaagcgcttacgtttttggtggaccccctcgaccatgtacgtaagcgcttacgtttttggtggaccccctcgaccatgtacgtaagcgcttacgtttttggtggaccccctcgacggatcccccctcgaccctagacgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcaatataaacaaagacggccacaagaaaaaaccaaaacaccgatattcattaatcttatctagtttctcaaaaaaattcatatcttccacaccctcgagatctagatatcgatgaattttggcgcgccttaattaaggaattcctcgagtttaaacggatccctgaaagcgacgttggatgttaacatctgcaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtagacctcaattgcgagctttctaatttcaaactattcgagctttctaattgaatatatccagggcccagcgtaagcaataccagccacaacaccctcaacctcagcaaccaaccaagggtatctatcttgcaacctctctagatcatcaatccactcttgtggtgtttgtggctctgtcctaaagttcactgtagacgtctcaatgtaatggttaacgatatcacaaaccgcggccatatcagctgctgtagctggcctaatctcaactggtctcctctccggagacatgtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagaggggggggggggggggaggacgagcggctgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgcttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagagatgcaatgctcattatctctagagaggggcacgaccgggtcacgctgcactgcagccaagtggcccccatttggacgtgaatgtagacacgtcgaaataaagatttccgaattagaataatttgcttattgctttcgcctataaatacgacggatcgtaatttgtcgctttatcagaatgtactttcattttataataacgctgcggacatctacatttttgaattgaaaaaaaattggtaattactctttctttttctccatattgaccatcatactcattgctgatccatgtagatttcccttacttgtctccctctaatctgactttattaacccaaagcaattgcttatttgttccccacgcccacaaagcccagcattgtccctaaggtattaatttgttgttcgattcttgttcttgaacccatttggagaatgcaacaagggttttcatgtcagcacggtaatggttctgtgtaaattccagtagtgctgcccaagtaaagtctgggtatttcctcgaatttgcggcattaactaagctagctgctggtgtcaccggtctctccagctcgggacatagaaagaatgcaagtgattttctcaccgtttcagtattcactactgcccaagcagctcttgtagataccgtttgtcaaagcctgtattcaaacaccacaacctcattttcgttaaaattttttgtatatacgtatgcatatatgttcagaatgtttattaccatgaatgtatcgcaatgttgacaacgaaagctcctgggatatgagcaacggagtgccacttttcatcggcaaacacttgaaggcctccaacttgatcctgatgaaggattgtcaaggaagtgggatcggtgtggggaccggttcccagggtcaactcaggcttttcgcatggagagtagtgattcagtctcaggattgaatcattttgttcgaaaaagtctttgaagtaggcttggtcaagccctagacttatccctagcagccccattatctctttggaaaccttgttcctggcttcacaatattcctggtaaagcctcctgtcaagtacgaaaagcaaaatcgagtgttagattcctatcctatggtaaaacttgtcgcaacattctacaaattaacataggttataaaagagaaacgcaaagtcaaaaaaggccatccagttataatgtgtattttttgttttaggagaaggggtatttaagcaatccatgttgaatggacttagggttcggtcaatgcaaggaatgatgtctattgaatttgtcaccccatctctttcaaacctaaaaaagatgtatatccaattgtcactatttgtacaaggcttgaaatacacatggggttaacaaatacgctgaaactgcaacatgtattttttgcattttggagactaaatcttgacgtaaaaataagctgatcatctcgtatattgactgaatgcaaattagcatttctcctatttaattaggaatggaagggtgagaagaatttatttacccaaaatctctaaaatcttcagtgccgaaaagacagtgtttctttccatggcaacttggaggaaaacctgccaacgaaactgcttgcatacccatagctttctcctacttttctcttagccttttgcttctcagagagttgcaggctgaagaagcggtccatgtactgatgagcttacaaccaaaaaaaacccatgcttcttgcatgcctcattcaccgcccctgccacttttgatacagcaagagagcccccaagcaagaaagctcccaagtcaatggcttgaattacaagttccgggtcatccaggcatggcttatcgtcgtcaggccatatgaactgagagggtatattggattcagatccaaggattgatgcatcgaaagctaaagggcgatgctcattctttgcgacagcagcagttggcggaagcatcggcttctcttgaatgacagaaactattggcagacaatgtaccattttctgtttcttgttttaggtggtcggagaggaaaaagaagacaagatatagaggagcaaaactaagagggtttaaataagagaagtagaaaaccaataatatttggaatctaattgtttgttttgaactgttgcctcatttcccctttaatttgtgttgtgttggcagcttaacgcttcctttgggttcgattgcaatatagtgcgttgaaatttttaccttgattttcaatctcactacaccacagtgacttgcttggttgtttggataatttttaccttgattttcaatctcactacaccacagtgacttgcttggttgtttggataatttttaccttgattttcaatctcactacaccacagtgacttgcctggttgtttggatatcttggtttccagcacagtgagacaagcctgctgcactcgacaaccttatcttcactggtgctaaaagctccagtcagctctagctagggcaataggtatttttggatgacaaaaatacccatactttaatttattattttaccattttatccttagatgttaaactaatttctttcccaatcttatttgttttcagatttgggaataaaacaatagtttctcccttgttcttgctggtttctcccttgttcttgctggtttctcccctcttttttgctttgttcttggttgtgggagcttaggatattctttttcttcaatcagactaacaagttagagatatcttgtgttttttcactttattttctcatgctcaacatttaccctttttctcaattaacaggggaagtctacattaattagtgcactctcagatagtaatctgtatagtgatgcaatgtatatatattctttaaaacagtttttcctcgaagttaaattctttgttaaaagtaaaaggctggatgtttttacctaattggaggtaatgtctttgtgtagattgtttgcaacattggatggttgattaaaaagtgttgttcttccttcaaggtgagatggtttgctgtcactcactattattattgttgttattgttattgcttttccattgaatagcctggttctaaatgatatacttaccttatcccataggcagcaacattttatcttttgatctttggacctatcatttagcatgctttacactctatttagtaatattattactaactataattttaagtcatacacaatgaaattacctaaacatctaaactcaaaaaattataacttaaagctt

SEQ ID NO: 19 - DNA, 9356 bp, Artificial
  synthetic
ctatagggcacgcgtggtcgacggcccgggctggtgtcgaaactactttgtaatatacaaccaccttttcagttaaattgcatccctaattctagccatgccatgcatttagatattacctgaatatttcaatcaaaatccatttccaaatcatgtaagtaccagcacacaaacaattccaactaagttcattgatgagctccactcaactattttaaagaaaatctaccccaatccttactgatgagtgaaagcacctagcagtgtgaaaagaaaaccaaatatgcattgatccatggacagactaatatgcaacaccttagcactagataaaatgcaagacttttcactctaaatatgaccatgttcttctagttaaaattgatgttaattgaacccagtgtctcttactttcgattctattagaaaacacacaacaatgccatacaaactgcatttttccttgaaaaaagaaaatcaaacagcaattgtataaggaaagtggccttaaatatatattaactgaagattaaatgaaaacagccaagtgttcaagtaattggaaacagctattccctgaccttaaatatataaaaaaactgtagattaaaggatatcaacctcatttaacactcaagatcaaacttaccagtaaacagagagtaggcttcccctaacatacctatatcttgacagttcagaaaattacagcataactttttcacattgtcctaatcaaatttctaaatacatcaaactttggcaacttagaacaaacctaataaactgctccaacttgggcatggacagcaaatgtagatatggacaactttgacccaaaattcaaagataaaggtccaaaagtggaaccactactagggtcttttagtcgtaagtgtggagctgccttatcctaagtttcccaaacccttttatgcttcatttgaggttagaatctcgggaaggcaggtcttttacaagcgttagcacaatttagttgcatcattgttggtgccaaaccatttttttctcaaccaacctattcttcctctctgttttaaggtactattcacagaagaagataggtagtttttaaggagaattactatccaacattagcaaatagaaacccaactatctgctggcttcaaaatgtagcgacagactaataccaaacaaaaccatgagattgtagagagataccttgggtttgatatgaatggccgacgtcctcaaaagagaaatcttcgttttctacataattaacaatgccaaagcaaaagatgagtaatttggattttttgaaaaataaaccaataatacaattcaaatatgaaactttgaaagaaaacactcattgtaagatcaaaaaaggcaaatattcaggtaaacaaattgacgcttagacaacttaataacacattgcggacgtttttaatgtacgccatgctggccgcccggggtacccaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcggggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaatgccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatcttattgcccccctagagtcgagatccacctgaaataaaacaatagaacaagtagaaaccaatcagcgaacatataccaaatcaaaagccgtaagagaaatcaaaacaacaccaaagagaaacggatctaaacataagaaacctaaaacagagagaatcgaacaaagaaaacacaaaaattgaatagatcgtccttgaaaatcctaatttcacaatcaagcaagaaattacacagatgtaaacactacgaatcgatatcttagtaatcaggacaaaatttagaagctggattgacgaaacgaacaatattgtcaaaagcaatttatacaaaagattcaataatccacataacaaaaattggagatcagatacgaatcaaaaacaaaaagaatcagaaaatataccttgaaagagagagtcgcgagagatttgcagagatcgctttaggctttgggagagattgaagagtcagaaaaagacgaaaggatgaattattatcttccacacgaaggtcttctttatatcgcaaaccaaaagcccaaaaccgtcttttctattaatgagaataaaatatctttagccaaaacaaaaaaaggaagatatcagttgaggattattatcacgaaactaaaggaaggaatcatatgatacgtgtcatattttccaccgtgcgtttttaaaagaccgactcaagtagaaacatcctatggtggtggttggattaggtcatccattacatctgcttcactgacatttttctatttttctttttgtatatacttttcctcaaataatttctttcttttctatagaagaatttaatcaataaggaaaaagttcaaaaaagattctttccattaagactatgtcttggttaacccaacccattaagaataagcaatcataatatatatagagaatactaatactatatatgagatttttcttttaatttcatgttgattatgatagtttatcttcttgatttaatttatcaatacttggcataaaagattctaatctactctaataaagaaaagaaaaaaaagtatctaccattgactaattaaaataaggaaacttatctaccaaatttgagtattttttagaacaatctttttggtttaattccaaaactctaaacctaattgttgggaaaaaggacctaatttttaagaaaagttaataattagaagatctgtatgtttttttttgatccaagtttttatttcttttctctttttttcatgataaaatctatgtttttttagtctacaattaaagtaattgttattattttctttatctttttttgttgttgttgttaattcccttttttttttttaacagcaacttcttaaaaaaaaaaacagttgggccttgaatttatttcaggcctgcgttattaagcccagataataactcaaaacaaaaaaaatgttgaaccggaataaacccgcgagattaaatgccggttttcaggtaacatagaagaagaatatatgaggattgaagaagtattcaagaggcggaacaattcacaagtccaagagcttaaatttctcctcactcttctgctacagactcggaactctttctctttgctaaaataagatgttcaggatttttgttgcccgacaattcatgtatctcacactctctctcttctctgttcttactactctgttacattaccaccaactcaagactttcttccacaatggcgtttatgagacttggctccaaatccggtaccggagctcgaattcgaagcttgcatgcctgcagtgatcaccatggtcgacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaaaggatccaccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgatgaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtggaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggccgcggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggagctctagatccccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgggtaccgagctcgaattcggcgcgcccaattgatttaaatggccgctgcggccaattcctgcagcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttaataaatatgggcaatctttccctacaccgactgtactgttactgtaatagactccggcctagactgattctgaattctgtctgtttactgactgttactctagtaaggggattacacactgagttttagtaaactcaccccgtttattaactgtgcaggtaatccccaacattaggtggatcggtgtcacagaaggactcggagacgaccacacaactgcacatgtttttttatttcgtttatttagtcaagcactttggtttttgatttgggttgtattaaggcctctttattttcttaaccttttatttgggaaatttatttagtatgcttaatatatgttagaagtagggcacggttttccaaaacaacaattggctttcaaaatatctcgtttccgtaactgtttaaaagtatgcttctgcagcaaataaggttttaagggaattaacgtttcacaagttttaaatggctagaggttttgagtagtaagaa

SEQ ID NO: 20 - DNA, 6369 bp, Artificial
  synthetic
cattatataaaaagaaaaattttaaatataaaaaaaatatattaaaacagtgaatcaacaatcatgtctcttaaaatgaaaaaaattatcatttaaaatttttaattaataaaagtaaaatgaaattaaaacattttacctgactttaacagtatagataattcaaaagtaaatatgatttcttctgatttttcaaaacaaatttagagattataaatgccattcgtcgatcattttgacacgaaagatttaagaaaagcaaatgtaacctttgacatgatgagcataatcacttcaaatagtatttttttttatttccaacgtcaatatattgaaatatttttttatagaataaactatagcaatttttgccatgatggttcaacttataccactatccttaaccaatatatttttgtttagacattaggagttaagtctgatgtcggatgttttgttccttatttattcaggtgttttataattttacttatgaaagaggttggtttttctcaatgcatttctaatatcatatatacacattgattgagcaaaaaaaaaaaaaactatcctcaaccaatgtagaagtgagttggttgcatgatcttttatttgaattttcaatattgaaaaaactaaaaccacatgtcttaatccatcgtaataatactgctattatttgaaaagttgatagcaaatattacaatagaaaatgtagatctataagaagaaagtatagctatgtgaaatctcatataactaatggtatgattaatgttgattatattcaaagtgttgataatctcacatattctttgacaaaagctttagttagagaaaaaaatatggatagtaataatgaggataagactaaagcctgaacaataataaaaatcaataataaggatactttacataatgattagagatcccaaaaattagattcaaaaggaaaaatgaatacatatttaaaaaatctctcttttcggtataatgaatgtatgtataacaataatagaagttgagtataactatactcttaatgaggtccataactagtttgagtgaaatgcagagttacaagaacatccttgataaccttatttatataaaaattaggacatattctcttaaggtagccaaagcccgggcttaattaaggcgcgccggccaagtcggccgcggccgcgttaacaagcttctgcagaattcgtcaacgagatcttgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgacccaacgagatcttgagccaatcaaagaggagtgatgtagacctaaagcaataatggagccatgacgtaagggcttacgcccatacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacggtcgagaagacacggccattctagttctcaacacaacatatacaaaacaaacgaatctcaagcaatcaagcattctacttctattgcagcaatttaaatcatttcttttaaagcaaaagcaattttctgaaaattttcaccatttacgaacgatagccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtgaggatccactagtaacggccgccagtgtgctggaattcgcccttgaattcaggcctgatcacctgtcgtacagtatttctacatttgatgtgtgatttgtgaagaacatcaaacaaaacaagcactggctttaatatgatgataagtattatggtaattaattaattggcaaaaacaacaatgaagctaaaattttatttattgagccttgcggttaatttcttgtgatgatctttttttttattttctaattatatatagtttcctttgctttgaaatgctaaaggtttgagagagttatgctctttttttcttcctctttcttttttaactttatcatacaaattttgaataaaaatgtgagtacattgagctctctgcaggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggcctcagaaaaatttgctacattgtttcacaaacttcaaatattattcatttatttgtcagctttcaaactctttgtttcttgtttgttgattgagaataggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagaattgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcctcgagtggaggcctcatcctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccttaattcaatattggctctagaacataacttgtttaacactaaatataagtttatgccattgacatatgtataatgcataactttctatcttcacttgaaaagagagatttactttcttcaaaatgtttgtgtattaatttagaagatttaaaccacgagacttgaagattttaagatatcgatagattcaagtacactttcgcatatattttatgttcttaatgatttgacatgataaaactcggtttatttagttttatattataattttatgattgtaacacttaattgataatgacatgatcaactattaatttgacaaaatcaaaagaaatagaaaccgacttttcattttatgtttagaatgaaaaattcagtgtctaatttagatgagtcaaatatttattcataattaggtcaggaatatacttaattacttgggtaaccaaaattggagtttgattttcaataattagatcaagttagtgttttaattacttataagaaaattttaatacccaaattactaatcaaatctattagaataaaattagatgaagatagaaaaattagctaatatatttaatgaaaatttaattctaggtatttactattgattgattattctttttgttagagttatgtgacctgaatcccgtttgatctgatctgaaaagttcgacgtacaattcatttgtcaaataaataaaataaagaggcaaaataggagatctgtataggactatatttcttcaattaaacattgattctaaaagggttgtttaatccttaaaatgcgtgtagtcgaaaacctcttgtattgtgattttttaatattagtgaatttctcctcttctgtccgtgatatttcccgataagggtttttcaaataaaacttgtgtattcttatttttctttcttattgttttacgatccttcctattgccattatcgaagtatagtataacacttttaaatcttttaaaaattatattatcaattatttttataagttaaaagaatataagttacaaataataatttttataccatttatcacgaaatatatacttgcgggtacgatatttgtccacaaacccttaacctcgtgttgagttgtgaacttcagctatataa

SEQ ID NO: 21 - DNA, 6126 bp, Artificial
  synthetic
ttatctatctttttttgtaggtcctaataaacgggctacagcactttgtgccaacaaaggtgaagccaaagttacagatagtcgaaacatttatcaaggtaagcaaaccagaaactcatatgaaagtatagcagacttgagatcataatatgctggtgatacacacttaaaaatcggaatcatcactcattttttttgcaggcatactatctgccagagacggaatatgtccactgggcaagagctcatccggtaaacaaacaaatctttcttaatctttcttaatctttataatgttttgcgtaaattaaatcgatgggatagaagactaatatgattaaaatgtgtaaacatacaggaatatacgaaagcacaggtcattggacttgtgaatttagtagccaccatgaaaagctggaagaggaaaacgcgtctagaagttgtggataagattgaatcagctgctgcatagatcaaatctaaaagcaacaacaaagtaatttttttacttcttttctctggtcttgttctgtctttgcttttggctcttacttttgcgttttgaaccgagtgtgtaaatttgaggataagcccttcttagttatcatctttcttttgcttaatggggtttgtgtaaaagatcctcctcaagttgtacagtcttgaagagattgtaacacacggtttcctacatttaaatacttaattaatgtctcagtatttgtattatcagttccttgaaccttattttatagtgcacaaaaccttttagtcatcatgttgtaccacttcaaacactgatagtttaaactgaaggcgggaaacgacaatctgatccccatcaagctctagctagagcggccgcgttatcaagcttctgcaggtcctgctcgagtggaagctaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaagaattcgatatcaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccagagatctaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttgatgagctcaagaattcgagctcggtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaccatcatactcaacttcaattttttttaatgtcattatgatagatgaataattccttttcttatcctgttgaccataataatgataaaacagtaatcatgataaatatatcaaaaagtgtattttaaaaattttctaatcattattcgagaaaaaaaacaaatcatttgtaagtttcactgttaactacagatgaaacatcttttgtttttaacgttttaatgatattgaaatcaatcagaataaaaggtgtctcatctcttgtgtactgtcatgtttgcgatgagaggctggaagaaagactagtcaaaagacttcaaagctgtggtgatttagttgtatctccaaccatttttaatgcaacgcatggttcatctactggtacctgttgcacaataaaacattcaaaaacatgttattttacaaatcttcactaaaagccttcaatttcatatgatgcgtgtcaatgtaaaccgacttcttattttcaaactgttgatgtgaaagagagaaaaaaatcagagaagtaaaatttatgaaggagatttactaagaagtaaattgttttataaataattatttttatataaaataagatttattattattttttcatgttaatgttaaaagacctttaaaaaatatgcattacgtttttataacagtagagatgtttttaagaattgatattaggtagctttttaaaattaatttagtaattatctaaaaaaaatgatctcttttataaaaagaaaaaatcacagagatccagatactgtcgtgtgaagtattaaagagatgtctttaaaaagtattaaagagacagtcgacagtttgctatctgttgtaataaataatagaaaaataaagaaactgcagcaggaagataaagaaaacatgagagacata

SEQ ID NO: 22 - DNA, 8557 bp, Artificial
  synthetic
actggtatttaatatactttaataaatattattagattcctcgtcaaagaactttttacaatatatctatttagaatcatatatgtcatagttttttttctaagagtctagtttactagtaaaatccgactcacatttttcgaacttgggatgcaacacttaaatagtacaaaaccttggtatgcagtattttacattgtaagattcaaaatttctaaagcagtatatatatgtttccagaaacttatagatatagaaaaaacagagagacgtatgcgaaaattcgataaaggtgtacattggattcgcaaggctaaatacatatttatcgtggatccatgcagagtttgggtaataaaattagatacttccaatcatgtgccacataatcacgtaacattagtaatttaaatgacattaccatgtccaactgatttaaaacacaaactcttcttgaaccatatagtttgacaaaccaaatatatataactggagctactagttatgaatcaattaaaaattactttgaagattcaacgtagtgccagtttggctctagcacatctaaccagaagggctaaggctggcttcaacaggaacagccaaatccgagatcgagccatttgccatttttgggtagttagtttaactttcatatatcttcccatccttttttgcctagcctaaatggctttgatgttgaagaccatattaatttgcttcagtggcactaggacaaccatattggctttggctgacccgttagagttagcctaatgggtggaaggggagggaaggggaggatcgatggtggcatgagagaggggttgacgatcacgatgatgatgcgagtgaggaggagagggtggcgacgacacaggggagaaaggagagggacgctaggagcgtcaagggcgtgggggaggggagggtcggagggatgaaggatgacctaaatattattgttgagtgatagagggttattcaactatccgacccgtcgattttgatggtatgttaaatttgtgtgtcatttgtttgatggatttagtaaaggttatgggtctagaggtgatttttgttgggtgggttttacagagtttaaactagcggattatatagtggtatagaagatatagttttattagaacatctccaaaatgtgactcgaaataatacccccaaaatttaaaatactacatcattttgataaaaaaggtaaagtagagcactgttggaacagtttttaaaagttgtgccctatattttaaaatagggtactgatttaaaatattgttgtgggggatagatatccccgggtccactagaaggcgagaaggcctcgcgtgtggccacgggccagttaccccgcaaggccatcccttcgtgggtcgagctagaattactggtagaatgggctgaccgaagaaggcaacagactcgagcccaaacaatccatcggctcgtgcgctatccacagaaactacccgactttccggcgcatggcatcctagaatatcggggcgtattagggatgagtcagcgagattttcggaagattagttcagtttgttcgctattatttaggagacatatgatcctcatgtacgtatggagtgccccacggtcgtgtatataaggtccagagggtaccccatcatttctatcgaccatctacctatctcatcagcttttctccattcaggagacctcgcttgtaacccaccacatatagatccatcccaagaagtagtgtattacgcctctctaagcggcccaaacttgcagaaaaccgcctatccctctctcgtgcgtccagcacgaaccattgagttacaatcaacagcaccgtaccttgaagcggaatacaatgaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtggccaccgcttaattaaggcgcgccatgcccgggcaagcggccgcttaattaaatttaaatgtttaaactaggaaatccaagcttgcatgcctgcagatccccggggatcctctagagtcgacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacccccggggtcgaccatggctcatgctgccctcagccctctctcccaacgctttgagagaatagctgtccagccactcactggtgtccttggtgctgagatcactggagtggacttgagggaaccacttgatgacagcacctggaatgagatattggatgccttccacacttaccaagtcatctactttcctggccaagcaatcaccaatgagcagcacattgcattctcaagaaggtttggaccagttgatccagtgcctcttctcaagagcattgaaggctatccagaggttcagatgatccgcagagaagccaatgagtctggaagggtgattggtgatgactggcacacagactccactttccttgatgcacctccagctgctgttgtgatgagggccatagatgttcctgagcatggcggagacactgggttcctttcaatgtacacagcttgggagaccttgtctccaaccatgcaagccaccatcgaagggctcaacgttgtgcactctgccacacgtgtgttcggttccctctaccaagcacagaaccgtcgcttcagcaacacctcagtcaaggtgatggatgttgatgctggtgacagagagacagtccatcccttggttgtgactcatcctggctctggaaggaaaggcctttatgtgaatcaagtctactgtcagagaattgagggcatgacagatgcagaatcaaagccattgcttcagttcctctatgagcatgccaccagatttgacttcacttgccgtgtgaggtggaagaaagaccaagtccttgtctgggacaacttgtgcaccatgcaccgtgctgttcctgactatgctggcaagttcagatacttgactcgcaccacagttggtggagttaggcctgcccgctgagtagttagcttaatcacctagagctcgtttaaactgagggcactgaagtcgcttgacgtgctgaattgtttgtgatgttggtggcgtattttgtttaaataagtaagcatggctgtgattttatcatatgatcgatctttggggttttatttaacacattgtaaaatgtgtatctattaataactcaatgtataagatgtgttcattcttcggttgccatagatctgcttatttgacctgtgatgttttgactccaaaaaccaaaatcacaactcaataaactcatggaatatgtccacctgtttcttgaagagttcatctaccattccagttggcatttatcagtgttgcagcggcgctgtgctttgtaacataacaattgttacggcatatatccaatagcggccggcctcctgcagggtttaaacttgccgtggcctattttcagaagaagttcccaatagtagtccaaaatttttgtaacgaagggagcataatagttacatgcaaaggaaaactgccattctttagaggggatgcttgtttaagaacaaaaaatatatcactttcttttgttccaagtcattgcgtatttttttaaaaatatttgttccttcgtatatttcgagcttcaatcactttatggttctttgtattctggctttgctgtaaatcgtagctaaccttcttcctagcagaaattattaatacttgggatatttttttagaatcaagtaaattacatattaccaccacatcgagctgcttttaaattcatattacagccatataggcttgattcattttgcaaaatttccaggatattgacaacgttaacttaataatatcttgaaatattaaagctattatgattaggggtgcaaatggaccgagttggttcggtttatatcaaaatcaaaccaaaccaactatatcggtttggattggttcggttttgccgggttttcagcattttctggttttttttttgttagatgaatattattttaatcttactttgtcaaatttttgataagtaaatatatgtgttagtaaaaattaattttttttacaaacatatgatctattaaaatattcttataggagaattttcttaataacacatgatatttatttattttagtcgtttgactaatttttcgttgatgtacactttcaaagttaaccaaatttagtaattaagtataaaaatcaatatgatacctaaataatgatatgttctatttaattttaaattatcgaaatttcacttcaaattcgaaaaagatatataagaattttgatagattttgacatatgaatatggaagaacaaagagattgacgcattttagtaacacttgataagaaagtgatcgtacaaccaattatttaaagttaataaaaatggagcacttcatatttaacgaaatattacatgccagaagagtcgcaaatatttctagatattttttaaagaaaattctataaaaagtcttaaaggcatatatataaaaactatatatttatattttttacccaaaagcaccgcaaggggtagccctgggtgtgcggacggactctaaacaccgacagctggcgcgccaggtagggggtgtgtctttgatctgagctagctcaatgaccattacctccaaatgcaagatcgcccttcgccccgggactatgttttgctttggaaccatctcatccatagcagatgaagagggaactctgcaccgcatagcagatctattggagaagaagctttcctcagaaatctcgaggggagccagggcagaacagcgggtggcaccatcacccgcacctcaagcgaagatgacctcttacaaaccgaaagtcgggagctcacctacccgaaaaactccgctgtccacttcgcccacaaaggagtggacacggattactcgaaagaaggaagcgagtgtcccgagtcaggggacgggaacacgccaagccatctttccgacgccttcgccctcaaatgaggatggaaagaagagcgccatcgcgctggctcctttctaccccgacgtcctcttcatcagggggagattggagttagcacccgtcttcaacgatgagccaaccatgcaaggggaagagcctccccagcgtgaggcgcgacgacggaggaatagaagccagaacgtgcggcgacatcacgaggctggggaacgggatccggcgcaacccgtatcccgggacgaagctttagaagtaggaaaaactcccgacgagtgggtacaccgagaaaggcggaactctcgccgccgtgatcgccgacaagcttaggaccgagaacgagagcaagccgagcaaggtgcaaggctgcgccgagagaatgctctctttgctcggaacctgtaccccgacttcgctcgtgcaatgaacacgccgagtgaagtcggaggggtactggcccagatagctgacggcctcccgcgaaccctagacacggaaggctaccggcggctgcttactcgagcagttaatcaccttctacccatcactaatcctccaagcgacctacgccatgccatcaacagccggcgagacacgcggagctccatcaacgcttcgcgcgaccgatgacacgaaagtgagatagggaaccgagaggagtatgtccgagatcatgccatcctggcatgaagtcatgccacccgagctgagtcggttgcggcctcgaccagtgtcccgttccagggacgatcaagatgacacacaactggctcccctccttgggaccgacctcacgaacgccgacatgaagacacgtgcggagtcttcgcacttactccgtgtctccgggccatccagtggcccctaacttcaaggtctccaacgtcagcaagtatgagcgcaagcaggacctgggtggctggttagccatctacacgattgtcacatgggccgccggagcgacggaggacgtgatgacagtgtattttcccattgtcctagggcaagacgcaatgcagtggctccgacatctaccccaacattgcatagacaattggagcgacttcagttggtgcttcatcgccaacttccagtccctctttgacaagccggcgcagccatgggacctaaaatccattgggcatcagggcgatgaaacgctccggttgtacctcaagaggttttagaccatgaggaaccacacccccgaagtcgccgaggcgggggtgattgaagacttctaccgaggatccaatgactcggctttcgtccgagccatactccagaaaagcgtcggccacctccgaacacttgttccgggaggcagacctctacatcaccacggattaacgggcccaggacctcatcggaggcacgaaagccgcgccacacgcgccacggtgtgacacgaaccagc

SEQ ID NO: 23 - DNA, 25250 bp, Artificial
  synthetic
ttctctggagtttcattatttcattgtacatatgaaaagaaacttacatcatataggagttttccaaaaatatgtatataatattgttgtataaaacataaaagcattattaaaccttgcagaggagcttccacatgattccaactgcatatttgattgaatttttcaagtcatctaaaaagaacaacatttaaagcaaaaactcgagtcaaattgatgtaacattagggattaccagatcccaatgcacgacgattgacacggtcgttggtgaggagtgacaaggtcagtggcttggggataggtgcgcttggttgatgtcagataattgagttcggcagctatgtgaggatcagtattgaggcacacaacctatggcggagcaagaacccgaagaaactcattctatttagtattgagacaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccaccggtggtaccgagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttatctatcaactttgtataataaagttgcccggtccttaggcggaccgggccatctaggccgcggccgcactgtcaagctattattagcttctttaataagtccaatgtgaacaaaccgtctagggttagatggattgctttcacagatttccttactggtctaggaatccctgtaaatatagagcacatagatggaaaaaataaccatctggctgatgctctgtccagattagtaactggttttgtttttgcagaaccacaatgtcaagacaagttccaggacgatttagggaaattggaagcagctcttcaggagaagaaagaggctccgcaagcaatgcacgtagaatatgtctccctgttgatcagatcagcggaccgcattacccgctcgctctgctttatgagggactcgtctcacagcagaatttactcatgcaggccaggcaaagaaccaatgaaggccttaatctgcgaacagaagtcatgccaatccaaaggcgacttagggaatacgaggactgtgcactccaagagtgcattcaatcagcaagacaactggtggccctccaccagcacaaactcgcttacatcagaagcaaagctacaagggacaacgcatatgccgataggctacccacatgcaatcgggaccacgagcaactgtgtgaagtggtcgagctattagaaggaatctcggaaagaatcagcgatacagctgtctaggacagctggcttcaattatggagcgtgatggacccccccgcaataatccaaagtttggtgtgcttttagtagtgcgtctttatggaccactactttattgtaataatcgatgctttttgtagtgcgctcttcgtgcgctctactttatgcttttgcttttgtaagtgcgctgtaagtgcgcctgtctttcttcagatgcttatcctttaagcatcttttgctttttgcgtggcatcctttagttcacaatttaaagaatgacgatggggcccaagatgtgcacccggttctctaaattgcctatataaggatatgccatagccttgtttttgcaagtcaggaatacctgagcataacttggctaagcaaaagtttgtaagtgttctaagctttcatttgtaaactttctgtttggttttaataaaatctctcgtcaatcgttgtgaacatatattgtttgtttgtattgttgtatcttatttgttgtggtgataaggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatggctgcgaccactctcacgagcgctctcccaggagcctttagcagctctcagagaccttcggctccgttcaacctccagaggagccctagagtcctcagacgcttcaaccgcaagaccggtagacagccacgcggtctcgtcagagctgctaaggctcagcgctctggtaccagatccatgggcaactccgttctcaattccggaaggactacgatctgtgatgcgtacaacgttgcagctcatgatccgttctcattccagcacaagtcacttgacactgttcagagggagtggactgagtggaagaagaacaaccattcgctgtatctcgatccgatcgttggaactgtggcttcattcctgctcaagaaggtcggttctctcgttggtaagaggattctctcggaactcaggaacttgatcttcccatctggtagcacaaacctcatgcaggacatacttagggaaactgagcagttcctgaaccaacgccttgacactgataccttggcaagggtcaatgctgagttgacaggtcttcaagcgaacgttgaggagttcaatcgccaagttgacaacttccttaaccctaaccggaatgccgttcctctgtctatcacgtcatctgtcaacacgatgcagcagctgttcttgaaccggcttcctcaattccagatgcaaggttaccaactgttgctccttccactgttcgctcaagctgctaatctgcatctgagcttcatcagggatgtcatcctgaatgccgacgaatggggtatatctgcagctacacttcgcacttacagggactacctgaagaactacacgcgcgactactcgaactactgcatcaacacctatcagtccgccttcaaaggcctgaacacgaggcttcatggtacgttggagtttcggacgtacatgttcctgaacgtgttcgagtatgtctccatctggtcactcttcaagtaccagtcattgctggtctcgtcaggtgctaacctgtacgcatcaggatcaggacctcaacagacgcaatcgttcacgtctcaagactggccattcctgtatagcttgttccaagtcaactccaactacgtgctgaacggcttctctggtgctaggttgtccaacactttcccaaacatcggtggacttccaggaagcactacgactcatgcactgcttgctgcaagggtcaactactctggaggtatctcatctggtgacattggagcttcaccgttcaaccagaacttcaactgcagcacattccttccacctttgcttacgccattcgttagatcatggcttgactctggatctgatagggaaggagtcgctactgtgaccaactggcagacagagtcattcgagacaacactcggtcttcgctcaggagcattcacagcaagaggcaacagcaactacttcccagactacttcattcgcaacatctctggagttcctcttgtcgttaggaacgaggaccttcgcagacctctgcactacaatgagatcaggaacattgcctcaccttcaggtacacctggtggagcaagggcttacatggtctcagttcacaaccgcaagaacaacatccatgcagttcatgagaacggatcgatgatccacttggcacctaacgactacactggattcacgatctcacctatccatgctactcaggtgaacaaccagactcgcactttcatcagcgagaagttcggcaaccaaggcgattctctgaggtttgagcagaacaacacgactgcaaggtacactctcagaggtaacggcaactcgtacaacctgtacttgcgcgtctccagcataggcaactcaacgatccgcgttaccatcaacggtcgcgtttacactgctacaaacgtcaacacgaccactaacaacgatggtgtcaacgacaatggtgctcgcttcagcgacatcaacatcggtaacgttgtcgcaagcagcaactctgacgttcctctggacatcaacgttacgttcaactctggaacacagttcgatttgatgaacaccatgctggttccgacgaacatcagcccattgtactgagttgcgtggaccgaagcttgcgcgcctaggtttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacatctccacattcaatatattccctgaattatctgacgattttgatggctcactgttttcccaagtcttgaattgtcttctgtgcgccagtcaaatgcatatgtgttgagtttatcttttaaatatcaagcttttgtttttaacttttgtttgtaaccaaaaactcacagtaggagtttgatcacataattttatgtttgcctttgcaatttctagtgagtctttgattaaaagcttgaaaagaaaatgcagccaagcttaccaagtaagttatgtgtattaaccagaggaagagagaatcttgcaaaatttcaacaaacacaaaaagaagtattactacgattggtggagaaagaaaacgattccaaatcttgaactgttgttgtaaaagcatagcagaaagtgggagacaaccgaaatagaaatgactataacttaatttaatgttatcattataatttcttctagcaaatatttagaaagtaaatatcacatcaacctttaatgtaattaagctttctctttttgattcatgtgagatgaaaagaaaaaaaagaagagaaaagtgtagaaaacacatcatttctaagctgaaggtacatagtacccttgtacttttggtttcacctgcatagagaaaacccacaagaatatgacagtctgatttgtcagtctcattctcaagcaacatttctctatccgttactttcatggtgaataacacaatccatcatcaatactttgtgttactcagaaactgaaagttattccgagtcttgcatatctttgggcctactcgtttttctaccattattgctgattgttaagctctcgctacttgaatcggcattgttggagtgggaaggttcaaaaaattggagttatgactagttgtctctttctatgtacgatggagaaaatgaataaacaactgagaaaatggctcttgtttagttgatgatgctcttaagctttccactggttgccatatatgatttgggcatttcactttgatcttaatgggccttgtaaggcccaagactcatgattatctttagttgatgctcttaattaggtgtgggcaaataattcaaactgtatgtacccgaccaaaaccaaagcaaaaataatcgaaccaaaccgaaaatttaaaaataaccgaatgaaaactaaatcctataactgaaagaactgaaaccgaatcaaaatatttaatgtaaccaaaaatatccgaaatataattatattgtcaaaaatattaataatttctagattaaataattaaaaatacttaaaaatttatataaaatagtaaaaatactcgaaaataaccacaaatattcaaaaacaaccgaaatatcccaaaatattcaaagcaaaataaccgaatggataccaaattttaaaaccgaaaaaactggaacaaaaccagaatcgaaccaaaatttcaaaaatcgaataaatactaaactttagaacaaaaaaaaacgataaccgaatgtatacgaaccaaagccgaattagataaccgaacgtccaggactactcttaatctttccgccacttatgatttgggctattactttgtttataatgagccttttcaagctcaagttcatgattgtccgtgagatgagaaactgacttgttggattcgaaaccctagctagtattggttaatacttaatacataaatgacctgcattgacatcatcatccaagaaaataaaaattgtatgcttgagatatttagttttcctagctaggttttctttattttagtaccgaatctttaggtgtgccacgttaatttagacccattttttcatacttaccaactgagtctagtttaatcatgactataatcgtataaaatgattcagtcgacgtcattgcgaacgtatataaaatcatccaaattgacgtcattccaaagaggtaagcatgcttatctaagagtccgagcatactaaacaagacgacattttatttgcactctaaatcaaattttgtattgcctaaagaaaaacaatcaaactcaagtttcttaaaattaatttcattcaaactaatcactttcaatatctcacatattattcatgccatttctatttgtctaaacatgatttaaaaaaaaagtaaaatacaaagattactatgcaaaaactctataaaaaaaaattcaaatttcttatttatttgtgacatcaaattttcaaaataatttttttaattatcggttgatccggtcagtcgataaaaacataaactttcagcgaccgttaaaactttcctactaccgatttagagaaaatcttagcttgaaacgtaattgtaacctgccttcatgcaagtcgcaagatatgtcatcctaagttgtatatgttttctcaaaagatgtatttacttgagaaaatacgtttcaacgttgatggacaaccaattaagaatcaagcacctttcgtaatcaatttaggcttatcgtctaaggtatactgatttacgacagttgactagacttataaggaacaaaataatagaataatttcgtcaagaaaaattgattttggactcatactttacataatattttactcttaaatttatttaagtggctcctcgcatgatcccaaagagcaagcctagactatatggaaaagtttctaaacacttcacctaatcatagagactaagatggtaattcgtaaacgacaaagcctagtgacactgtccattgtaaaattccacatcatattagtattaaacatatacatgtagtttcctgaacacatgtagtatcaaacacacttcgtggcttcttcctcgaaatcgaggcctaggcttaaggtttaaacagcccgggcgcgcccggaccgggccatctaggccccttagggagctctcgcgacgtcaatcgagtacgtacgtaagggcgacaccccctaattagcccgggtctagagtcgacagatctccatggatccgttaacggccactttgtacaagaaagctgggtgcccgggaataagtgactagggtcacgtgaccctagtcacttaggtgaccaagcttcggccgcaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatccccggccctcttctctataagaggagcccttgcaatcagttgcaagcatgcaagtttcccactgcaagcttacttctgagtttgagttcaagttcaataaaattcaagctttcctcttacattctgttcttgaaaggttcgatctaatcgagcgagtagagaacaagatcttttgggatttccgccgttccggatcttcgatatcccggactggcgccaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatttcttttatatccttcactcccatgaaaaggctagtaatctttctcgatgtaacatcgtccagcactgctattaccgtgtggtccatccgacagtctggctgaacacatcatacgatattgagcaaagatcgatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggcgccttgggatccatgggccacaacaacccgaacatcaacgagtgcatcccgtacaactgcctgtccaacccggaggtggaggtgcttggaggcgagagaatcgagaccggctacactcccatcgacatcagcctcagccttacccagttcctgctctcggagttcgtgccaggagcaggtttcgtgctgggactggtcgacgtgatctggggcatcttcggtccgtcccaatgggatgcgttcctggttcagatcgagcagctgatcaaccagcgcatcgaggagttcgccaggaaccaggccatctctagggtcgagggcctcagcaacctgtaccagatctacgcagagtccttcagagagtgggaggccgatccgaccaatccagcgctcaaggaggagatgcgcacgcagttcaacgacatgaactccgctctgacgacagccattccgctgtttgcggtccagaactaccaggtgccgctgcttagcgtgtacgtccaggctgctaacctccacctgtcggttcttcgggacgtgtcagtgttcggccagaggtggggattcgacgctgcgacgatcaactcgcgctacaacgacctcaccaggctcatcgggaactacacagaccacgcagtgcgctggcacaacaccgggttggagcggatatggggcccggactcgagagattggattcggtacaaccagttccgccgcgagctgaccctcacggtgctggacatcgtgtcgctgttcccgaactacgactcgcgcacgtacccgatccgcacggcgagccaactgaccagggagatctacaccaacccggttctcgagaacttcgacggcagctttcgcggaagcgcgcaaggcatcgaaggttcgatccgctcgccgcacctgatggacatactcaacagcatcaccatctacacggacgcgcacagaggcgagtactactggagcggacaccagatcatggcgagccctgtcggcttctctggaccagagttcacattcccgctgtacggcacgatgggtaacgctgctccgcaacagaggatcgttgctcagctcggccaaggcgtctacagaaccctgtcctcgactctgtaccggaggccgttcaacatcggcatcaacaaccagcagctttccgtccttgacggtacggagttcgcgtatggcacctcatccaacctgccttccgccgtttaccggaagtccgggacagtggacagcctcgacgagatcccgccgcagaacaacaacgtgcctccaaggcaaggcttctctcacaggctctcacacgtgtcgatgttccgctctgggttcagcaactcctccgtctccatcatccgcgctcccatgttctcgtggattcacaggagcgccgagttcaacaacacgatcgacccggagcgcatcaaccagatcccgctgaccaagagcacgaacctcggctcaggcacctctgtggtcaaaggacccggtttcactggcggcgacatcttgaggaggacaagcccagggcagatctccacgcttcgcgtcaacatcacagctccgctgtcccagcgctaccgcgttcggatcaggtacgcctcgacgaccaacctccaattccacacctcgatcgatgggaggccgatcaaccagggcaacttctccgcgacaatgtcctccggcagcaacttgcagagcggttccttccgcaccgtgggcttcaccacgccgttcaacttcagcaacgggtcctctgtcttcaccctgtcggcacatgtgttcaacagcgggaacgaggtctacatcgaccgcatcgagtttgtgccagccgaggttacgtttgaagcggagtacgacctggagcgcgcgcagaaagtggtcaacgcgctgttcacgtcctcgaaccagatcgggctcaagaccgacgtgacggactaccacatcgaccaggtgtccaacctcgtggactgcctgtccgacgagttctgcctcgacgagaagcgcgaactgtccgagaaggtgaagcacgcgaagcggctgtctgacgagcggaaccttctgcaagacccgaacttcagaggtatcaacaggcaacctgaccgcgggtggcgcggatcgacggacatcacgatccagggcggcgacgacgtgttcaaggagaactacgttacactgcccggcacagtggacgagtgttacccgacctacctgtaccagaagatcgacgagtcgaagctcaaggcgtacacgaggtacgagcttcgcggctacatcgaggactcgcaagacctggagatctacctgatccgctacaacgccaagcacgagatcgtgaacgtgcctggtactggttcactgtggccactgagcgcgcaaagcccgattgggaagtgcggtgaacccaacaggtgcgctcctcacctggaatggaatccggacctggattgttcttgccgcgatggcgagaaatgcgcgcaccactcccaccacttcaccctggacatcgacgtcggttgcaccgatctcaacgaggacttgggcgtgtgggtgatcttcaagatcaagacccaggatgggcacgccaggctcggcaacctggagttcctggaggagaagcctctgcttggtgaagcgcttgccagagtcaagagggcggagaagaagtggcgcgacaagcgcgagaagctccagctggagacgaacatcgtctacaaggaggccaaggagtccgtcgacgccctctttgtgaacagccagtacgaccggctccaggtggacacgaacatcgccatgatccatgcagccgacaagcgggttcacaggatcagggaggcttatcttccggagctgagcgtcataccgggcgtgaacgctgcgatcttcgaggagcttgagggccggatcttcacggcttacagcctctacgacgcgaggaacgtgatcaagaacggcgacttcaacaacggcctgctctgctggaacgtcaagggccacgttgacgtcgaggagcagaacaatcaccggagcgtgctggtgatccctgagtgggaagccgaggtgtctcaggaggtcagggtctgtcctggacgcggatacatccttcgcgtcacagcctacaaggagggctatggcgagggctgcgtcaccattcacgagatcgaggacaacaccgacgagctgaagttcagcaattgcgtcgaggaggaggtgtacccgaacaacaccgtcacctgcaacaactacacgggcacacaggaggagtatgagggcacctacacctctcgcaaccagggctacgatgaagcgtacggcaacaacccatcagttcccgccgactacgcctccgtctacgaggagaagtcgtacaccgacggcagacgcgagaatccttgtgagtccaacagaggctacggcgactacacgccactgccggctggatatgtgaccaaggacctggagtacttcccggagaccgacaaggtgtggatcgagatcggcgagaccgagggaaccttcatcgtcgacagcgtcgagctgctcctgatggaggagtaggttaattcgattactagtgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatgggaattcctaggcttaaggtttaaacagccccctccggcggtgtcccccactgaagaaactatgtgctgtagtatagccgctggctagctagctagttgagtcatttagcggcgatgattgagtaataatgtgtcacgcatcaccatgcatgggtggcagtctcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacgaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgctgcagaaacttatctctgttatgaatcagaagaagttcatgtctcgtttcatttaaaactttggtggtttgtgttttggggccttgtaaagcccctgatgaataattgttcaactatgtttccgttcctgtgttatacctttctttctaatgagtaatgacatcaaacttcttctgtattgaaattatgtccttgtgagtctctttatcatcgtttcgtctttacattatatgtgctacttttgtctaatgagcctgaaaagtggctccaatggtacgcactggaagatttgttggcttctggtagatatagcgacagtgttgagcttgtaatatcatgtctcttattgctaaattagttcctttcttaacagaaaccttcaaagtttttgtttttgttttcatttacctaatgtacacatacgctggccatgactaacaacatgtccaggcttagagcatatttttttctagcttaaattgttaacttgtcattcagtaaaatccgagaattgtgaagctctaattgaagctaattcgttttataaagtcagttaaaaagtatactaaattatccaacttttcttcaaaatctcaaaattctatgacaaaacgatagtctttgtttatgtcagtaccacaaagaggtggaaaaaaacaccaaaaaaacaataagcaaactatacactgagaagaaaaataaaagagagctcaatagatgttttatactaacggtagattagatcaaagatccaagctttactctacatagagcagaacccagaatcccttcatatctcttttattctagcaccgataatctactgaaaagaagacacttagagctctgtctctttgtcaaagaagtcccagccgtcatccagaagctccttacgttcattaacagagaattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcggggtgaccaagcttggcgcgccgtcccattctggccgaatttaagtgactagggtcacgtgaccctagtcacttaccggattctggccggagcctgcttttttgtacaaacttgaagctggccttctaggcccggaccgggtgaccaagcttgggccgcgtttaaacttcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatgaacaagaacaacaccaagctgagcacccgcgccctgccgagcttcatcgactacttcaacggcatctacggcttcgccaccggcatcaaggacatcatgaacatgatcttcaagaccgacaccggcggcgacctgaccctggacgagatcctgaagaaccagcagctgctgaacgacatcagcggcaagctggacggcgtgaacggcagcctgaacgacctgatcgcccagggcaacctgaacaccgagctgagcaaggagatccttaagatcgccaacgagcagaaccaggtgctgaacgacgtgaacaacaagctggacgccatcaacaccatgctgcgcgtgtacctgccgaagatcaccagcatgctgagcgacgtgattaagcagaactacgccctgagcctgcagatcgagtacctgagcaagcagctgcaggagatcagcgacaagctggacatcatcaacgtgaacgtcctgatcaacagcaccctgaccgagatcaccccggcctaccagcgcatcaagtacgtgaacgagaagttcgaagagctgaccttcgccaccgagaccagcagcaaggtgaagaaggacggcagcccggccgacatcctggacgagctgaccgagctgaccgagctggcgaagagcgtgaccaagaacgacgtggacggcttcgagttctacctgaacaccttccacgacgtgatggtgggcaacaacctgttcggccgcagcgccctgaagaccgccagcgagctgatcaccaaggagaacgtgaagaccagcggcagcgaggtgggcaacgtgtacaacttcctgatcgtgctgaccgccctgcaggcccaggccttcctgaccctgaccacctgtcgcaagctgctgggcctggccgacatcgactacaccagcatcatgaacgagcacttgaacaaggagaaggaggagttccgcgtgaacatcctgccgaccctgagcaacaccttcagcaacccgaactacgccaaggtgaagggcagcgacgaggacgccaagatgatcgtggaggctaagccgggccacgcgttgatcggcttcgagatcagcaacgacagcatcaccgtgctgaaggtgtacgaggccaagctgaagcagaactaccaggtggacaaggacagcttgagcgaggtgatctacggcgacatggacaagctgctgtgtccggaccagagcgagcaaatctactacaccaacaacatcgtgttcccgaacgagtacgtgatcaccaagatcgacttcaccaagaagatgaagaccctgcgctacgaggtgaccgccaacttctacgacagcagcaccggcgagatcgacctgaacaagaagaaggtggagagcagcgaggccgagtaccgcaccctgagcgcgaacgacgacggcgtctacatgccactgggcgtgatcagcgagaccttcctgaccccgatcaacggctttggcctgcaggccgacgagaacagccgcctgatcaccctgacctgtaagagctacctgcgcgagctgctgctagccaccgacctgagcaacaaggagaccaagctgatcgtgccaccgagcggcttcatcagcaacatcgtggagaacggcagcatcgaggaggacaacctggagccgtggaaggccaacaacaagaacgcctacgtcgaccacaccggcggcgtgaacggcaccaaggccctgtacgtgcacaaggacggcggcatcagccagttcatcggcgacaagctgaagccgaagaccgagtacgtgatccagtacaccgtgaagggcaagccatcgattcacctgaaggacgagaacaccggctacatccactacgaggacaccaacaacaacctggaggactaccagaccatcaacaagcgcttcaccaccggcaccgacctgaagggcgtgtacctgatcctgaagagccagaacggcgacgaggcctggggcgacaacttcatcatcctggagatcagcccgagcgagaagctgctgagcccggagctgatcaacaccaacaactggaccagcaccggcagcaccaacatcagcggcaacaccctgaccctgtaccagggcggcaggggcatcctgaagcagaacctgcagctggacagcttcagcacctaccgcgtgtacttcagcgtgagcggcgacgccaacgtgcgcatccgcaactcccgcgaggtgctgttcgagaagaggtacatgagcggcgccaaggacgtgagcgagatgttcaccaccaagttcgagaaggacaacttctacatcgagctgagccagggcaacaacctgtacggcggcccgatcgtgcacttctacgacgtgagcatcaagtaggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgacctcgaggcctaggcttaaggtttaaacagcccgggcgcgccggtaccgagctcgaattcggtaacccggtccgggccattctggccgtaccgagctcgaattcggcccaacttttctatacaaagttgatagcgataaatcctgaggatctggtcttcctaaggacccgggatatcggaccgattaaactttaattcggtccgataacttcgtatagcatacattatacgaagttatacctggtggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttaaaagatgaaaattaatgagacaatttttgttgaaacttatctaaaataaagcaaatatttgactaaatattgtgtgcgaaacatatccaaaataaataaaatatttcatgttaatttggactaaacatttccttactaaatccttaatccataaaggttaaaccctaaacatttgactaaacatttccctactaaatccttaatccataaatgtaacactctaaaattctattatgagatttagggaaattcttccaaaagttttggattaaaatatatttttataaaaaatctctgaaatagataaatggtgttagtattttttttgtaatataaaagttatacttaaagcatttaaatgggaaatgttatttgaaactccaaaagaaataaaacagggtcatctttaattggtaattgatgaaatacgcgtttagaaaccttctataaagaagatttaaaacaaaattaattcttaataaaagttagacaatataagctcttaaattaaataaataataaatacctaatttttaaatgaacttttaactagactccacattcaaattgaaattttattaagatgaatgacgtttgtgtattgca

SEQ ID NO: 24 - DNA, 13998 bp, Artificial
  synthetic
cgagacttcactgccagttgatcgtcccatgaggttgtcagaaattcatgttgcagctgagcatgcactgaaacaaacgatatcagatcctgattttatgacatcactttcatcactagaagaatttgaggtagtacattcttgcacatcacaattttttttgaaacaatggcaagagttctgcctttcaaccaaggcgaaatgaattttttatacaagaaggggtagccaaacgtgatgcctaccacaagacactcaaatggctaacccacccacgcgacgtggcagagtacaaaactactatgagtcattgttgctgagcatggttgcatagcagccaacatgtccggcttaccatggctgatccagacgacccactctaatgatgttgggcactcgagccagaattaagagtccactacaggtcatcgttgccaatgtcaagcgtgccacataagagcagctttgactttttatttcgaacccttggtgccgcataagagcagcctattgtggttcctgctgatgtgccatttcagattgcataatcttgctcctattcctggtactgagtcttttatgggatggtgggagaagattgacaaaagttctggagattggctatgaggggtctcaattctcttattgccttgggcgcctggattctttggaatcattggaatcgaattgtctttgatggattatcccctacccctagtgtttccgcagctctctgtcaggcaagggagaaacaacagttgtgggaaatggcaggagctaagggtctctcctttcttgccgccaccaacagtgtcccatagctgcttgttgtagggttgtatttgtttatcatggatcgttttctttttctttgttttaaacttccttagggtttttttttgtgtgtactggggtctattttggaccttcttttcctaatacttaatataatgatgcgtagttctcctgcgctttcgagaaaaaaaatcaccactattgtcatcattgtcctatcaccctgtagaagtctctctagagggccatcaacggtagctgcacacaacaaacgcatagctgcatcggctgccagcccctccaatcgggcatcacatggaatcagatttgatcctgctgcacacgtacaccaccacacccatagccattgtcctcacatgtgcgccccgccacagagggcaccaccccctgcccttgtgtcgccggattttcccgctaggtcctcggcctcggcgccccggatcgatgccgctgccatcgcctgggcatggacaccagtgcgccgtgcatccggtcctgagactgaattttggaccccagacaaggaagcagccatgggcaccgtcctggtggcgcctgagccgccgcagcgacagcatacgacagatccataccaagggccatcggacccacactcaaggatggcagaatccattccgctggcgctagagccaatgatggcaatgtcccggcaggccggcatagctgcgcttgcgtgctctcatcgtgacccccagcctttggtggcatgctccggcggtggcgagggggaggggtagggaggaggctcaacggtgcgtgcgatgggcttcgctctaaccgtctaggcaggcaacgcaggggttggggggggaattgcgatgctcatcacaatatatacacattatggactttaattttcgtaataatgcttctgtgttttctttgaactatttttgtgttacagaaaagatatatggagcttactaaaggtgcagctgacaattaccaccgatcttggtggaaaagacatggagttgttcttgatggagagatcgcagctctgttctttaagcatggaaattatgacctggctgtgaaatcctatgagaaagtttgtgctctctattctgcagaaggctgggaagagctgttggcagatgttcttcctgatcttgcagaatgccagaagattcttaatgatgaagctggttatttggcttcttgtgtaaagttactatcgctggacagtggcttgttttcatctaaagagcggcaaggtttccagtcagaagttgttcgacttgctcacagtgaaatctgatcatgagcggagaattaaggcgggaaacgacaatctgatcatgagcggagaattaagggagtcacgttatgacccccgccgatgacgcgggacaagccgttttacgtttggaactgacagaaccgcaacgttgaaggagccactcagcaagcttgatatcgaattcctgcagccctatgatttagaataatatacaaatatattacataaaaaatatattaattgaattagtgttgtctaatttataattattagaatgtaattcaattccaacgaaacaacggggccttaggtttaatatcttccttacactgcgaaaatgttgttacacttgccaaaaaaaatcaatcgcatatttaccttacaaggacatattttagcaaaatgctatagacatgaatccaacgtaatcaatagagtgagatttactggtaaactaccaattgctcatctgctcggtaccaaccagcctttcctattaccatgcacatgttgcctctcaactgcagcatctttcaagccgtgagcagacatgttgcagatcgaagtaaggtatatatgtgcatagtctcctaattcttcatcttcaacctctagctgattgatctctggtatttaccactctttccttccttccttccttcaattctaaataccacaaatcaaagttgctttgccatggagaagaggaacctgcagtggcggcgagggcgtgatggcatcgtgcagtaccctcacctcttcttcgcggccctggcgctggccctcctagtcgcggacccgttcggcctcagtccgctggccgaggtcgactaccggccggtgaagcacgagctcgcgccgtacggggaggtcatgggcagctggcccagagacaatgccagccggctcaggcgcgggaggctggagttcgtcggcgaggtgttcgggccggagtctatcgagttcgatctccagggccgcgggccgtacgccggcctcgccgacggccgcgtcgtgcggtggatgggcgaggaggccgggtgggagacgttcgccgtcatgaatcctgactggtaagtgctcgatatcgctccggcgtccactcgttacatgctataatatagtagtactaagatattttgatctgattttttgcattcttgggagaaacgtcatgcaaaatttgttgtttcttggcaaaggtcagaagaagtctgtgccaatggagtgaactcaacgacgaggaagcagcacgagaaggaggagttctgcggccggccgctcggcctgaggttccacggggagaccggcgagctctacgtcgccgacgcgtactacggtctcatggtcgttggccagagcggcggcgtggcgtcctccgtcgcgagggaagccgacggggaccccatccggttcgcgaacgacctcgatgtgcacaggaatggatccgtattcttcactgacacgagcatgagatacagcagaaagtgagcaaagcgacgtaacaatccggcttctcattttcaaacgcctctgtattctctgctgaaagagtagctcaccagacaagagctgaatttgcagggaccatctgaacatcctgttagaaggagaaggcaccgggaggctgctcaggtatgatccagaaacaagcggtgtccatgtcgtgctcaaggggctggtgttcccaaacggcgtgcagatctcagaggaccatcagtttcttctcttctccgagacaacaaactgcaggtaacaaaaatactatctgacgatgctcatgattctaccgtatccatagtcatgaacacaaaccacacgaatctggccttgaccaggataatgaggtactggctggaaggcccaagagcgggcgaggtagaggtgttcgcgaacctgccgggcttccccgacaacgtgcgctccaacggcaggggccagttctgggtggcgatcgactgctgccggacgccggcgcaggaggtgttcgccaagaggccgtggctccggaccctgtacttcaagttcccgctgtcgctcaaggtgctcacttggaaggccgccaggaggatgcacacggtgctcgcgctcctcgacggcgaagggcgcgtcgtggaggtgctcgaggaccggggccacgaggtgatgaagctggtgagcgaggtgcgggaggtgggccgcaagctgtggatcggaaccgtggcgcacaaccacatcgccaccatcccctaccctttagaggactaaccatgatctatgctgtttcaatgcctcctaatctgtgtacgtctataaatgtctaatgcagtcactggttgtaatcttgtttgtgtttggcaaattggcataataatggacagattcaatgggcattggtgctgtagtcgcatcacactaattgaatgggatcatgttgagctctcactttgctacaatttgctccagcttgtacggttgtaccctcttgctcgtctatagtaagggccatctaaaaaaaactcaaattagatctgcaatacaagtatgattgggccgaatttggattgtcacgggtccgcgaccgcgaattgggctcggtttgatttagccgacatagtagtgaccgacccgagccggcggcgagccaaaccgagcggacgccgccatgatcaagctatcggacggccgctctagaactagtggatcagcttgcatgcctgcaggtcgactctagaggatctgcaccggacactgtctggtggcataccagacagtccggtgtgccagatcagggcacccttcggttcctttgctcctttgcttttgaaccctaactttgatcgtttattggtttgtgttgaacctttatgcacctgtggaatatataatctagaacaaactagttagtccaatcatttgtgttgggcattcaaccaccaaaattatttataggaaaaggttaaaccttatttccctttcaatctccccctttttggtgattgatgccaacacaaaccaaagaaaatatataagtgcagaattgaactagtttgcataaggtaagtgcataggttacttagaattaaatcaatttatacttttacttgatatgcatggttgctttcttttattttaacattttggaccacatttgcaccacttgttttgttttttgcaaatctttttggaaattctttttcaaagtcttttgcaaatagtcaaaggtatatgaataagattgtaagaagcattttcaagatttgaaatttctccccctgtttcaaatgcttttcctttgactaaacaaaactccccctgaataaaattctcctcttagctttcaagagggttttaaatagatatcaattggaaatatatttagatgctaattttgaaaatataccaattgaaaatcaacataccaatttgaaattaaacataccaatttaaaaaatttcaaaaagtggtggtgcggtccttttgctttgggcttaatatttctccccctttggcattaatcgccaaaaacggagactttgtgagccatttatactttctccccattggtaaatgaaatatgagtgaaagattataccaaatttggacagtgatgcggagtgacggcgaaggataaacgataccgttagagtggagtggaagccttgtcttcgccgaagactccatttccctttcaatctacgacttagcatagaaatacacttgaaaacacattagtcgtagccacgaaagagatatgatcaaaggtatacaaatgagctatgtgtgtaatgtttcaatcaaagtttcgagaatcaagaatatttagctcattcctaagtttgctaaaggttttatcatctaatggtttggtaaagatatcgactaattgttctttggtgctaacataagcaatctcgatatcacccctttgttggtgatccctcaaaaagtgataccgaatgtctatgtgcttagtgcggctgtgttcaacgggattatccgccatgcagatagcactctcattgtcacataggagagggactttgctcaatttgtagccatagtccctaaggttttgcctcatccaaagtaattgcacacaacaatgtcctgcggcaatatacttggcttcggcggtagaaagagctattgagttttgtttctttgaagtccaagacaccagggatctccctagaaactgacaagtccctgatgtgctcttcctatcaattttacaccctgcccaatcggcatctgaatatcctattaaatcaaaggtggatcccttggggtaccaaagaccaaatttaggagtgtaaactaaatatctcatgattcttttcacggccctaaggtgaacttccttaggatcggcttggaatcttgcacacatgcatatagaaagcatactatctggtcgagatgcacataaatagagtaaagatcctatcatcgaccggtataccttttggtctacggatttacctcccgtgtcgaggtcgagatgcccattagttcccatgggtgtcctgatgggcttggcatccttcattccaaacttgttgagtatgtcttgaatgtactttgtttggctgatgaaggtgccatcttggagttgcttgacttgaaatcctagaaaatatttcaacttccccatcatagacatctcgaatttcggaatcatgatcctactaaactcttcacaagtagatttgttagtagacccaaatataatatcatcaacataaatttggcatacaaacaaaacttttgaaatggttttagtaaagagagtaggatcggctttactgactctgaagccattagtgataagaaaatctcttaggcattcataccatgctgttggggcttgcttgagcccataaagcgcctttgagagtttataaacatggttagggtactcactatcttcaaagccgagaggttgctcaacatagacctattcaccccatttgatcacttttttggtccttcaggatctaatagttatgtataatttagagtctcttgtttaatggccagatatttctaattaatctaagaatttatgatattttttaattttttatcatgtctgatgagaattaacataaaggctcaattgggtcctgaattaataatagagtgaaaattaatccagaggctctattagaaccttcaattagtaataccaagatatatataagatagtagagtatagtttaaatgttggcattgttcattctttcttttgttatttaatttatgctttccacggtggttagtggttacttctgaagggtccaaataatgcatgaagagtttgaggacaagaagtctgccctaaaaatagcgatgcaaaggcatggtgtccaagccatacatatagcgcactaattttatcagcagaacaatggtatttataggtcctagtgcccaggcaacaagagacacgaataaagcatcgatcacgacaccatggcggcgacaatggcagtgacgacgatggtgacgaggagcaaggagagctggtcgtcattgcaggtcccggcggtggcattcccttggaagccacgaggtggcaagaccggcggcctcgagttccctcgccgggcgatgttcgccagcgtcggcctcaacgtgtgcccgggcgtcccggcggggcgcgacccgcgggagcccgatcccaaggtcgtccgggcggcctgcggcctggtccaggcacaagtcctcttccaggggtttaactgggagtcgtgcaagcagcagggaggctggtacaacaggctcaaggcccaggtcgacgacatcgccaaggccggcgtcacgcacgtctggctgcctccaccctcgcactccgtctcgccacaaggctacatgccaggccgcctatacgacctggacgcgtccaagtacggcacggcggcggagctcaagtccctgatagcggcgttccacggcaggggcgtgcagtgcgtggcggacatcgtcatcaaccaccggtgcgcggaaaagaaggacgcgcgcggcgtgtactgcatcttcgagggcgggactcccgacgaccgcctggactggggccccgggatgatctgcagcgacgacacgcagtactcggacgggacggggcaccgcgacacgggcgaggggttcgcggcggcgcccgacatcgaccacctcaacccgcgcgtgcagcgggagctctccgcctggctcaactggctcaggtccgacgccgtggggttcgacggctggcgcctcgacttcgccaagggctactcgccggccgtcgccagaatgtacgtggagagcacggggccgccgagcttcgtcgtcgcggagatatggaactcgctgagctacagcggggacggcaagccggcgcccaaccaggaccagtgccggcaggagctgctggactggacgcgggccgtcggcgggcccgccatggcgttcgacttccccaccaagggcctgctgcaggcgggcgtgcagggggagctgtggcggctgcgcgacagctccggcaacgcggccggcctgatcgggtgggcgcccgagaaggccgtcaccttcgtcgacaaccatgacaccgggtcgacgcagaagctctggccgttcccatccgacaaggtcatgcagggctacgcctacatcctcacccatccaggagtcccctgcattttctacgaccacatgttcgactggaacctgaagcaggagatatccacgctgtctgccatcagggcgcggaacggcatccgcgccgggagcaagctgcggatcctcgtggcggacgcggacgcgtacgtggccgtcgtcgacgagaaggtcatggtgaagatcgggacaaggtacggcgtgagcagcgtggtcccgtcggatttccacccggcggcgcacggcaaggactactgcgtctgggagaaagcgagcctccgcgtcccggcggggcgccacctctagcagctcagattgctcagtcttgtgctgcattgcaaacacagcagcacgacactgcataacgtcttttccttgagatctgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagctactttagactgtgttcggcgttccccctaaatttctccccctatatctcactcacttgtcacatcagcgttctctttcccctatatctccacgtcgacgcggccgatcccccgggctgcaggaattcccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactaagctgaccgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcccccgggctgcaggtcgactctagaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattcagtacattaaaaacgtccgcaatgtgttattaagttgtctaagcgtcaatttgtgatggagatcctggtacactatctgtagcagtttggaatggcttcccagatgacatcacactggagtctctcagtttaaggttgtcagcttcttctagtgcagatgaaggtatcaaggtacaagtactgtgttagcattactgattgctacccttttcctctgtggcatgtcttattctgttattcatggtttggatgcactaatatgttttcaagtaaaattcagttccatccttgcagaacagatttcataaatcaaaactactatgataaagtatatcaaactctagagaaatgaccgcacataacatattcttcagtcacctaatggtttaatgttgtcatccatgcaggcaattaaaagttcagattctcatgttctagtaccaggtagaaatatcatctcttttgacattcctcgtcaaaagcctggctcctatgtgttgggtgctctcactggacagattggcaagctgtcattcagatcacatggattttcccaagatggtccagttgaaactgatgaatttatgagctttgagaaaccgacaagacctgttttgaaggtaattgctaaaatgagctgaagattacttagaagtttttcgtggggcatgactggagctagatcccttagaagtttctggccttaagttttttggggtttccccttaagattttttttttctttttccccatttttccataactggcaccttgtattgggtctattttaggcctttctttctcttcttaatatattgatgtacagttctcctacagttcgagaaaaaaagagctgacgatctgaactgttttataaaatcgtagccctaccccatgtcactaaactgccataagagatactatccatactttcaaaacaaaaccggagcatagtttcctctaattagtgaacatggtcagttatattgtttaagacagatatgttctcattgcatttgtttctctgttgtaggtgagaaaaccaagggctttagttgatattacacctgctgtgtcctctgctttgcttatgaatgagctccaatggattggattaatcgttaagcctatagactattctttaaaaggtggaatattgcatatcgatgctggcgctgaactgaaaattgaggagtctcagatgattgaaatagaaatttacgtaagtgatatggagtgtgctaattctgccaacagctccatcaaagctggaaaggttgaaaaggtacctattgaaaatggaaagatagaacttcctgattgggctagtgatgtgactactcttgtttggtttcctgttcgtgctattgatgacacaattgcaagaggagaatccctaggtctgtatgtaacaagtgataaattcactatgttatgtttcaatcagttagttgacctttctttgtccttgtttcagtgtctcagaaacagagcgttgtagatgggatgagaatgattgctctcaagcttgagtttggagttttccgtaaccaagtgtttgaaaggtatctctctgtttttcccctcaaacatgcagaagagctgtgtcatgtctgccattgtataagaaaagatgagatacttcgatacattgaactttagatatagatactcaacaaatttattattatgattttcaggaccattgcagttcattttactaacccattccatgtaagcacacgcgtcgtggacaagtgcaatgatggagctctacttctacaggtaaagttctttcttgtctggcacatgattgtcattgttgtattcctagatttaaaacacacacacacacacacatatatatacagggatattgggagccctgctcttccaaacatatatactagaagccccagccgtgccctgcaccacccgaccggcttggccacctagatcccgtgcacacgtccatcccctgcatgcggatatttgtttccatttgcatgtgcgaaaaatttggaaggtgtatctgtttcctgctgcatgcgcataa

SEQ ID NO: 25 - DNA, 6755 bp, Artificial
  synthetic
atcgattgcaaccttcaaattctcttcgatcttccttccaaattcatctaatatttcatcctcggcaaggaaatcttctaacgtgtaaacttcactgggatccaactcgaaaggatcaaactctccctctggttcgtttgacattgtggatggagtgactaacctgctaacaccctgcaacaatttatacaggagcatatcctcatgcacacgcaaaactgatgttgtccacaagacacgcacaggacacgcacaggacacgcaaacagtttcagactcatgcacacgcacatcagtttcagactcaggcacacgcacatcaaatcaccttcgcttgtcgatgagtcgcagccgcatcgtacaatggcgattttaccgacgataaggcatgggagcacgagccgtcgccgtcgccttgcgagacgacgggagcgatctctcccttcatttaatctcttccacgtcaggttattttgctgagatggcagtatacagacggcaaagttaatgccgttgtacatgcccttagactcttccgtcaccaactcacttagatttttacaacggaacataaggttcgcttgcagacttacatataaggtatagttgcataataatcgccttatgctgtacattgcgacacccgtaaatattcgatgaaatattagtacacaatattaaataagaacgaacaatacatatattatcattgatcttagtatctccttttgctcctcgtagaacaattctgtgtaaattatgcgtaaaattcgaggaccaaaacattggctagaaaaatacctaaaatcagttttgcaattgtttctgattttcctcatattttcttgcttataaagttttccaaaagtaccattttggatgaaaaaacggaaaacaacgctggtctacttgtaaatttggtagtgacatttgggaccgtctagacacgacctaaaaatagtagtctaaaacatagtctgacacgatgccttaaaaatagacgacaaagcacaacacgattagatgtgtcgtgttttgaccgacacgacacaaagtaaggcacgatttaaaacccaataaataatattttaatggttattttatgttccaataattttcatctcttcaaaaaaatgttatagaaatcattgatacttagttgaatatcctaacacaatatatatatatatattaatatatatatatatcaattttaagtcactttgctagacatagtaatatattttaaatattttctctttcttgtatatttttaaaatacacatcagtttttatatgtgtcgtgcttgaaccgacacgatataatcatcggttcgccgtacttctagatcatgatgttcctaggttttaatattaagagacggtctatattaactcaaaactatttcgtgaaaggctaactcgaaaaaaaaatgaatgtaatcacggcccgtcctggattcgagattctaacgtttcattcgtgtccagtgtgcacacttgtggaaaaggaagacgaagaaaaaaaccaacaactaactccggcccgccggatgcgcccacctacttccccctcgcccctctcatggtctctctcgcgcccagatctgctactagacggcaccgctgcagcgcgtcgtgtcgcgggggttggtggcaggcagcgagagcttgccgttcctctctctcagttgtcaggtcctaggctcacctcaccggctcccagcccgcttctatttcttcctccccgaccccgtgcaggtggcagtccagtccacgccaccaaccgcgaggcgaaccaaaccaacccactctccccaaccccgcgcgcccaggccgcccgccctaccaaccatcggcgtcggcaatggcggccatggcgaccaaggccgccgcgggcaccgtgtcgctggacctcgccgcgccgtcgcgccgccaccaccgcccgagctcggcgcgcccgcccgcccgccccgccgtccgcgggctgcgggcgcctgggcgccgcgtgatcgccgcgccgccggcggcggcagcggcggcggcggtgcaggcgggtgccgaggagatcgtgctgcagcccatcaaggagatctccggcaccgtcaagctgccggggtccaagtcgctttccaaccggatcctcctgctcgccgccctgtccgaggtgagcgattttggtgcttgctgcgctgccctgtctcactgctacctaaatgttttgcctgtcgaataccatggattctcggtgtaatccatctcacgatcagatgcaccgcatgtcgcatgcctagctctctctaatttgtctagtagtttgtatacggattaatattgataaatcggtaccgcaaaagctaggtgtaaataaacactagaaaattggatgttcccctatcggcctgtactcggctactcgttcttgtgatggcatgctgtctcttcttggtgtttggtgaacaaccttatgaaatttgggcgcaaagaactcgccctcaagggttgatcttatgccatcgtcatgataaacagtggagcacggacgatcctttacgttgtttttaacaaactttgtcagaaaactagcatcattaacttcttaatgacgatttcacaacaaaaaaaggtaacctcgctactaacataacaaaatacttgttgcttattaattatatgttttttaatctttgatcaggggacaacagtggttgataacctgttgaacagtgaggatgtccactacatgctcggggccttgaggactcttggtctctctgtcgaagcggacaaagctgccaaaagagctgtagttgttggctgtggtggaaagttcccagttgaggattctaaagaggaagtgcagctcttcttggggaatgctggaattgcaatgcggtcattgacagcagctgttactgctgctggtggaaatgcaacgtatgtttcctctctttctctctacaatacttgctggagttagtatgaaacccatgggtatgtctagtggcttatggtgtattggtttttgaacttcagttacgtgcttgatggagtaccaagaatgagggagagacccattggcgacttggttgtcggattgaagcagcttggtgcagatgttgattgtttccttggcactgactgcccacctgttcgtgtcaatggaatcggagggctacctggtggcaaggttagctactaagggccacatgttacattcttctgtaaatggtacaactattgtcgagcttttgcatttgtaaggaaagcattgattgatctgaatttgatgctacaccacaaaatatcctacaaatggtcatccctaactagcaaacaatgaagtaatacttggcatgtgtttatcaaattaatttccatcttctggggcattgcctgttttctagtctaatagcatttgtttttagcattaattagctcttacaattgttatgttctacaggtcaagctgtctggctccatcagcagtcagtacttgagtgccttgctgatggctgctcctttggctcttggggatgtggagattgaaatcattgataaattaatctccattccctacgtcgaaatgacattgagattgatggagcgttttggtgtgaaagcagagcattctgatagctgggacagattctacattaagggaggtcaaaaatacaagtaagctctgtaatgtatttcactactttgatgccaatgtttcagttttcagttttccaaacagtcgcatcaatatttgaatagatgcactgtagaaaaaaaatcattgcagggaaaaactagtactgagtattttgactgtaaattattttaccagtcggaatatagtcagtctattggagtcaagagcgtgaaccgaaatagccagttaattatcccattatacagaggacaaccatgtatactattgaaacttggtttataagagaatctaggtagctggactcgtagctgcttggcatggataccttcttatctttaggaaaagacacttgattttttttttctgtggccctctatgatgtgtgaacctgcttctctattgctttagaaggatatatctatgtcgttatgcaacatgcttcccttagccatttgtactgaaatcagtttcataagttcgttagtggttccctaaacgaaaccttgtttttctttgcaatcaacaggtcccctaaaaatgcctatgttgaaggtgatgcctcaagcgcaagctatttcttggctggtgctgcaattactggagggactgtgactgtggaaggttgtggcaccaccagtttgcaggtaaagatttcttggctggtgctacaataactgcttttgtctttttggtttcagcattgttctcagagtcactaaataacattatcatctgcaaatgtcaaatagacatacttaggtgaattcatgtaaccgtttccttacaaatttgctgaaacctcagggtgatgtgaagtttgctgaggtactggagatgatgggagcgaaggttacatggaccgagactagcgtaactgttactggcccaccgcgggagccatttgggaggaaacacctcaaggcgattgatgtcaacatgaacaagatgcctgatgtcgccatgactcttgctgtggttgccctctttgccgatggcccgacagccatcagagacggtaaaacattctcagccctacaaccatgcctcttctacatcactacttgacaagactaaaaactattggctcgttggcagtggcttcctggagagtaaaggagaccgagaggatggttgcgatccggacggagctaaccaaggtaaggctacatacttcacatgtctcacgtcgtctttccatagctcgctgcctcttagcggcttgcctgcggtcgctccatcctcggttgctgtctgtgttttccacagctgggagcatctgttgaggaagggccggactactgcatcatcacgccgccggagaagctgaacgtgacggcgatcgacacgtacgacgaccacaggatggccatggccttctcccttgccgcctgtgccgaggtccccgtgaccatccgggaccctgggtgcacccggaagaccttccccgactacttcgatgtgctgagcactttcgtcaagaattaataaagcgtgcgatactaccacgcagcttgattgaagtgataggcttgtgctgaggaaatacatttcttttgttctgttttttctctttcacgggattaagttttgagtctgtaacgttagttgtttgtagcaagtttctatttcggatcttaagtttgtgcactgtaagccaaatttcatttcaagagtggttcgttggaataataagaataataaattacgtttcagtggctgtcaagcctgctgctacgttttaggagatggcattagacattcatcatcaacaacaataaaaccttttagcctcaaacaataatagtgaagttattttttagtcctaaacaagttgcattaggatatagttaaaacacaaaagaagctaaagttagggtttagacatgtggatattgttttccatgtatagtatgttctttctttgagtctcatttaactacctctacacataccaactttagttttttttctacctcttcatgttactatggtgccttcttatcccactgagcattggtatatttagaggtttttgttgaacatgcctaaatcatctcaatcaacgatggacaatcttttcttcgattgagctgaggtacgtcatctacaggataggaccttgagaatatgtgtccgtcaatagctaaccctctactaattttttcaatcaagcaacctattggcttgactttaattcgtaccggcttctactacttctacagtattttgtctctataaattgcagctacaacagtcagaacggctggctttaaaatcaaatggcctaaggatcattgaaaggcatcttagcaatgtctaaaattattaccttctctagacgttgatatcgtcttatagggcttggacatgacacagacataatttatatagtattagattgagtgagactgggtgggtttaaattccaagcaagtcaaacttgttcttaattttttccaatcccattcaatccatgggtaacgggattaaccgaacaatgtcttatagggcttggacatgacacagacataatttatatagtattagtgccggtaccacacgggtctaatgtcgtgtttaggcctccatccggcatgatgattggcataagcacgacatgattaggtggtcgacacgattaagcacggcttaaaccagctagtagtatgtagatgtgattgtgatatgtatatgtgattgtgaattgtgatttattatggttttaagtataatcaatttactttataatggtataaatgtttaaattctaaaactgtatatataattttaaagattttctataattctagactgacacaaatactattagttgttaatgttatatagtgccggtatgatacaattaaatattatagtgtagtgtctagatctacactagtgccgacacgacacaatataattagtaatagtgcttaataatacttagtgtacctactgggcggttcgtctggctatctataatcatgaatactcgtgtgctcgtcaccggtcacggccaggagatatgacgacggctgttgttttcttctggcgtggggctgcgcgaagggataagaaggcaccatagtaacat

SEQ ID NO: 26 - DNA, 6624 bp, Artificial
  synthetic
ataaaccagctcccttcgaggggcttcaagctcatgaacaaggcagacgggaagcacaggaacttcggtatatctcgaatctctggcgaggcagttgctactcgacggaactagtcagctgaaggtctttttgctattggaattcaataggaggcgggagctaactgacaccatgccgccctagttttgttgaactgatagtggtgctagccttgtgcatagtaggggccaggggagtaataatatttcccttgtctaaggaagtgattaaggatgtaggagcacagcgcaaatgattagcaaaacgatcacgttcgcagtgcggcaggcattgcgcagctggttcgtttgttcaatcagtgctctcgctcacagtgacctgacagcgtgatcactaaacctagtagctgggcgcgcctttctagatcagaacggaacattggcactggattagattccagtgcggcatgcttcgaaagcgaggacacgggtgctccctaagtacatgttttgaatggatgtggagtggcccgtccattccgcattccaatgggcctgtttggttcagcttttttctgaccagcttttctgagaatctggctgtggggagaatctggctgtgtagagaatctgagtatcattaggattacgtgcggaggaagataaaggtgtccataggactcaggatgtagaaagtgacggattcctactattgcaacgactcaaccgattatgtgtttatgttgattttgaatggtttttacccaaacgaattttatagaagttgactgaaaagctgagcgtttggcagtccgcaacagcttttggtggccagaagctccaaaaagctgaaacaaacaggggcaatagttgacgctttggacgtctcaagcctggccgtcaggcgctgtaccccagctggctgttcagagctacaacactactcgggagtttttttttgtctcacctataaaaaagaacataaaataaagaactactgctacgactcacaggccagaagtcagaacgacgacacagtggcaactctcgtccatcgtcattttctgcgtggcggtacgcatgcatagggtcgccgttgattcctactcgtatactttacctgaacaccgctgcatgcacgcctggacaaagataacgaggaaacgtccagtcgctagctctgacagtgttggacgttttaccgctgcaatcactattacgggatgcatttgatatctggagagtacgttgcgcgaagacgcagtttgcgaagcctggtcgtgctcgtgcacctgcattgcagttcatttcagtcgctcgagcactcctcatcccccaccatcgtcaccacttttcgggtttggtttggtattttcccgcacgcacgggggccccgaggtgtgtgacactgtgactggacacccaaagcctagccaagcaagcggaagaacgcaacgcaaagcaggtcagctcctccgtccgaattcagcctgttactcgttcgctcactttcctctccctttctctccccgccatcttccttaaatcttttcttccagtaacctttcgccgcaaaatccgtgcttccgagtcgagtattggactcgctgcctggtttcgttttttttatgatgagagaaagagagagaggaaaggtaatccgtcctgttttgatgatgtgcctgctgagatttaaggctgcattgtgggttcagtctgcgaatgtttgggggtgggggccaatccctgcgcagaatctcaggatccgagcggagtttatgggtcgacggtatcgataagcttgatatcgaattcctgcagcccgggccgagtgccatccttggacactcgataaagtatattttattttttttattttgccaaccaaactttttgtggtatgttcctacactatgtagatctacatgtaccattttggcacaattacatatttacaaaaatgttttctataaatattagatttagttcgtttatttgaatttcttcggaaaattcacatttaaactgcaagtcactcgaaacatggaaaaccgtgcatgcaaaataaatgatatgcatgttatctagcacaagttacgaccgatttcagaagcagaccagaattttcaagcaccatgctcactaaacatgaccgtgaacttgttatctagttgtttaaaaattgtataaaacacaaataaagtcagaaattaatgaaacttgtccacatgtcatgatatcatatatagaggttgtgataaaaatttgataatgtttcggtaaagttgtgacgtactatgtgtagaaacctaagtgacctacacataaaatcatagagtttcaatgtagttcactcgacaaagactttgtcaagtgtccgataaaaagtactcgacaaagaagccgttgtcgatgtactgttcgtcgagatctctttgtcgagtgtcacactaggcaaagtctttacggagtgtttttcaggctttgacactcggcaaagcgctcgattccagtagtgacagtaatttgcatcaaaaatagctgagagatttaggccccgtttcaatctcacgggataaagtttagcttcctgctaaactttagctatatgaattgaagtgctaaagtttagtttcaattaccaccattagctctcctgtttagattacaaatggctaaaagtagctaaaaaatagctgctaaagtttatctcgcgagattgaaacagggccttaaaatgagtcaactaatagaccaactaattattagctattagtcgttagcttctttaatctaagctaaaaccaactaatagcttatttgttgaattacaattagctcaacggaattctctgtttttttaaaaaaaaactgcccctctcttacagcaaattgtccgctgcccgtcgtccagatacaatgaacgtacctagtaggaactcttttacacgctcggtcgctcgccgcggatcggagtccccggaacacgacaccactgtggaacacgacaaagtctgctcagaggcggccacaccctggcgtgcaccgagccggagcccggataagcacggtaaggagagtacggcgggacgtggcgacccgtgtgtctgctgccacgcagccttcctccacgtagccgcgcggccgcgccacgtaccagggcccggcgctggtataaatgcgcgccacctccgctttagttctgcatacagccaacccaaggatcccctcgaaattcggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagctttgttcatcggtagtttttcttttcatgatttgtgacaaatgcagcctcgtgcggagcttttttgtaggtagccaccatggtttcgccgacgaatctcctcccggcgcggaagatcacccctgtctcaaatggcggcgcagcgacggcgagcccctcttctccctcggtggccgcacggccacggcgactcccttcaggcctccaatctgtgactggtagagggaaggtttccttggcagccatcactagtacaggtttaacagctaagaccggagtagagcacttcggcaccgttggagtagcaatggttactccattcacggaatccggagacatcgatatcgctgctggccgcgaagtcgcggcttatttggttgataagggcttggattctttggttctcgcgggcaccactggtgaatccccaacgacaaccgccgctgaaaaactagaactgctcaaggccgttcgtgaggaagttggggatcgggcgaagctcatcgccggtgtcggaaccaacaacacgcggacatctgtggaacttgcggaagctgctgcttctgctggcgcagacggccttttagttgtaactccttattactccaagccgagccaagagggattgctggcgcacttcggtgcaattgctgcagcaacagaggttccaatttgtctctatgacattcctggtcggtcaggtattccaattgagtctgataccatgagacgcctgagtgaattacctacgattttggcggtcaaggacgccaagggtgacctcgttgcagccacgtcattgatcaaagaaacgggacttgcctggtattcaggcgatgacccactaaaccttgtttggcttgctttgggcggatcaggtttcatttccgtaattggacatgcagcccccacagcattacgtgagttgtacacaagcttcgaggaaggcgacctcgtccgtgcgcgggaaatcaacgccaaactatcaccgctggtagctgcccaaggtcgcttgggtggagtcagcttggcaaaagctgctctgcgtctgcagggcatcaacgtaggagatcctcgacttccaattatggctccaaatgagcaggaacttgaggctctccgagaagacatgaaaaaagctggagttctataatgatctagagcaagccgaattcctgcagcccgggggatccactagttctagagcggccgccaccgcggtggagctcgccaaaacgagcaggaagcaacgagagggtggcgcgcgaccgacgtgcgtacgtagcatgagcctgagtggagacgttggacgtgtatgtatatacctctctgcgtgttaactatgtacgtaagcggcaggcagtgcaataagtgtggctctgtagtatgtacgtgcgggtacgatgctgtaagctactgaggcaagtccataaataaataatgacacgtgcgtgttctataatctcttcgcttcttcatttgtccccttgcggagtttggcatccattgatgccgttacgctgagaacagacacagcagacgaaccaaaagtgagttcttgtatgaaactatgacccttcatcgctaggctcaaacagcaccccgtacgaacacagcaaattagtcatctaactattagcccctacatgtttcagacgatacataaatatagcccatccttagcaattagctattggccctgcccatcccaagcaatgatctcgaagtatttttaatatatagtatttttaatatgtagcttttaaaattagaagataattttgagacaaaaatctccaagtatttttttgggtattttttactgcctccgtttttctttatttctcgtcacctagtttaattttgtgctaatcggctataaacgaaacagagagaaaagttactctaaaagcaactccaacagattagatataaatcttatatcctgcctagagctgttaaaaagatagacaactttagtggattagtgtatgcaacaaactctccaaatttaagtatcccaactacccaacgcatatcgttcccttttcattggcgcacgaactttcacctgctatagccgacgtacatgttcgttttttttgggcggcgcttactttcttccccgttcgttctcagcatcgcaactcaatttgttatggcggagaagcccttgtatcccaggtagtaatgcacagatatgcattattattattcataaaagaattcgagggggatccataacttcgtatagcatacattatacgaagttattctagagcggccgccaccgcggtggagctcggatccactagtaacggccgccataattattgcccagtctttcagggtattacagtgtacaatgtttcagtattttagactgcactactataatatcacaagaactacaaatggagcattcaatactactagtacaatgagtgtgcattgcgacggcacacaaattattcaataaaatattagtggacaataattacatgaaaacgaaccaattaagatgtccgcttgaagtgccttttgggtcaattacatagctcccctgcaaatggagcattcaatactttcctgtccgcttgaaatgccttttgggtcaattacatagctcccctgcaaattaagatgtcaaatatactatcacataaccatcccaaaggataaaccaggatcaccaaacacacaataccaattgatctcagacaggcacatccaacattgcttgaatctcaagtaaaacaattccagatctatcttgtataagtaaaagagctttctgatccaagacatgcttttctgaatctcaagtaaaacaataccagacaggcacattacttgctttctgatccaagacatgctttgactcagaatccactcaagtataacaccatatatttatatatcttttagttgagtaaaacatgcaatcatcaatgaagataaaccccctgcttataatgaatcaggcttataac

SEQ ID NO: 27 - DNA, 5744 bp, Artificial
  synthetic
ctactattttggaacagagggaatagtaggtagtaatgtttcacaataaatcacagtgctaaaatgagatatgaccttagatcccatagctcattgcataaaagtacttgatcagttggtcactgtctttcagaaaaaccgcagtttaattaaacttaatggctgctggatcagatgctagatagcactatatcttaattcgttttgcatgaacaccttataatacagaaccttttcttttcttccagcaactagactctgcatcccttgtggaaatgtgaaccttggttcgtacatgacaaataatacaacatatcatgctacctatgcaacactgacttgatttcaatccttggtttacttgccacatcatacttatggtatatttgggtgaaaagtagatgtttcaagcagtttgtaaatcctaaatacatcatggtttactctggtgctacaactactgttttaaggagaaactattagctttgggatcatctagaatattagcatagcatcaacttcaggcacaactacaagagcagcacttgaaggggtaaaattcagtgctacctacgtaaatcataaaaaaaattatttgtttgaattcaagtgttttgggggttcaacaatgattctatgtagctataaatttttttgttgatttcatgtcatattgattattggtttgatttcacctgttttcttattgtcagccagaaccagcttactatttcggatgttgcaaggaaggcattccttcacagtgtatgtgatagttctctttatacatgtaagacagtgtacaagggcaaaactttgtatgaaattactctgtactgtttacaatgattacagggcatacttgtagtgtttgaggagcaagagacaaaactctacattttagatatataaagcaatagttctttaactcaaaccaaaatctggattagtcttgagcactttctaataatcatataatgctcatgtacctcaataactgcttcagttaatcacatcttttagtttagcactatgctatgatagtttacaaagtgcattgttagcttattttccccataggttctttaatcatatctattgtgctagattttactctgtcccattaggagacaaacaagttcgttcaatagcatatgacacttgtatgctggtaaaatgattgaatattttgtttgacacctattgtttgaaggatgtaagcaatctcatgtacctgttgttgctcgcaaggttcatccctcacaagtacaagatcaaagactcgaaagcttaggtatgaacccataaaactgaatacttaatatgggcacacagtgatgtttttactgaaggcgggaaacgacaatctggcgcgcctgcctgcaggcatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgaggcctcatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgaagcttactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaacccttctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagcggaccggtaccatgataaaactactatttacgtacatatgcacatacacatataaactatatgctctatatcatatggattacgcatgcgtgtgtatgtataaatataaaggcatcgtcacgcttcaagtttgtctcttttatattaaactgagagttttcctctcaaactttaccttttcttcttcgatcctagctcttaagaaccctaataattcattgatcaaaataatggcgattttgccggaaaactcttcaaacttggatcttactatctccgttccaggcttctcttcatcccctctctccgatgaaggaagtggcggaggaagagaccagctaaggctagacatgaatcggttaccgtcgtctgaagacggagacgatgaagaattcagtcacgatgatggctctgctcctccgcgaaagaaactccgtctaaccagagaacagtcacgtcttcttgaagatagtttcagacagaatcatacccttaatcccaaacaaaaggaagtacttgccaagcatttgatgctacggccaagacaaattgaagtttggtttcaaaaccgtagagcaaggagcaaattgaagcaaaccgagatggaatgcgagtatctcaaaaggtggtttggttcattaacggaagaaaaccacaggctccatagagaagtagaagagcttagagccataaaggttggcccaacaacggtgaactctgcctcgagccttactatgtgtcctcgctgcgagcgagttacccctgccgcgagcccttcgagggcggtggtgccggttccggctaagaaaacgtttccgccgcaagagcgtgatcgttgagtcggggcccgggctgcatgcgtttggacgtatgctcattcaggttggagccaatttggttgatgtgtgtgcgagttcttgcgagtctgatgagacatctctgtattgtgtttctttccccagtgttttctgtacttgtgtaatcggctaatcgccaacagattcggcgatgaataaatgagaaataaattgttctgattttgagtgcaaaaaaaaaggaattaagcttactcgagatccactagtgattaagcggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgcgatccacatttccctacatggtggaggatacagagcactcattccggagtataatcttgtcttgtgttgccacctatttatcacttgagcatatacaatgaatgtttcttaagatgtcaatatttgcttataatatagtgacttcccagtatatgtttcagttttgtttcttgcttagtgactagaacaaaaaaacaagtgttctattggtttaattaatctaaactgcattgctcaagcaataattgagtttattccctataacttgcaaagtgtggccatgttatgccctgtggctgtgcgtggccatctgacctccacatctatgtttcccactgctaggttgctttgagtcgtggtggatctcccggtgtgacgacatggggctgggtctgggagctcttctcaaatccgcgggctctcggtctgctcttctcaatttagtgttatctttttcttttagaacaatgccaaagaaacatgacagactgctttgcttgtttttgacatgatgcaaaagcaatggagtctttacttttaaacctttgtatttttgttttgattcaattctgatactgctgaaatgtacgaatatctattattttgatgcaatagtggtgttttgcgatgtttaacagcttgtgtaatccaattattaaccctgctttgtatagaagagtttcttcagaatatatcaattcatgtattctcattatggatattatgtggatattttctatctatgaatggcccctgcactttgaatcgcttatgtcattgttagcaatcacataatatttttcttttctcttagatttaatttgtataatcatataatcattcatatattgttacaacaggatttggttcagcttctcactttgttttaattctgatactgctggttctcttcttgccaggtgcgtgcggccacccggccgtggaccaagcgccgccgctgctcttcttgccaggtgcgtgcgtctcttcagctggccaaaatcaatcatcccaatgcttgtttctgaagggcagcatgtagctcgaggtgctggtcaaacggaggaatcattctggtttggtttctcttttgtttttgtacatagatgacgacgacgtgtctcccgatgcctccccgtcactggcacaccttggcatcgatcctcgaatctctacccccacacccacatctccccgctgccgaaccagccccgatttgccaccatggacgtcgatgacgtatcacccaatgtctccctgtctctgg

SEQ ID NO: 28 - DNA, 9259 bp, Artificial
  synthetic
gttgatattttgacccaaagacatgccattctcattgttttcagttttatcctaccaatgtagtaacccaaacagtttaattacaagtgaatcaactggaaacaagatctacagactgcacacagcacattaacaattctgcgccaaatgaatgacagagtgatttataggcactaagctggttttcatcatgaattttgatcagagcataaaccttgttgacaacagaatagaatcaaacaagctgctcagctcagatggaccaaaataatgaacgcgcgcacagtatattgaaccaaaatagtgaacgcacagtagaaaacagacggagaagctagtgcacactaaaaatagaactcactgatgcatttttaaccgaatcattaaaaaacagaccaacatgattagaccagctatcaaaagacacactgctgagaatcaacaactgctatatgtgtgatctacctgaaataaacagatgcagttttttgtccctacagaccaacatgttttaatcagaatgccaatgataaatccttagagatctggagagcaaactaattaaccactgagaatcaacaactgctataatccttagagatctggagagcacactaattaaccactgagcctctttggtcagcatgcagaaatccccaaaactatactgtaattcgaaacccataagcgcacatcaaagcaactccattgttgatggttgggaatgttaaaaatatggttgtggacaccaattgccgtaaacaaacccacagggagaacatgagtatgaacaatcaaaactaacatgaataagtggatgttcagattaagtaaggaaaatggtgcacagattaagaacagagcaatgtatagcacaagtggaaattacaaaccttttttcacaaaattacaatctctagacagcacacagcaagatcttggaatggatgagggtgatgaatcggtgaaaaaagacagcacacagcaagatctctagaacagaacatgaaatgttggagagtggggatgggttgtagacctaacctctgtccatccttttaatagccgtcgctgccaggtatggaatagggcgccgtgagtgccgtcgagggcgcgcgcaggggcgcggtttggtgtgggcgggatacccggcgcggtcgggaaaccccacggcggtcatcacggcgagccgggggaggggggggttggcggggtggcaggcggtcgctgccaggtattgatgtgcgccagtcagcatcatcacaccaaaagttaggcccgaatagtttgaaattagaaagctcgcaattgaggtctgtcgaggaggttaacctaggtactgaattaccctgttatccctaactagatatcgaattctaactataacggtcctaaggtagcgaatcgttcttcttggcgcgccaagacgcaaactcggaccggttcaagccgtcaaggcacttctatgcaaccacagtcaacttgaatgccgcttgagtgccttctcaagtttttttttcttgcaaaaatcatttcttttttttaaaaaaagtataatttggatcgtgcaaatttctctctaggtgtgtgtgtgactgtgtgagtaacaatttctctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttccccaacctcatcttctctcgtgttgttcggcacaacccgatcgatccccaactccctcgtcgtctctcctcgcgagcctcgtcgatcccccgcttcaaggtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcaggtgcaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgattaattaactaggctactgtagctagctgtgcatgtatgtggtgtggttactaaaataattagtgttttccttttgtttggaagcatatgtgtggtgaataaatgatgaactccgatgttcctctctataaatcttgatgattcgctagctatccgtacgtcgttgttctttgatttgatgatgagattgaaaaatggaatgtcatgctaaggagggtgccgcggcccggccgtgacggccacgagcgaactcctgcaggacaacggagcagcctcctcagcaaatcctaccacctcatttaaatagagtgaggttgatttgctgaggtagcggccgcgttaacaagcttctgcagaattcgtcaacgagatcttgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgacccaacgagatcttgagccaatcaaagaggagtgatgtagacctaaagcaataatggagccatgacgtaagggcttacgcccatacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacggtcgagaagacacggccattctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagtgatcaaccatggcccagatcaacaacatggcccagggcatccagaccctgaaccctaactctaacttccacaagccgcaagtgcccaagtctagctccttcctcgtgttcggctccaagaagctcaagaatagcgccaattccatgctggtcctgaagaaagactcgatcttcatgcagaagttctgctcctttcgcatcagtgcttcggttgcgactgcctgcatgctcaccttcgttaggaacgcctggtacgtcgccgctctccctgaggagctgagcgagaagcccttgggtcgcaccatcctagacactccgttagccctttaccgccagcctgacggcgtagtggcggccctgcttgacatctgcccgcataggttcgctccgctcagcgacggcatcctcgtcaacgggcatcttcagtgcccgtaccacgggctggaatttgacggcggtgggcagtgtgtccacaacccgcacggcaacggcgcacggccagcttccctcaacgttaggtcgttccctgttgtcgagcgcgacgcactgatctggatctggcctggcgacccagctctggccgatccaggagccattcccgacttcggttgccgcgtggacccagcctatcggacggtcggcggttacgggcacgtcgattgtaactataagctccttgtggacaaccttatggatttgggccacgctcagtacgtgcaccgggctaacgctcagactgacgcctttgaccgtctcgaaagggaggtcatcgtcggcgacggagagattcaggcgctgatgaagatccctggaggcacgccctctgtgctcatggcgaagtttctcagaggcgcgaacacgcccgtggacgcctggaacgacatccgctggaataaggtctccgcgatgctgaacttcatcgccgttgcgcccgagggcacacccaaagagcagtcaatccacagcagagggacccatattcttacaccggaaaccgaggctagttgccactacttcttcggctcgtcacggaatttcgggatagacgatccggagatggacggtgttcttcgatcttggcaagcgcaagctctcgtcaaggaagataaggtggtcgtggaggctatcgagcgtaggcgcgcctacgttgaggcgaacggtattaggcccgcgatgctgtcctgcgacgaggccgcagttagagtgtcgcgcgagatagaaaagctggagcagctagaggccgcctgaggtaccgagctcgtcaatcactagtgaattctgcatgcgtttggacgtatgctcattcaggttggagccaatttggttgatgtgtgtgcgagttcttgcgagtctgatgagacatctctgtattgtgtttctttccccagtgttttctgtacttgtgtaatcggctaatcgccaacagattcggcgatgaataaatgagaaataaattgttctgattttgagtgcaaaaaaaaaggaattagatctgtgtgtgttttttggatccccggggcggccgcacaacaaacgaatacgtcctgcttggcctactaggccaacgcaggcgctggccgtgacggccacgagcgaactgatatcgaattctagggataacagggtaatccacgtgtagctaaacgcgccctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatagcattcgcaatacagttagatgcgagtgaagcacgataagtcacaaccataatacatactattagaatccggctctttgccgagtgctttttttgggcactcggcaaagacttctttaccggcaaagtcctactctcggtaacgaccacgtttaccgagagcaggacgctcggtacagggagacactcggcaaagacctctttgtcgagtgccaaacgctcggcaaagggccgtcagcagccgtctatagcagatgtttattatctttgccgagcgccaagcgttggcactcggcaaaacaagtgttgccgagtgcttaaattggacactcggcaaaatatatttttcttttttctttttgcaaccaaactttctgtggtttgttcctacactatgtagacatacatgttccattttggcacaattataaaagtgtttgctataaatattagattttgttcgtttaattgaattttctcggataattcagatttgaactacaagtcactcaaaaaatggaaaaccatgaatgcaaaaatgatatccatgttatttagcacaagttacggccgatttcaggagtagacccgaatttttgagcaccatgctcacgaaacatgactgtgaacttgtcatccagttgttttaaaattgtataaaacacaaacaaaatcaaaaaatcatgaaacttgtccacatgtcatgatatcatcatatgtagaggttgtgataaaaagttgaaaatatttcgtgaaagttgtccatagttcactgagcctctttgtcgagtgtcacactcggcaaagcctttgtcgagtgcttcagacactcggcaaataagttgtttccagtagtgacattaatagaaaaagagtgcatagttcgttcacatagctaactaattggttcattacaaagaaacattagaacaagcataatgatgacttggacataatgaaataaaatacataggttgagggaaccaaacataatggaacacagaacaaatagaggacaacatcatagaaaacgacataacttcacatgggttttacgccaattgctccacgccaagtccataagtgctcgatgagatcatgttgaagttggttgtatcatttgagactagtgatgttgtggtgggagcatagccactctgtacgaacttcagggattgtgatgagagatccacgtgcactccaattgcggcaaactcaacattacttgcacgtgggaccttgtcctcgacaa

SEQ ID NO: 29 - DNA, 5629 bp, Artificial
  synthetic
gaggtcctgtatggaatcgtgttcgtttatttcccgggggccggggcaaaaaagacggcaatggattgttggacttgacatgtgcggtgcgtgggtccaacccggcctggcttttgggccagcgcgggccgacatagtgaggcccaaaatttaaaaagcacagctgcaggcccacggaaccagtggttatgaaaaacggaaacaaagatagtaaattttacctccttaaattgccaccgtatccaatatccaaattcaggtaatctattcctataaagcaccaatttccgctcttttcatctatcgtctgtcatgcgctctgttcctctccatcgtgtatcgcagataaaaggttacatgcatttccatgcatgtgatgggataaaaacaagaaaaaaggttgacatgcatttccatgcagataaaaggttacatggatttccttggagaaagtataataagactaaatgctgaggcggaggagagagagagaggagatgtgggtagtaaacttttagtcatctttgacacaagatcaaagaagatttgtgaaattatgcattaaaatatcgaagagctaactactacacgaataagctaaatggtaggctgcaaaggtgattacagctagcagttgactctattattaaacttcctcttagggcaacagtagttggaaaggtttttttggtgctgcccagatgcaaactaaaatccatgcatcctctctcaacctggaaggtgggcctaaaaaagatgatctaccatccacggatccacctgtcagctcaagttattgggtttaggaaacagggacctacgtggagatgtgtgctggacgggcgggcctcccacctgtcacgccgcaggcggaacggtgcgaaacgacgcacgcttttgctgtgcgcctgtgcgtctggcggtcagcgcgagcgtgactgcgttttcgtttgcgttagacgacgatcatcgctggaaatttggtattctctcacgttgaaggaaaatggattggagggagtatgtagataaattttcaaagcgttagacggctgtctttgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaccatggtagaaggtaaagtaaaatggttcaactctgaaaaaggtttcggattcatcgaagtagaaggtcaagacgatgtattcgttcatttctctgctattcaaggcgaaggcttcaaaactttagaagaaggccaagctgtttcttttgaaatcgttgaaggaaaccgcggaccacaagctgctaacgttactaaagaagcgtgaatttaaatgggcccgggggatccactagttctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatatcattctttacaagtatacgtgtttaaattgaataccataaatttttatttttcaaatacatgtaaaattatgaaatgggagtggtggcgaccgagctcaagcacacttcaattcctataacggaccaaatcgcaaaaattataataacatattatttcatcctggattaaaagaaagtcaccggggattattttgtgacgccgattacatacggcgacaataaagacattggaaatcgtagtacatattggaatacactgattatattaatgatgaatacatactttaatatccttacgtaggatcgatccgaattcgcgacacgcggccgctctagaactagtggatcccccccttaattaagggggctgcaggaattcataacttcgtataatgtatgctatacgaagttatagcttggtcgagtggaagctagctttccgatcctacctgtcacttcatcaaaaggacagtagaaaaggaaggtggcacctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatccttcgcaagacccttcctctatataaggaagttcatttcatttggagaggacacgctgaaatcaccagtctctctctacaagatcggggatctctagctagacgatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacgcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgatccccaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatcgggccactcgaccaagctataacttcgtataatgtatgctatacgaagttatcgcgccaaatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcacgttgaagaaaaatggatggagggaggaagtagataaagttttttgttgtatattgtgattttaatttgaaatcaagcttggtcaaaccgtggccgaaatttggcctggccactaatggccatgaaccaagcgtagtttgccgattaccccgtcccgacggtacgactttctctaatcgctcggttactgtccctgcaacctgcatctcatgactccaggccggcccaacaccagcagcgaccgcgaccaggctcctcctcctcctccagccacgggcaagaggccgcgcgcatgctctcgctcctgttcccggtaatccggcccagtaccttggtaccgcaccgtacctgtaatctctatctctagttctctagtacatattaagtcaatagtgtagactgtaacactaccatgacttcatcctcccttacctcgctctctgcgcacgcacaaaccacccttccgccccatataggagccgatatcgtgccccccgtcctggccgcacgcttccctaacccctcgtggactaggcttcccctccacgacgaggccacgacaatggttgcccccgcacgacgaggccgcggtgtgggcgaaggaggcgacgtgacctacagtccaaggcctcacatccacatacatgcgtcatctaattgattaatctatagcctggtcgcgctgtgctgctactgcttgatcgacgagtgctgttgcgacccgtctgtcatcttcgtcagctagacgaagcatccgagtacaactctaaacatacgaacattttaataacgagagcatataacgataaatagtgcttctacattaatgtatgttatcaatacttattgactcagtgacaaagcacggacatacatctagtagttaataataaaaataaataattaccttattaaacgatcatttattatataaatgtatttattttttatgtacatataataagttattacaatctgacaatatatataagtgatagaacataaagtagaggaacaaacggaacgtaaaggaaaacgaagctagtcaggtagatgctcccgaggacaaaaaaaaaggggcatagttgtcaagtttaatcttcccaagttttatcttacgtagtagtagagcgagagcggtccaattaagggcacgcacagttgcagcaggtgcagggctccagtagccgcggcgggtacgctcgcagtcgcagggcgccgcgcctagttctgctgcccggcccgggtcatgaaccaac

SEQ ID NO: 30 - DNA, 9920 bp, Artificial
  synthetic
atgggtctagaaatcccattcatctctaccgaggagacgagacagggtccactacgaggcctttataaagttctactagctttagaaaacccgctatgatttatacgagaatggggcgatatatgaatccctcgtctgaaaagtcatcgcaacatgatcaacccgagaacctccttatatcggtaactcctcacaccgcggttgcccctttcgggtaaggtagttctcaactagcattcctaaatagtcagtcaagtgcgtcacataccacctttgtggtagcactgttttcctgtgtggttctccatgtttcgattaacaaaatagttttatcatgaacaataaataatccaacaataatgatagagtatgatcataattcagatataacattaataccaaaaccaggtagagtaatagcaaaactacacaatagttcatttgtttgcaaggtgtgggacaaacaaaactaggtaatacttcaaacactgatagtttaaactggcactagcctaacggtgttgactaactaggccgcttccctaattagctaaccatggcccgggactgaggcgcgccgggacccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactattgcgtttgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgccggaccctggaaaaccaaaacaacgaataagcaaactgcaggaaaagtatgcagtggaaaccaacccagattcggacgataggaaagtatcaagtgaatgatttgccaggaaaaggagaggggtaaaaaggggcgaagatttagaagatctaaagcacaagaaccagagattagattgaacaatagggaacttggagcatcctttttttcttcagggaaaaactgaaaatccaaaccatgttgagcaaaaccgagtgggattggaaaccaaaaaacccgagataaagaaactcgagaaaaagcatgaaatcgaaaccaacttcagtaaaacaaaaggaggacagaaaagaaagtcggaaggtataaagaatacattaacattcagtgaaacagcatgctgtcttcttctttttttatgcacaacagagcatacatatataccttcccaggctgaggacttggcggaggagagccgcggagaggttggcggtgcagacggtctggacgggcccgaagacggagacgaacagcgggcccttcctgcccaggcaccacgcttggaacgccaagcacgcgccaaccgcggccccgccgaggacgacgatccccgcgacaagcgtggcgtcgatcctgggcgaccccaagccgaggaacctcccttccaggacgagccgcaggacggcggtgagagaggcacccgtcgcggaggtggcgcagcacaaggtgagcagcgcggggaaggcggcgagcgtggcggcctgcaggacggtgacgagcgcgaagacggtgacgccggcgacgaggcagcagcagccgaggatccagtcgtaggaggaagccggaccaaaccgggcaatgcaacctgcagatgcactagacggaggaaacgaggaggaggagaaaacagagcaagagcaggcggagagaagatagagcaaaacacgagtgaggcacagcgtaagcactcggtagaagtctccagaggcgaggtgcgcacaggagaacagatgagtaaagtcagccaaggatccacgatccaacggctacgaatttttggagtgacgtggataggctcaaaggcgccatttccatccggctttatagtattttaaaaaaattcattttcctccctctagtgtgtgcggaggcgtgagcccgtttaacggcgttgagaagtctaacggacaccaaccacaaccaggaaccagcgccggccgcgccgccgagtgaagcagactgcatacggcacggcgcggcatctctctggctgcctctcgagagttccgcccccaccttcccgcggtagcgtggtggtttcgctttccgctgtcggcatccggaagttgcgtggcagagtggacggagacgaggccgggtcctccagctcctctcaaacgtcacggcaccggcatccggcagccagcgcggtccttcccaaccactcgttcccaacccatcccccttcctcgcccgccgtcataaatagccagccccatccccagcttctttccccaacctcatcttctctccttttgctctgaacgcacacaccgcccggtctccgatctccgatccccgatcccctcgtcgatcctaggtacggcgaccatcctcccccccccccccccccctctctctctgccttctctagatcggcgatccgatccatgcttacttggttagggcctgctaactatgttcatgtttgcgttagatccgtgcatggacgcgatctgtacacaccagacgcgttctgattgctagctaactcgccagtacctgggaatcctgggatggctgtagccggccccgcacgcagacgggaccgatttcatgattctctatttttttctttgtttcgttgcctagggtttcgttcgatcgatccgcgttattctttatttccatatattctggtacgatgttgatacggttcgaccgtgctgcttacgttctgtgcgcttgtttgccgggtcatttttaccttgccttttttgtatggtttggttgtggcgatgtggtctggtcgggctgtcgttctagatcggagtagagtgctgtttcaaactgtctagcggatctattagatttggatctgcatgtgtgacatatatcttcgtagttaagatgatgcatctgtatgtgtgacatgcggatctattagatttggatctgtatgtgtgacatatatcttcgtagttgagatgatgcatctgtatgtgtgacatatatcttcgtagttaagattatgcatggaaatatcaatcctttagataaggacgggtatacttgttgctgtgggttttactggtacttcgatagatgcatatacatgatctaacatgcttagatacatgaagtaacatgctgctacggtttaataattcttgagttgatttttactggtacttagatagatgtatatacatgcttagatacatgaagtaacatgctcctacagttcctttaatcattattgagtacctatatattctaataaatcagtatgttttaaattattttgattttactggtacttagatagatgtatatatacatgctcaaacatgcttagatacatgaagtaacatgctgctacggtttagtcattattgagtgcctatatattctaataaatcagtatgttttaaattattttgattttactggtacttagatagatgtatatatacatgctcaaacatgcttagatacatgaagtaatatgctactacggtttaattgttcttgagtacctatatattctaataaatcagtatgttttaaattatttcgattttactggtacttagatagatgtatatatacatgcttagatacatgaagtaacatgctactacggtttaattgttcttgaatacctatatattctaataaatcagtatgttttaaattatttcgattttactggtacttagatagatgtatatatacatgctcgaacatgcttagatacatgaagtaacatgctacatatatattataataaatcagtatgtcttaaattattttgattttactggtacttagatagatgtatatacatgctcaaacatgcttagatacatgaagtaacatgctactacggtttaatcattattgagtacctatatattctaataaatcagtatgttttcaattgttttgattttactggtacttagatatatgtatatatacatgctcgaacatgcttagatacgtgaagtaacatgctactatggttaattgttcttgagtacctatatattctaataaatcagtatgttttaaattatttcgattttactggtacttagatagatgtatatatacatgctcgaacatgcttagatacatgaagtaacatgctactacggtttaatcgttcttgagtacctatatattctaataaatcagtatgtcttaaattatcttgattttactggtacttagatagatgtatatacatgcttagatacatgaagtaacatgctactatgatttaatcgttcttgagtacctatatattctaataaatcagtatgtttttaattattttgattttactggtacttagatagatgtatatatacatgctcgaacatgcttagatacatgaagtaacatgctactacggtttaatcattcttgagtacctatatattctaataaatcagtatgtttttaattattttgatattactggtacttaacatgtttagatacatcatatagcatgcacatgctgctactgtttaatcattcgtgaatacctatatattctaatatatcagtatgtcttctaattattatgattttgatgtacttgtatggtggcatatgctgcagctatgtgtagattttgaatacccagtgtgatgagcatgcatggcgccttcatagttcatatgctgtttatttcctttgagactgttcttttttgttgatagtcaccctgttgtttggtgattcttatgcagatccagatcttcgtatttttacaacaattaccaacaacaacaaacaacaaacaacattacaattactatttacataaaccatggcttcgatctcctcctcagtcgcgaccgttagcaggaccgcccctgctcaggccaacatggtggctccgttcaccggccttaagtccaacgccgccttccccaccaccaagaaggctaacgacttctccacccttcccagcaacggtggaagagttcagtgtatgcaggtgtggccggcctacggcaacaagaagttcgagacgctgtcgtacctgccgccgctgtctatggcgcccaccgtgatgatggcctcgtcggccaccgccgtcgctccgttccaggggctcaagtccaccgccagcctccccgtcgcccgccgctcctccagaagcctcggcaacgtcagcaacggcggaagaatccggtgcatggccggtgccgaggagatcgtgctgcagccgatcaaggagatcagcggcaccgtgaagctgccgggcagcaagagcctgagcaaccgcatcctgctgctggccgccctgagcgagggcaccaccgtggtggacaacctgctgaacagcgaggacgtgcactacatgctgggcgccctgaggaccctgggcctgagcgtggaggccgacaaggccgccaagagggccgtggtggtgggctgcggcggcaagttcccggtggaggacgccaaggaggaggtgcagctgttcctgggcaacgccggcatcgccatgaggagcctgaccgccgccgtgaccgccgccggcggcaacgccacctacgtgctggacggcgtgccgaggatgagggagaggccgatcggcgacctggtggtgggcctgaagcagctgggcgccgacgtggactgcttcctgggcaccgactgcccgccggtgagggtgaacggcatcggcggcctgccgggcggcaaggtgaagctgagcggcagcatcagcagccagtacctgagcgccctgctgatggccgccccgctggccctgggcgacgtggagatcgagatcatcgacaagctgatcagcatcccgtacgtggagatgaccctgaggctgatggagaggttcggcgtgaaggccgagcacagcgacagctgggacaggttctacatcaagggcggccagaagtacaagagcccgaagaacgcctacgtggagggcgacgccagcagcgccagctacttcctggccggcgccgccatcaccggcggcaccgtgaccgtggagggctgcggcaccaccagcctgcagggcgacgtgaagttcgccgaggtgctggagatgatgggcgccaaggtgacctggaccgagaccagcgtgaccgtgaccggcccgccgagggagccgttcggcaggaagcacctgaaggccatcgacgtgaacatgaacaagatgccggacgtggccatgaccctggccgtggtggccctgttcgccgacggcccgaccgccatcagggacgtggccagctggagggtgaaggagaccgagaggatggtggccatcaggaccgagctgaccaagctgggcgccagcgtggaggagggcccggactactgcatcatcaccccgccggagaagctgaacgtgaccgccatcgacacctacgacgaccacaggatggcgatggccttcagcctggccgcctgcgccgaggtgccggtgaccatcagagacccgggctgcaccaggaagaccttcccggactacttcgacgtgctgagcaccttcgtgaagaactaaagagctcgccaaggttcaattaagctgctgctgtacctgggtatctgcgtcgtctggtgccctctggtgtacctctatatggatgtcgtcgtctaataaacatctgtggtttgtgtgtcatgaatcgtggttgtggcttcgttggtttaatggacctgttgtgtcctctgtgttgtacccaaaactcttctgcagcagtatggcttgaatccttatgaagtttgatatttgaacttaaaagtctgctcattatgtttttttctggttatatctcctaattaactgcctgggatcaaatttgattcgctggtgtttattggacccctcccaggttcttgctttctaccgtttcttgctgaatgttaacttgattctgtcaggctcagtttcccactatggcttacagcttaacgtgtttggtttgttgaatgttaacttggttttgtcaagctcagttttttactctggcttacagcataacatgtttgacttttggttttgctgctttgttattgggttctgggtagttcttgatgaatccaaaagatcatgtgcacagccatattatctatttaagcgatccaggttattactatgaaaggatgccttctagctaaggagtagttaggttttttcttcaaggttaaattttctcgatgctctagtgttcctgtgaccataatcataataattcctttgaaagctctatggtccctggaagcagggcatacaatgcaagacagcaacttgatcacatcaactgaagtatacagggttctcttaactcttggtgacttcggtttaatggaccggttgtactcgtgttctatccgtaaccgttgtgatgtcttgtgtgtttggttgcgggatagctgggaccacgacgtttccgtctaattctgatggatagctatagacggcactgagatggttatattataacctctgatcctgaactctacgagatcgtctcatccgtcattgccaccaaatacaccattaaattacggacccgatttaaataatattcggaccgctagtgcatgcactgactaattagctaaagtcaaagattcaaatagaggacctaacagaactcgccgtaaagactggcgaacagttcatacagagtctcttacgactcaatgacaagaagaaaatcttcgtcaacttggtggagcacgacacgctagtctactccaaaaatatcaaagatacagtctcagaagaccaaagggcaattgagacttttcaacaaagggtaatatccggaaacctcctcggattccattgcccagctatctgtcacttaattgtgaagatagtggaaaaggaaggtggctcctacaaatgccatcattgcgataaaggaaaggccatcgttgaagatgcctctgccgacagtggtcccaaagatggacccccacccacgaggagcatcgtggtaaaagaagacgttccaaccacgtcttcaaagcaagtggattgatgtgatatctccactgacgtaagggatgacgcacaatcccactatccttcgcaagacccttcctctatataaggaagttcatttcatttggagaggataattatccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggctccacattgtacacacatttgcttaagtctatggaggcgcaaggttttaagtcagtggttgctgttataggccttccaaacgatccatctgttaggttgcatgaggctctaggatacacagcgcggggtacactgcgcgcagctggatacaagcatggtggatggcatgatgttggtttttggcaaagggattttgagttgccagctcctccaaggccagtgaggccagttacccagatctgactaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcattagcctgcaggcccgggttagtccatggctaattagctaacggccaggatcgccgcgtgagcctttagcaactagctagattaattaacgcaatctgttattaagttgtctaagcgtcaatttgtttgtcccacaccttgcaaacaaatgaactattgtgtagttttgctaagcatgtcacggtgattaataggaacgttattaggtaaagaatgtgatcaagagcacaacttgcctttttggtgcaactcgaggtacttctccaagttgagactcaaattcctcgtgacatcgcttctatttgtaacaatacatatacgtagacaaataataggtaaaaataacattacaccaaagatggaaacaaccaaatcaataatattctacgcgccgctacgagatcgtaagtttgagaatcactaaaattaaagctaaaataaaatagttttgaattttctaagtttctggatttatttttgtactaaaaatcattttctcgtcttttattttatatatataaataatccagactgtgggtagatatatgctaaagcgcaggttctgatttgaaatgttttggaccaatttgtaataatttgtatatattgttggactacaggttcatttataaaatcggtagaggctcttacacaatt

SEQ ID NO: 31 - DNA, 8487 bp, Artificial
  synthetic
aacggagctggtttaaactggcactagcctaacggtgttgactaagtaggccgcttccctaattagctaagggacccgggtcattgagcggagaattaagggagtcacgttatgacccccgccgatgacgcgggacaagccgttttacgtttggaactgacagaaccgcaacgaatattggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccaccatgactagtaacggccgccagtgtgctggtattcgcccttatgacggccgacaacaacaccgaggcctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgaacgagctgttcaccagcagcaaccagatcggcctgaagaccgacgtgaccgactaccacatcgatcaggtgtaggagctgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggacccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaccatgacggccgacaacaacaccgaggccctggacagcagcaccaccaaggacgtgatccagaagggcatcagcgtggtgggcgacctgctgggcgtggtgggcttccccttcggcggcgccctggtgagcttctacaccaacttcctgaacaccatctggcccagcgaggacccctggaaggccttcatggagcaggtggaggccctgatggaccagaagatcgccgactacgccaagaacaaggcactggccgagctacagggcctccagaacaacgtggaggactatgtgagcgccctgagcagctggcagaagaaccccgctgcaccgttccgcaacccccacagccagggccgcatccgcgagctgttcagccaggccgagagccacttccgcaacagcatgcccagcttcgccatcagcggctacgaggtgctgttcctgaccacctacgcccaggccgccaacacccacctgttcctgctgaaggacgcccaaatctacggagaggagtggggctacgagaaggaggacatcgccgagttctacaagcgccagctgaagctgacccaggagtacaccgaccactgcgtgaagtggtacaacgtgggtctagacaagctccgcggcagcagctacgagagctgggtgaacttcaaccgctaccgccgcgagatgaccctgaccgtgctggacctgatcgccctgttccccctgtacgacgtgcgcctgtaccccaaggaggtgaagaccgagctgacccgcgacgtgctgaccgaccccatcgtgggcgtgaacaacctgcgcggctacggcaccaccttcagcaacatcgagaactacatccgcaagccccacctgttcgactacctgcaccgcatccagttccacacgcgtttccagcccggctactacggcaacgacagcttcaactactggagcggcaactacgtgagcacccgccccagcatcggcagcaacgacatcatcaccagccccttctacggcaacaagagcagcgagcccgtgcagaaccttgagttcaacggcgagaaggtgtaccgcgccgtggctaacaccaacctggccgtgtggccctctgcagtgtacagcggcgtgaccaaggtggagttcagccagtacaacgaccagaccgacgaggccagcacccagacctacgacagcaagcgcaacgtgggcgccgtgagctgggacagcatcgaccagctgccccccgagaccaccgacgagcccctggagaagggctacagccaccagctgaactacgtgatgtgcttcctgatgcagggcagccgcggcaccatccccgtgctgacctggacccacaagagcgtcgacttcttcaacatgatcgacagcaagaagatcacccagctgcccctggtgaaggcctacaagctccagagcggcgccagcgtggtggcaggcccccgcttcaccggcggcgacatcatccagtgcaccgagaacggcagcgccgccaccatctacgtgacccccgacgtgagctacagccagaagtaccgcgcccgcatccactacgccagcaccagccagatcaccttcaccctgagcctggacggggcccccttcaaccaatactacttcgacaagaccatcaacaagggcgacaccctgacctacaacagcttcaacctggccagcttcagcacccctttcgagctgagcggcaacaacctccagatcggcgtgaccggcctgagcgccggcgacaaggtgtacatcgacaagatcgagttcatccccgtgaactagatctgaggggtaccagctcttgacgacctgctaagatcgttcaaacatttggcaataaagtttcttaagattgaatcctgttgccggtcttgcgatgattatcaatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctattgttactagatctaattgacggacccggcgcgccatttaaatggtaccggtccggcatgcatgcagggatccacatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcactagtccaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcgcggggtacattgcgcgcagctggatacaagcatggtggatggcatgatgttggtttttggcaaagggattttgagttgccagctcctccaaggccagttaggccagttacccagatctgaatagtgatatcggcgcctgggtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgtagccctgcaggaaatttaccggtgcccgggcggccagcatggccgtatccgcaatgtgttattaagttgtctaagcgtcaatttgtttacaccacaataccaggcccag

SEQ ID NO: 32 - DNA, 5092 bp, Artificial
  synthetic
tttcctcattttctttttcccgcttttgtttcaatttttcttgggtaatgtacagtgagtatattttttcttgttctttttctcatggccaaaatccacaatggatcgatgaattagctgtcgttgttgccaacaacaacaacagaacaaaatcacgtgacgtactagcacaatgcaagtagccaaactgagcttccgggcaccgacgaacggttgcacgccatcggcgggaaggaacaggccgggctgtcaatggacaaacgggccgccaagctggagggagtgtcatgggctttgagaaccatcgtcagggtccagtttattcttttgtttttattaaaggcggtaaactcggggaacgaatatactaggaaaaacactagccagtcagagtcagtcaaagtggactgagttaaaattgcaacgacacacacgcagcagtcagggcgtcgggaatgaacaatggatgaatttattataatctgaagaaaacgaagggacacagccactacgaacactggggagtggggagtgaatgaatgaatgcattccactggaccgttccagcgcttcgtgtgcctcgctagatgcgctgaacactcgaacgccatggacctcgctccgctctctatatatagagggaaggccttcagtctactcctcgggatataccactgaacgtcaccaagaagatcagtactcaaacactgatagtttaaactgaagaagcttaatttaaatccctcgagtagcggccgctagcccgggcatagcttaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtcggagaaattcatcgaagcgaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatggcacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagctgcagaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgatccccgcaagggatccggcatggaaactgatcgccttgtgatcccaggatcgaaaagcatcaccaaccgggctttgcttttggctgccgcagcgaagggcacgtcggtcctggtgagaccattggtcagcgccgatacctcagcattcaaaactgcaatccaggccctcggtgccaacgtctcagccgacggtgacgattgggtcgttgaaggcctgggtcaggcacccaacctcgacgccgacatctggtgcgaggacgcaggtactgtggcccggttcctccctccattcgtagccgcaggtcaggggaagttcaccgtcgacggatcagagcagctgcggcggcgcccgcttcggcccgtggtcgacggcatccgccacctgggcgcccgcgtctcctccgagcagctgccccttacaattgaagcgagcgggctggcaggcggggagtacgaaattgaagcccatcagagcagccagttcgcctccggcctgatcatggccgccccgtacgcgagacaaggcctgcgtgtgaagataccaaatcccgtgtcacagccctacctcacgatgacactgcggatgatgagggacttcggcattgagaccagcaccgacggagccaccgtcagcgtccctccagggcgctacacagcccggcggtatgaaatagaaccggatgcgtcaactgcgtcgtacttcgccgccgcttccgccgtctctggcaggcgcttcgaatttcaaggccttggcacagacagcatccaaggcgacacgtcattcttcaatgtacttgggcggctcggtgcggaggtccactgggcatccaactcggtcaccatacggggaccggaaaggctgaccggcgacattgaagtggatatgggcgagatttcggacaccttcatgacactcgcggcgattgcccctttggccgatggacccatcacgataaccaacattggtcatgcacggttgaaggaatccgaccgcatctcagcgatggaaagcaacctgcgcacgctcggtgtacaaaccgacgtcggacacgactggatgagaatctacccctctaccccgcacggcggtagagtgaattgccaccgggaccacaggatcgctatggcgttttcaatcctgggactgagagtggacgggattaccctcgacgaccctcaatgcgtcgggaagacctttcctggcttcttcgactaccttggacgccttttccccgaaaaggcgcttacgctccccggctagggcgcgcctccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcacgtgttgagcatataagaaacccttagtatgtatttgtatttgtaaaatacttctatcaataaaatttctaattcctaaaaccaaaatccagtactaaaatccactcgagacgcgtgaattcagtacattaaaaacgtccgcaatgtgttattaagttgtctaagcgtcaatttgtttacaccgttctcagagggagatgggcggcaagggcggcgggggtggtggcaagggcggcggcgggggtggtggcaagggcggaggaggttttggtggcaagagcggcggcgggggtggtggcaagggcggaggaggtgttggtggcaagagcggcggcggcaagtcaggcggcggcggcggtgggggctatggtggtggagggaagtcaggctccggcggcagtggcggcgacggaatgatgaaggcgcccggcggcagtggcgagtacatctcccgctctgtcttcgaggccagcccgcaggtgttcttccatggcctccaccagggaggaggcaacaacaagtagatccatctagctagactgctgctgctacttcacaagcttgggacgatgtgtgatcatgcatgcttggactggcatcagtctctatgtagcttctgaataaaataaaatgtaacgatgctcgattgtgtttcacttgctcgcttgtttcagccaagttattatatatcatcaggctcgtacgtcagctatatatatatatatatatatatatatatatatatatatatatatatatatatatatatatatatatatatatatatatatatatatacacacacacacatatgcaggtgcatggattgtgcaacgcgaatgtgtgattgtgctaatccgttagttgatgccgtttgttgctt

SEQ ID NO: 33 - DNA, 9425 bp, Artificial
  synthetic
  misc_feature (9377)..(9382): n is a, c, g, or t
atgaaaaagtccaagtcgagcaagggtacgtaccgcggccggcggctaattacggaggacatgtcgtagtagctggtagtaaattaacacacgcgtacgagtagcggagttaaatgggggcatgcatgcagcaggacgtggtattagtaagcttactactctagctttatccatccatccatcgcgctagctggctgcaggcacgggttatcttatcttgtcgtccagaggacgacacacggccggccggtgaagtaaaagggagtaatcttattttgccaggacgaggggcggtacatgatattacacacgtaccatgcatgcatatatgcatggacaaggtacgtcgtcgtcgatcgacgtcgatgcatatgtgtgtatgtatgtacgtgcataatgcatggtaccagctgctggcttatatatatttgtcaccgatcgatgcatgctgctgctctacacggtttgacactttaatttgactcatcgatgaccttgctagatagtagcggctcgtcaattaatgagccatcaagttaacaagagggcacgggcttgcgcgactgattccaccttattaacatacgccctgcgcccgcgcgtgctgtacgtacgagaatttcgaattacattaattcaaagctgtgtatgtatgtatatatatatgtgcgtttttttgtgtgtgtatgtctctttgcttggtctttctctatcgatccccctctttgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctgggccccccctcgaggtcggccgcattcgcaaaacacacctagactagatttgttttgctaacccaattgatattaattatatatgattaatatttatatgtatatggatttggttaatgaaatgcatctggttcatcaaagaattataaagacacgtgacattcatttaggataagaaatatggatgatctctttctcttttattcagataactagtaattacacataacacacaactttgatgcccacattatagtgattagcatgtcactatgtgtgcatccttttatttcatacattaattaagttggccaatccagaagatggacaagtctaggttaactgactagctagtcagtacacagtcctgccatcaccatccaggatcatatccttgaaagccccaccactagggatcataggcaacacatgctcctggtgtgggacgattatatccaagaggtacggccctggagtctcgagcatcttctttatcgctgcgcggacttcgttcttctttgtcacacggaccgctggaatgttgaaccctttggcgatcgtcacgaaatctggatatatctcactttcattctctgggtttcccaagtatgtgtgcgctctgttggccttatagaacctgtcctccaactgcaccaccatccccaggtgctggttgtttagcacaaagaccttcaccgggaggttctcaattcggatcatagctagctcctgaacgttcatgagaaagctaccatctccatcgatgtcaacaacagtgacacctgggttggccacagaagcaccagcagcagccggcaaaccaaatcccatagccccaagaccagctgaagacaaccactgccttggccgcttgtaagtgtagtactgtgccgcccacatctggtgctgcccaacacctgtgccgatgatggcctcgcctttcgtcagctcatcaagaacctgaatagcatattgtggctggatctcctcattagatgttttatacccaagggggaattccctcttctgctgatccaactcatcgttccatgagccaaagtcaaagctcttctttgatgtgcttccttcaagaagagcattcatgccctgcaaagcaagcttaacatctgcacagatggacacatgtggctgcttgttcttgccaatctcagccggatcaatatcaacgtgcacaatcttagccctgcttgcaaaagcctcaatcttccctgtcacgcgatcatcaaaccgcacaccaagtgcaagcaacagatcggccttatccactgcataatttgcatacaccgtcccatgcatacctagcatgcgcagagacagtgggtcgtcgctggggaagttgccgaggcccataagagtagttgtgaccgggattccagtcagctccacaaagcgtcgcaactcctcaccagatgctgcgcagccaccgcccacataaagaacagggcgccgcgattcaccaacaagacgcagcacctgctcaagcaactcagtcgcagggggcttgggaaggcgcgcaatgtacccaggcagactcatgggcttgtcccagacaggcaccgccatctgctgctggatgtccttggggatgtcgacaagcaccggccctggtcgaccagaggaggcgaggaagaaagcctcctgcacgacgcgggggatgtcgtcgacgtcgaggaccaggtagttgtgcttggtgatggagcgggtgacctcgacgatgggcgtctcctggaaggcgtcggtgccaatcatgcgtcgcgccacctgtcccgtgatggcgaccatggggacggaatcgagcagcgcgtcggcgagcgcggagactaggttggtggcgccggggccggaggtggcgatgcagacgccgacgcggcccgaggagcgcgcgtagccggaggcggcaaaggcctccccttgctcgtggcggaagaggtggttggcgatgacgggggagcgggtgagtgcctggtggatctccatggacgcgccgccggggtaggcgaagacgtcgcggacgccgcagcgctcgagggactcgacgaggatgtcagcacccttgcggggctcggtggggccccacggccggagcggggtggccgggggagccatcggcatggcgggtgacgccgctgagcacctgatgggcgcggcgagggcgcggcgggtggccaggaggtgcgcccggcgcctcgccttgggcgcagcggtagtggcgccagtgagcgcggtagacgcggcggcggcggtggccatggttgcggcggctgtctcggaggcggcgcgagggtttggggtgggtgccacggacacggagtgggagaaagggggatgtgcgtggaggcctccctgcttttgttcagaggatgtgtggctcagatggtgatgggaatgggactcgcaagacgacgacgacacgtccgtcgcccgaatacgtacacgctacagaccggacggtggggcctgtcgacgtgggaccacgtgtcggcctggattacaaacagtggtgtccaccgagtgctggtacacgacagcgtgcgtcaaggaaggttttgaactgttccgttaaaaaaagaggggagattttggacttgactgtggacgacggtgcatgtcatcggagtacagacggtactgacacaaggggcccagacaagggaatccaaacgggtcgcacccacctgccaggctgccacccgcaatccgcaacagggaaaccgggcacagcccacaaccacaagatgagcagctgcggcgacagcgtcaggcccggtgtcggtgttagggatggcaccctttggctccccgtatccgtccccgcgacaaaaaaatttcccgcggggattcccacgaactcttgcgagagacatttcttccccatccccgttccccacggggataaatccccatcggggatcctctagagtcgacctgcaggcatgcaagcttcggtccgcggccagcttgctaacccgggccccccctcgaggtcatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacgaagattttcttcttgtcattgagtcgtaagagactctgtatgaactgttcgccagtctttacggcgagttctgttaggtcctctatttgaatctttgactccatggacggtatcgataagctagcttgatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacgaagattttcttcttgtcattgagtcgtaagagactctgtatgaactgttcgccagtctttacggcgagttctgttaggtcctctatttgaatctttgactccatgatcgaattatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacgaagattttcttcttgtcattgagtcgtaagagactctgtatgaactgttcgccagtctttacggcgagttctgttaggtcctctatttgaatctttgactccatgggaattcctgcagcccgggatctaggagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaccgccggggatccacacgacaccatggctattgaggttaagcctatcaacgcagaggatacctatgaccttaggcatagagtgctcagaccaaaccagcctatcgaagcctgcatgtttgagtctgaccttactaggagtgcatttcaccttggtggattctacggaggtaaactgatttccgtggcttcattccaccaagctgagcactctgaacttcaaggtaagaagcagtaccagcttagaggtgtggctaccttggaaggttatagagagcagaaggctggttccagtctcgtgaaacacgctgaagagattctcagaaagagaggtgctgacatgatctggtgtaatgccaggacatctgcttcaggatactacaggaagttgggattcagtgagcaaggagaggtgttcgatactcctccagttggacctcacatcctgatgtataagaggatcacataactagctagtcagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattcagtacattaaaaacgtccgcaatgtgttattaagttgtctaagcgtcaatttgctgtacatgtaaccagctttcctagaaaaaaaacacagtaatatttacagtatacaataatgctcattgattagatccaaaattatgaaatgtatatattttttgccataacttttattacagtcttatactgaagtaaagttgtatcacgtcatgaacatagaataagactgagatagttgactaatccagtcatatcctatagtacactagtaaggtgcatgtgctaacaatatgacaaatctagtatttctttatagtttatcatccataaatcgcaaaatacctaagcataattttatttgaatagagagagtactgcccgttattgtatgaggtaatgacttccatatatacaacatagtcataagaacacaattatgaaaaaaatctatttaaaattgaaatgcgttgagatcttgtcttgcatactaaacatagtaaatataaattaatgcataaatgactgttataacagacaatgctggtgacaatagacaatgtactgaatctaactggatacgtaggatgctgctatcttattcactcatagttattcagatagtggtcattctttttgacccatagaaaactgtgtgctataatacaccaaaaggaaagcaaagtgaaaaggaaactttgaatagccaagaagactcggagtgcttcacgccttcacctatcccacataggtgatgagctaagagtaaaatgtagattctctcgagtactgaatattgcctgcacttttccttgcagtaaatacacctttaatccatgacgagagtccactctttgagtccgtcttgagattcttccattgatcatacaacatgacctcgaagtcctgatggagaacaacttatataattaaaactacaatacagaaagttcctgacaattaaaacctttggtggtggcatgccgtaggttaaaaaaaatagataatgacaacacaactggagacacgctctttgccgagtgctcacacgtttgctgagagcgagcactcggcaaatatatgatttgccgaataccaccctcctcggcaaaacaatacactaggcaaaaaggtagtttcccatcaccatgatgcccgccgttaatgtaccttctatgccgagtatgttggcgctcagcaaagagatcgttaccggcgtttgtttcaccaagagctctttgacgagtgtggcacacgacaaaaccttttgccgagtgtaattagtcgtttgccaagtgactggtgcagttggcaaaggagtnnnnnnttatgtgtgggcaaaatgatatatggtgccagttagggctagc

SEQ ID NO: 34 - DNA, 6466 bp, Artificial; identical to SEQ ID NO: 16
  synthetic

SEQ ID NO: 35 - DNA, 23 bp, Artificial
  synthetic
gaagaaaatcttcgtcaacatgg

SEQ ID NO: 36 - DNA, 23 bp, Artificial
  synthetic
tccagggcgagctcggtacccgg

SEQ ID NO: 37 - DNA, 23 bp, Artificial
  synthetic
ggccgcggaccgaattcccatgg

SEQ ID NO: 38 - DNA, 23 bp, Artificial
  synthetic
atcgtggcctcttgctcttcagg

SEQ ID NO: 39 - DNA, 23 bp, Artificial
  synthetic
cgaggcaagcttgtcgaaaatgg

SEQ ID NO: 40 - DNA, 23 bp, Artificial
  synthetic
aaacactgatagtttaaacgcgg

SEQ ID NO: 41 - DNA, 23 bp, Artificial
  synthetic
tgcactgcaggcatgcaagctgg

SEQ ID NO: 42 - DNA, 23 bp, Artificial
  synthetic
tgtactgaattgtctagacccgg

SEQ ID NO: 43 - DNA, 23 bp, Artificial
  synthetic
cgcgttaacaagcttactcgagg

SEQ ID NO: 44 - DNA, 23 bp, Artificial
  synthetic
agatcggggatagcttctgcagg

SEQ ID NO: 45 - DNA, 23 bp, Artificial
  synthetic
tgcactgcaggcatgcaagctgg

SEQ ID NO: 46 - DNA, 23 bp, Artificial
  synthetic
ggcaccggtaaatttcctgcagg

SEQ ID NO: 47 - DNA, 23 bp, Artificial
  synthetic
tgcactgcaggcatgcaagctgg

SEQ ID NO: 48 - DNA, 23 bp, Artificial
  synthetic
actagatctgctagccctgcagg

SEQ ID NO: 49 - DNA, 23 bp, Artificial
  synthetic
cgcggccgcttaattaaggccgg

SEQ ID NO: 50 - DNA, 23 bp, Artificial
  synthetic
cgggtttctagtcaccggttagg

SEQ ID NO: 51 - DNA, 27 bp, Artificial
  synthetic
tttggactgagaattagcttccactcg

SEQ ID NO: 52 - DNA, 27 bp, Artificial
  synthetic
tttctcatctaagcccccatttggacg

SEQ ID NO: 53 - DNA, 23 bp, Artificial
  synthetic
ttctgcaggtcctgctcgagtgg

SEQ ID NO: 54 - DNA, 23 bp, Artificial
  synthetic
cggccgcttcgagtggctgcagg

SEQ ID NO: 55 - DNA, 23 bp, Artificial
  synthetic
gtacgccatgctggccgcccggg

SEQ ID NO: 56 - DNA, 23 bp, Artificial
  synthetic
cttggctccaaatccggtaccgg

SEQ ID NO: 57 - DNA, 23 bp, Artificial
  synthetic
ttctgcaggtcctgctcgagtgg

SEQ ID NO: 58 - DNA, 23 bp, Artificial
  synthetic
atcgatgcggccgcttcgagtgg

SEQ ID NO: 59 - DNA, 27 bp, Artificial
  synthetic
tttaaactatcagtgtttgagcgcttt

SEQ ID NO: 60 - DNA, 27 bp, Artificial
  synthetic
tttaaacgctcttcaactggaagagcg

SEQ ID NO: 61 - DNA, 27 bp, Artificial
  synthetic
tttctaattcctaaaaccaaaatccag

SEQ ID NO: 62 - DNA, 27 bp, Artificial
  synthetic
tttggaacaagtggctatcgccagata

SEQ ID NO: 63 - DNA, 27 bp, Artificial
  synthetic
tttcccgccttcagtttaaactatcag

SEQ ID NO: 64 - DNA, 27 bp, Artificial
  synthetic
tttggaactgacagaaccgcaacgttg

SEQ ID NO: 65 - DNA, 27 bp, Artificial
  synthetic
tttcccgccttcagtttaaactatcag

SEQ ID NO: 66 - DNA, 27 bp, Artificial
  synthetic
tttaaatcaattgggcgcgccgaattc

SEQ ID NO: 67 - DNA, 27 bp, Artificial
  synthetic
tttctccatattgaccatcatactcat

SEQ ID NO: 68 - DNA, 27 bp, Artificial
  synthetic
tttgatgaagtgacaggtaggatcgga

SEQ ID NO: 69 - DNA, 27 bp, Artificial
  synthetic
tttaaaaataaatttttaatttagttg

SEQ ID NO: 70 - DNA, 27 bp, Artificial
  synthetic
tttaatcgcggcccatgatcacaccgg

SEQ ID NO: 71 - DNA, 27 bp, Artificial
  synthetic
tttgacacacacactaagcgtgcctgg

SEQ ID NO: 72 - DNA, 27 bp, Artificial
  synthetic
tttgaattcgagctcggtacccgggga

SEQ ID NO: 73 - DNA, 27 bp, Artificial
  synthetic
tttaaactatcagtgtttggagcttga

SEQ ID NO: 74 - DNA, 27 bp, Artificial
  synthetic
tttggactgagaattagcttccactcg

SEQ ID NO: 75 - DNA, 27 bp, Artificial
  synthetic
tttaaatggccgctgcggccaattcct

SEQ ID NO: 76 - DNA, 27 bp, Artificial
  synthetic
tttaataaatatgggcaatctttccct

SEQ ID NO: 77 - DNA, 27 bp, Artificial
  synthetic
tttcccgccttcagtttaaactatcag

SEQ ID NO: 78 - DNA, 27 bp, Artificial
  synthetic
tttggactgagaattagcttccactcg

SEQ ID NO: 79 - DNA, 27 bp, Artificial
  synthetic
tttaaacgtgcaagcgctcaattcgcc

SEQ ID NO: 80 - DNA, 27 bp, Artificial
  synthetic
tttaatgtactgaattgcgtacgattg

SEQ ID NO: 81 - DNA, 27 bp, Artificial
  synthetic
tttgtagcacttgcacgtagttacccg

SEQ ID NO: 82 - DNA, 27 bp, Artificial
  synthetic
tttaaacgctcttcaactggaagagcg

SEQ ID NO: 83 - DNA, 27 bp, Artificial
  synthetic
tttgtgattctctaaacactgatagtt

SEQ ID NO: 84 - DNA, 27 bp, Artificial
  synthetic
tttaatcatattgttaaggatataatt

SEQ ID NO: 85 - DNA, 27 bp, Artificial
  synthetic
tttctactattataaaagcttggtacc

SEQ ID NO: 86 - DNA, 27 bp, Artificial
  synthetic
tttgagtggatcctgttatctcttctc

SEQ ID NO: 87 - DNA, 27 bp, Artificial
  synthetic
tttaccggtgcccgggcggccagcatg

SEQ ID NO: 88 - DNA, 27 bp, Artificial
  synthetic
tttgtttacaccacaatatatttcaag

SEQ ID NO: 89 - DNA, 24 bp, Artificial
  synthetic
tttaccggtgcccgggcggccagc

SEQ ID NO: 90 - DNA, 27 bp, Artificial
  synthetic
tttgccagtgggcccagcctggcccag

SEQ ID NO: 91 - DNA, 27 bp, Artificial
  synthetic
tttggacgtgaatgtagacacgtcgaa

SEQ ID NO: 92 - DNA, 27 bp, Artificial
  synthetic
tttccggggatgcaatgagtatgatgg

SEQ ID NO: 93 - DNA, 27 bp, Artificial
  synthetic
tttctagtcaccggttaggatccgttt

SEQ ID NO: 94 - DNA, 27 bp, Artificial
  synthetic
tttaattcttaacaatcaatattttaa

SEQ ID NO: 95 - DNA, 27 bp, Artificial
  synthetic
tttccgaattagaataatttgtttatt

SEQ ID NO: 96 - DNA, 27 bp, Artificial
  synthetic
tttcctaaattagtcctactttttgat

SEQ ID NO: 97 - DNA, 27 bp, Artificial
  synthetic
tttctcatctaagcccccatttggacg

SEQ ID NO: 98 - DNA, 27 bp, Artificial
  synthetic
tttatcaaaatgtactttcattttata

SEQ ID NO: 99 - DNA, 27 bp, Artificial
  synthetic
tttgtttacctgaatatttgccttttt

SEQ ID NO: 100 - DNA, 27 bp, Artificial
  synthetic
tttaatgtacgccatgctggccgcccg

SEQ ID NO: 101 - DNA, 27 bp, Artificial
  synthetic
tttctcatctaagcccccatttggacg

SEQ ID NO: 102 - DNA, 27 bp, Artificial
  synthetic
tttacaattgaccatcatactcaactt

SEQ ID NO: 103 - DNA, 23 bp, Artificial
  synthetic
gggacggaagaaagagtgaaggg

SEQ ID NO: 104 - DNA, 23 bp, Artificial
  synthetic
acgctcttcaactggaagagcgg

SEQ ID NO: 105 - DNA, 23 bp, Artificial
  synthetic
agcacttgcacgtagttacccgg

SEQ ID NO: 106 - DNA, 23 bp, Artificial
  synthetic
ccggggcccatcgatatccgcgg

SEQ ID NO: 107 - DNA, 23 bp, Artificial
  synthetic
ctgatagtttaaactgaaggcgg

SEQ ID NO: 108 - DNA, 23 bp, Artificial
  synthetic
gaaggagccactcagcaagctgg

SEQ ID NO: 109 - DNA, 23 bp, Artificial
  synthetic
gccttgtagcggcccacgcgtgg

SEQ ID NO: 110 - DNA, 23 bp, Artificial
  synthetic
cgcgttaacaagcttactcgagg

SEQ ID NO: 111 - DNA, 23 bp, Artificial
  synthetic
cagtttaaactatcagtgtttgg

SEQ ID NO: 112 - DNA, 23 bp, Artificial
  synthetic
gggcgcgccgaattcgagctcgg

SEQ ID NO: 113 - DNA, 23 bp, Artificial
  synthetic
tggatcagcaatgagtatgatgg

SEQ ID NO: 114 - DNA, 23 bp, Artificial
  synthetic
ctacctgtcacttcatcaaaagc

SEQ ID NO: 115 - DNA, 23 bp, Artificial
  synthetic
acaagagcaagtagcggatacgg

SEQ ID NO: 116 - DNA, 23 bp, Artificial
  synthetic
gatcctaaccggtgtgatcatgg

SEQ ID NO: 117 - DNA, 23 bp, Artificial
  synthetic
ccgctctagcgcttcaatcgtgg

SEQ ID NO: 118 - DNA, 23 bp, Artificial
  synthetic
ttctgcaggtcctgctcgagtgg

SEQ ID NO: 119 - DNA, 23 bp, Artificial
  synthetic
atacatgcttagcatgccccagg

SEQ ID NO: 120 - DNA, 23 bp, Artificial
  synthetic
gggatccactagttctagagcgg

SEQ ID NO: 121 - DNA, 23 bp, Artificial
  synthetic
atcaaaaaaggcaaatattcagg

SEQ ID NO: 122 - DNA, 23 bp, Artificial
  synthetic
gtacgccatgctggccgcccggg

SEQ ID NO: 123 - DNA, 23 bp, Artificial
  synthetic
taaactatcagtgtttgaagtgg

SEQ ID NO: 124 - DNA, 23 bp, Artificial
  synthetic
cctgcagaagcttgataacgcgg

SEQ ID NO: 125 - DNA, 23 bp, Artificial
  synthetic
tccagggcgagctcggtacccgg

SEQ ID NO: 126 - DNA, 23 bp, Artificial
  synthetic
aaacaaacgggaccatagaaggg

SEQ ID NO: 127 - DNA, 23 bp, Artificial
  synthetic
atcgtggcctcttgctcttcagg

SEQ ID NO: 128 - DNA, 23 bp, Artificial
  synthetic
aagcgtcaatttggaacaagtgg

SEQ ID NO: 129 - DNA, 23 bp, Artificial
  synthetic
aaacactgatagtttaaacgcgg

SEQ ID NO: 130 - DNA, 23 bp, Artificial
  synthetic
acatatgtatgtatataatttgg

SEQ ID NO: 131 - DNA, 23 bp, Artificial
  synthetic
tgtactgaattgtctagacccgg

SEQ ID NO: 132 - DNA, 23 bp, Artificial
  synthetic
attttatagatcatacaaaaagg

SEQ ID NO: 133 - DNA, 23 bp, Artificial
  synthetic
ggggatatccccggggaattcgg

SEQ ID NO: 134 - DNA, 23 bp, Artificial
  synthetic
tagagtggaagtgtgtcgcgtgg

SEQ ID NO: 135 - DNA, 23 bp, Artificial
  synthetic
ggcaccggtaaatttcctgcagg

SEQ ID NO: 136 - DNA, 23 bp, Artificial
  synthetic
agatgacttgaaatatattgtgg

SEQ ID NO: 137 - DNA, 23 bp, Artificial
  synthetic
actagatctgctagccctgcagg

SEQ ID NO: 138 - DNA, 23 bp, Artificial
  synthetic
cccagcctggcccagggaagagg

SEQ ID NO: 139 - DNA, 23 bp, Artificial
  synthetic
gggaattcggtaccaagctttgg

SEQ ID NO: 140 - DNA, 24 bp, Artificial
  synthetic
ccggggatgcaatgagtatgatgg

SEQ ID NO: 141 - DNA, 23 bp, Artificial
  synthetic
ttacgatccgtcgtatttatagg

SEQ ID NO: 142 - DNA, 22 bp, Artificial
  synthetic
acagaagccatcaaaaagtagg

SEQ ID NO: 143 - DNA, 23 bp, Artificial
  synthetic
cggccgcttcgagtggctgcagg

SEQ ID NO: 144 - DNA, 23 bp, Artificial
  synthetic
gaaatgcttgaggagagtgaagg

SEQ ID NO: 145 - DNA, 23 bp, Artificial
  synthetic
attgatttaaatggccgctgcgg

SEQ ID NO: 146 - DNA, 23 bp, Artificial
  synthetic
gtaacagtacagtcggtgtaggg

SEQ ID NO: 147 - DNA, 23 bp, Artificial
  synthetic
atcgatgcggccgcttcgagtgg

SEQ ID NO: 148 - DNA, 23 bp, Artificial
  synthetic
aaattgaagttgagtatgatggt

SEQ ID NO: 149 - protein, 1307 aa, Unknown
  bacterial
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

(19) United States
(12) Patent Application Publication
(10) Pub. No.: US 2024/0294937 A1
(43) Pub. Date: Sep. 5, 2024

(54) GENOME EDITING OF TRANSGENIC CROP PLANTS WITH MODIFIED TRANSGENIC LOCI

(71) Applicant: INARI AGRICULTURE TECHNOLOGY, INC., Cambridge, MA (US)

(72) Inventors: Michael Andreas Kock, Rheinfelden (DE); Michael Lee Nuccio, Salem, NH (US); Frédéric Van Ex, Kortrijk (BE); Alexandra Elata, Arlington, MA (US); Daniel Rodriguez Leal, Raleigh, NC (US); Joshua L. Price, Cambridge, MA (US)

(21) Appl. No.: 18/007,164

(22) PCT Filed: Jul. 26, 2021
(86) PCT No.: PCT/US2021/043161

§ 371 Date: Jan. 27, 2023

Related U.S. Application Data
(63) Continuation of application No. 17/248,936, filed on Feb. 12, 2021, now Pat. No. 11,359,210.
Application No. 17/248,936 is a continuation of application No. 17/249,640, filed on Mar. 8, 2021, now Pat. No. 11,214,811.
Application No. 17/249,640 is a continuation of application No. 17/302,110, filed on Apr. 23, 2021.
Application No. 17/302,110 is a continuation of application No. 17/302,121, filed on Apr. 23, 2021, now Pat. No. 11,242,534.
Application No. 17/302,121 is a continuation of application No. 17/302,739, filed on May 11, 2021, now Pat. No. 11,326,177.
Application No. 17/302,739 is a continuation of application No. 17/303,116, filed on May 20, 2021, now Pat. No. 11,369,073.
(60) Provisional application No. 63/059,813, filed on Jul. 31, 2020.
(60) Provisional application No. 63/059,860, filed on Jul. 31, 2020.
(60) Provisional application No. 63/059,916, filed on Jul. 31, 2020.
(60) Provisional application No. 63/059,963, filed on Jul. 31, 2020.
(60) Provisional application No. 63/199,930, filed on Feb. 3, 2021.
(60) Provisional application No. 63/199,949, filed on Feb. 4, 2021.
(60) Provisional application No. 63/199,951, filed on Feb. 4, 2021.
(60) Provisional application No. 63/201,029, filed on Apr. 9, 2021.
(60) Provisional application No. 63/201,030, filed on Apr. 9, 2021.
(60) Provisional application No. 63/202,569, filed on Jun. 16, 2021.
(60) Provisional application No. 63/203,137, filed on Jul. 9, 2021.

(51) Int. Cl. C12N 15/82

(52) U.S. Cl. CPC C12N 15/8286; C12N 15/8213

(57) ABSTRACT

Methods of selectively excising selectable marker genes and/or non-essential DNAs from transgenic loci from transgenic plants as well as making targeted genetic changes with genome editing techniques are provided. Also provided are transgenic plants comprising the transgenic loci lacking the selectable markers and/or non-essential DNAs and use of such methods and plants to facilitate plant breeding are disclosed.

### SEQUENCE LISTING

The sequence listing contained in the file named “Replacement_Sequence_Listing_18007164”, which is 486,243 bytes as measured in the Windows operating system, and which was created on Aug. 15, 2023 and electronically filed via EFS-Web, is incorporated herein by reference in its entirety.

### BACKGROUND

Transgenes which are placed into different positions in the plant genome through non-site specific integration can exhibit different levels of expression (Weising et al., 1988, Ann. Rev. Genet. 22:421-477). Such transgene insertion sites can also contain various undesirable rearrangements of the foreign DNA elements that include deletions and/or duplications. Furthermore, many transgene insertion sites can also comprise selectable or scoreable marker genes which in some instances are no longer required once a transgenic plant event containing the linked transgenes which confer desirable traits are selected.

Commercial transgenic plants typically comprise one or more independent insertions of transgenes at specific locations in the host plant genome that have been selected for features that include expression of the transgene(s) of interest and the transgene-conferred trait(s), absence or minimization of rearrangements, and normal Mendelian transmission of the trait(s) to progeny. Examples of selected transgenic corn, soybean, cotton, and canola plant events which confer traits such as herbicide tolerance and/or pest tolerance are disclosed in U.S. Pat. Nos. 7,323,556; 8,575,434; 6,040,497; 10,316,330; 8,618,358; 8,212,113; 9,428,765; 8,455,720; 7,897,748; 8,273,959; 8,093,453; 8,901,378; 8,466,346; RE44962; 9,540,655; 9,738,904; 8,680,363; 8,049,071; 9,447,428; 9,944,945; 8,592,650; 10,184,134; 7,179,965; 7,371,940; 9,133,473; 8,735,661; 7,381,861; 8,048,632; and 9,738,903.

Methods for removing selectable marker genes and/or duplicated transgenes in transgene insertion sites in plant genomes involving use of site-specific recombinase systems (e.g., cre-lox) as well as for insertion of new genes into transgene insertion sites have been disclosed (Srivastava and Ow; Methods Mol Biol, 2015,1287:95-103; Dale and Ow, 1991, Proc. Natl Acad. Sci. USA 88, 10558-10562; Srivastava and Thomson, Plant Biotechnol J, 2016;14(2):471-82). Such methods typically require incorporation of the recombination site sequences recognized by the recombinase at particular locations within the transgene.

### SUMMARY

Methods of producing an elite crop plant comprising at least one modification of an approved transgenic locus and at least one targeted genetic change conferring a desirable trait, said method comprising the steps of: (a) transforming a plant cell or plant tissue of an elite crop plant with a marker-based transformation system comprising at least one selectable marker and one or more transgenes encoding at least one genome editing molecule, wherein the elite crop plant comprises a modification of the approved transgenic locus comprising a deletion of a segment comprising, consisting essentially of, or consisting of a selectable marker gene and wherein said genome editing molecules are designed to induce said at least one targeted genetic change, (b) selecting plants which comprise a stable integration of said transformation system in the genomic DNA of the plant by utilizing said selectable marker, and (c) selecting plants which comprise said modification of an approved transgenic locus and at least one targeted genetic change conferring a desirable trait are provided herein.

Methods of producing an elite crop plant comprising a targeted genetic change conferring a desirable trait and at least one modification of an approved transgenic locus comprising steps of: (i) inducing at least one targeted genetic change in the genome of the maize plant with one or more genome editing molecules in an elite crop plant comprising the modification of the approved transgenic locus; and (ii) selecting an elite crop plant comprising the modification of the approved transgenic locus wherein the targeted genetic change are provided herein.

Elite crop plants comprising a modification of an original approved transgenic locus, wherein the modification comprises a deletion of a segment comprising, consisting essentially of, or consisting of a selectable marker gene of an original approved transgenic locus, and wherein the modification of the approved transgenic locus does not comprise a site-specific recombination system DNA recognition site are provided herein.

Methods for obtaining the elite crop plants comprising the steps of: (a) obtaining a crop plant comprising the modification of an original approved transgenic locus comprising a deletion of a segment comprising, consisting essentially of, or consisting of a selectable marker gene of the original approved transgenic locus, wherein the plant does not comprise germplasm of the elite crop plant; and (b) introgressing the modified transgenic locus into the germplasm of the elite crop plant are provided.

Methods for obtaining a bulked population of inbred seed for commercial seed production comprising selfing the elite crop plants provided herein and harvesting seed from the selfed elite crop plants are provided.

Methods of obtaining hybrid seed comprising crossing a first plant comprising edited transgenic plant genomes comprising the modified transgenic loci set forth herein and optionally targeted genetic changes to a second plant and harvesting seed from the cross are provided.

DNA comprising a selectable marker gene excision site wherein a segment comprising, consisting essentially of, or consisting of a selectable marker gene of an original approved transgenic locus is deleted are provided.

Nucleic acid markers adapted for detection of genomic DNA or fragments comprising a selectable marker gene excision site wherein a segment comprising, consisting essentially of, or consisting of a selectable marker gene of an original approved transgenic locus is deleted and wherein the nucleic acid marker does not detect an original approved transgenic locus wherein the selectable marker gene has not been deleted are provided.

Biological samples comprising plant genomic DNA or fragments thereof, said genomic DNA or fragments comprising a selectable marker gene excision site wherein a segment comprising, consisting essentially of, or consisting of a selectable marker gene of an original approved transgenic locus is deleted are provided.

Methods of identifying the elite crop plants, DNA, or biological samples set forth herein comprising detecting with a nucleic acid detection assay a polynucleotide comprising a selectable marker gene excision site wherein a segment comprising, consisting essentially of, or consisting of a selectable marker gene of an original approved transgenic locus is deleted are provided.

Methods of enhancing functionality of an approved transgenic locus comprising deleting a segment of the original approved transgenic locus with one or more gene editing molecules, the segment comprising, consisting essentially of, or consisting of: a duplication of a transgene; a duplication of a transgene element; and/or a fragment of a transgene; optionally, wherein the duplication and/or fragment of a transgene element is a duplication and/or fragment of a promoter or a polyadenylation signal are provided.

### BRIEF DESCRIPTION OF THE DRAWINGS/FIGURES

FIG. 1 shows a diagram of transgene expression cassettes and selectable markers in the DAS-59122-7 transgenic locus set forth in SEQ ID NO: 1.

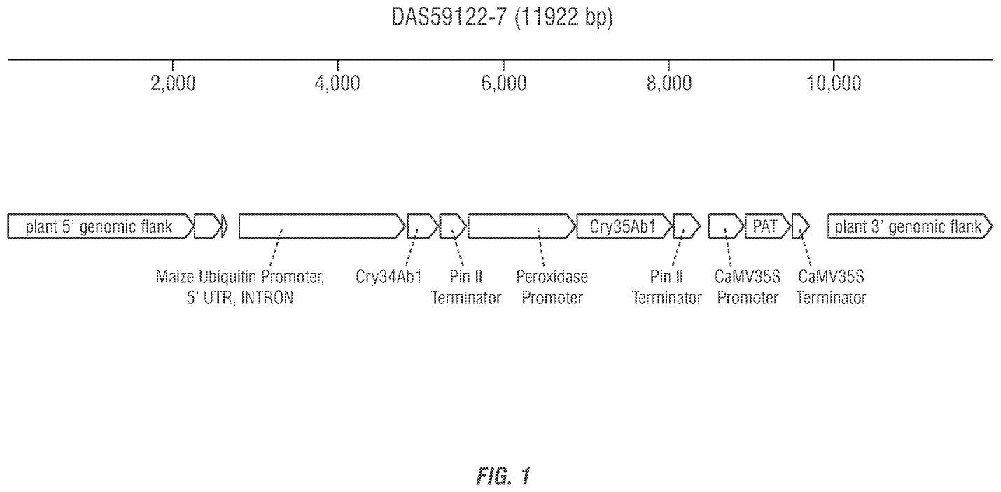

FIG. 2 shows a diagram of transgene expression cassettes and selectable markers in the DP-4114 transgenic locus set forth in SEQ ID NO: 2.

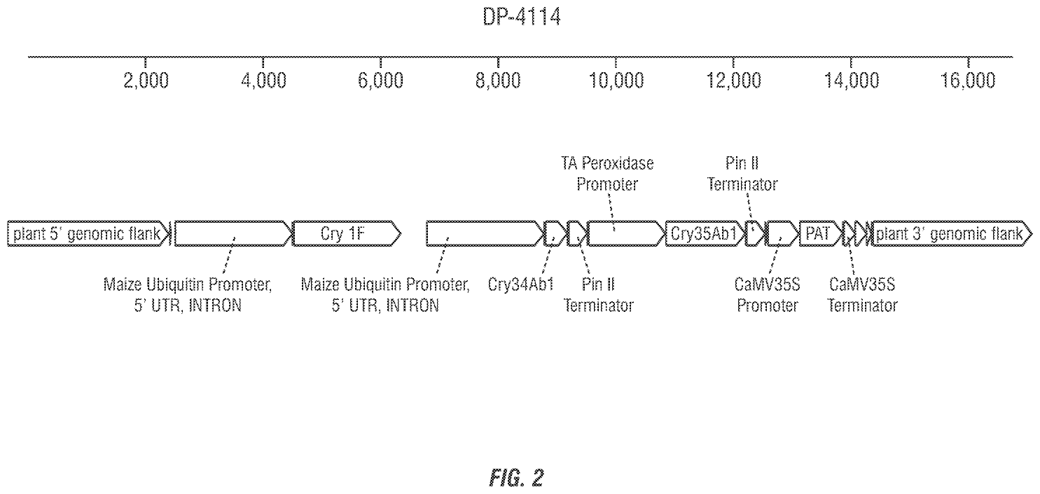

FIG. 3 shows a diagram of transgene expression cassettes and selectable markers in the MON87411 transgenic locus set forth in SEQ ID NO: 3.

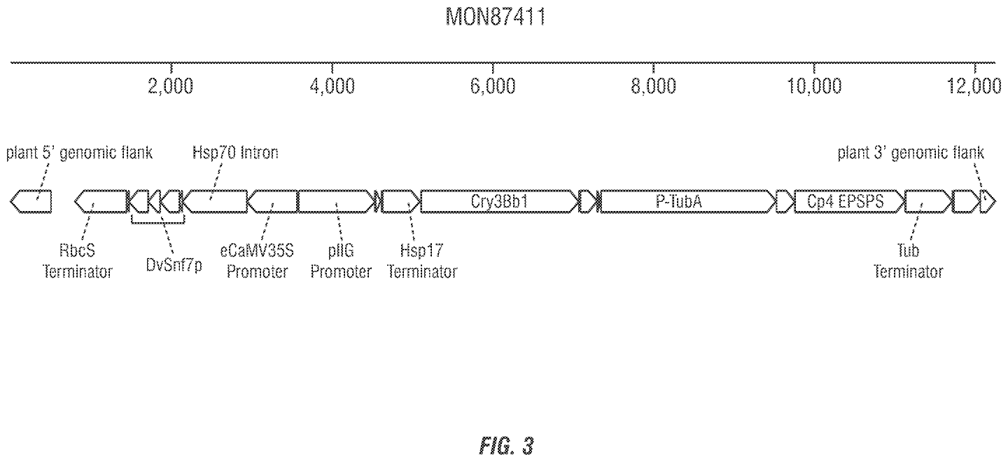

FIG. 4 shows a diagram of transgene expression cassettes in the MON89034 transgenic locus.

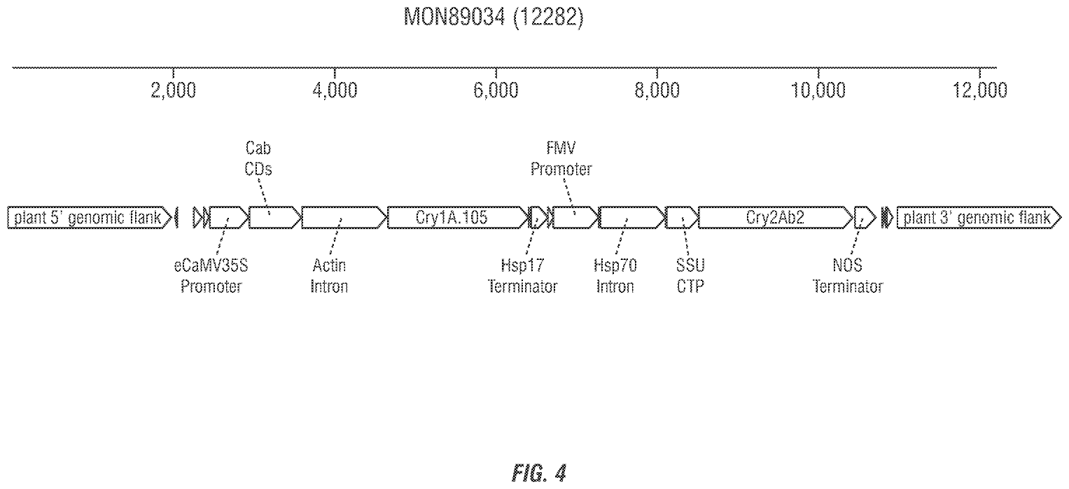

FIG. 5 shows a diagram of transgene expression cassettes and selectable markers in the MIR162 transgenic locus.

FIG. 6 shows a diagram of transgene expression cassettes and selectable markers in the MIR604 transgenic locus set forth in SEQ ID NO: 6.

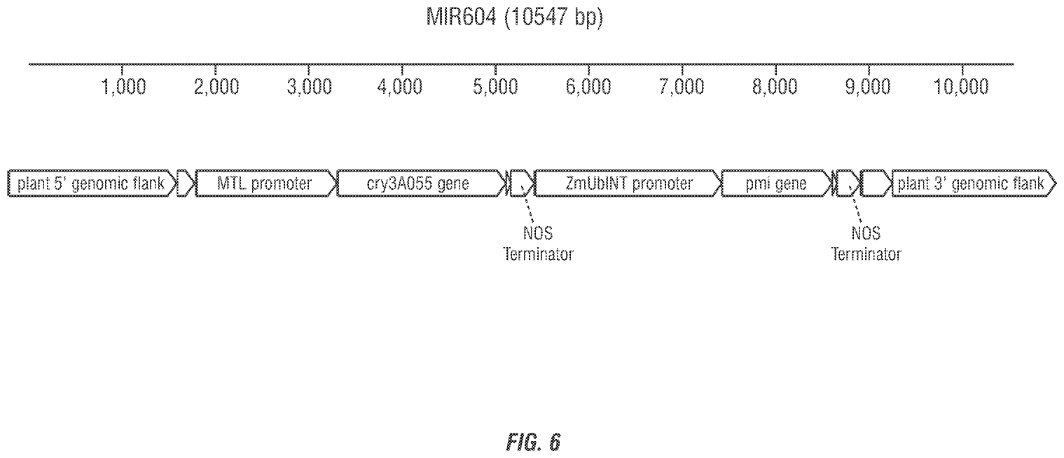

FIG. 7 shows a diagram of transgene expression cassettes and selectable markers in the NK603 transgenic locus set forth in SEQ ID NO: 7.

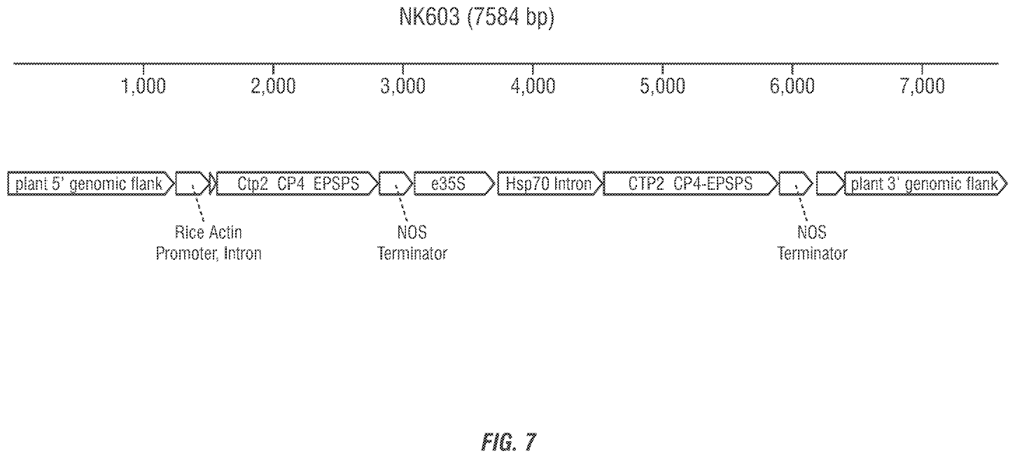

FIG. 8 shows a diagram of transgene expression cassettes and selectable markers in the SYN-E3272-5 transgenic locus set forth in SEQ ID NO: 8.

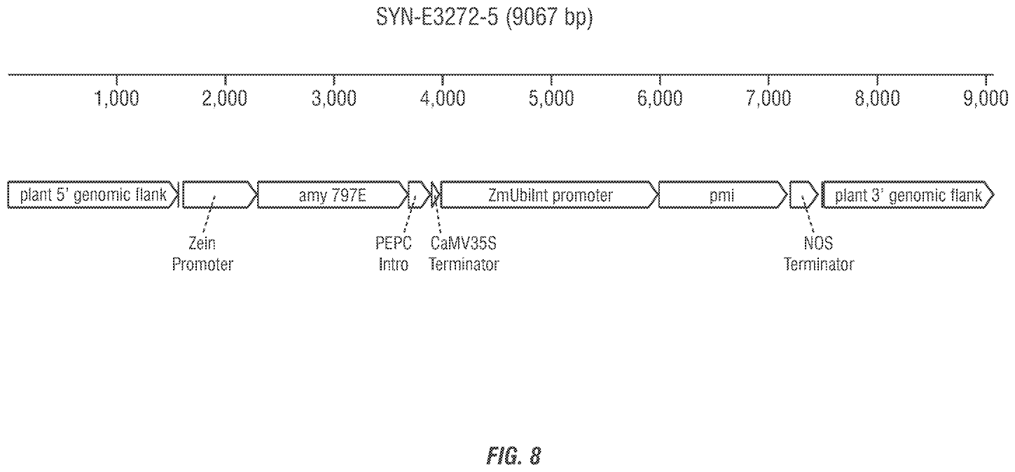

FIG. 9 shows a diagram of transgene expression cassettes and selectable markers in the TC1507 transgenic locus set forth in SEQ ID NO: 8.

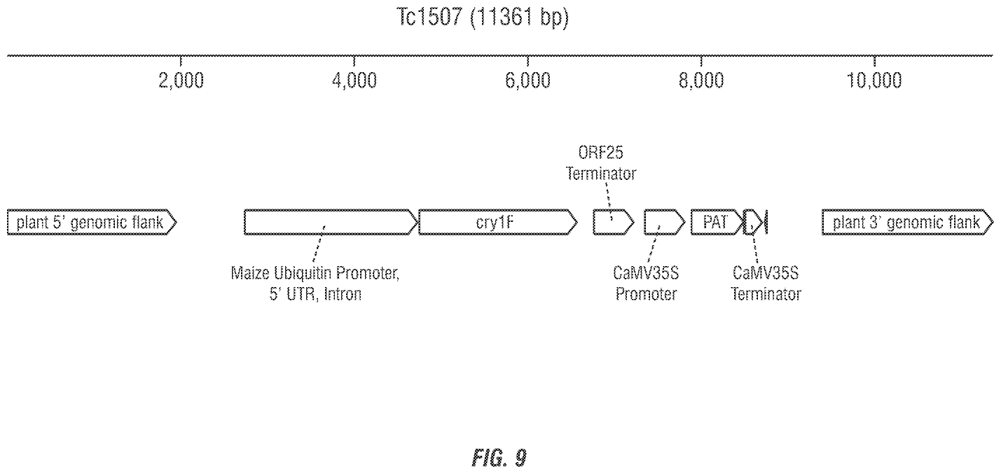

FIG. 10 shows a diagram of transgene expression cassettes and selectable markers in the 5307 transgenic locus set forth in SEQ ID NO: 10.

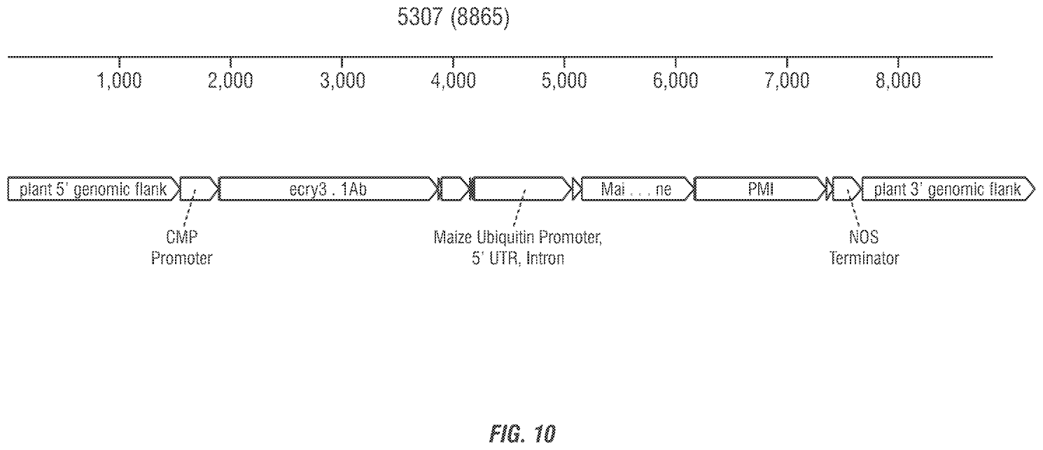

FIG. 11 shows a schematic diagram which compares current breeding strategies for introgression of transgenic events (i.e., transgenic loci) to alternative breeding strategies for introgression of transgenic events where the transgenic events (i.e., transgenic loci) can be removed following introgression to provide different combinations of transgenic traits.

FIG. 12 shows a diagram of transgene expression cassettes and selectable markers in the DAS68416-4 transgenic locus set forth in SEQ ID NO: 12.

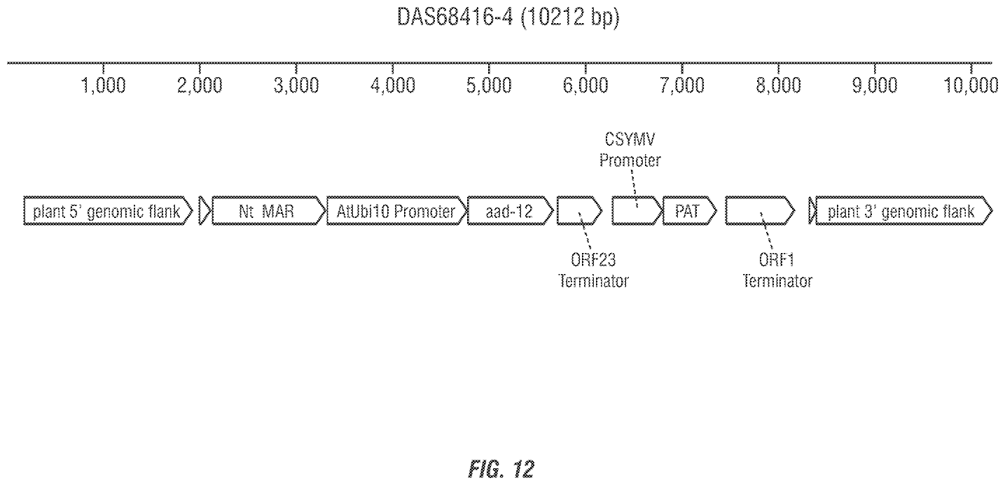

FIG. 13 shows a diagram of transgene expression cassettes and selectable markers in the MON87701 transgenic locus set forth in SEQ ID NO: 14.

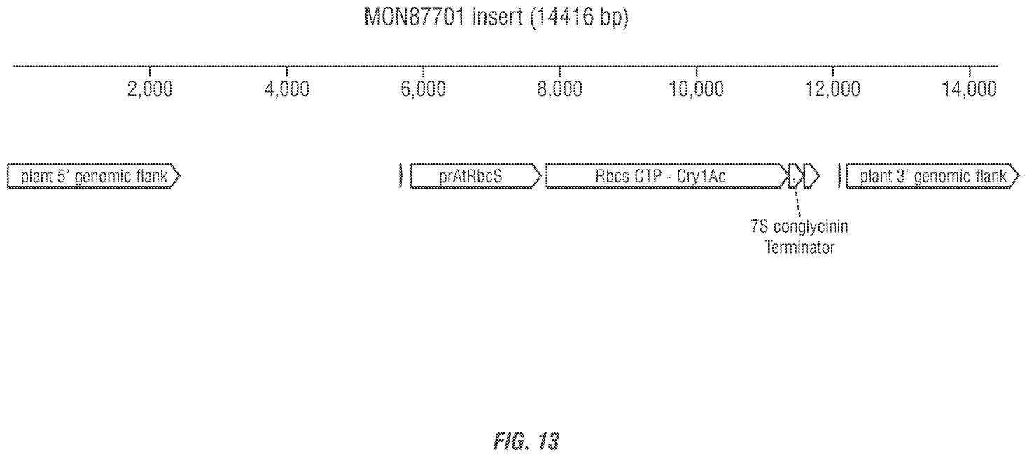

FIG. 14 shows a diagram of transgene expression cassettes and selectable markers in the MON89788 transgenic locus set forth in SEQ ID NO: 16.

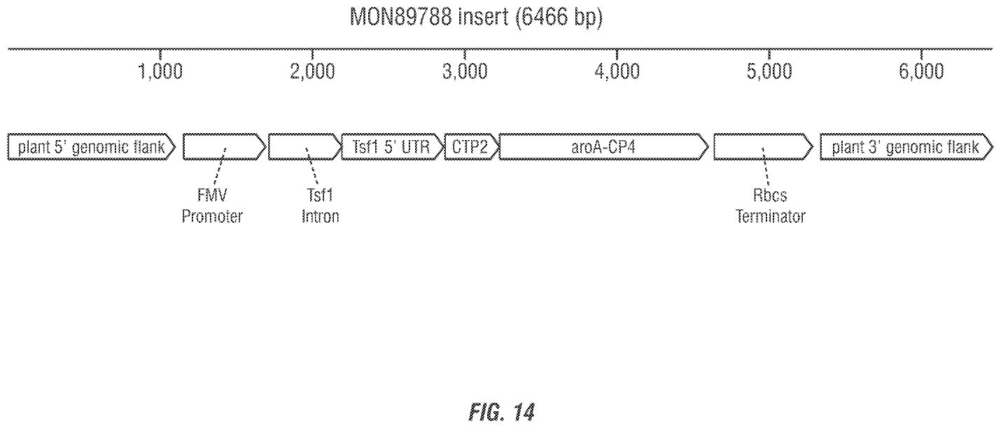

FIG. 15 shows a diagram of transgene expression cassettes and selectable markers in the COT102 transgenic locus set forth in SEQ ID NO: 19.

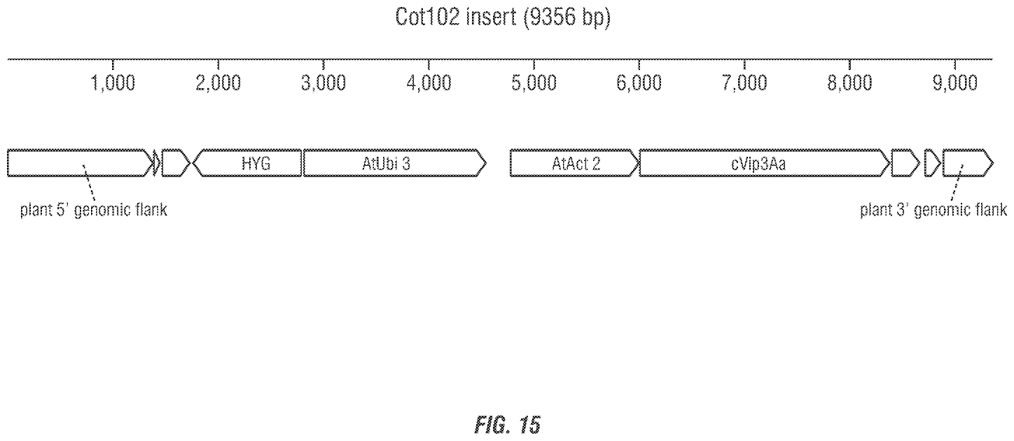

FIG. 16 shows a diagram of transgene expression cassettes and selectable markers in the MON88302 transgenic locus set forth in SEQ ID NO: 21.

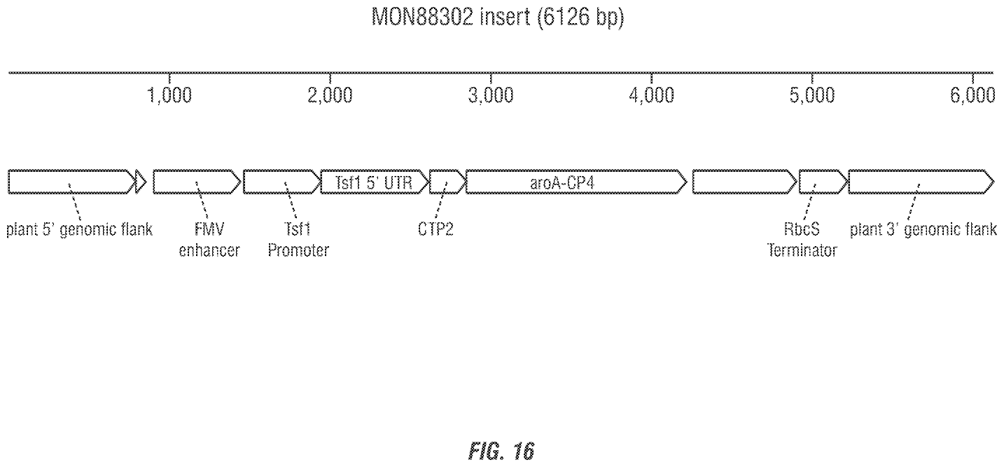

### DETAILED DESCRIPTION

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as well as necessarily defines the exact complements, as is known to one of ordinary skill in the art.

Where a term is provided in the singular, the inventors also contemplate embodiments described by the plural of that term.

The term “about” as used herein means a value or range of values which would be understood as an equivalent of a stated value and can be greater or lesser than the value or range of values stated by 10 percent. Each value or range of values preceded by the term “about” is also intended to encompass the embodiment of the stated absolute value or range of values.

The phrase “allelic variant” as used herein refers to a polynucleotide or polypeptide sequence variant that occurs in a different strain, variety, or isolate of a given organism.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrase “approved transgenic locus” is a genetically modified plant event which has been authorized, approved, and/or de-regulated for any one of field testing, cultivation, human consumption, animal consumption, and/or import by a governmental body. Illustrative and non-limiting examples of governmental bodies which provide such approvals include the Ministry of Agriculture of Argentina, Food Standards Australia New Zealand, National Biosafety Technical Committee (CTNBio) of Brazil, Canadian Food Inspection Agency, China Ministry of Agriculture Biosafety Network, European Food Safety Authority, US Department of Agriculture, US Department of Environmental Protection, and US Food and Drug Administration.

The term “backcross”, as used herein, refers to crossing an F1 plant or plants with one of the original parents. A backcross is used to maintain or establish the identity of one parent (species) and to incorporate a particular trait from a second parent (species). The term “backcross generation”, as used herein, refers to the offspring of a backcross.

As used herein, the phrase “biological sample” refers to either intact or non-intact (e.g. milled seed or plant tissue, chopped plant tissue, lyophilized tissue) plant tissue. It may also be an extract comprising intact or non-intact seed or plant tissue. The biological sample can comprise flour, meal, syrup, oil, starch, and cereals manufactured in whole or in part to contain crop plant by-products. In certain embodiments, the biological sample is “non-regenerable” (i.e., incapable of being regenerated into a plant or plant part). In certain embodiments, the biological sample refers to a homogenate, an extract, or any fraction thereof containing genomic DNA of the organism from which the biological sample was obtained, wherein the biological sample does not comprise living cells.

As used herein, the terms “correspond,” “corresponding,” and the like, when used in the context of an nucleotide position, mutation, and/or substitution in any given polynucleotide (e.g., an allelic variant of SEQ ID NO: 1-34) with respect to the reference polynucleotide sequence (e.g., SEQ ID NO: 1-34) all refer to the position of the polynucleotide residue in the given sequence that has identity to the residue in the reference nucleotide sequence when the given polynucleotide is aligned to the reference polynucleotide sequence using a pairwise alignment algorithm (e.g., CLUSTAL O 1.2.4 with default parameters).

As used herein, the terms “Cpf1” and “Cas12a” are used interchangeably to refer to the same RNA dependent DNA endonuclease (RdDe). Cas12a proteins include the protein provided herein as SEQ ID NO: 149.

The term “crossing” as used herein refers to the fertilization of female plants (or gametes) by male plants (or gametes). The term “gamete” refers to the haploid reproductive cell (egg or pollen) produced in plants by meiosis from a gametophyte and involved in sexual reproduction, during which two gametes of opposite sex fuse to form a diploid zygote. The term generally includes reference to a pollen (including the sperm cell) and an ovule (including the ovum). When referring to crossing in the context of achieving the introgression of a genomic region or segment, the skilled person will understand that in order to achieve the introgression of only a part of a chromosome of one plant into the chromosome of another plant, random portions of the genomes of both parental lines recombine during the cross due to the occurrence of crossing-over events in the production of the gametes in the parent lines. Therefore, the genomes of both parents must be combined in a single cell by a cross, where after the production of gametes from the cell and their fusion in fertilization will result in an introgression event.

As used herein, the phrases “DNA junction polynucleotide” and “junction polynucleotide” refers to a polynucleotide of about 18 to about 500 base pairs in length comprised of both endogenous chromosomal DNA of the plant genome and heterologous transgenic DNA which is inserted in the plant genome. A junction polynucleotide can thus comprise about 8, 10, 20, 50, 100, 200, or 250 base pairs of endogenous chromosomal DNA of the plant genome and about 8, 10, 20, 50, 100, 200, or 250 base pairs of heterologous transgenic DNA which span the one end of the transgene insertion site in the plant chromosomal DNA. Transgene insertion sites in chromosomes will typically contain both a 5′ junction polynucleotide and a 3′ junction polynucleotide. In embodiments set forth herein in SEQ ID NO: 1-34, the 5′ junction polynucleotide is located at the 5′ end of the sequence and the 3′ junction polynucleotide is located at the 3′ end of the sequence.

The term “donor”, as used herein in the context of a plant, refers to the plant or plant line from which the trait, transgenic event, or genomic segment originates, wherein the donor can have the trait, introgression, or genomic segment in either a heterozygous or homozygous state.

As used herein, the terms “excise” and “delete,” when used in the context of a DNA molecule, are used interchangeably to refer to the removal of a given DNA segment or element (e.g., transgene element) of the DNA molecule.

As used herein, the phrase “elite crop plant” refers to a plant which has undergone breeding to provide one or more trait improvements. Elite crop plant lines include plants which are an essentially homozygous, e.g. inbred or doubled haploid. Elite crop plants can include inbred lines used as is or used as pollen donors or pollen recipients in hybrid seed production (e.g. used to produce F1 plants). Elite crop plants can include inbred lines which are selfed to produce non-hybrid cultivars or varieties or to produce (e.g., bulk up) pollen donor or recipient lines for hybrid seed production. Elite crop plants can include hybrid F1 progeny of a cross between two distinct elite inbred or doubled haploid plant lines.

As used herein, an “event.” “a transgenic event,” “a transgenic locus” and related phrases refer to an insertion of one or more transgenes at a unique site in the genome of a plant as well as to DNA fragments, plant cells, plants, and plant parts (e.g., a seed, leaf, tuber, stem, root, or boll) comprising genomic DNA containing the transgene insertion. Such events typically comprise both a 5′ and a 3′ DNA junction polynucleotide and confer one or more useful traits including herbicide tolerance, insect resistance, male sterility, and the like.

As used herein, the phrases “endogenous sequence,” “endogenous gene,” “endogenous DNA” and the like refer to the native form of a polynucleotide, gene or polypeptide in its natural location in the organism or in the genome of an organism.

The term “exogenous DNA sequence” as used herein is any nucleic acid sequence that has been removed from its native location and inserted into a new location altering the sequences that flank the nucleic acid sequence that has been moved. For example, an exogenous DNA sequence may comprise a sequence from another species.

As used herein, the term “F1” refers to any offspring of a cross between two genetically unlike individuals.

The term “gene,” as used herein, refers to a hereditary unit consisting of a sequence of DNA that occupies a specific location on a chromosome and that contains the genetic instruction for a particular characteristics or trait in an organism. The term “gene” thus includes a nucleic acid (for example, DNA or RNA) sequence that comprises coding sequences necessary for the production of an RNA, or a polypeptide or its precursor. A functional polypeptide can be encoded by a full length coding sequence or by any portion of the coding sequence as long as the desired activity or functional properties (e.g., enzymatic activity, pesticidal activity, ligand binding, and/or signal transduction) of the RNA or polypeptide are retained.

The term “identifying,” as used herein with respect to a plant, refers to a process of establishing the identity or distinguishing character of a plant, including exhibiting a certain trait, containing one or more transgenes, and/or containing one or more molecular markers.

The term “isolated” as used herein means having been removed from its natural environment.

As used herein, the terms “include,” “includes,” and “including” are to be construed as at least having the features to which they refer while not excluding any additional unspecified features.

As used herein, the phrase “introduced transgene” is a transgene not present in the original transgenic locus in the genome of an initial transgenic event or in the genome of a progeny line obtained from the initial transgenic event. Examples of introduced transgenes include exogenous transgenes which are inserted in a resident original transgenic locus.

As used herein, the terms “introgression”, “introgressed” and “introgressing” refer to both a natural and artificial process, and the resulting plants, whereby traits, genes or DNA sequences of one species, variety or cultivar are moved into the genome of another species, variety or cultivar, by crossing those species. The process may optionally be completed by backcrossing to the recurrent parent. Examples of introgression include entry or introduction of a gene, a transgene, a regulatory element, a marker, a trait, a trait locus, or a chromosomal segment from the genome of one plant into the genome of another plant.

The phrase “marker-assisted selection”, as used herein, refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The phrase “molecular marker”, as used herein, refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), microsatellite markers (e.g., SSRs), sequence-characterized amplified region (SCAR) markers, Next Generation Sequencing (NGS) of a molecular marker, cleaved amplified polymorphic sequence (CAPS) markers or isozyme markers or combinations of the markers described herein which defines a specific genetic and chromosomal location.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

The term “offspring”, as used herein, refers to any progeny generation resulting from crossing, selfing, or other propagation technique.

The phrase “operably linked” refers to a juxtaposition wherein the components so described are in a relationship permitting them to function in their intended manner. For instance, a promoter is operably linked to a coding sequence if the promoter affects its transcription or expression. When the phrase “operably linked” is used in the context of a PAM site and a DNA segment, it refers to a PAM site which permits cleavage of at least one strand of DNA in the DNA segment with an RNA dependent DNA endonuclease, RNA dependent DNA binding protein, or RNA dependent DNA nickase which recognizes the PAM site when a guide RNA complementary to sequences adjacent to the PAM site is present.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In contrast, some plant cells are not capable of being regenerated to produce plants and are referred to herein as “non-regenerable” plant cells.

The term “purified,” as used herein defines an isolation of a molecule or compound in a form that is substantially free of contaminants normally associated with the molecule or compound in a native or natural environment and means having been increased in purity as a result of being separated from other components of the original composition. The term “purified nucleic acid” is used herein to describe a nucleic acid sequence which has been separated from other compounds including, but not limited to polypeptides, lipids and carbohydrates.

The term “recipient”, as used herein, refers to the plant or plant line receiving the trait, transgenic event or genomic segment from a donor, and which recipient may or may not have the have trait, transgenic event or genomic segment itself either in a heterozygous or homozygous state.

As used herein the term “recurrent parent” or “recurrent plant” describes an elite line that is the recipient plant line in a cross and which will be used as the parent line for successive backcrosses to produce the final desired line.

As used herein the term “recurrent parent percentage” relates to the percentage that a backcross progeny plant is identical to the recurrent parent plant used in the backcross. The percent identity to the recurrent parent can be determined experimentally by measuring genetic markers such as SNPs and/or RFLPs or can be calculated theoretically based on a mathematical formula.

The terms “selfed,” “selfing,” and “self,” as used herein, refer to any process used to obtain progeny from the same plant or plant line as well as to plants resulting from the process. As used herein, the terms thus include any fertilization process wherein both the ovule and pollen are from the same plant or plant line and plants resulting therefrom. Typically, the terms refer to self-pollination processes and progeny plants resulting from self-pollination.

The term “selecting”, as used herein, refers to a process of picking out a certain individual plant from a group of individuals, usually based on a certain identity, trait, characteristic, and/or molecular marker of that individual.

As used herein, the phrase “selectable marker gene excision site” refers to the DNA which remains in a modified transgenic locus wherein a segment comprising, consisting essentially of, or consisting of a selectable marker gene of an original transgenic locus has been deleted. A selectable marker gene (SMG) excision site can thus comprise a contiguous segment of DNA comprising at least 10 base pairs of the DNA located 5′ to the SMG promoter and 10 base pairs of DNA located 3′ to the SMG terminator.

As used herein, the phrase “transgene element” refers to a segment of DNA comprising, consisting essentially of , or consisting of a promoter, a 5′ UTR, an intron, a coding region, a 3′UTR, or a polyadenylation signal. Polyadenylation signals include transgene elements referred to as “terminators” (e.g., NOS, pinII, rbcs, Hsp17, TubA).

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Genome editing molecules can permit introduction of targeted genetic change conferring desirable traits in a variety of crop plants (Zhang et al. Genome Biol. 2018; 19: 210; Schindele et al. FEBS Lett. 2018; 592(12): 1954). Desirable traits introduced into crop plants such as maize and soy bean include herbicide tolerance, improved food and/or feed characteristics, male-sterility, and drought stress tolerance. Nonetheless, full realization of the potential of genome editing methods for crop improvement will entail efficient incorporation of the targeted genetic changes in germplasm of different elite crop plants adapted for distinct growing conditions. Such elite crop plants will also desirably comprise useful transgenic loci which confer various traits including herbicide tolerance, pest resistance (e.g.; insect, nematode, fungal disease, and bacterial disease resistance), conditional male sterility systems for hybrid seed production, abiotic stress tolerance (e.g., drought tolerance), improved food and/or feed quality, and improved industrial use (e.g., biofuel). Provided herein are elite crop plants that are improved and/or adapted for rapid incorporation of targeted genetic changes by genome editing that comprise modified transgenic loci, and methods of making and using such crop plants. Also provided are DNA molecules obtained from the modified transgenic loci and/or plants comprising the same, biological samples containing the DNA, nucleic acid markers adapted for detecting the isolated DNA molecules, and related methods of identifying the elite crop plants comprising modified transgenic loci that are improved and/or adapted for rapid incorporation of targeted genetic changes by genome editing.

Provided herein are methods for the directed or targeted excision of selectable marker genes or scoreable marker genes from transgenic loci in transgenic plants. In certain embodiments, methods for the excision of the selectable marker genes or scoreable marker genes from transgenic loci include targeted excision of a given selectable marker genes or scoreable marker genes in a transgenic locus in certain breeding lines or crosses of transgenic loci lacking the selectable or scoreable marker genes to other plants. Other useful applications of the methods for the excision of the selectable marker genes or scoreable marker genes from transgenic loci include removal of the selectable traits from certain breeding lines when it is desirable to replace the selectable trait in the breeding line without disrupting other transgenic loci and/or non-transgenic loci. In certain embodiments, excision of selectable marker genes or scoreable marker genes from transgenic loci can be accompanied or followed by insertion of new transgenes that confer a replacement or other desirable trait at the genomic location of the excised selectable marker genes or scoreable marker genes (i.e., the excision site which remains in the genome following excision of the selectable marker gene or scoreable marker gene). Transgenic plants comprising edited genomes containing transgenic loci where the selectable marker gene or scoreable marker gene has been excised are also provided. In certain embodiments, the transgenic loci where the selectable marker gene has been excised do not contain any site-specific recombinase recognition sites (e.g., lox or FRT sites). In certain embodiments, the methods result in plants, genomic DNA, biological samples, and/or DNA containing a selectable marker gene excision site wherein a segment comprising, consisting essentially of, or consisting of a selectable marker gene of a transgenic locus is deleted.

Also provided herein are methods for the directed or targeted excision (e.g., resulting in a deletion) of polynucleotide segments from transgenic loci contained in the genomes of transgenic plants and the resulting edited transgenic plant genomes and plant cells, plant parts, and plants comprising such edited genomes. In certain embodiments, an original transgenic locus is modified by deleting a segment of DNA which comprises, consists essentially of, or consists of a segment of DNA that is non-essential for expression of any transgene in the locus. In some cases, such non-essential DNA can be considered undesirable or even detrimental to the function or purpose of the transgenic event and/or transgene and thus its removal can result in a recognizable improvement of the transgenic locus and/or of a transgenic plant comprising such an edited genome. In certain embodiments, removal of the detrimental DNA can provide for enhanced functionality of the modified transgenic locus in comparison to a transgenic locus lacking the deletion. In certain embodiments, the enhanced functionality comprises decreased silencing of an intact transgene of the modified transgenic locus comprising the deletion and/or increased expression of an intact transgene of the approved transgenic locus comprising the deletion. The generation of transgenic events by various methods can lead to the inclusion of extraneous and/or non-essential DNA sequences within transgenic loci in addition to the inserted transgenes. Non-limiting examples of non-essential DNA in a transgenic locus include synthetic cloning site sequences, duplications or other repetitions of entire transgenes, transgene elements, fragments of transgenes or transgene elements, bacterial antibiotic resistance genes (e.g., beta-lactamase (bla)), bacterial vector backbone sequences, and Agrobacterium right and/or left border sequences. Plant transformation performed by particle bombardment can in particular result in duplications and fragments of transgene sequences. Duplicate promoter sequences or fragments of promoter sequences within a transgenic locus that are in addition to the promoter sequence driving expression of a transgene may interfere with, hinder, or otherwise alter expression of the transgene or potentially other gene expression in the region of the non-essential promoter sequences as well. In certain embodiments, the non-essential DNA does not comprise DNA encoding a selectable marker gene, that is, the non-essential DNA and any selectable marker gene of a transgenic locus are considered for purposes of such an embodiment to be separate elements. In certain embodiments, methods for the excision of the segments of the transgenic loci include targeted excision of a non-essential DNA, or targeted excision of a non-essential DNA along with targeted excision of a selectable marker gene, such as in a transgenic locus in certain breeding lines. In certain embodiments, methods for the excision of the segments of the transgenic loci include crosses of plants comprising transgenic loci modified by deletion of non-essential DNA, or by deletion of non-essential DNA and a selectable marker gene, to other plants. Other useful applications of the methods for the excision of the non-essential DNA or the non-essential DNA and selectable marker gene from transgenic loci include removal of the non-essential DNA or non-essential DNA and selectable marker gene from certain breeding lines (e.g., inbred lines). For example, it is sometimes desirable to excise or replace the non-essential DNA and/or the non-essential DNA and selectable marker gene in the breeding line without disrupting other transgenic loci and/or non-transgenic loci. In certain embodiments, excision of the non-essential DNA or excision of the non-essential DNA and selectable marker gene from transgenic loci can be accompanied or followed by insertion of an introduced DNA sequence, such as new transgenes, that confer a replacement or other desirable functionality or trait at the location of the excised segment or segments (i.e., the excision site which remains in the genome following excision of the deleted polynucleotide segment). Edited transgenic plants genomes containing transgenic loci where the non-essential DNA has, or non-essential DNA and selectable marker gene have been excised are also provided. Transgenic plants comprising such edited genomes containing modified transgenic loci where non-essential DNA has, or non-essential DNA and selectable marker gene have been excised are also provided. In certain embodiments, the transgenic loci where the non-essential DNA has or non-essential DNA and selectable marker gene have been excised do not contain any site-specific recombinase recognition sites (e.g., lox or FRT sites).

Methods provided herein can be used to excise any selectable marker gene and/or non-essential DNA from transgenic loci where the DNA sequences flanking and/or comprising the selectable marker gene and/or non-essential DNA are or can be determined. Such DNA sequences are readily identified in new transgenic events by sequencing and PCR techniques. In certain embodiments, such sequences are published. Examples of transgenic loci which can be improved and used in the methods provided herein include certain corn (maize), soybean, cotton, and canola transgenic loci set forth in Tables 1, 2, 3, and 4, respectively. DNA sequences including selectable marker genes, non-essential DNA segments, and their flanking regions of certain events are also depicted in the Figures and provided herewith.

Methods provided herein can be used to excise any selectable marker genes from transgenic loci where the 5′ and 3′ DNA sequences comprising the 5′ and 3′ ends of the expression cassette comprising the selectable marker gene (e.g., a DNA segment comprising a promoter which is operably linked to DNA encoding the protein which confers the selectable trait which is in turn operably linked to DNA encoding a termination or polyadenylation signal) are known or have been determined. Such 5′ and 3′ DNA sequences flanking the selectable marker gene are readily identified in new transgenic events by sequencing and PCR techniques. In certain embodiments, the 5′ and 3′ DNA sequences flanking the selectable marker gene are published. Examples of transgenic loci which can be improved and used in the methods provided herein include certain corn (maize), soybean, cotton, and canola transgenic loci set forth in Tables 1, 2, 3, and 4, respectively. Transgenic 5′ and 3′ DNA sequences flanking the selectable marker gene for certain events are also depicted in the Figures. Such transgenic loci set forth in Tables 1-4 are found in crop plants which have in some instances been cultivated, been placed in commerce, and/or have been described in a variety of publications by various governmental bodies. Databases which have compiled descriptions of approved transgenic loci including the loci set forth in Tables 1-4 include the International Service for the Acquisition of Agri-biotech Applications (ISAAA) database (available on the world wide web internet site “isaaa.org/gmapprovaldatabase/event”), the GenBit LLC database (available on the world wide web internet site “genbitgroup.com/en/gmo/gmodatabase”), and the Biosafety Clearing-House (BCH) database (available on the http internet site “bch.cbd.int/database/organisms”).

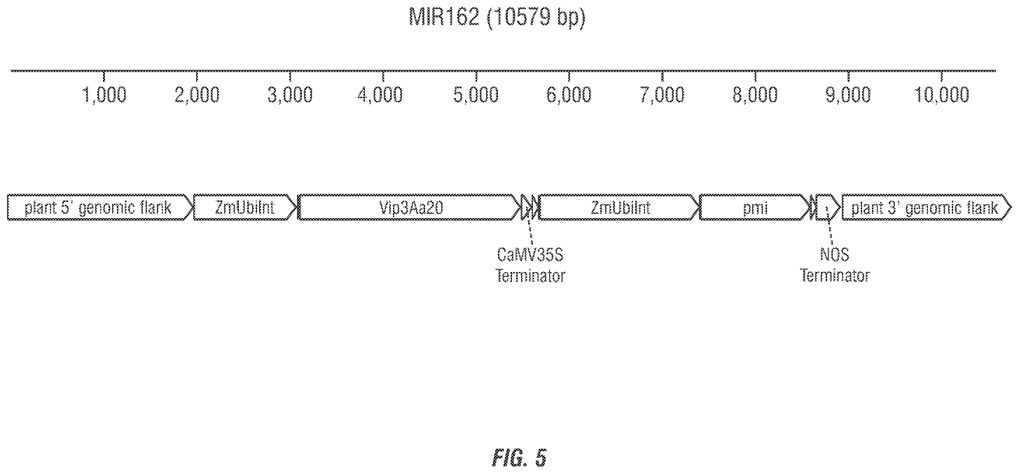

TABLE 1
Corn Events (transgenic loci)

| | Patent or Patent | ATCC or | | |
|---|---|---|---|---|
| Event Name | Application | NCIMB Deposit | Trait expression | |
| (traits)¹ | Number(s)² | Designation | cassette(s) | SEQ ID NO |
| BVLA430101 | CN2013103194381A | | phyA2 | |
| (Q) | | | | |
| Bt10 (IR, HT) | | | Cry1Ab, PAT | |
| Bt11 (IR, HT) | US 6,342,660; | ATCC 209671 | Cry1Ab and PAT | |
| | US 6,403,865; | | | |
| | US 6,943,282 | | | |
| Bt176 | | | Cry1Ab, PAT | |
| CBH-351 | JP 2006197926 A | | PAT, Cry9c | |
| (HT, IR) | | | | |
| DAS-59122-7 | US 6127180; | PTA-11384 | cry34Ab1, | SEQ ID NO: 1 |
| (IR, HT) | US 6340593; | | cry35Ab1, PAT | |
| | US 6548291; | | | |
| | US 6624145; | | | |
| | US 6893872; | | | |
| | US 6900371; | | | |
| | US 7323556 (Event); | | | |
| | US 7695914 (Event); | | | |
| | US 7696341; | | | |
| | US 7956246 (Event); | | | |
| | US 8592653 (Event); | | | |
| | US 8952223 (Event); | | | |
| | RE 43,373; | | | |
| | US 9878321 | | | |
| | (Event) | | | |
| DAS-40278 (HT) | US 20120244533 | PTA-10244 | aad-1 | SEQ ID NO: 22 |
| DBT418 (IR, HT) | | | Cry1Ac, PAT, | |
| | | | pinII | |
| DP-4114 | US 8,575,434; | PTA-11506 | Cry1Ab, | SEQ ID NO: 2 ( |
| (IR, HT) | US 10,190,179; | | cry34Ab1, | |
| | US 20190136331 | | cry35Ab1, PAT | |
| DP-32138 | US 20130031674 | PTA-9158 | Zm Ms45, Zm aa1 | SEQ ID NO: 24 |
| (MS, MSR) | US 20090038026 | | gene, DsRed2 | |
| | US 20060288440 | | | |
| DP-33121 | US 20150361446 | PTA-13392 | Cry2A.127, | SEQ ID NO: 23 |
| (IR. HT) | | | Cry1A.88, | |
| | | | VIP3 Aa20, PAT | |
| GA21 (HT) | US 2005086719; | ATCC 209033 | EPSPS | |
| | US 6,040,497; | | | |
| | US 6,762,344; | | | |
| | US 7,314,970 | | | |
| HCEM485 (HT) | US 8759618 B2 | PTA-12014 | zmEPSPS | SEQ ID NO: 25 |
| LY038 (Q) | US 7157281 | PTA-5623 | cordapA | SEQ ID NO: 26 |
| MON810 | US 6,852,915 | PTA-6260 | Cry1Ab, goxv247, | |
| (IR, HT, AR) | | | cp4epsps | |
| MON832 (HT) | | | Goxv247, cp4 | |
| | | | epsps, nptII | |
| MON863 (IR) | US 7705216 | PTA-2605 | Cry3Bb1 | |
| MON87403 (YG) | US 20170088904 | PTA-13584 | athb17 | SEQ ID NO: 27 |
| MON87411 | US 10,316,330 | PTA-12669 | cry3Bb1, cp4epsps, | SEQ ID NO: 3 |
| (IR, HT) | | | dvsnf7 | |
| MON87419 (HT) | US 2015/0267221 | PTA-120860 | DMO, PAT | SEQ ID NO: 28 |
| MON87427 | US 8,618,358 | PTA-7899 | cp4epsps | |
| (HT/MS)³ | | | | |
| MON87460 | US 8450561 | PTA-8910 | cspB | SEQ ID NO: 29 |
| (AST) | | | | |
| MON88017 | US 8,212,113; | PTA-5582 | cry3Bb1, cp4epsps | |
| (IR, HT) | US 8,686,230 | | | |
| MON89034 | US 9,428,765 | PTA-7455 | cry2Ab2, | SEQ ID NO: 4 |
| (IR)⁴ | | | cry1A.105 | |
| MIR162 | US 8,455,720 | PTA-8166 | VIP3Aa20 | SEQ ID NO: 5 |
| (IR, MU) | | | | (FIG. 5) |
| MIR604 | US 7,897,748 | none | cry3A055 | SEQ ID NO: 6 |
| (IR, MU) | | | | |
| MS3 | | | Barnase, PAT | |
| MS6 | | | barnase | |
| MZHGOJG (HT) | US_201662346688_ | PTA-122835 | ZmEPSPS, PAT | SEQ ID NO: 30 |
| | PWO 2017214074 | | | |
| MZIR098 | US 20200190533 | PTA-124143 | ecry3.1Ab, | SEQ ID NO: 31 |
| (IR, HT) | | | mcry3A, PAT | |
| MYDT09Y | | | | |
| DP-E29 | | | | |
| NK603 (HT) | US 8,273,959 | PTA-2478 | cp4epsps | SEQ ID NO: 7 |
| SYN-E3272-5 | US 8,093,453 | PTA-9972 | amy797E | SEQ ID NO: 8 |
| (BF, MU) | | | | |
| T14 (HT) | | | PAT | |
| T25 (HT) | | | PAT | |
| TC1507 (IR, HT) | US 8,901,378; | PTA-5448 | cry1Fa2, PAT | SEQ ID NO: 9 |
| | US 8,502,047 | (Inbred | | |
| | | BE1146BMR); | | |
| | | PTA-8519 | | |
| | | (LLD06BM) | | |
| TC6275 (IR, HT) | | | PAT, moCry1F | |
| VCO-Ø1981-5 | US 9,994,863 | NCIMB41842 | EPSPS | SEQ ID NO: 32 |
| (HT) | | | | |
| 676 (MS, HT) | | | dam, PAT | |
| 678 (MS, HT) | | | dam, PAT | |
| 680 (MS,HT) | | | dam, PAT | |
| 98140 (HT) | US 7,928,296 | PTA-8296 | zm-hra, GAT | SEQ ID NO: 33 |
| 5307 (IR, MU) | US 8,466,346 | PTA-9561 | ecry3.1Ab | SEQ ID NO: 10 |

¹Traits:
IR = Insect Resistance;
HT = Herbicide Tolerance;
AR = Antibiotic Resistance;
MU = mannose utilization;
BF = Biofuel;
MS = Male Sterility;
MSR = Male Sterility Restoration;
Q = Food and/or Feed Quality;
AST = Abiotic Stress Tolerance;
YG = Yield/Growth.
²Each US Patent or Patent Application Publication is incorporated herein by reference in its entirety.
³A single transgene confers vegetative tolerance to glyphosate and exhibits glyphosate-induced male sterility.
⁴Resistance to coleopteran and lepidopteran insect pests.

TABLE 2
Soybean Events (transgenic loci)

| | | ATCC;³ | | |
|---|---|---|---|---|
| | | NCIMB⁴ | | |
| | | Deposit | | |
| | Patent or Patent | Number; or | Trait | |
| | Application | Commercial | expression | |
| Event Name (traits)¹ | Number(s)² | Source | cassette(s) | SEQ ID NO |
| A5547-127 (HT) | US 20080196127 | NCIMB | PAT | |
| | RE44962 | 41660 | | |
| DAS44406-6 (HT)⁵ | US 9,540,655 | PTA-11336 | Aad-12, | SEQ ID NO: 11 |
| | US 10,400,250 | | 2mepsps, | |
| | | | PAT | |
| DAS68416-4 | US 9,738,904 | PTA-10442 | Aad-12, PAT | SEQ ID NO: 12 |
| (IR, HT)⁶ | | PTA-12006 | | |
| DAS81419-2 (IR, HT) | US 8680363 | PTA-12006 | cry1Ac, | SEQ ID NO: 13 |
| | US 8632978 | | | |
| | US 9695441 | | | |
| | US 9738904 | | cry1F, PAT | |
| GTS 40-3-2 (HT) | US 20070136836 | M690GT \| 0.9 | cp4epsps | |
| | | RM Soybean⁷ | | |
| MON87701 (IR) | US 8049071 | PTA-8194 | cry1Ac | SEQ ID NO: 14 |
| MON87708 (HT)⁸ | US 9447428 | PTA-9670 | DMO | SEQ ID NO: 15 |
| MON89788 (HT) | US 9944945 | PTA-6708 | cp4epsps | SEQ ID NO: 16 |
| MST-FGØ72-3 (HT)⁹ | US 8592650 | NCIMB | hppdPF | SEQ ID NO: 34 |
| | | 41659 | W336, | |
| | | | 2mepsps | |
| SYHT0H2¹⁰ | US 10,184,134 | PTA-11226 | CAvHPPD-03 | |

¹Traits:
IR = Insect Resistance;
HT = Herbicide Tolerance;
AR = Antibiotic Resistance;
MU = mannose utilization;
BF = Biofuel;
MS = Male Sterility.
²Each US Patent or Patent Application Publication is incorporated herein by reference in its entirety.
³ATCC is the American Type Culture Collection, 10801 University Boulevard Manassas, VA 20110 USA (for “PTA-XXXXX” deposits).
⁴NCIMB is the National Collection of Industrial, Food and Marine Bacteria, Ferguson Building, Craibstone Estate, Bucksburn, Aberdeen AB9YA, Scotland.
⁵HT to 2,4-D; glyphosate, and glufosinate; also refered to as pDAB8264.44.06.1.
⁶Independent IR/HT and HT events combined by breeding. IR/HT event (Cry 1F, Cry1Ac synpro (Cry1Ac), and PAT) is DAS81419-2, deposited with ATCC under PTA-12006, also referred to as DAS81419-2.
⁷Elk Mound Seed, 308 Railroad Street Elk Mound, WI, USA 54739.
⁸HT to dicamba.
⁹HT to both glyphosate and isoxaflutole herbicides.
¹⁰HT to glufosinate and mesotrione herbicides.

TABLE 3
Cotton Events (transgenic loci)

| Event Name | Patent | | Trait | |
|---|---|---|---|---|
| (traits)¹ | Number(s) | ATCC Deposit | expression cassette(s) | SEQ ID NO |
| DAS-21023-5 | US 7,179,965 | PTA-6233 | Cry1Ac, PAT | SEQ ID NO: 17 |
| (IR, HT) ¹ | | | | |
| DAS-24236-5 | US 7,179,965 | PTA-6233 | Cry1F, PAT | SEQ ID NO: 18 |
| (IR, HT) ¹ | | | | |
| COT102 (IR, AR) ² | US 7,371,940 | | Vip3A(a), | SEQ ID NO: 19 |
| LLcotton25 (HT) | US | PTA-3343 | PAT | |
| | 20030097687 | | | |
| MON15985 | US 9,133,473 | PTA-2516 | cry1Ac, | |
| (IR, AR, SM) ³ | | | cry2Ab2 | |
| MON88701 (HT)⁴ | US 8,735,661 | PTA-11754 | DMO, PAT | SEQ ID NO: 20 |
| MON88913 (HT) | US 7,381,861 | PTA-4854 | cp4 epsps | |

¹ Traits:
IR = Insect Resistance;
HT = Herbicide Tolerance;
AR = Antibiotic Resistance;
SM= Screenable Marker.
² Both cry1Ac cotton event 3006-210-23 and cry1F cotton event 281-24-236 described in US 7,179,965; seed comprising both events deposited with ATCC as PTA-6233.
³ Contains both the MON531 chimeric Cry1A and MON15985X Cry2Ab insertions.
⁴Tolerance to dicamba and glufosinate herbicides.

TABLE 4
Canola Events (transgenic loci)

| | Patent or Patent | | | |
|---|---|---|---|---|
| | Application | | Trait | |
| Event Name | Publication | ATCC | Expression | |
| (traits)¹ | Number(s) | Deposit | cassette | SEQ ID NO |
| GT73 (HT) | US 8,048,632 | PTA- | cp4 epsps | |
| | US 9,474,223 | 121409 | | |
| HCN28/ | | | | |
| T45 (HT) | | | | |
| MON88302 | US 9,738,903 | PTA-10955 | cp4 epsps | SEQ ID NO: 21 |
| (HT) | | | | |
| MS8 (MS) | US2003188347 | PTA-730 | | |
| RF3 (HT) | US2003188347 | PTA-730 | | |

¹Traits:
HT = Herbicide Tolerance;
MS = Male Sterility

Sequences of certain transgenic loci are set forth in Tables 1-4 (e.g., SEQ ID NO: 1-34). the patent references set forth therein and incorporated herein by reference, and elsewhere in this disclosure. Such sequences include the 5′ and 3′ DNA sequences flanking the selectable marker genes, non-essential DNA sequences, the selectable marker gene cassette sequences, as well as the sequences of other expression cassettes that confer useful traits (e.g., herbicide tolerance, insect resistance, biofuel use). Allelic or other variant sequences corresponding to the sequences set forth in Tables 1-4 and elsewhere in this disclosure which may be present in certain variant transgenic plant loci can also be improved by identifying sequences in the variants that correspond to the sequences of Tables 1-4 (e.g., SEQ ID NO: 1-34), the patent references set forth therein and incorporated herein by reference, and elsewhere in this disclosure by performing a pairwise alignment (e.g., using CLUSTAL O 1.2.4 with default parameters) and making corresponding changes in the allelic or other variant sequences. Such allelic or other variant sequences include sequences having at least 85%, 90%, 95%, 98%, or 99% sequence identity across the entire length or at least 20, 40, 100, or 500, 1,000, 2,000, 4,000, 8,000, 10,000, or 12,000 nucleotides of the sequences set forth in Tables 1-4 (e.g., SEQ ID NO: 1-34), the patent references set forth therein and incorporated herein by reference, and elsewhere in this disclosure. Also provided are plants, genomic DNA, and/or isolated DNA obtained from the plants set forth in Tables 1-4 comprising modifications of their transgenic loci comprising a selectable marker gene excision site wherein a segment comprising, consisting essentially of, or consisting of a selectable marker gene of a transgenic locus is deleted. Also provided herein are plants, genomic DNA, and/or isolated DNA obtained from the plants set forth in Tables 1-4 comprising modifications of their transgenic loci which enhance functionality of the transgenic locus including deletions of non-essential DNA from the transgenic locus. In certain embodiments, the functionality enhancing modification can comprise a deletion of the segment comprising. consisting essentially of, or consisting of: a duplication of a transgene; a duplication of a transgene element; and/or a fragment of a transgene; optionally, wherein the duplication and/or fragment of a transgene element is a duplication and/or fragment of a promoter or a polyadenylation signal.

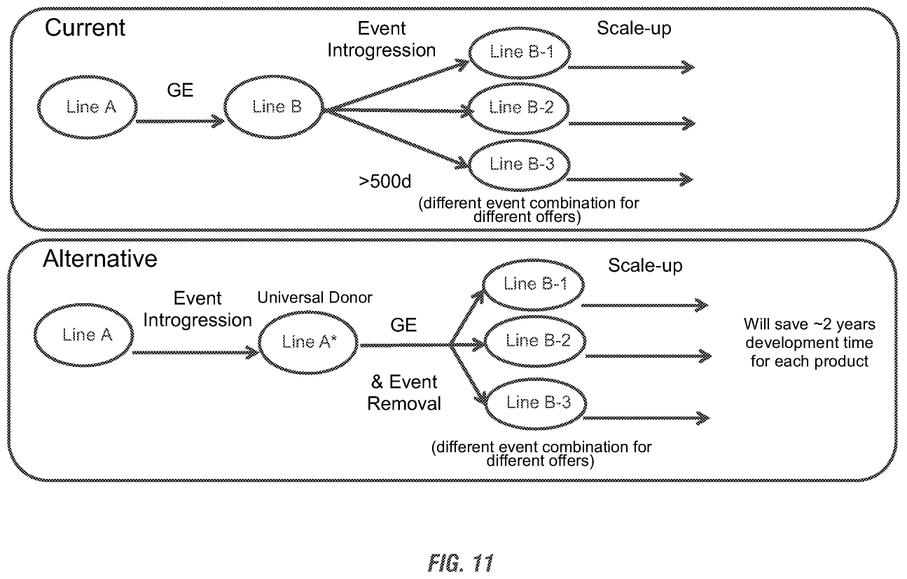

Modified transgenic loci provided herein can be used in a variety of breeding schemes to obtain or use the elite crop plants comprising the modified transgenic loci and, in certain aspects, targeted genetic changes. Such elite crop plants can be inbred plant lines or can be hybrid plant lines. In certain embodiments, one or more modified transgenic loci (e.g., transgenic loci in Tables 1-4 which have been subjected to genome editing) are introgressed into a desired donor line comprising elite crop plant germplasm and then optionally subjected to genome editing molecules to recover plants comprising both the modified transgenic loci and targeted genetic changes introduced by the genome editing molecules. Introgression can be achieved by backcrossing plants comprising the modified transgenic locus to a recurrent parent comprising the desired elite germplasm and selecting progeny with the modified transgenic locus and recurrent parent germplasm. Such backcrosses can be repeated and/or supplemented by molecular assisted breeding techniques using SNP or other nucleic acid markers to select for recurrent parent germplasm until a desired recurrent parent percentage is obtained (e.g., at least 95%, 96%, 97%, 98%, or 99% recurrent parent percentage). A non-limiting, illustrative depiction of a scheme for obtaining plants with both modified transgenic loci and the targeted genetic changes is shown in the FIG. 11 (bottom “Alternative” panel), where one or more of the modified transgenic loci (“Event” in FIG. 11) are present in Line A and then moved into elite crop plant germplasm by introgression. In the non-limiting FIG. 11 illustration, introgression can be achieved by crossing a “Line A” comprising one or more of the modified transgenic loci to the elite germplasm and then backcrossing progeny of the cross comprising the modified transgenic loci to the elite germplasm as the recurrent parent) to obtain a “Universal Donor” (e.g. Line A+ in FIG. 11) comprising one or more of the modified transgenic loci. This elite germplasm containing the modified transgenic loci (e.g. “Universal Donor” of FIG. 11) can then be subjected to genome editing molecules which introduce other targeted genetic changes in the genomes of the elite crop plants containing the modified transgenic loci. In certain embodiments where more than one modified transgenic locus is present in the elite crop plant (e.g. “Universal Donor” of FIG. 11), a modified transgenic locus (“Event” in FIG. 11) can be removed to obtain an elite crop plant having a subset of modified transgenic loci and a targeted genetic change. In certain embodiments, it is also desirable to bulk up populations of inbred elite crop plants or their seed comprising the modified transgenic loci by selfing. Such inbred progeny of the selfed plants can be used either as is for commercial sales where the crop can be grown a varietal, non-hybrid crop (e.g., commonly though not always in soybean, cotton, or canola). In certain embodiments, inbred progeny of the selfed plants can be used as a pollen donor or recipient for hybrid seed production (e.g., most commonly in maize but also in cotton, soybean, and canola).

Hybrid plant lines comprising elite crop plant germplasm, the modified transgenic loci, and in certain aspects, additional targeted genetic changes are also provided herein. Methods for production of such hybrid seed can comprise crossing elite crop plant lines where at least one of the pollen donor or recipient comprises at least the modified transgenic loci and/or additional targeted genetic changes. In certain embodiments, the pollen donor and recipient will comprise germplasm of distinct heterotic groups and provide hybrid seed and plants exhibiting heterosis. In certain embodiments, the pollen donor and recipient can each comprise a distinct modified transgenic locus which confers either a distinct trait (e.g., herbicide tolerance or insect resistance), a different type of trait (e.g., tolerance to distinct herbicides or to distinct insects such as coleopteran or lepidopteran insects), or a different mode-of-action for the same trait (e.g., resistance to coleopteran insects by two distinct modes-of-action or resistance to lepidopteran insects by two distinct modes-of-action). In certain embodiments, the pollen recipient will be rendered male sterile or conditionally male sterile. Methods for inducing male sterility or conditional male sterility include emasculation (e.g., detasseling), cytoplasmic male sterility, chemical hybridizing agents or systems, a transgenes or transgene systems, and/or mutation(s) in one or more endogenous plant genes. Descriptions of various male sterility systems that can be adapted for use with the elite crop plants provided herein are described in Wan et al. Molecular Plant; 12, 3, (2019):321-342 as well as in U.S. Pat. No. 8,618,358; US 20130031674; and US 2003188347. In certain embodiments, it will be desirable to use genome editing molecules to effect modifications of transgenic loci and/or make targeted genetic changes in elite crop plant or other germplasm. Techniques for effecting genome editing in crop plants (e.g., maize,) include use of morphogenic factors such as Wuschel (WUS), Ovule Development Protein (ODP), and/or Babyboom (BBM) which can improve the efficiency of recovering plants with desired genome edits. In some aspects, the morphogenic factor comprises WUS1, WUS2, WUS3, WOX2A, WOX4, WOX5, WOX9, BBM2, BMN2, BMN3, and/or ODP2. In certain embodiments, compositions and methods for using WUS, BBM, and/or ODP, as well as other techniques which can be adapted for effecting genome edits in elite crop plant and other germplasm, are set forth in US 20030082813, US 20080134353, US 20090328252, US 20100100981, US 20110165679, US 20140157453, US 20140173775, and US 20170240911, which are each incorporated by reference in their entireties. In certain embodiments, the genome edits can be effected in regenerable plant parts (e.g., plant embryos) of elite crop plants by transient provision of gene editing molecules or polynucleotides encoding the same and do not necessarily require incorporating a selectable marker gene into the plant genome (e.g., US 20160208271 and US 20180273960, both incorporated herein by reference in their entireties; Svitashev et al. Nat Commun. 2016; 7:13274).

Provided for herein is a modified version of an approved transgenic locus which in its unmodified form (in certain embodiments, the “unmodified form” is the “original form,” “original transgenic locus,” etc.) comprises at least one selectable marker gene. In the modified version, at least one selectable marker has been deleted with genome editing molecules as described elsewhere herein from the unmodified approved transgenic locus. In certain embodiments, the targeted genome editing and deletion of the selectable marker gene does not affect any other functionality of the approved transgenic locus. In certain embodiments, the selectable marker gene that is deleted confers resistance to an antibiotic, tolerance to an herbicide, or an ability to grow on a specific carbon source, for example, mannose. In certain embodiments, the selectable marker gene comprises a DNA encoding a phosphinothricin acetyl transferase (PAT), a glyphosate tolerant 5-enol-pyruvylshikimate-3-phosphate synthase (EPSPS), a glyphosate oxidase (GOX), neomycin phosphotransferase (npt), a hygromycin phosphotransferase (hyg), an aminoglycoside adenyl transferase, or a phosphomannose isomerase (pmi). In certain embodiments, the modified locus does not contain a site-specific recombination system DNA recognition site, for example, in certain embodiments, the modified locus does not contain a lox or FRT site. In certain embodiments, the selectable marker gene to be deleted is flanked by operably linked protospacer adjacent motif (PAM) sites in the unmodified form of the approved transgenic locus. Thus, in certain embodiments of the modified locus, PAM sites flank the excision site of the deleted selectable marker gene. In certain embodiments, the PAM sites are recognized by an RNA dependent DNA endonuclease (RdDe); for example, a class 2 type II or class 2 type V RdDe. In certain embodiments, the deleted selectable marker gene is replaced in the modified approved transgenic locus by an introduced DNA sequence as discussed in further detail elsewhere herein. For example, in certain embodiments, the introduced DNA sequence comprises a trait expression cassette such as a trait expression cassette of another transgenic locus. In addition to the deletion of a selectable marker gene, in certain embodiments at least one copy of a repetitive sequence has also been deleted with genome editing molecules from an unmodified approved transgenic locus. In certain embodiments, deletion of the repetitive sequence enhances the functionality of the modified approved transgenic locus. In certain embodiments, the approved transgenic locus which is modified is: (i) a Bt11, DAS-59122-7, DP-4114, GA21, MON810, MON87411, MON87427, MON88017, MON89034, MIR162, MIR604, NK603, SYN-E3272-5, 5307, DAS-40278, DP-32138, DP-33121, HCEM485, LY038, MON863, MON87403, MON87403, MON87419, MON87460, MZHG0JG, MZIR098, VCO-Ø1981-5, 98140, and/or TC1507 transgenic locus in a transgenic maize plant genome; (ii) an A5547-127, DAS44406-6, DAS68416-4, DAS81419-2, GTS 40-3-2, MON87701, MON87708, MON89788, MST-FGØ72-3, and/or SYHT0H2 transgenic locus in a transgenic soybean plant genome; (iii) a DAS-21023-5, DAS-24236-5, COT102, LLcotton25, MON15985, MON88701, and/or MON88913 transgenic locus in a transgenic cotton plant genome; or (iv) a GT73, HCN28, MON88302, and/or MS8 transgenic locus in a transgenic canola plant genome. Also provided herein are plants comprising any of aforementioned modified transgenic loci.

Excision of selectable marker genes and/or non-essential DNA can be achieved by using suitable gene editing molecules which can introduce blunt or staggered double stranded DNA breaks in 5′ and 3′ DNA sequences flanking or comprising the selectable marker genes and/or non-essential DNA of transgenic loci. Such blunt or staggered dsDNA breaks can be introduced in or adjacent to the promoter and terminator or polyadenylation signal of the selectable marker gene. Typically, the breaks are introduced at or just 5′ to the DNA comprising the promoter and at or just 3′ to the DNA comprising the terminator or polyadenylation signal. However, such breaks can also be introduced within DNA comprising the promoter and the terminator or polyadenylation signal of the selectable marker gene. In certain embodiments, the gene editing molecules can comprise zinc finger nucleases, zinc finger nickases, TALENs, and/or TALE nickases which introduce double stranded breaks in DNA segments flanking a sequence to be deleted from the genome (e.g., selectable marker gene cassettes and/or non-essential DNA). In certain embodiments, the gene editing molecules comprise RdDe and guide RNAs directed to DNA targets comprising pre-existing PAM sites in DNA flanking or comprising the promoter and the terminator or polyadenylation signal of the selectable marker gene in the transgenic plant genome. Such PAM sites can be recognized by RdDe and suitable guide RNAs directed to DNA sequences adjacent to the PAM to provide for cleavage within or near the DNA sites targeted for cleavage. In certain embodiments, the PAMs are recognized by the same class and/or type of RdDe (e.g., class 2 type II or class 2 type V) or by the same RdDe (e.g., both PAMs recognized by the same Cas9 or Cas 12 RdDe). Guide RNAs can be directed to the DNA sites targeted for cleavage by using pre-existing PAM sites (e.g., located within or adjacent to a DNA segments flanking a selectable marker gene cassette and/or non-essential DNA). Non-limiting examples of such pre-existing PAM sites present in polynucleotides which can be used by suitable guide RNAs to direct RdDe or RNA dependent nickases in a DNA segments flanking selectable marker gene cassettes of certain transgenic loci are set forth in Table 5, Table 6, Table 7, Table 8, and Table 9 of the Examples. In certain embodiments, a selectable marker gene conferring herbicide tolerance or antibiotic resistance is excised from a transgenic locus having a primary functionality of conferring insect resistance, male sterility, or biofuel use. In certain embodiments, the selectable marker gene which confers antibiotic resistance is excised from a transgenic locus having a primary functionality of conferring herbicide tolerance.

In certain embodiments, edited transgenic plant genomes, transgenic plant cells, parts, or plants containing those genomes, and DNA molecules obtained therefrom can lack one or more non-essential DNAs and/or selectable and/or scoreable markers found in an original event (transgenic locus) and comprise a selectable marker gene excision site or a scoreable marker gene excision site. When a segment comprising a selectable marker gene (SMG) of an original transgenic locus has been deleted, the selectable marker gene excision site can comprise a contiguous segment of DNA comprising at least 10 base pairs of the DNA located 5′ to the SMG promoter and 10 base pairs of DNA located 3′ to the SMG terminator, wherein the entire selectable marker gene (e.g., an expression cassette in the original transgenic locus comprising a promoter which is operably linked to DNA encoding the selectable marker protein which operably linked to a terminator) has been deleted. In certain embodiments where a segment comprising a selectable marker gene of an original transgenic locus has been deleted, the selectable marker gene excision site can comprise a contiguous segment of DNA comprising at least 10 base pairs of the DNA located 5′ to the excision site and 10 base pairs of DNA located 3′ to an excision site wherein the entire selectable marker gene (e.g., an expression cassette in the original transgenic locus comprising a promoter which is operably linked to DNA encoding the selectable marker protein which operably linked to a polyadenylation sequence) and at least 1, 2, 5, 10, 20, 50, or more base pairs of DNA located 5′ to the SMG promoter and/or 3′ to the SMG polyadenylation signal in the original transgenic locus has been deleted. In such embodiments where DNA comprising the selectable marker gene or scoreable marker gene is deleted, a selectable marker excision site can comprise at least 10 base pairs of the DNA located 5′ to the excision site and 10 base pairs of DNA located 3′ to an excision site (e.g., DNA located 5′ to the SMG promoter and/or 3′ to the SMG polyadenylation signal prior to deletion of the fragment) wherein all of the selectable marker gene sequences are absent and either all or less than all of the DNA flanking the selectable marker gene or scoreable marker gene sequences are present. In any of the aforementioned embodiments or in other embodiments, the continuous segment of DNA comprising the selectable marker gene excision site can further comprise an insertion of 1 to about 2, 5, 10, 20, or more nucleotides between the DNA located 5′ and 3′ to the excision site. Such insertions can result either from endogenous DNA repair and/or recombination activities at the double stranded breaks introduced at the excision site and/or from deliberate insertion of an oligonucleotide. In certain embodiments where a segment consisting essentially of a selectable marker gene of an original transgenic locus has been deleted. the selectable marker gene excision site can be a contiguous segment of at least 10 base pairs of the DNA located 5′ to the excision site and 10 base pairs of DNA located 3′ to an excision site wherein less than the entire selectable marker gene (e.g., an expression cassette in the original transgenic locus comprising a promoter which is operably linked to DNA encoding the selectable marker protein which operably linked to a polyadenylation signal sequence) has been deleted. In certain aforementioned embodiments where a segment consisting essentially of a selectable marker gene of an original transgenic locus has been deleted. the selectable marker excision site can thus contain at least 1 base pair of DNA or 1 to about 2 or 5, 8, 10, 20, or 50 base pairs of DNA comprising the 5′ end and/or 3′ end of the selectable marker gene cassette (e.g., DNA comprising fragments of the selectable marker gene cassette promoter and/or polyadenylation signal). In certain embodiments where a segment consisting of a selectable marker gene of an original transgenic locus has been deleted. the selectable marker gene excision site can contain a contiguous segment of DNA comprising at least 10 base pairs of the DNA located 5′ to the excision site and 10 base pairs of DNA located 3′ to an excision site wherein the entire selectable marker gene (e.g., an expression cassette in the original transgenic locus comprising a promoter which is operably linked to DNA encoding the selectable marker protein which operably linked to a polyadenylation signal sequence) has been deleted. In such embodiments where DNA consisting of the selectable marker gene is deleted, a selectable marker excision site can comprise at least 10 base pairs of the DNA located 5′ to the excision site and 10 base pairs of DNA located 3′ to an excision site wherein all of the selectable marker gene sequences are absent and all the DNA flanking the selectable marker sequences are present. Deletions of DNA segments comprising, consisting essentially of, or consisting of scoreable marker genes from transgenic loci can provide scoreable marker gene excision sites with features analogous to those of the aforementioned selectable marker gene excision sites. Original transgenic loci (events), including those set forth in Tables 1-4 and depicted in the drawings. can contain selectable transgenes markers conferring herbicide tolerance, antibiotic resistance, or an ability to grow on a carbon source. Selectable marker transgenes which can confer herbicide tolerance include genes encoding a phosphinothricin acetyl transferase (PAT), a glyphosate tolerant 5-enol-pyruvylshikimate-3-phosphate synthase (EPSPS), and a glyphosate oxidase (GOX). Selectable marker transgenes which can confer antibiotic resistance include genes encoding a neomycin phosphotransferase (npt), a hygromycin phosphotransferase, an aminoglycoside adenyl transferase. Transgenes encoding a phosphomannose isomerase (pmi) can confer the ability to grow on mannose. Original transgenic loci (events), including certain events set forth in Tables 1-4, can contain scoreable transgenic markers which can be detected by enzymatic, histochemical, nucleic acid detection (e.g., sequencing, amplification, hybridization, SNP), or other assays. Scoreable marker genes can include genes encoding beta-glucuronidase (uid) or fluorescent proteins (e.g., a GFP, RFP, or YFP). Such selectable or scoreable marker transgenes can be excised from an original transgenic locus by contacting the transgenic locus with one or more gene editing molecules which introduce double stranded breaks in the transgenic locus at the 5′ and 3′ end of the expression cassette comprising the selectable marker transgene (e.g., an RdDe and guide RNAs directed to PAM sites located at the 5′ and 3′ end of the expression cassette comprising the selectable marker transgenes) and selecting for plant cells, plant parts, or plants wherein the selectable or scoreable marker has been excised in whole or in part. Plants, edited plant genomes, biological samples, and DNA molecules (e.g., including isolated or purified DNA molecules) comprising the selectable marker gene excision sites are provided herein. Nucleic acid markers adapted for detecting the selectable marker gene excision sites and/or scorable marker gene excision sites as well as methods for detecting the presence of DNA molecules comprising the selectable marker excision sites and/or scorable marker gene excision sites are also provided herein.

Methods and reagents (e.g., nucleic acid markers including nucleic acid probes and/or primers) for detecting plants, edited plant genomes, and biological samples containing DNA molecules comprising the selectable marker gene excision sites and/or non-essential DNA deletions are also provided herein. Detection of the DNA molecules can be achieved by any combination of nucleic acid amplification (e.g., PCR amplification), hybridization, sequencing, and/or mass-spectrometry based techniques. Methods set forth for detecting junction nucleic acids in unmodified transgenic loci set forth in US 20190136331 and U.S. Pat. No. 9,738,904, both incorporated herein by reference in their entireties, can be adapted for use in detection of the nucleic acids provided herein. In certain embodiments, such detection is achieved by amplification and/or hybridization-based detection methods using a method (e.g., selective amplification primers) and/or probe (e.g., capable of selective hybridization or generation of a specific primer extension product) which specifically recognizes the target DNA molecule (e.g., selectable marker gene excision site) but does not recognize DNA from an unmodified transgenic locus. In certain embodiments, the hybridization probes can comprise detectable labels (e.g., fluorescent, radioactive, epitope, and chemiluminescent labels). In certain embodiments, a single nucleotide polymorphism detection assay can be adapted for detection of the target DNA molecule (e.g., selectable marker gene excision site).

In certain embodiments, the selectable or scoreable marker transgene can be inactivated. Inactivation can be achieved by modifications including insertion, deletion, and/or substitution of one or more nucleotides in a promoter element, 5′ or 3′ untranslated region (UTRs), intron, coding region, and/or 3′ terminator and/or polyadenylation signal of the selectable marker transgene. Such modifications can inactivate the selectable or scoreable marker transgene by eliminating or reducing promoter activity, introducing a missense mutation, and/or introducing a pre-mature stop codon. In certain embodiments, the selectable and/or scoreable marker transgene can be replaced by an introduced transgene. In certain embodiments, an original transgenic locus that was contacted with gene editing molecules which introduce double stranded breaks in the transgenic locus at the 5′ and 3′ end of the expression cassette comprising the selectable marker and/or scoreable transgene can also be contacted with a suitable donor DNA template comprising an expression cassette flanked by DNA homologous to remaining DNA in the transgenic locus located 5′ and 3″ to the selectable marker excision site. In certain embodiments, a coding region of the selectable and/or scoreable marker transgene can be replaced with another coding region such that the replacement coding region is operably linked to the promoter and 3′ terminator or polyadenylation signal of the selectable and/or scoreable marker transgene.

In certain embodiments, edited transgenic plant genomes provided herein can comprise additional new introduced DNA sequences including transgenes (e.g., expression cassettes) inserted into the transgenic locus of a given event. Introduced transgenes inserted at the transgenic locus of an event subsequent to the event's original isolation can be obtained by inducing a double stranded break at a site within an original transgenic locus (e.g., with genome editing molecules including an RdDe and suitable guide RNA(s); a suitable engineered zinc-finger nuclease; a TALEN protein and the like) and providing an exogenous transgene in a donor DNA template which can be integrated at the site of the double stranded break (e.g. by homology-directed repair (HDR) or by non-homologous end-joining (NHEJ). In certain embodiments, introduced transgenes can be integrated in a selectable marker gene excision site created by using a suitable RdDe, guide RNA, and either a pre-existing PAM site in the DNA segments that flank or comprise the 5′ end or 3′ end of the selectable marker gene. In certain embodiments, such deletions and replacements are effected by introducing dsDNA breaks in DNA segments that flank or comprise the 5′ end or 3′ end of the selectable marker gene and providing the new expression cassettes on a donor DNA template or other DNA template suitable for integration by NHEJ or MMEJ (microhomology mediated end joining). Suitable expression cassettes for insertion include DNA molecules comprising promoters which are operably linked to DNA encoding proteins and/or RNA molecules which confer useful traits which are in turn operably linked to polyadenylation signal or terminator elements. In certain embodiments, such expression cassettes can also comprise 5′ UTRs, 3′ UTRs, and/or introns. Useful traits include biotic stress tolerance (e.g., insect resistance, nematode resistance, or disease resistance), abiotic stress tolerance (e.g., heat, cold, drought, and/or salt tolerance), herbicide tolerance, and quality traits (e.g., improved fatty acid compositions, protein content, starch content, and the like). Suitable expression cassettes for insertion include expression cassettes contained in any of the events (transgenic loci) listed in Table 1 or set forth in the drawings which confer insect resistance, herbicide tolerance, biofuel use, male sterility, or other useful traits.

In certain embodiments, plants provided herein, including plants with one or more modified transgenic loci comprising selectable marker gene excision sites and/or deletions of one or more non-essential DNAs can further comprise one or more targeted genetic changes introduced by one or more of gene editing molecules or systems. Also provided are methods where the targeted genetic changes are introduced into plants which include plants with one or more modified transgenic loci comprising selectable marker gene excision sites and/or deletions of one or more non-essential DNAs. Such targeted genetic changes include those conferring traits such as improved yield, improved food and/or feed characteristics (e.g., improved oil, starch, protein, or amino acid quality or quantity), improved nitrogen use efficiency, improved biofuel use characteristics (e.g., improved ethanol production), male sterility/conditional male sterility systems (e.g., by targeting endogenous MS26, MS45 and MSCAl genes), herbicide tolerance (e.g., by targeting endogenous ALS, EPSPS, HPPD, or other herbicide target genes), delayed flowering, non-flowering, increased biotic stress resistance (e.g., resistance to insect, nematode, bacterial, or fungal damage), increased abiotic stress resistance (e.g., resistance to drought, cold, heat, metal, or salt), enhanced lodging resistance, enhanced growth rate, enhanced biomass, enhanced tillering, enhanced branching, delayed flowering time, delayed senescence, increased flower number, improved architecture for high density planting, improved photosynthesis, increased root mass, increased cell number, improved seedling vigor, improved seedling size, increased rate of cell division, improved metabolic efficiency, and increased meristem size in comparison to a control plant lacking the targeted genetic change. Types of targeted genetic changes that can be introduced include insertions, deletions, and substitutions of one or more nucleotides in the crop plant genome. Sites in endogenous plant genes for the targeted genetic changes include promoter, coding, and non-coding regions (e.g., 5′ UTRs, introns, splice donor and acceptor sites and 3′ UTRs). In certain embodiments, the targeted genetic change comprises an insertion of a regulatory or other DNA sequence in an endogenous plant gene. Non-limiting examples of regulatory sequences which can be inserted into endogenous plant genes with gene editing molecules to effect targeted genetic changes which confer useful phenotypes include those set forth in US Patent Application Publication 20190352655, which is incorporated herein by example. such as: (a) auxin response element (AuxRE) sequence; (b) at least one D1-4 sequence (Ulmasov et al. (1997) Plant Cell, 9:1963-1971), (c) at least one DR5 sequence (Ulmasov et al. (1997) Plant Cell, 9:1963-1971); (d) at least one m5-DR5 sequence (Ulmasov et al. (1997) Plant Cell, 9:1963-1971); (e) at least one P3 sequence; (f) a small RNA recognition site sequence bound by a corresponding small RNA (e.g., an siRNA, a microRNA (miRNA), a trans-acting siRNA as described in U.S. Pat. No. 8,030,473, or a phased sRNA as described in U.S. Pat. No. 8,404,928; both of these cited patents are incorporated by reference herein); (g) a microRNA (miRNA) recognition site sequence; (h) the sequence recognizable by a specific binding agent includes a microRNA (miRNA) recognition sequence for an engineered miRNA wherein the specific binding agent is the corresponding engineered mature miRNA; (i) a transposon recognition sequence; (j) a sequence recognized by an ethylene-responsive element binding-factor-associated amphiphilic repression (EAR) motif; (k) a splice site sequence (e.g., s donor site, a branching site, or an acceptor site; see, for example, the splice sites and splicing signals set forth in the internet site lemur[dot]amu[dot]edu[dot]pl/share/ERISdb/home.html); (l) a recombinase recognition site sequence that is recognized by a site-specific recombinase; (m) a sequence encoding an RNA or amino acid aptamer or an RNA riboswitch, the specific binding agent is the corresponding ligand, and the change in expression is upregulation or downregulation; (n) a hormone responsive element recognized by a nuclear receptor or a hormone-binding domain thereof; (o) a transcription factor binding sequence; and (p) a polycomb response element (see Xiao et al. (2017) Nature Genetics, 49:1546-1552. doi: 10.1038/ng.3937). Non limiting examples of target maize genes that can be subjected to targeted gene edits to confer useful traits include: (a) ZmIPK1 (herbicide tolerant and phytate reduced maize; Shukla et al., Nature. 2009; 459:437-41); (b) ZmGL2 (reduced epicuticular wax in leaves; Char et al. Plant Biotechnol J. 2015; 13:1002); (c) ZmMTL (induction of haploid plants; Kelliher et al. Nature. 2017; 542:105); (d) Wx1 (high amylopectin content; US 20190032070; incorporated herein by reference in its entirety); (e) TMS5 (thermosensitive male sterile; Li et al. J Genet Genomics. 2017; 44:465-8); (f) ALS (herbicide tolerance; Svitashev et al.; Plant Physiol. 2015; 169:931-45); and (g) ARGOS8 (drought stress tolerance; Shi et al., Plant Biotechnol J. 2017; 15:207-16). Non-limiting examples of target soybean genes that can be subjected to targeted gene edits to confer useful traits include: (a) FAD2-1A, FAD2-1B (increased oleic acid content; Haun et al.; Plant Biotechnol J. 2014; 12:934-40); (b) FAD2-1A, FAD2-1B, FAD3A (increased oleic acid and decreased linolenic content; Demorest et al., BMC Plant Biol. 2016; 16:225); and (c) ALS (herbicide tolerance; Svitashev et al.; Plant Physiol. 2015; 169:931-45). A non-limiting examples of target Brassica genes that can be subjected to targeted gene edits to confer useful traits include: (a) the FRIGIDA gene to confer early flowering (Sun Z. et al.. J Integr Plant Biol. 2013; 55:1092-103); and (b) ALS (herbicide tolerance; US 20160138040, incorporated herein by reference in its entirety). Non-limiting examples of target genes in crop plants including corn and soy bean which can be subjected to targeted genetic changes which confer useful phenotypes include those set forth in US Patent Application Nos. 20190352655, 20200199609, 20200157554, and 20200231982, which are each incorporated herein in their entireties; and Zhang et al. (Genome Biol. 2018; 19: 210).

Gene editing molecules of use in methods provided herein include molecules capable of introducing a double-strand break (“DSB”) or single-strand break (“SSB”) in double-stranded DNA, such as in genomic DNA or in a target gene located within the genomic DNA as well as accompanying guide RNA or donor DNA template polynucleotides. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a class 2 type II Cas nuclease, a Cas9, a nCas9 nickase, a class 2 type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas14, an engineered nuclease. a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for an RNA-guided nuclease, or a DNA encoding a gRNA for an RNA-guided nuclease; (d) donor DNA template polynucleotides; and (e) other DNA templates (dsDNA, ssDNA, or combinations thereof) suitable for insertion at a break in genomic DNA (e.g., by non-homologous end joining (NHEJ) or microhomology-mediated end joining (MMEJ).

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, genome-inserted CRISPR elements are useful in plant lines adapted for use in the methods provide herein. In certain embodiments, plants or plant cells used in the systems, methods, and compositions provided herein can comprise a transgene that expresses a CRISPR endonuclease (e.g., a Cas9), a Cpf1-type or other CRISPR endonuclease). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas12a can also recognize a 5′-CTA PAM motif. Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites. The Cpf1 based editing system may or may not comprise a tracrRNA. Introduction of one or more of a wide variety of CRISPR guide RNAs that interact with CRISPR endonucleases integrated into a plant genome or otherwise provided to a plant is useful for genetic editing for providing desired phenotypes or traits, for trait screening, or for gene editing mediated trait introgression (e.g., for introducing a trait into a new genotype without backcrossing to a recurrent parent or with limited backcrossing to a recurrent parent). Multiple endonucleases can be provided in expression cassettes with the appropriate promoters to allow multiple genome site editing.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Other CRISPR nucleases useful for editing genomes include Cas12b and Cas12c (see Shmakov et al. (2015) Mol. Cell, 60:385-397; Harrington et al. (2020) Molecular Cell doi:10.1016/j.molcel.2020.06.022) and CasX and CasY (see Burstein et al. (2016) Nature, doi:10.1038/nature21059; Harrington et al. (2020) Molecular Cell doi:10.1016/j.molcel.2020.06.022), or Cas 12j (Pausch et al, (2020) Science 10.1126/science.abb1400). Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application No. 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application No. 62/023,246). All of the patent publications referenced in this paragraph are incorporated herein by reference in their entirety. In certain embodiments, an RNA-guided endonuclease that leaves a blunt end following cleavage of the target site is used. Blunt-end cutting RNA-guided endonucleases include Cas9, Cas12c, and Cas 12h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e.

The methods can also use sequence-specific endonucleases or sequence-specific endonucleases and guide RNAs that cleave a single DNA strand in a dsDNA target site. Such cleavage of a single DNA strand in a dsDNA target site is also referred to herein and elsewhere as “nicking” and can be effected by various “nickases” or systems that provide for nicking. Nickases that can be used include nCas9 (Cas) comprising a D10A amino acid substitution), nCas12a (e.g., Cas12a comprising an R1226A amino acid substitution; Yamano et al., 2016), Cas12i (Yan et al. 2019), a zinc finger nickase e.g., as disclosed in Kim et al., 2012), a TALE nickase (e.g., as disclosed in Wu et al., 2014), or a combination thereof. In certain embodiments, systems that provide for nicking can comprise a Cas nuclease (e.g., Cas9 and/or Cas12a) and guide RNA molecules that have at least one base mismatch to DNA sequences in the target editing site (Fu et al., 2019). In certain embodiments, genome modifications can be introduced into the target editing site by creating single stranded breaks (i.e., “nicks”) in genomic locations separated by no more than about 10, 20, 30, 40, 50, 60, 80, 100, 150, or 200 base pairs of DNA. In certain illustrative and non-limiting embodiments, two nickases (i.e., a CAS nuclease which introduces a single stranded DNA break including nCas9, nCas12a, Cas12i, zinc finger nickases, TALE nickases, combinations thereof, and the like) or nickase systems can directed to make cuts to nearby sites separated by no more than about 10, 20, 30, 40, 50, 60, 80 or 100 base pairs of DNA. In instances where an RNA guided nickase and an RNA guide are used, the RNA guides are adjacent to PAM sequences that are sufficiently close (i.e., separated by no more than about 10, 20, 30, 40, 50, 60, 80, 100, 150, or 200 base pairs of DNA). For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least 16 or 17 nucleotides of gRNA sequence are required by Cas9 for DNA cleavage to occur: for Cpf1 at least 16 nucleotides of gRNA sequence are needed to achieve detectable DNA cleavage and at least 18 nucleotides of gRNA sequence were reported necessary for efficient DNA cleavage in vitro; see Zetsche et al. (2015) Cell, 163:759-771. In practice, guide RNA sequences are generally designed to have a length of 17-24 nucleotides (frequently 19, 20, or 21 nucleotides) and exact complementarity (i.e., perfect base-pairing) to the targeted gene or nucleic acid sequence; guide RNAs having less than 100% complementarity to the target sequence can be used (e.g., agRNA with a length of 20 nucleotides and 1-4 mismatches to the target sequence) but can increase the potential for off-target effects. The design of effective guide RNAs for use in plant genome editing is disclosed in US Patent Application Publication 2015/0082478 A1, the entire specification of which is incorporated herein by reference. More recently, efficient gene editing has been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in genome editing: see, for example, Hendel et al. (2015) Nature Biotechnol., 985-991. The design of effective gRNAs for use in plant genome editing is disclosed in US Patent Application Publication 2015/0082478 A1, the entire specification of which is incorporated herein by reference.

Genomic DNA may also be modified via base editing. Both adenine base editors (ABE) which convert A/T base pairs to G/C base pairs in genomic DNA as well as cytosine base pair editors (CBE) which effect C to T substitutions can be used in certain embodiments of the methods provided herein. In certain embodiments, useful ABE and CBE can comprise genome site specific DNA binding elements (e.g., RNA-dependent DNA binding proteins including catalytically inactive Cas9 and Cas12 proteins or Cas9 and Cas12 nickases) operably linked to adenine or cytidine deaminases and used with guide RNAs which position the protein near the nucleotide targeted for substitution. Suitable ABE and CBE disclosed in the literature (Kim, Nat Plants, 2018 March; 4(3): 148-151) can be adapted for use in the methods set forth herein. In certain embodiments, a CBE can comprise a fusion between a catalytically inactive Cas9 (dCas9) RNA dependent DNA binding protein fused to a cytidine deaminase which converts cytosine (C) to uridine (U) and selected guide RNAs, thereby effecting a C to T substitution: see Komor et al. (2016) Nature, 533:420-424. In other embodiments, C to T substitutions are effected with Cas9 nickase [Cas9n(D10A)] fused to an improved cytidine deaminase and optionally a bacteriophage Mu dsDNA (double-stranded DNA) end-binding protein Gam; see Komor et al., Sci Adv. 2017 August; 3(8):eaao4774. In other embodiments, adenine base editors (ABEs) comprising an adenine deaminase fused to catalytically inactive Cas9 (dCas9) or a Cas9 D10A nickase can be used to convert A/T base pairs to G/C base pairs in genomic DNA (Gaudelli et al., (2017) Nature 551(7681):464-471.

In certain embodiments. zinc finger nucleases or zinc finger nickases can also be used in the methods provided herein. Zinc-finger nucleases are site-specific endonucleases comprising two protein domains: a DNA-binding domain. comprising a plurality of individual zinc finger repeats that each recognize between 9 and 18 base pairs, and a DNA-cleavage domain that comprises a nuclease domain (typically Fokl). The cleavage domain dimerizes in order to cleave DNA; therefore, a pair of ZFNs are required to target non-palindromic target polynucleotides. In certain embodiments, zinc finger nuclease and zinc finger nickase design methods which have been described (Urnov et al. (2010) Nature Rev. Genet., 11:636-646; Mohanta et al. (2017) Genes vol. 8, 12: 399; Ramirez et al. Nucleic Acids Res. (2012); 40(12): 5560-5568; Liu et al. (2013) Nature Communications, 4: 2565) can be adapted for use in the methods set forth herein. The zinc finger binding domains of the zinc finger nuclease or nickase provide specificity and can be engineered to specifically recognize any desired target DNA sequence. The zinc finger DNA binding domains are derived from the DNA-binding domain of a large class of eukaryotic transcription factors called zinc finger proteins (ZFPs). The DNA-binding domain of ZFPs typically contains a tandem array of at least three zinc “fingers” each recognizing a specific triplet of DNA. A number of strategies can be used to design the binding specificity of the zinc finger binding domain. One approach, termed “modular assembly”, relies on the functional autonomy of individual zinc fingers with DNA. In this approach, a given sequence is targeted by identifying zinc fingers for each component triplet in the sequence and linking them into a multifinger peptide. Several alternative strategies for designing zinc finger DNA binding domains have also been developed. These methods are designed to accommodate the ability of zinc fingers to contact neighboring fingers as well as nucleotide bases outside their target triplet. Typically, the engineered zinc finger DNA binding domain has a novel binding specificity, compared to a naturally-occurring zinc finger protein. Engineering methods include, for example, rational design and various types of selection. Rational design includes, for example, the use of databases of triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, e.g., U.S. Pat. Nos. 6,453,242 and 6,534,261, both incorporated herein by reference in their entirety. Exemplary selection methods (e.g., phage display and yeast two-hybrid systems) can be adapted for use in the methods described herein. In addition, enhancement of binding specificity for zinc finger binding domains has been described in U.S. Pat. No. 6,794,136, incorporated herein by reference in its entirety. In addition, individual zinc finger domains may be linked together using any suitable linker sequences. Examples of linker sequences are publicly known, e.g., see U.S. Pat. Nos. 6,479,626; 6,903,185; and 7,153,949, incorporated herein by reference in their entirety. The nucleic acid cleavage domain is non-specific and is typically a restriction endonuclease, such as Fokl. This endonuclease must dimerize to cleave DNA. Thus, cleavage by Fokl as part of a ZFN requires two adjacent and independent binding events, which must occur in both the correct orientation and with appropriate spacing to permit dimer formation. The requirement for two DNA binding events enables more specific targeting of long and potentially unique recognition sites. Fokl variants with enhanced activities have been described and can be adapted for use in the methods described herein; see, e.g., Guo et al. (2010) J. Mol. Biol., 400:96-107.

Transcription activator like effectors (TALEs) are proteins secreted by certain Xanthomonas species to modulate gene expression in host plants and to facilitate the colonization by and survival of the bacterium. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site has been found. The simple relationship between amino acid sequence and DNA recognition of the TALE binding domain allows for the design of DNA binding domains of any desired specificity. TALEs can be linked to a non-specific DNA cleavage domain to prepare genome editing proteins, referred to as TAL-effector nucleases or TALENs. As in the case of ZFNs, a restriction endonuclease, such as Fokl, can be conveniently used. Methods for use of TALENs in plants have been described and can be adapted for use in the methods described herein, see Mahfouz et al. (2011) Proc. Natl. Acad. Sci. USA, 108:2623-2628; Mahfouz (2011) GM Crops, 2:99-103; and Mohanta et al. (2017) Genes vol. 8,12: 399). TALE nickases have also been described and can be adapted for use in methods described herein (Wu et al.; Biochem Biophys Res Commun. (2014); 446(1):261-6; Luo et al; Scientific Reports 6, Article number: 20657 (2016)).

Embodiments of the donor DNA template molecule having a sequence that is integrated at the site of at least one double-strand break (DSB) in a genome include double-stranded DNA, a single-stranded DNA, a single-stranded DNA/RNA hybrid, and a double-stranded DNA/RNA hybrid. In embodiments, a donor DNA template molecule that is a double-stranded (e.g., a dsDNA or dsDNA/RNA hybrid) molecule is provided directly to the plant protoplast or plant cell in the form of a double-stranded DNA or a double-stranded DNA/RNA hybrid, or as two single-stranded DNA (ssDNA) molecules that are capable of hybridizing to form dsDNA, or as a single-stranded DNA molecule and a single-stranded RNA (ssRNA) molecule that are capable of hybridizing to form a double-stranded DNA/RNA hybrid; that is to say, the double-stranded polynucleotide molecule is not provided indirectly, for example, by expression in the cell of a dsDNA encoded by a plasmid or other vector. In various non-limiting embodiments of the method, the donor DNA template molecule that is integrated (or that has a sequence that is integrated) at the site of at least one double-strand break (DSB) in a genome is double-stranded and blunt-ended; in other embodiments the donor DNA template molecule is double-stranded and has an overhang or “sticky end” consisting of unpaired nucleotides (e.g., 1, 2, 3, 4, 5, or 6 unpaired nucleotides) at one terminus or both termini. In an embodiment, the DSB in the genome has no unpaired nucleotides at the cleavage site, and the donor DNA template molecule that is integrated (or that has a sequence that is integrated) at the site of the DSB is a blunt-ended double-stranded DNA or blunt-ended double-stranded DNA/RNA hybrid molecule, or alternatively is a single-stranded DNA or a single-stranded DNA/RNA hybrid molecule. In another embodiment, the DSB in the genome has one or more unpaired nucleotides at one or both sides of the cleavage site, and the donor DNA template molecule that is integrated (or that has a sequence that is integrated) at the site of the DSB is a double-stranded DNA or double-stranded DNA/RNA hybrid molecule with an overhang or “sticky end” consisting of unpaired nucleotides at one or both termini, or alternatively is a single-stranded DNA or a single-stranded DNA/RNA hybrid molecule; in embodiments, the donor DNA template molecule DSB is a double-stranded DNA or double-stranded DNA/RNA hybrid molecule that includes an overhang at one or at both termini, wherein the overhang consists of the same number of unpaired nucleotides as the number of unpaired nucleotides created at the site of a DSB by a nuclease that cuts in an off-set fashion (e.g., where a Cas12 nuclease effects an off-set DSB with 5-nucleotide overhangs in the genomic sequence, the donor DNA template molecule that is to be integrated (or that has a sequence that is to be integrated) at the site of the DSB is double-stranded and has 5 unpaired nucleotides at one or both termini). In certain embodiments, one or both termini of the donor DNA template molecule contain no regions of sequence homology (identity or complementarity) to genomic regions flanking the DSB; that is to say, one or both termini of the donor DNA template molecule contain no regions of sequence that is sufficiently complementary to permit hybridization to genomic regions immediately adjacent to the location of the DSB. In embodiments, the donor DNA template molecule contains no homology to the locus of the DSB, that is to say, the donor DNA template molecule contains no nucleotide sequence that is sufficiently complementary to permit hybridization to genomic regions immediately adjacent to the location of the DSB. In embodiments, the donor DNA template molecule is at least partially double-stranded and includes 2-20 base-pairs, e.g., 2, 3, 4, 5, 6, 7, 8, 9, 10, 11, 12, 13, 14, 15, 16, 17. 18, 19, or 20 base-pairs; in embodiments, the donor DNA template molecule is double-stranded and blunt-ended and consists of 2-20 base-pairs, e.g., 2, 3, 4, 5, 6, 7, 8, 9, 10, 11, 12, 13, 14, 15, 16, 17, 18, 19, or 20 base-pairs; in other embodiments, the donor DNA template molecule is double-stranded and includes 2-20 base-pairs, e.g., 2, 3, 4, 5, 6, 7, 8, 9, 10, 11, 12, 13, 14, 15, 16, 17, 18, 19, or 20 base-pairs and in addition has at least one overhang or “sticky end” consisting of at least one additional, unpaired nucleotide at one or at both termini. In an embodiment, the donor DNA template molecule that is integrated (or that has a sequence that is integrated) at the site of at least one double-strand break (DSB) in a genome is a blunt-ended double-stranded DNA or a blunt-ended double-stranded DNA/RNA hybrid molecule of about 18 to about 300 base-pairs, or about 20 to about 200 base-pairs, or about 30 to about 100 base-pairs, and having at least one phosphorothioate bond between adjacent nucleotides at a 5′ end, 3′ end, or both 5′ and 3′ ends. In embodiments, the donor DNA template molecule includes single strands of at least 11, at least 18, at least 20, at least 30, at least 40, at least 60, at least 80, at least 100, at least 120, at least 140, at least 160, at least 180, at least 200, at least 240, at about 280, or at least 320 nucleotides. In embodiments, the donor DNA template molecule has a length of at least 2, at least 3, at least 4, at least 5, at least 6, at least 7, at least 8, at least 9, at least 10, or at least 11 base-pairs if double-stranded (or nucleotides if single-stranded), or between about 2 to about 320 base-pairs if double-stranded (or nucleotides if single-stranded), or between about 2 to about 500 base-pairs if double-stranded (or nucleotides if single-stranded), or between about 5 to about 500 base-pairs if double-stranded (or nucleotides if single-stranded), or between about 5 to about 300 base-pairs if double-stranded (or nucleotides if single-stranded), or between about 11 to about 300 base-pairs if double-stranded (or nucleotides if single-stranded), or about 18 to about 300 base-pairs if double-stranded (or nucleotides if single-stranded), or between about 30 to about 100 base-pairs if double-stranded (or nucleotides if single-stranded). In embodiments, the donor DNA template molecule includes chemically modified nucleotides (see, e.g., the various modifications of internucleotide linkages, bases, and sugars described in Verma and Eckstein (1998) Annu. Rev. Biochem., 67:99-134); in embodiments, the naturally occurring phosphodiester backbone of the donor DNA template molecule is partially or completely modified with phosphorothioate, phosphorodithioate, or methylphosphonate internucleotide linkage modifications, or the donor DNA template molecule includes modified nucleoside bases or modified sugars, or the donor DNA template molecule is labelled with a fluorescent moiety (e.g., fluorescein or rhodamine or a fluorescent nucleoside analogue) or other detectable label (e.g., biotin or an isotope). In another embodiment, the donor DNA template molecule contains secondary structure that provides stability or acts as an aptamer. Other related embodiments include double-stranded DNA/RNA hybrid molecules, single-stranded DNA/RNA hybrid donor molecules, and single-stranded DNA donor molecules (including single-stranded, chemically modified DNA donor molecules), which in analogous procedures are integrated (or have a sequence that is integrated) at the site of a double-strand break.

Donor DNA template molecules used in the methods provided herein include DNA molecules comprising, from 5′ to 3′, a first homology arm, a replacement DNA, and a second homology arm, wherein the homology arms containing sequences that are partially or completely homologous to genomic DNA (gDNA) sequences flanking a target site-specific endonuclease cleavage site in the gDNA. In certain embodiments, the replacement DNA can comprise an insertion, deletion, or substitution of 1 or more DNA base pairs relative to the target gDNA. In an embodiment, the donor DNA template molecule is double-stranded and perfectly base-paired through all or most of its length, with the possible exception of any unpaired nucleotides at either terminus or both termini. In another embodiment, the donor DNA template molecule is double-stranded and includes one or more non-terminal mismatches or non-terminal unpaired nucleotides within the otherwise double-stranded duplex. In an embodiment, the donor DNA template molecule that is integrated at the site of at least one double-strand break (DSB) includes between 2-20 nucleotides in one (if single-stranded) or in both strands (if double-stranded), e.g., 2, 3, 4, 5, 6, 7, 8, 9, 10, 11, 12, 13, 14, 15, 16, 17, 18, 19, or 20 nucleotides on one or on both strands, each of which can be base-paired to a nucleotide on the opposite strand (in the case of a perfectly base-paired double-stranded polynucleotide molecule). Such donor DNA templates can be integrated in genomic DNA containing blunt and/or staggered double stranded DNA breaks by homology-directed repair (HDR). In certain embodiments, a donor DNA template homology arm can be about 20, 50, 100, 200, 400, or 600 to about 800, or 1000 base pairs in length. In certain embodiments, a donor DNA template molecule can be delivered to a plant cell) in a circular (e.g., a plasmid or a viral vector including a geminivirus vector) or a linear DNA molecule. In certain embodiments, a circular or linear DNA molecule that is used can comprise a modified donor DNA template molecule comprising, from 5′ to 3′, a first copy of the target sequence-specific endonuclease cleavage site sequence, the first homology arm, the replacement DNA, the second homology arm, and a second copy of the target sequence-specific endonuclease cleavage site sequence. Without seeking to be limited by theory, such modified donor DNA template molecules can be cleaved by the same sequence-specific endonuclease that is used to cleave the target site gDNA of the eukaryotic cell to release a donor DNA template molecule that can participate in HDR-mediated genome modification of the target editing site in the plant cell genome. In certain embodiments, the donor DNA template can comprise a linear DNA molecule comprising, from 5° to 3′, a cleaved target sequence-specific endonuclease cleavage site sequence, the first homology arm, the replacement DNA, the second homology arm, and a cleaved target sequence-specific endonuclease cleavage site sequence. In certain embodiments, the cleaved target sequence-specific endonuclease sequence can comprise a blunt DNA end or a blunt DNA end that can optionally comprise a 5′ phosphate group. In certain embodiments, the cleaved target sequence-specific endonuclease sequence comprises a DNA end having a single-stranded 5′ or 3′ DNA overhang. Such cleaved target sequence-specific endonuclease cleavage site sequences can be produced by either cleaving an intact target sequence-specific endonuclease cleavage site sequence or by synthesizing a copy of the cleaved target sequence-specific endonuclease cleavage site sequence. Donor DNA templates can be synthesized either chemically or enzymatically (e.g., in a polymerase chain reaction (PCR)).

Various treatments are useful in delivery of gene editing molecules and/or other molecules to a plant cell. In certain embodiments, one or more treatments is employed to deliver the gene editing or other molecules (e.g., comprising a polynucleotide, polypeptide or combination thereof) into a eukaryotic or plant cell, e.g., through barriers such as a cell wall, a plasma membrane, a nuclear envelope, and/or other lipid bilayer. In certain embodiments, a polynucleotide-, polypeptide-, or RNP-containing composition comprising the molecules are delivered directly, for example by direct contact of the composition with a plant cell. Aforementioned compositions can be provided in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof can be applied directly to a plant, plant part, plant cell, or plant explant (e.g., through abrasion or puncture or otherwise disruption of the cell wall or cell membrane, by spraying or dipping or soaking or otherwise directly contacting, by microinjection). For example, a plant cell or plant protoplast is soaked in a liquid genome editing molecule-containing composition, whereby the agent is delivered to the plant cell. In certain embodiments, the agent-containing composition is delivered using negative or positive pressure, for example, using vacuum infiltration or application of hydrodynamic or fluid pressure. In certain embodiments, the agent-containing composition is introduced into a plant cell or plant protoplast, e.g., by microinjection or by disruption or deformation of the cell wall or cell membrane, for example by physical treatments such as by application of negative or positive pressure, shear forces, or treatment with a chemical or physical delivery agent such as surfactants, liposomes, or nanoparticles; see, e.g., delivery of materials to cells employing microfluidic flow through a cell-deforming constriction as described in US Published Patent Application 2014/0287509, incorporated by reference in its entirety herein. Other techniques useful for delivering the agent-containing composition to a eukaryotic cell, plant cell or plant protoplast include: ultrasound or sonication: vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation. In certain embodiments, the agent-containing composition is provided by bacterially mediated (e.g., Agrobacterium sp., Rhizobium sp., Sinorhizobium sp., Mesorhizobium sp., Bradyrhizobium sp., Azobacter sp., Phyllobacterium sp.) transfection of the plant cell or plant protoplast with a polynucleotide encoding the genome editing molecules (e.g., RNA dependent DNA endonuclease, RNA dependent DNA binding protein, RNA dependent nickase, ABE, or CBE, and/or guide RNA); see, e.g., Broothaerts et al. (2005) Nature, 433:629-633). Any of these techniques or a combination thereof are alternatively employed on the plant explant, plant part or tissue or intact plant (or seed) from which a plant cell is optionally subsequently obtained or isolated; in certain embodiments, the agent-containing composition is delivered in a separate step after the plant cell has been isolated.

In some embodiments, one or more polynucleotides or vectors driving expression of one or more genome editing molecules or trait-conferring genes (e.g.; herbicide tolerance, insect resistance, and/or male sterility) are introduced into a plant cell. In certain embodiments, a polynucleotide vector comprises a regulatory element such as a promoter operably linked to one or more polynucleotides encoding genome editing molecules and/or trait-conferring genes. In such embodiments, expression of these polynucleotides can be controlled by selection of the appropriate promoter, particularly promoters functional in a eukaryotic cell (e.g., plant cell); useful promoters include constitutive, conditional, inducible, and temporally or spatially specific promoters (e.g., a tissue specific promoter, a developmentally regulated promoter, or a cell cycle regulated promoter). Developmentally regulated promoters that can be used in plant cells include Phospholipid Transfer Protein (PLTP), fructose-1,6-bisphosphatase protein, NAD(P)-binding Rossmann-Fold protein, adipocyte plasma membrane-associated protein-like protein, Rieske [2Fe-2S] iron-sulfur domain protein, chlororespiratory reduction 6 protein, D-glycerate 3-kinase, chloroplastic-like protein, chlorophyll a-b binding protein 7, chloroplastic-like protein, ultraviolet-B-repressible protein, Soul heme-binding family protein, Photosystem I reaction center subunit psi-N protein, and short-chain dehydrogenase/reductase protein that are disclosed in US Patent Application Publication No. 20170121722, which is incorporated herein by reference in its entirety and specifically with respect to such disclosure. In certain embodiments, the promoter is operably linked to nucleotide sequences encoding multiple guide RNAs, wherein the sequences encoding guide RNAs are separated by a cleavage site such as a nucleotide sequence encoding a microRNA recognition/cleavage site or a self-cleaving ribozyme (see. e.g., Ferré-D'Amaré and Scott (2014) Cold Spring Harbor Perspectives Biol., 2:a003574). In certain embodiments, the promoter is an RNA polymerase III promoter operably linked to a nucleotide sequence encoding one or more guide RNAs. In certain embodiments, the RNA polymerase III promoter is a plant U6 spliceosomal RNA promoter, which can be native to the genome of the plant cell or from a different species, e.g., a U6 promoter from maize, tomato, or soybean such as those disclosed US Patent Application Publication 2017/0166912, or a homologue thereof; in an example, such a promoter is operably linked to DNA sequence encoding a first RNA molecule including a Cas12a gRNA followed by an operably linked and suitable 3′ element such as a U6 poly-T terminator. In another embodiment, the RNA polymerase III promoter is a plant U3, 7SL (signal recognition particle RNA), U2, or U5 promoter, or chimerics thereof, e.g., as described in U.S. Patent Application Publication 20170166912. In certain embodiments, the promoter operably linked to one or more polynucleotides is a constitutive promoter that drives gene expression in eukaryotic cells (e.g., plant cells). In certain embodiments, the promoter drives gene expression in the nucleus or in an organelle such as a chloroplast or mitochondrion. Examples of constitutive promoters for use in plants include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and the nopaline synthase (NOS) and octopine synthase (OCS) promoters from Agrobacterium tumefaciens. In certain embodiments, the promoter operably linked to one or more polynucleotides encoding elements of a genome-editing system is a promoter from figwort mosaic virus (FMV), a RUBISCO promoter, or a pyruvate phosphate dikinase (PPDK) promoter, which is active in photosynthetic tissues. Other contemplated promoters include cell-specific or tissue-specific or developmentally regulated promoters, for example, a promoter that limits the expression of the nucleic acid targeting system to germline or reproductive cells (e.g., promoters of genes encoding DNA ligases, recombinases, replicases, or other genes specifically expressed in germline or reproductive cells). In certain embodiments, the genome alteration is limited only to those cells from which DNA is inherited in subsequent generations, which is advantageous where it is desirable that expression of the genome-editing system be limited in order to avoid genotoxicity or other unwanted effects. All of the patent publications referenced in this paragraph are incorporated herein by reference in their entirety.

Expression vectors or polynucleotides provided herein may contain a DNA segment near the 3′ end of an expression cassette that acts as a signal to terminate transcription and directs polyadenylation of the resultant mRNA and may also support promoter activity. Such a 3′ element is commonly referred to as a “3′-untranslated region” or “3′-UTR” or “terminator” or a “polyadenylation signal.” In some cases, plant gene-based 3′ elements (or terminators) consist of both the 3′-UTR and downstream non-transcribed sequence (Nuccio et al., 2015). Useful 3′ elements include: Agrobacterium tumefaciens nos 3′, tml 3′, tmr 3′, tms 3′, ocs 3′, and tr7 3′ elements disclosed in U.S. Pat. No. 6,090,627, incorporated herein by reference, and 3′ elements from plant genes such as the heat shock protein 17, ubiquitin, and fructose-1,6-biphosphatase genes from wheat (Triticum aestivum), and the glutelin, lactate dehydrogenase, and beta-tubulin genes from rice (Oryza sativa), disclosed in US Patent Application Publication 2002/0192813 A1. All of the patent publications referenced in this paragraph are incorporated herein by reference in their entireties.

In certain embodiments, the plant cells can comprise haploid, diploid, or polyploid plant cells or plant protoplasts, for example, those obtained from a haploid, diploid, or polyploid plant, plant part or tissue, or callus. In certain embodiments, plant cells in culture (or the regenerated plant, progeny seed, and progeny plant) are haploid or can be induced to become haploid; techniques for making and using haploid plants and plant cells are known in the art, see, e.g., methods for generating haploids in Arabidopsis thaliana by crossing of a wild-type strain to a haploid-inducing strain that expresses altered forms of the centromere-specific histone CENH3, as described by Maruthachalam and Chan in “How to make haploid Arabidopsis thaliana”. protocol available at www[dot]openwetware[dot]org/images/d/d3/Haploid_Arabidopsis_protocol[dot]pdf; (Ravi et al. (2014) Nature Communications, 5:5334, doi: 10.1038/ncomms6334). Haploids can also be obtained in a wide variety of monocot plants (e.g., maize, wheat, rice, sorghum, barley) or dicot plants (e.g., soy bean, Brassica sp. including canola, cotton, tomato) by crossing a plant comprising a mutated CENH3 gene with a wildtype diploid plant to generate haploid progeny as disclosed in U.S. Pat. No. 9,215,849, which is incorporated herein by reference in its entirety. Haploid-inducing maize lines that can be used to obtain haploid maize plants and/or cells include Stock 6, MHI (Moldovian Haploid Inducer), indeterminate gametophyte (ig) mutation, KEMS, RWK, ZEM, ZMS, KMS, and well as transgenic haploid inducer lines disclosed in U.S. Pat. No. 9,677,082, which is incorporated herein by reference in its entirety. Examples of haploid cells include but are not limited to plant cells obtained from haploid plants and plant cells obtained from reproductive tissues, e.g., from flowers, developing flowers or flower buds, ovaries, ovules, megaspores, anthers, pollen, megagametophyte, and microspores. In certain embodiments where the plant cell or plant protoplast is haploid, the genetic complement can be doubled by chromosome doubling (e.g., by spontaneous chromosomal doubling by meiotic non-reduction, or by using a chromosome doubling agent such as colchicine, oryzalin, trifluralin, pronamide, nitrous oxide gas, anti-microtubule herbicides, anti-microtubule agents, and mitotic inhibitors) in the plant cell or plant protoplast to produce a doubled haploid plant cell or plant protoplast wherein the complement of genes or alleles is homozygous; yet other embodiments include regeneration of a doubled haploid plant from the doubled haploid plant cell or plant protoplast. Another embodiment is related to a hybrid plant having at least one parent plant that is a doubled haploid plant provided by this approach. Production of doubled haploid plants provides homozygosity in one generation, instead of requiring several generations of self-crossing to obtain homozygous plants. The use of doubled haploids is advantageous in any situation where there is a desire to establish genetic purity (i.e. homozygosity) in the least possible time. Doubled haploid production can be particularly advantageous in slow-growing plants or for producing hybrid plants that are offspring of at least one doubled-haploid plant.

In certain embodiments, the plant cells used in the methods provided herein can include non-dividing cells. Such non-dividing cells can include plant cell protoplasts, plant cells subjected to one or more of a genetic and/or pharmaceutically-induced cell-cycle blockage, and the like.

In certain embodiments, the plant cells in used in the methods provided herein can include dividing cells. Dividing cells can include those cells found in various plant tissues including leaves, meristems, and embryos. These tissues include but are not limited to dividing cells from young maize leaf, meristems and scutellar tissue from about 8 or 10 to about 12 or 14 days after pollination (DAP) embryos. The isolation of maize embryos has been described in several publications (Brettschneider, Becker, and Lörz. 1997; Leduc et al. 1996; Frame et al. 2011; K. Wang and Frame 2009). In certain embodiments, basal leaf tissues (e.g., leaf tissues located about 0 to 3 cm from the ligule of a maize plant; Kirienko, Luo, and Sylvester 2012) are targeted for HDR-mediated gene editing. Methods for obtaining regenerable plant structures and regenerating plants from the HDR-mediated gene editing of plant cells provided herein can be adapted from methods disclosed in US Patent Application Publication No. 20170121722, which is incorporated herein by reference in its entirety and specifically with respect to such disclosure. In certain embodiments, single plant cells subjected to the HDR-mediated gene editing will give rise to single regenerable plant structures. In certain embodiments, the single regenerable plant cell structure can form from a single cell on, or within, an explant that has been subjected to the HDR-mediated gene editing.

In some embodiments, methods provided herein can include the additional step of growing or regenerating a plant from a plant cell that had been subjected to the improved HDR-mediated gene editing or from a regenerable plant structure obtained from that plant cell. In certain embodiments, the plant can further comprise an inserted transgene, a target gene edit, or genome edit as provided by the methods and compositions disclosed herein. In certain embodiments, callus is produced from the plant cell, and plantlets and plants produced from such callus. In other embodiments, whole seedlings or plants are grown directly from the plant cell without a callus stage. Thus, additional related aspects are directed to whole seedlings and plants grown or regenerated from the plant cell or plant protoplast having a target gene edit or genome edit, as well as the seeds of such plants. In certain embodiments wherein the plant cell or plant protoplast is subjected to genetic modification (for example, genome editing by means of, e.g., an RdDe), the grown or regenerated plant exhibits a phenotype associated with the genetic modification. In certain embodiments, the grown or regenerated plant includes in its genome two or more genetic or epigenetic modifications that in combination provide at least one phenotype of interest. In certain embodiments, a heterogeneous population of plant cells having a target gene edit or genome edit, at least some of which include at least one genetic or epigenetic modification, is provided by the method; related aspects include a plant having a phenotype of interest associated with the genetic or epigenetic modification, provided by either regeneration of a plant having the phenotype of interest from a plant cell or plant protoplast selected from the heterogeneous population of plant cells having a target gene or genome edit, or by selection of a plant having the phenotype of interest from a heterogeneous population of plants grown or regenerated from the population of plant cells having a target gene edit or genome edit. Examples of phenotypes of interest include herbicide resistance, improved tolerance of abiotic stress (e.g., tolerance of temperature extremes, drought, or salt) or biotic stress (e.g., resistance to nematode, bacterial, or fungal pathogens), improved utilization of nutrients or water, modified lipid, carbohydrate, or protein composition, improved flavor or appearance, improved storage characteristics (e.g., resistance to bruising, browning, or softening), increased yield, altered morphology (e.g., floral architecture or color, plant height, branching, root structure). In an embodiment, a heterogeneous population of plant cells having a target gene edit or genome edit (or seedlings or plants grown or regenerated therefrom) is exposed to conditions permitting expression of the phenotype of interest; e.g., selection for herbicide resistance can include exposing the population of plant cells having a target gene edit or genome edit (or seedlings or plants grown or regenerated therefrom) to an amount of herbicide or other substance that inhibits growth or is toxic, allowing identification and selection of those resistant plant cells (or seedlings or plants) that survive treatment. Methods for obtaining regenerable plant structures and regenerating plants from plant cells or regenerable plant structures can be adapted from published procedures (Roest and Gilissen, Acta Bot. Neerl., 1989, 38(1), 1-23; Bhaskaran and Smith, Crop Sci. 30(6):1328-1337; Ikeuchi et al., Development, 2016, 143: 1442-1451). Methods for obtaining regenerable plant structures and regenerating plants from plant cells or regenerable plant structures can also be adapted from US Patent Application Publication No. 20170121722, which is incorporated herein by reference in its entirety and specifically with respect to such disclosure. Also provided are heterogeneous or homogeneous populations of such plants or parts thereof (e.g., seeds), succeeding generations or seeds of such plants grown or regenerated from the plant cells or plant protoplasts, having a target gene edit or genome edit. Additional related aspects include a hybrid plant provided by crossing a first plant grown or regenerated from a plant cell or plant protoplast having a target gene edit or genome edit and having at least one genetic or epigenetic modification, with a second plant, wherein the hybrid plant contains the genetic or epigenetic modification; also contemplated is seed produced by the hybrid plant. Also envisioned as related aspects are progeny seed and progeny plants, including hybrid seed and hybrid plants, having the regenerated plant as a parent or ancestor. The plant cells and derivative plants and seeds disclosed herein can be used for various purposes useful to the consumer or grower. In other embodiments, processed products are made from the plant or its seeds, including: (a) corn, soy, cotton, or canola seed meal (defatted or non-defatted); (b) extracted proteins, oils, sugars, and starches; (c) fermentation products; (d) animal feed or human food products (e.g., feed and food comprising corn, soy, cotton, or canola seed meal (defatted or non-defatted) and other ingredients (e.g., other cereal grains, other seed meal, other protein meal, other oil, other starch, other sugar, a binder, a preservative, a humectant, a vitamin, and/or mineral; (e) a pharmaceutical; (f) raw or processed biomass (e.g., cellulosic and/or lignocellulosic material); and (g) various industrial products.

### Embodiments

Various embodiments of the plants, genomes, methods, biological samples, and other compositions described herein are set forth in the following sets of numbered embodiments.

### Embodiment List One

1. An edited transgenic plant genome comprising a modification of an original transgenic locus, wherein the modification comprises a deletion of a segment of the original transgenic locus, the segment comprising, consisting essentially of, or consisting of: a synthetic cloning site sequence; a duplication of a transgene sequence; a fragment of a transgene sequence; Agrobacterium right and/or left border sequences;
   and/or a segment of DNA that is non-essential for a primary functionality of the transgenic locus and wherein optionally the primary functionality is herbicide tolerance, insect resistance, biofuel use, or male sterility.
2. The edited transgenic plant genome of embodiment 1, wherein the modification of the original transgenic locus further comprises deletion of a selectable marker gene and optionally wherein the primary functionality is insect resistance, biofuel use, or male sterility.
3. The edited transgenic plant genome of embodiment 1 or 2, wherein the modification comprises: (a) a deletion of the segment comprising, consisting essentially of, or consisting of the synthetic cloning site, the duplication of a transgene sequence, the fragment of a transgene sequence, and/or Agrobacterium right and/or left border sequences: or (b) a deletion of a selectable marker gene.
4. The edited transgenic plant genome of embodiment 3, wherein the selectable marker gene confers resistance to an antibiotic, tolerance to an herbicide, or an ability to grow on a specific carbon source, wherein the specific carbon source is optionally mannose and optionally wherein the primary functionality is insect resistance, biofuel use, or male sterility.
5. The edited transgenic plant genome of embodiment 4, wherein the selectable marker gene comprises a DNA encoding a phosphinothricin acetyl transferase (PAT), a glyphosate tolerant 5-enol-pyruvylshikimate-3-phosphate synthase (EPSPS), and a glyphosate oxidase (GOX), neomycin phosphotransferase (npt), a hygromycin phosphotransferase, an aminoglycoside adenyl transferase, or a phosphomannose isomerase (pmi).
6. The edited transgenic plant genome of any one of embodiments 1 to 5, wherein the deleted segment of the original transgenic locus is at least two, three, four, five, six, seven, eight, nine, 10, 15, 20, 25, 30, 35, 40, 45, 50, 60, 70, 80, 90, 100, 125, 150, 175, 200, 250, 300, 400, 500, 600, 700, 800, 900, or 1000 base pairs of DNA in length.
7. The edited transgenic plant genome of any one of clams 1 to 5, wherein the deleted segment of the original transgenic locus is between any of two, three, four, five, six, seven, eight, nine, 10, 15, 20, 25, 30, 35, 40, 45, or 50 to 60, 70, 80, 90, 100, 125, 150, 175, 200, 250, 300, 400, 500, 600, 700, 800, or 900 base pairs of DNA in length and any of three, four, five, six, seven, eight, nine, 10, 15, 20, 25, 30, 35, 40, 45, or 50 to 60, 70, 80, 90, 100, 125, 150, 175, 200, 250, 300, 400, 500, 600, 700, 800, 900, or 1,000 base pairs of DNA in length: optionally, wherein the segment of the original transgenic locus that is deleted is between 10 and 500 base pairs of DNA in length.
8. The edited transgenic plant genome of any one of embodiments 1 to 7, wherein the deleted segment is flanked by operably linked protospacer adjacent motif (PAM) sites in the original transgenic locus and/or wherein the deleted segment encompasses an operably linked PAM site in the original transgenic locus.
9. The edited transgenic plant genome of any one of embodiments 1 to 8 comprising PAM sites flanking the deletion site in the modified transgenic locus.
10. The edited transgenic plant genome of any one of embodiments 1 to 9, wherein the PAM sites are recognized by an RNA dependent DNA endonuclease (RdDe); optionally wherein the RdDe is a class 2 type II or class 2 type V RdDe.
11. The edited transgenic plant genome of any one of embodiments 1 to 10, wherein the modification comprises two or more separate deletions.
12. The edited transgenic plant genome of any one of embodiments 1 to 11, wherein there is a modification in two or more original transgenic plant loci.
13. The edited transgenic plant genome of any one of embodiments 1 to 12, wherein the deleted segment of the original transgenic locus is replaced in the modified transgenic locus by an introduced DNA sequence.
14. The edited transgenic plant genome of any one of embodiments 1 to 13, wherein the modification comprises a modification of a Bt11, DAS-59122-7, DP-4114, GA21, MON810, MON87411, MON87427, MON88017, MON89034, MIR162, MIR604, NK603, SYN-E3272-5, 5307, DAS-40278, DP-32138, DP-33121, HCEM485, LY038, MON863, MON87403, MON87403, MON87419, MON87460, MZHG0JG, MZIR098, VCO-Ø1981-5, 98140, or TC1507 original transgenic locus in a transgenic corn plant genome.
15. The edited transgenic plant genome of any one of embodiments 1 to 13, wherein the modification comprises a modification of an A5547-127, DAS44406-6, DAS68416-4, DAS81419-2, GTS 40-3-2, MON87701, MON87708, MON89788, MST-FGØ72-3, or SYHT0H2 original transgenic locus in a transgenic soy bean plant genome.
16. The edited transgenic plant genome of any one of embodiments 1 to 13, wherein the modification comprises a modification of a DAS-21023-5, DAS-24236-5, COT102, LLcotton25, MON15985, MON88701, or MON88913 original transgenic locus in a transgenic cotton plant genome.
17. The edited transgenic plant genome of any one of embodiments 1 to 13, wherein the modification comprises a modification of an GT73, HCN28, MON88302, or MS8 original transgenic locus in a transgenic canola plant genome.
18. A transgenic plant cell comprising the edited transgenic plant genome of any one of embodiments 1 to 17.
19. A transgenic plant comprising the edited transgenic plant genome of any one of embodiments 1 to 17.
20. A transgenic plant part comprising the edited transgenic plant genome of any one of embodiments 1 to 17.
21. The transgenic plant part of embodiment 20, wherein the plant part is a seed, leaf, tuber, stem, root, or boll.
22. A processed transgenic plant product obtained from the transgenic plant part of embodiment 20 or 21, wherein the processed plant product contains a polynucleotide comprising a portion of the modified transgenic locus comprising the deletion site of the segment of the original transgenic locus.
23. A biological sample obtained from the transgenic plant cell of embodiment 18, the transgenic plant of embodiment 19, or the transgenic plant part of embodiment 20 or 21, wherein the biological sample contains a polynucleotide comprising a portion of the modified transgenic locus comprising the deletion site of the segment of the original transgenic locus.
24. A method of editing a transgenic plant genome to obtain a plant cell, plant part, or plant containing a modification of an original transgenic locus, the method comprising: (a) contacting a transgenic plant genome with one or more gene editing molecules which introduce one or more single or double-stranded breaks providing for excision of a segment of the original transgenic locus comprising, consisting essentially of, or consisting of: a synthetic cloning site sequence; a duplication of a transgene sequence; a fragment of a transgene sequence; Agrobacterium right and/or left border sequences; and/or a segment of DNA that is non-essential for a primary functionality of the transgenic locus and wherein optionally the primary functionality is herbicide tolerance, insect resistance, biofuel use, or male sterility, and (b) selecting a plant cell, plant part, or plant containing a modified transgenic locus, wherein a segment comprising or consisting of the synthetic cloning site sequence, duplication of a transgene sequence, fragment of a transgene sequence, Agrobacterium right and/or left border sequences, and/or segment of DNA that is non-essential for expression of any transgene in the locus has been deleted, thereby obtaining a plant cell, plant part, or plant containing a modified transgenic locus: optionally, wherein the transgenic plant genome is contacted in step (a) by introducing one or more compositions comprising or encoding the gene editing molecules into a plant cell comprising the transgenic plant genome.
25. The method of embodiment 24, wherein the method further comprises excising DNA comprising a selectable marker gene from the original transgenic locus.
26. The method of embodiment 24 or 25, wherein the segment comprising the synthetic cloning site sequence, the duplication of a transgene sequence, the fragment of a transgene sequence, and/or Agrobacterium right/and or left border sequences further comprises a selectable marker gene.
27. The method of any one of embodiments 24 to 26, further comprising contacting the genome with one or more gene editing molecules which introduce one or more single or double-stranded breaks providing for excision of a selectable marker gene, wherein the segment comprising, consisting essentially of, or consisting of the synthetic cloning site sequence, the duplication of a transgene sequence, the fragment of a transgene sequence, and/or Agrobacterium right and/or left border sequences is deleted: optionally in step (b) selecting a plant cell, plant part, or plant containing a modified transgenic locus, wherein a selectable marker gene and the segment comprising or consisting of the synthetic cloning site sequence, duplication of a transgene sequence, fragment of a transgene sequence, Agrobacterium right and/or left border sequences, and/or segment of DNA that is non-essential for expression of any transgene in the locus have been deleted.
28. The method of embodiment 26 or 27, wherein the selectable marker gene confers resistance to an antibiotic, tolerance to an herbicide, or an ability to grow on a specific carbon source, wherein the specific carbon source is optionally mannose.
29. The method of embodiment 28, wherein the selectable marker gene comprises a DNA encoding a phosphinothricin acetyl transferase (PAT), a glyphosate tolerant 5-enol-pyruvylshikimate-3-phosphate synthase (EPSPS), and a glyphosate oxidase (GOX), neomycin phosphotransferase (npt), a hygromycin phosphotransferase, an aminoglycoside adenyl transferase, or a phosphomannose isomerase (pmi).
30. The method of any one of embodiments 24 to 29, wherein the transgenic plant cell is in tissue culture, in a callus culture, a plant part, or in a whole plant.
31. The method of any one of embodiments 24 to 30, wherein the transgenic plant cell is a haploid plant cell, optionally, wherein the plant is a haploid plant.
32. The method of any one of embodiments 24 to 31, wherein the deleted segment of the original transgenic locus is at least two, three, four, five, six, seven, eight, nine, 10, 15, 20, 25, 30, 35, 40, 45, 50, 60, 70, 80, 90, 100, 125, 150, 175, 200, 250, 300, 400, 500, 600, 700, 800, 900, or 1000 base pairs of DNA in length.
33. The method of any one of embodiments 24 to 31, wherein the deleted segment of the original transgenic locus is between any of two, three, four, five, six, seven, eight, nine, 10, 15, 20, 25, 30, 35, 40, 45, or 50 to 60, 70, 80, 90, 100, 125, 150, 175, 200, 250, 300, 400, 500, 600, 700, 800, or 900 base pairs of DNA in length and any of three, four, five, six, seven, eight, nine, 10, 15, 20, 25, 30, 35, 40, 45, or 50 to 60, 70, 80, 90, 100, 125, 150, 175, 200, 250, 300, 400, 500, 600, 700, 800, 900, or 1,000 base pairs of DNA in length, optionally, wherein the deleted segment of the original transgenic locus is between 10 and 500 base pairs of DNA in length.
34. The method of any one of embodiments 24 to 33, wherein the one or more gene editing molecules is selected from the group consisting of RNA dependent DNA endonucleases (RdDe) and/or guide RNAs, RNA dependent nickases and/or guide RNAs, Zinc Finger nucleases or nickases, and TALE nucleases or nickases.
35. The method of any one of embodiments 24 to 34, wherein the deleted segment is flanked by operably linked protospacer adjacent motif (PAM) sites in the original transgenic locus and/or wherein the deleted segment encompasses an operably linked PAM site in the original transgenic locus.
36. The method of any one of embodiments 24 to 35, wherein the modified transgenic locus comprises PAM sites flanking the deletion site.
37. The method of any one of embodiments 24 to 36, wherein the PAM sites are recognized by an RNA dependent DNA endonuclease (RdDe); optionally wherein the RdDe is a class 2 type II or class 2 type V RdDe.
38. The method of any one of embodiments 24 to 37, wherein the modification comprises two or more deletions.
39. The method of any one of embodiments 24 to 38, wherein two or more original transgenic plant loci are modified.
40. The method of any one of embodiments 24 to 39, wherein the deleted segment of the original transgenic locus is replaced in the modified transgenic locus by an introduced DNA sequence.
41. The method of embodiment 40, wherein the gene editing molecules include a donor DNA template containing the introduced DNA sequence,
optionally, wherein the transgenic plant cell, transgenic plant part, or transgenic plant is selected for integration of the introduced DNA sequence at the deletion site of the deleted segment of the original transgenic locus.
42. The method of any one of embodiments 24 to 41, wherein the modification comprises a modification of a Bt11, DAS-59122-7, DP-4114, GA21, MON810, MON87411, MON87427, MON88017, MON89034, MIR162, MIR604, NK603, SYN-E3272-5, 5307, DAS-40278, DP-32138, DP-33121, HCEM485, LY038, MON863, MON87403, MON87403, MON87419, MON87460, MZHG0JG, MZIR098, VCO-Ø1981-5, 98140, or TC1507 original transgenic locus in a transgenic corn plant genome.
43. The method of any one of embodiments 24 to 41, wherein the modification comprises a modification of an A5547-127, DAS44406-6, DAS68416-4, DAS81419-2, GTS 40-3-2, MON87701, MON87708, MON89788, MST-FGØ72-3, or SYHT0H2 original transgenic locus in a transgenic soy bean plant genome.
44. The method of any one of embodiments 24 to 41, wherein the modification comprises a modification of a DAS-21023-5, DAS-24236-5, COT102, LLcotton25, MON15985, MON88701, or MON88913 original transgenic locus in a transgenic cotton plant genome.
45. The method of any one of embodiments 24 to 41, wherein the modification comprises a modification of an GT73, HCN28, MON88302, or MS8 original transgenic locus in a transgenic canola plant genome.
46. A method of obtaining a plant breeding line comprising: (a) crossing a transgenic plant comprising an edited transgenic plant genome of any one of embodiments 1 to 17 with a second plant; and (b) selecting from the cross a progeny plant comprising the modified transgenic locus of the edited transgenic genome, thereby obtaining a plant breeding line.
47. The method of embodiment 46, wherein the second plant also comprises an edited transgenic plant genome of any one of embodiments 1 to 17; and (b) selecting from the cross a progeny plant comprising the modified transgenic locus of the first plant and the modified transgenic locus of the second plant, thereby obtaining a plant breeding line.
48. The method of embodiment 46 or 47, wherein the first plant or second plant further comprises an additional third modified transgenic locus; and (b) selecting from the cross a progeny plant comprising the modified transgenic locus of the first plant, the modified transgenic locus of the second plant, and the third modified transgenic locus, thereby obtaining a plant breeding line.

### EXAMPLES

The following Examples are provided for purposes of illustration only and are not intended to be limiting.

Example 1. Excision of Selectable Marker Genes from Transgenic Loci

Transgenic plant genomes containing one or more of the following transgenic loci (events) with selectable marker genes are contacted with a class 2 type II or class 2 type V RdDe and guide RNAs which recognize the indicated target DNA sites (guide RNA coding plus PAM site) in the DNA segments that flank the selectable marker gene. Plant cells, callus, parts, or whole plants comprising a deletion of the selectable marker gene from the transgenic loci in the transgenic plant genome are selected.

TABLE 5
Pre-existing genomic DNA target and PAM sites in DNA flanking
selectable marker genes of different events (transgenic loci)

| | | Selectable Marker Gene | |
|---|---|---|---|
| | | Flanking DNA 1 | |
| | | polynucleotide target DNA | Selectable Marker Gene Flanking |
| | Selectable | (Guide RNA coding | DNA 2 polynucleotide target DNA |
| | Marker | sequence + PAM for Class 2 | (Guide RNA coding sequence + |
| | Gene | type II) | PAM for Class 2 type II) |
| CORN | | | |
| EVENT | | | |
| NAME | | | |
| DAS- | PAT | GAAGAAAATCTTCGTCAA | TCCAGGGCGAGCTCGGTACCC |
| 59122-7 | | CATGG (SEQ ID NO: 35) | GG (SEQ ID NO: 36) |
| DP-4114 | PAT | GGCCGCGGACCGAATTCC | ATCGTGGCCTCTTGCTCTTCAG |
| | | CATGG (SEQ ID NO: 37) | G (SEQ ID NO: 38) |
| MON87411 | CP4 | CGAGGCAAGCTTGTCGAA | AAACACTGATAGTTTAAACGC |
| | EPSPS | AATGG (SEQ ID NO: 39) | GG (SEQ ID NO: 40) |
| MIR162 | PMI | TGCACTGCAGGCATGCAA | TGTACTGAATTGTCTAGACCC |
| | | GCTGG (SEQ ID NO: 41) | GG (SEQ ID NO: 42) |
| NK603 | CP4 | CGCGTTAACAAGCTTACT | AGATCGGGGATAGCTTCTGCA |
| | EPSPS | CGAGG (SEQ ID NO: 43) | GG (SEQ ID NO: 44) |
| SYN- | PMI | TGCACTGCAGGCATGCAA | GGCACCGGTAAATTTCCTGCA |
| E3272-5 | | GCTGG (SEQ ID NO: 45) | GG (SEQ ID NO: 46) |
| 5307 | PMI | TGCACTGCAGGCATGCAA | ACTAGATCTGCTAGCCCTGCA |
| | | GCTGG(SEQ ID NO: 47) | GG(SEQ ID NO: 48) |
| SOYBEAN | | | |
| EVENT | | | |
| NAME | | | |
| DAS68416- | | CGCGGCCGCTTAATTAAG | CGGGTTTCTAGTCACCGGTTA |
| 4 | | GCCGG (SEQ ID NO: 49) | GG (SEQ ID NO: 50) |
| MON89788 | EPSPS | TTTGGACTGAGAATTAGC | TTTCTCATCTAAGCCCCCATTT |
| | | TTCCACTCG ((SEQ ID NO: | GGACG (SEQ ID NO: 52); |
| | | 51; CLASS 2 TYPE V | CLASS 2 TYPE V PAM + GRNA |
| | | PAM + GRNA CODING) | CODING) |
| MON89788 | EPSPS | TTCTGCAGGTCCTGCTCGA | CGGCCGCTTCGAGTGGCTGCA |
| | | GTGG (SEQ ID NO: 53; | GG (SEQ ID NO: 54; CLASS 2 |
| | | CLASS 2 TYPE 2 GRNA | TYPE II GRNA CODING + PAM) |
| | | CODING + PAM) | |
| COTTON | | | |
| EVENT | | | |
| NAME | | | |
| COT102 | aph4 (hpt) | GTACGCCATGCTGGCCGC | CTTGGCTCCAAATCCGGTACC |
| | | CCGGG (SEQ ID NO: 55) | GG (SEQ ID NO: 56) |
| CANOLA | | | |
| EVENT | | | |
| NAME | | | |
| MON88302 | EPSPS | TTCTGCAGGTCCTGCTCGA | ATCGATGCGGCCGCTTCGAGT |
| | | GTGG (SEQ ID NO: 57) | GG (SEQ ID NO: 58) |

Example 2. Excision of Agrobacterium Right and Left Border Sequences from Transgenic Loci

Transgenic plant genomes containing one or more of the following transgenic loci (events) with Agrobacterium right and left border sequences are contacted with a class 2 type V RdDe and guide RNAs which recognize the indicated target DNA sites (guide RNA coding plus PAM site) in the DNA segments that flank the Agrobacterium right or left border sequences. Plant cells, callus, parts, or whole plants comprising a deletion of the selectable marker gene from the transgenic loci in the transgenic plant genome are selected.

TABLE 6
Class 2 type V Cas Nuclease Pre-existing genomic DNA target and
PAM sites in DNA flanking Agrobacterium Right Border Sequence
of different events (transgenic loci)

| | Agrobacterium Right Border | Agrobacterium Right Border |
|---|---|---|
| | Sequence Flanking DNA 1 | Sequence Flanking DNA 2 |
| | polynucleotide target DNA | polynucleotide target DNA |
| | (PAM + Guide RNA coding | (PAM + Guide RNA coding |
| | sequence) | sequence) |
| CORN | | |
| EVENT | | |
| NAME | | |
| DAS-59122-7 | TTTAAACTATCAGTGTTTGAGCGCTTT | TTTAAACGCTCTTCAACTGGAAGAGCG |
| | (SEQ ID NO: 59) | (SEQ ID NO: 60) |
| DP-4114 | TTTCTAATTCCTAAAACCAAAATCCAG | TTTGGAACAAGTGGCTATCGCCAGATA |
| | (SEQ ID NO: 61) | (SEQ ID NO: 62) |
| MON87411 | NO RB | NO RB |
| MIR162 | TTTCCCGCCTTCAGTTTAAACTATCAG | TTTGGAACTGACAGAACCGCAACGTTG |
| | (SEQ ID NO: 63) | (SEQ ID NO: 64) |
| SYN-E3272-5 | TTTCCCGCCTTCAGTTTAAACTATCAG | TTTAAATCAATTGGGCGCGCCGAATTC |
| | (SEQ ID NO: 65) | (SEQ ID NO: 66) |
| 5307 | NO RB | NO RB |
| MON89034 | TTTCTCCATATTGACCATCATACTCAT | TTTGATGAAGTGACAGGTAGGATCGGA |
| | (SEQ ID NO: 67) | (SEQ ID NO: 68) |
| SOYBEAN | | |
| EVENT | | |
| NAME | | |
| DAS68416-4 | TTTAAAAATAAATTTTTAATTTAGTTG | TTTAATCGCGGCCCATGATCACACCGG |
| | (SEQ ID NO: 69) | (SEQ ID NO: 70) |
| MON87701 | TTTGACACACACACTAAGCGTG | TTTGAATTCGAGCTCGGTACCCGG |
| | CCTGG (SEQ ID NO: 71) | GGA (SEQ ID NO: 72) |
| MON89788 | TTTAAACTATCAGTGTTTGGAG | TTTGGACTGAGAATTAGCTTCCAC |
| | CTTGA (SEQ ID NO: 73) | TCG (SEQ ID NO: 74) |
| COTTON | | |
| EVENT | | |
| NAME | | |
| COT102 | TTTAAATGGCCGCTGCGGCCAA | TTTAATAAATATGGGCAATCTTTC |
| | TTCCT (SEQ ID NO: 75) | CCT (SEQ ID NO:76 ) |
| CANOLA | | |
| EVENT | | |
| NAME | | |
| MON88302 | TTTCCCGCCTTCAGTTTAAACTA | TTTGGACTGAGAATTAGCTTCCAC |
| | TCAG (SEQ ID NO: 77) | TCG (SEQ ID NO: 78) |

TABLE 7
Class 2 type V Cas Nuclease Pre-existing genomic DNA target and
PAM sites in DNA flanking Agrobacterium Left Border Sequences
of different events (transgenic loci)

| | Agrobacterium Left Border | Agrobacterium Left Border |
|---|---|---|
| | Sequence Flanking DNA 1 | Sequence Flanking DNA 2 |
| | polynucleotide target DNA | polynucleotide target DNA |
| | (PAM + Guide RNA coding | (PAM + Guide RNA coding |
| | sequence) | sequence) |
| CORN | | |
| EVENT | | |
| NAME | | |
| DAS-59122-7 | TTTAAACGTGCAAGCGCTCAA | TTTAATGTACTGAATTGCGTACGA |
| | TTCGCC (SEQ ID NO: 79) | TTG (SEQ ID NO: 80) |
| DP-4114 | TTTGTAGCACTTGCACGTAGTT | TTTAAACGCTCTTCAACTGGAAGA |
| | ACCCG (SEQ ID NO: 81) | GCG (SEQ ID NO: 82) |
| MON87411 | TTTGTGATTCTCTAAACACTGA | TTTAATCATATTGTTAAGGATATA |
| | TAGTT (SEQ ID NO: 83) | ATT (SEQ ID NO: 84) |
| MIR162 | NO LB | NO LB |
| NK603 | TTTCTACTATTATAAAAGCTTG | TTTGAGTGGATCCTGTTATCTCTTC |
| | GTACC (SEQ ID NO: 85) | TC (SEQ ID NO: 86) |
| SYN-E3272-5 | TTTACCGGTGCCCGGGCGGCC | TTTGTTTACACCACAATATATTTCA |
| | AGCATG (SEQ ID NO: 87) | AG (SEQ ID NO: 88) |
| 5307 | TTTACCGGTGCCCGGGCGGCC | TTTGCCAGTGGGCCCAGCCTGGCC |
| | AGC (SEQ ID NO: 89) | CAG (SEQ ID NO: 90) |
| MON89034 | TTTGGACGTGAATGTAGACAC | TTTCCGGGGATGCAATGAGTATGA |
| | GTCGAA (SEQ ID NO: 91) | TGG (SEQ ID NO: 92) |
| SOYBEAN | | |
| EVENT | | |
| NAME | | |
| DAS68416-4 | TTTCTAGTCACCGGTTAGGATC | TTTAATTCTTAACAATCAATATTTT |
| | CGTTT (SEQ ID NO: 93) | AA (SEQ ID NO: 94) |
| MON87701 | TTTCCGAATTAGAATAATTTGT | TTTCCTAAATTAGTCCTACTTTTTG |
| | TTATT (SEQ ID NO: 95) | AT (SEQ ID NO: 96) |
| MON87708 | | |
| MON89788 | TTTCTCATCTAAGCCCCCATTT | TTTATCAAAATGTACTTTCATTTTA |
| | GGACG (SEQ ID NO: 97) | TA (SEQ ID NO: 98) |
| COTTON | | |
| EVENT | | |
| NAME | | |
| COT102 | TTTGTTTACCTGAATATTTGCC | TTTAATGTACGCCATGCTGGCCGC |
| | TTTTT (SEQ ID NO: 99) | CCG (SEQ ID NO: 100) |
| CANOLA | | |
| EVENT | | |
| NAME | | |
| MON88302 | TTTCTCATCTAAGCCCCCATTT | TTTACAATTGACCATCATACTCAA |
| | GGACG (SEQ ID NO: 101) | CTT (SEQ ID NO: 102) |

Transgenic plant genomes containing one or more of the following transgenic loci (events) with Agrobacterium right and left border sequences are contacted with a class 2 type II RdDe and guide RNAs which recognize the indicated target DNA sites (guide RNA coding plus PAM site) in the DNA segments that flank the Agrobacterium right or left border sequences. Plant cells, callus, parts, or whole plants comprising a deletion of the selectable marker gene from the transgenic loci in the transgenic plant genome are selected.

TABLE 8
Class 2 type II Cas Nuclease Pre-existing genomic DNA target and
PAM sites in DNA flanking Agrobacterium Right Border Sequences
of different events (transgenic loci)

| | Agrobacterium Right Border | Agrobacterium Right Border |
|---|---|---|
| | Sequence Flanking DNA 1 | Sequence Flanking DNA 2 |
| | polynucleotide target DNA | polynucleotide target DNA |
| | (Guide RNA coding sequence + | (Guide RNA coding sequence + |
| | PAM) | PAM) |
| CORN | | |
| EVENT | | |
| NAME | | |
| DAS- | GGGACGGAAGAAAGAGTGAAGG | ACGCTCTTCAACTGGAAGAGCG |
| 59122-7 | G (SEQ ID NO: 103) | G (SEQ ID NO: 104) |
| DP-4114 | AGCACTTGCACGTAGTTACCCGG | CCGGGGCCCATCGATATCCGCG |
| | (SEQ ID NO: 105) | G (SEQ ID NO: 106) |
| MIR162 | CTGATAGTTTAAACTGAAGGCGG | GAAGGAGCCACTCAGCAAGCTG |
| | (SEQ ID NO: 107) | G (SEQ ID NO: 108) |
| NK603 | GCCTTGTAGCGGCCCACGCGTGG | CGCGTTAACAAGCTTACTCGAG |
| | (SEQ ID NO: 109) | G (SEQ ID NO: 110) |
| SYN- | CAGTTTAAACTATCAGTGTTTGG | GGGCGCGCCGAATTCGAGCTCG |
| E3272-5 | (SEQ ID NO: 111) | G (SEQ ID NO: 112) |
| MON89034 | TGGATCAGCAATGAGTATGATGG | CTACCTGTCACTTCATCAAAAGC |
| | (SEQ ID NO: 113) | (SEQ ID NO: 114) |
| SOYBEAN | | |
| EVENT | | |
| NAME | | |
| DAS68416- | ACAAGAGCAAGTAGCGGATACG | GATCCTAACCGGTGTGATCATG |
| 4 | G (SEQ ID NO:115) | G (SEQ ID NO: 116) |
| MON87701 | CCGCTCTAGCGCTTCAATCGTGG | TTCTGCAGGTCCTGCTCGAGTGG |
| | (SEQ ID NO: 117) | (SEQ ID NO: 118) |
| CORN | | |
| EVENT | | |
| NAME | | |
| MON89788 | ATACATGCTTAGCATGCCCCAGG | GGGATCCACTAGTTCTAGAGCG |
| | (SEQ ID NO: 119) | G (SEQ ID NO: 120) |
| COTTON | | |
| EVENT | | |
| NAME | | |
| COT102 | ATCAAAAAAGGCAAATATTCAGG | GTACGCCATGCTGGCCGCCCGG |
| | (SEQ ID NO: 121) | G (SEQ ID NO: 122) |
| CANOLA | | |
| EVENT | | |
| NAME | | |
| MON88302 | TAAACTATCAGTGTTTGAAGTGG | CCTGCAGAAGCTTGATAACGCG |
| | (SEQ ID NO: 123) | G (SEQ ID NO: 124) |

TABLE 9
Class 2 type II Cas Nuclease Pre-existing genomic DNA target and
PAM sites in DNA flanking Agrobacterium Left Border Sequences
of different events (transgenic loci)

| | Agrobacterium Left Border | Agrobacterium Left Border |
|---|---|---|
| | Sequence Flanking DNA 1 | Sequence Flanking DNA 2 |
| | polynucleotide target | polynucleotide target |
| | DNA (Guide RNA coding | DNA (Guide RNA coding |
| | sequence + PAM) | sequence + PAM) |
| CORN | | |
| EVENT | | |
| NAME | | |
| DAS- | TCCAGGGCGAGCTCGGTACCCG | AAACAAACGGGACCATAGAAGG |
| 59122-7 | G (SEQ ID NO: 125) | G (SEQ ID NO: 126) |
| DP-4114 | ATCGTGGCCTCTTGCTCTTCAGG | AAGCGTCAATTTGGAACAAGTGG |
| | (SEQ ID NO: 127) | (SEQ ID NO: 128) |
| MON87411 | AAACACTGATAGTTTAAACGCG | ACATATGTATGTATATAATTTGG |
| | G (SEQ ID NO: 129) | (SEQ ID NO: 130) |
| MIR162 | TGTACTGAATTGTCTAGACCCG | ATTTTATAGATCATACAAAAAGG |
| | G (SEQ ID NO: 131) | (SEQ ID NO: 132) |
| NK603 | GGGGATATCCCCGGGGAATTCG | TAGAGTGGAAGTGTGTCGCGTGG |
| | G (SEQ ID NO: 133) | (SEQ ID NO: 134) |
| SYN- | GGCACCGGTAAATTTCCTGCAG | AGATGACTTGAAATATATTGTGG |
| E3272-5 | G (SEQ ID NO: 135) | (SEQ ID NO: 136) |
| 5307 | ACTAGATCTGCTAGCCCTGCAG | CCCAGCCTGGCCCAGGGAAGAGG |
| | G (SEQ ID NO: 137) | (SEQ ID NO: 138) |
| MON89034 | GGGAATTCGGTACCAAGCTTTG | CCGGGGATGCAATGAGTATGATG |
| | G (SEQ ID NO: 139) | G (SEQ ID NO: 140) |
| SOYBEAN | | |
| EVENT | | |
| NAME | | |
| MON87701 | TTACGATCCGTCGTATTTATAGG | ACAGAAGCCATCAAAAAGTAGG |
| | (SEQ ID NO: 141) | (SEQ ID NO: 142) |
| MON89788 | CGGCCGCTTCGAGTGGCTGCAG | GAAATGCTTGAGGAGAGTGAAGG |
| | G (SEQ ID NO: 143) | (SEQ ID NO: 144) |
| COTTON | | |
| EVENT | | |
| NAME | | |
| COT102 | ATTGATTTAAATGGCCGCTGCG | GTAACAGTACAGTCGGTGTAGGG |
| | G (SEQ ID NO: 145) | (SEQ ID NO: 146) |
| CANOLA | | |
| EVENT | | |
| NAME | | |
| MON88302 | ATCGATGCGGCCGCTTCGAGTG | AAATTGAAGTTGAGTATGATGGT |
| | G (SEQ ID NO: 147) | (SEQ ID NO: 148) |

The breadth and scope of the present disclosure should not be limited by any of the above-described embodiments.

1.-78. (canceled)

79. An elite crop plant comprising a modification of an original approved transgenic locus, wherein the modification comprises a deletion of a segment comprising, consisting essentially of, or consisting of:
a duplication of a transgene;
a duplication of a transgene element;
Agrobacterium right and/or left border sequences; and/or
a fragment of a transgene;

80. The elite crop plant of claim 79, wherein the duplication a transgene element and/or fragment of a transgene is a duplication and/or fragment of a promoter or a polyadenylation signal.

81. The elite crop plant of claim 79, wherein the selectable marker gene (SMG) and 20, 50 or more base pairs of DNA located 5′ to the SMG promoter and/or 3′ to the SMG polyadenylation signal in the original transgenic locus has been deleted.

82. (canceled)

83. The elite crop plant of any one of claims 79, wherein the original approved transgenic locus comprises a Bt11, DAS-59122-7, DP-4114, GA21, MON810, MON87411, MON87427, MON88017, MON89034, MIR162, MIR604, NK603, SYN-E3272-5, 5307, DAS-40278, DP-32138, DP-33121, HCEM485, LY038, MON863, MON87403, MON87403, MON87419, MON87460, MZHG0JG, MZIR098, VCO-Ø1981-5, 98140, and/or TC1507 transgenic locus in a transgenic maize plant genome.

84. The elite crop plant of claim 79, wherein the original approved transgenic locus comprises an A5547-127, DAS44406-6, DAS68416-4, DAS81419-2, GTS 40-3-2, MON87701, MON87708, MON89788, MST-FGØ72-3, and/or SYHT0H2 transgenic locus in a transgenic soybean plant genome.

85. (canceled)

86. The elite crop plant of claim 79, wherein the crop plant is a soybean plant, wherein the approved transgenic locus is a DAS44406-6 transgenic locus, and wherein a segment of the transgenic comprising the Agrobacterium left border sequence is deleted.

87. The elite crop plant of claim 79, wherein the crop plant is a maize plant, wherein the original approved transgenic locus is a DP-4114 transgenic locus of the maize plant, and wherein a segment comprising the Agrobacterium left border sequence is deleted.

88. The elite crop plant of claim 79, wherein the crop plant is a maize plant, wherein the original approved transgenic locus is a 1507 transgenic locus of the maize plant, and wherein the modification comprises a deletion of a fragment of a transgene element comprising a PAT gene, a fragment of a maize ubiquitin promoter, a fragment of an Agrobacterium mannopine synthase polyadenylation signal, and/or a truncated cry1F coding fragment in the 1507 transgenic locus of the maize plant.

89. The elite crop plant of claim 79, wherein the crop plant is a maize plant, wherein the approved transgenic locus is an MIR604 transgenic locus of a maize plant, and wherein the modification comprises a deletion of a transgene element comprising the NOS polyadenylation signal which is operably linked to DNA encoding phosphomannose isomerase (PMI) in the MIR604 transgenic locus of the maize plant.

90. The elite crop plant of claim 79, wherein the crop plant is a maize plant, wherein the approved transgenic locus is a GA21 transgenic locus of the maize plant, and wherein the modification comprises a deletion of one or more duplications of a transgene encoding EPSPS and/or a deletion of a fragment of the transgene encoding the EPSPS in the GA21 transgenic locus of the maize plant.

91. The elite crop plant of claim 79, wherein the modification to the approved transgenic locus further comprises a deletion of a segment comprising, consisting essentially of, or consisting of a selectable marker gene of an original approved transgenic locus.

92.-100. (canceled)

101. A method of editing a transgenic plant genome to obtain a plant cell, plant part, or plant containing a modification of an original transgenic locus, the method comprising: (a) contacting a transgenic plant genome with one or more gene editing molecules which introduce one or more single or double-stranded breaks providing for excision of a segment of the original transgenic locus comprising, consisting essentially of, or consisting of: a synthetic cloning site sequence; a duplication of a transgene sequence; a fragment of a transgene sequence; Agrobacterium right and/or left border sequences; and/or a segment of DNA that is non-essential for a primary functionality of the transgenic locus, and (b) selecting a plant cell, plant part, or plant containing a modified transgenic locus, wherein a segment comprising or consisting of the synthetic cloning site sequence, duplication of a transgene sequence, fragment of a transgene sequence, Agrobacterium right and/or left border sequences, and/or segment of DNA that is non-essential for expression of any transgene in the locus has been deleted, thereby obtaining a plant cell, plant part, or plant containing a modified transgenic locus.

102. The method of claim 101, wherein the method further comprises excising DNA comprising a selectable marker gene from the original transgenic locus.

103. The method of claim 101, wherein the segment comprising the synthetic cloning site sequence, the duplication of a transgene sequence, the fragment of a transgene sequence, and/or Agrobacterium right/and or left border sequences further comprises a selectable marker gene.

104. The method of claim 101, further comprising contacting the genome with one or more gene editing molecules which introduce one or more single or double-stranded breaks providing for excision of a selectable marker gene, wherein the segment comprising, consisting essentially of, or consisting of the synthetic cloning site sequence, the duplication of a transgene sequence, the fragment of a transgene sequence, and/or Agrobacterium right and/or left border sequences is deleted.

105. The method of claim 104, wherein step (b) comprises selecting a plant cell, plant part, or plant containing a modified transgenic locus, wherein a selectable marker gene and the segment comprising or consisting of the synthetic cloning site sequence, duplication of a transgene sequence, fragment of a transgene sequence, Agrobacterium right and/or left border sequences, and/or segment of DNA that is non-essential for expression of any transgene in the locus have been deleted.

106. The method of claim 101, wherein the modification comprises a modification of a Bt11, DAS-59122-7, DP-4114, GA21, MON810, MON87411, MON87427, MON88017, MON89034, MIR162, MIR604, NK603, SYN-E3272-5, 5307, DAS-40278, DP-32138, DP-33121, HCEM485, LY038, MON863, MON87403, MON87403, MON87419, MON87460, MZHG0JG, MZIR098, VCO-Ø1981-5, 98140, or TC1507 original transgenic locus in a transgenic corn plant genome.

107. The method of claim 101, wherein the modification comprises a modification of an A5547-127, DAS44406-6, DAS68416-4, DAS81419-2, GTS 40-3-2, MON87701, MON87708, MON89788, MST-FGØ72-3, or SYHT0H2 original transgenic locus in a transgenic soybean plant genome.
